(19) United States
(12) Patent Application Publication
(10) Pub. No.: US 2026/0162764 A1
(43) Pub. Date: Jun. 11, 2026

(54) METHODS AND SYSTEMS OF MULTI-OMIC APPROACH FOR MOLECULAR PROFILING OF TUMORS

(71) Applicant: Cedars-Sinai Medical Center, Los Angeles, CA (US)

(72) Inventors: Dan Theodorescu, Los Angeles, CA (US); Arsen Osipov, Los Angeles, CA (US); Ognjen Nikolic, Menlo Park, CA (US); Arkadiusz Gertych, El Segundo, CA (US); Sarah Parker, Los Angeles, CA (US); Jennifer E. Van Eyk, Los Angeles, CA (US)

(73) Assignee: Cedars-Sinai Medical Center, Los Angeles, CA (US)

(21) Appl. No.: 19/123,191

(22) PCT Filed: Oct. 27, 2023
(86) PCT No.: PCT/US2023/078070

§ 371 Date: Apr. 22, 2025

Related U.S. Application Data
(60) Provisional application No. 63/420,450, filed on Oct. 28, 2022.

(51) Int. Cl. G16B 20/00; G16B 25/10; G16B 40/20; G16H 50/30

(52) U.S. Cl. CPC G16B 20/00; G16B 25/10; G16B 40/20; G16H 50/30

(57) ABSTRACT

The present invention provides computer computer-implemented methods for determining available medical tests at a medical institution, and training machine learning models with single-omic and multi-omic combinations of plurality of features. The present invention also provides systems for performing these methods. The present invention further provides a method of prognosticating prostate cancer, as well as selecting treatment and administering treatment.

### CROSS-REFERENCE TO RELATED APPLICATIONS

This application includes a claim of priority under 35 U.S.C. § 119(e) to U.S. provisional patent application No. 63/420,450, filed Oct. 28, 2022, the entirety of which is hereby incorporated by reference.

### FIELD OF INVENTION

This invention relates to profiling tumors using artificial intelligence-based integration of multi-omic and computational pathology features.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Pancreatic ductal adenocarcinoma (PDAC) is one of the most aggressive malignancies, accounting for 47,830 deaths in 2022. Unfortunately, therapeutic advances with targeted agents and immunotherapy seen in other cancers have not translated to PDAC and thus it is expected to become the second leading cause of cancer related death in the US by 2030. While only 30-40% of PDAC patients present with localized disease and undergo potentially curative surgical resection either after diagnosis or following neoadjuvant chemotherapy, most fail and succumb to their disease. Thus, improvements in markers aimed at identifying patients cured or undergo reoccurrence by surgery by surgery and/or systemic therapies are urgently needed.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a computer-implemented method comprising: determining available medical tests at a medical institution, the available medical tests being at least a subset of known medical tests performed at various medical institutions; selecting, from the available medical tests, selected medical tests based on a trained parsimonious model for pancreatic cancer; obtaining one or more biological samples from a subject for the selected medical tests; assaying the one or more biological samples via the selected medical tests to obtain one or more factors; and prognosticating the subject as having a higher likelihood of survival, the subject as having a higher likelihood of recurrence, or a combination thereof based on the trained parsimonious model and the one or more factors.

In various embodiments, the method can further comprise weighting each factor of the one or more factors based on the selected medical tests. In various embodiments, the method can further comprise selecting a pancreatic cancer treatment method from among a plurality of pancreatic cancer treatment methods based on the trained parsimonious model and the one or more factors. In various embodiments, the method can further comprise administering the pancreatic cancer treatment method.

Various embodiments of the invention provide for a computer-implemented method comprising: processing a plurality of analytes from a plurality of individuals with cancer to obtain a plurality of features; training one or more machine learning models with single-omic and multi-omic combinations of the plurality of features to predict binary survival and disease recurrence outcomes of the plurality of individuals; evaluating the one or more machine learning models for positive predictive value and accuracy in predicting the survival and disease recurrence outcomes and feature proportions; and recursively eliminating features from the plurality of features based on the evaluating of the one or more machine learning models to develop a parsimonious machine learning model for predicting survival and disease recurrence outcome.

In various embodiments, the plurality of analytes can be derived from serum, plasma, blood, and tissue samples subjected to targeted NGS DNA sequencing, whole transcriptome RNA sequencing, paired tissue proteomics, unpaired serum proteomics, lipidomics, surgical pathology, and/or computational pathology.

In various embodiments, the plurality of analytes can include plasma or serum or blood proteins, RNA fusions, tissue proteins, plasma or serum lipids, RNA gene expressions, CNVs, INDELS, SNVs, and or tumor nuclei characteristics.

In various embodiments, the feature proportions can be evaluated using a leave-one-patient-out cross-validation strategy.

In various embodiments, the one or more machine learning models can be Support Vector Machine (SVM), Principal Component Analysis (PCA)+Logistic Regression, L1-Normalized SVM, L1-Normalized Random Forest, 5-hidden-layer Deep Neural Network, Recursive Feature Elimination (RFE) Logistic Regression and/or RFE Random Forest.

Various embodiments of the invention provide for a system comprising: memory storing computer-executable instructions; and one or more processors, the one or more processors being configured to execute the computer-executable instructions to: determine available medical tests at a medical institution, the available medical tests being at least a subset of known medical tests performed at various medical institutions; select, from the available medical tests, selected medical tests based on a trained parsimonious model for pancreatic cancer; obtain one or more biological samples from a subject for the selected medical tests; assay the one or more biological samples via the selected medical tests to obtain one or more factors; and prognosticate the subject as having a higher likelihood of survival, the subject as having a higher likelihood of recurrence, or a combination thereof based on the trained parsimonious model and the one or more factors.

In various embodiments, the one or more processors can be configured to execute the computer-executable instructions to weight each factor of the one or more factors based on the selected medical tests. In various embodiments, the one or more processors can be configured to execute the computer-executable instructions to select a pancreatic cancer treatment method from among a plurality of pancreatic cancer treatment methods based on the trained parsimonious model and the one or more factors. In various embodiments, the one or more processors can be configured to execute the computer-executable instructions to cause, at least on part, an administering of the pancreatic cancer treatment.

Various embodiments provide for a system comprising: memory storing computer-executable instructions; and one or more processors, the one or more processors being configured to execute the computer-executable instructions to: receive a plurality of features from a plurality of analytes obtained from a plurality of individuals with cancer; train one or more machine learning models with single-omic and multi-omic combinations of the plurality of features to predict binary survival and disease recurrence outcomes of the plurality of individuals; evaluate the one or more machine learning models for positive predictive value and accuracy in predicting the survival and disease recurrence outcomes and feature weights; and recursively eliminate features from the plurality of features based on the evaluating of the one or more machine learning models to develop a parsimonious machine learning model for predicting survival and disease recurrence outcome.

In various embodiments, the plurality of analytes can be derived from serum (or plasma or blood) and tissue tumor samples subjected to targeted NGS DNA sequencing, whole transcriptome RNA sequencing, paired tissue proteomics, unpaired serum proteomics, lipidomics, surgical pathology, and/or computational pathology. In various embodiments, the plurality of analytes can include plasma, or serum, or blood proteins, RNA fusions, tissue proteins, plasma or serum lipids, RNA gene expressions, CNVs, INDELS, SNVs, and tumor nuclei characteristics.

In various embodiments, the feature weights can be evaluated using a leave-one-patient-out cross-validation strategy.

In various embodiments, the one or more machine learning models can comprise Support Vector Machine (SVM), Principal Component Analysis (PCA)+Logistic Regression, L1-Normalized SVM, L1-Normalized Random Forest, 5-hidden-layer Deep Neural Network, Recursive Feature Elimination (RFE) Logistic Regression or RFE Random Forest.

Various embodiments of the invention provide for a method of prognosticating prostate cancer in a subject, comprising: assaying a plurality of analytes to detect a presence of a plurality of features, wherein the plurality of analytes (i) can be derived from serum, plasma, blood, and/or tissue samples subjected to targeted NGS DNA sequencing, whole transcriptome RNA sequencing, paired tissue proteomics, unpaired serum proteomics, lipidomics, surgical pathology, computational pathology, or a combination thereof, or (ii) can include plasma, or serum, or blood proteins, RNA fusions, tissue proteins, plasma or serum lipids, RNA gene expressions, CNVs, INDELS, SNVs, tumor nuclei characteristic, or a combination thereof, or (iii) both (i) and (ii), wherein the plurality of features can be selected from Tables 4A-4C, Tables 5A-5B, Tables 6A-6B, Tables 7A-7B, Table 8, Table 9, Tables 13A-13B, Table 14, Table 15, Tables 18A-18B or a combination thereof, and prognosticate the subject as having a higher likelihood of survival or the subject as having a lower likelihood of recurrence based on presence of the plurality of features, or prognosticate the subject as having a lower likelihood of survival or the subject as having a higher likelihood of recurrence based on presence of the plurality of features.

In various embodiments, the method can further comprise selecting a pancreatic cancer treatment method from among a plurality of pancreatic cancer treatment methods based on the likelihood of survival or the likelihood of recurrent.

In various embodiments, the method can further comprise administering the pancreatic cancer treatment method.

In various embodiments, the plurality of features can comprise at least 202 features. In various embodiments, the plurality of features can comprise at least 250 features. In various embodiments, the plurality of features can comprise at least 500 features. In various embodiments, the plurality of analytes can comprise at least four analytes. In various embodiments, the at least four analytes can comprise protein (plasma, serum, or blood protein), lipid (plasma or serum lipid), pathology and clinical. In various embodiments, the plurality of features can be selected from Table 15.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels A-E) shows a Study Classification Methodology Overview. (A) Combined multi-omic dataset of 6363 processed features spanning Clinical & Surgical Pathology, SNV, CNV, INDEL, RNA, Fusion, Tissue Proteins, Plasma Proteins, Lipids and Computational Pathology analytes. (B) Construction of all possible analyte combinations (n=1024) via Drop-Column Importance approach to simulate availability of various combinations of analytes. (C) For each analyte combination, 7 independent machine learning (ML) models were trained for model evaluation including: Support Vector Machine (SVM), Principal Component Analysis (PCA)+Logistic Regression, L1-Normalized SVM, L1-Normalized Random Forest, 5-hidden-layer Deep Neural Network, Recursive Feature Elimination (RFE) Logistic Regression, and RFE Random Forest. (D) Input analyte combinations (n=1024) with 7 modeling strategies per analyte combination produced 7168 resulting grid search runs that were subsequently analyzed for predictive power, analyte composition, and feature contributions. (E) Each unique analyte combination and ML strategy was trained via leave-one-patient-out cross-validation approach. Single-omic and multi-omic models were validated using testing sets from four separate cohorts, TCGA, JHU Cohort 1, JHU Cohort 2 and MGH cohort.

FIG. 2 (panels A-F) show a Computational Pathology Pipeline. (A) Images of random tumor nests selected by pathologist in digital H&E slides are sent for (B) processing by deep learning models to provide a mask of tumor nuclei. (C) Downstream nuclear feature extraction and formation of order statistics of morphology and H&E staining features in nuclei under the mask in patients from the cohort. (D) Patient-level visualization of extracted features by the clustergram (right) and UMAP feature embeddings (left) plots. (E) Feature learning by multiple machine learning (ML) models using leave one out (LOO) cross-validation strategy to identify the models that can predict survival with the highest accuracy. (F) Visualization of top features learned by top survival prediction models. The top features were selected based on the feature importance learned by the models.

FIG. 3 (panels A-C) show a Multi-omic Performance by Number of Analytes and Contribution. (A) Asymmetric violin plots showing accuracy and PPV distributions for multi-omic survival models, segmented by number of analytes in the multi-omic combinations. (B) Multi-omic grid search model results for Disease Survival (DS); number of analytes 1-10 represent plasma protein, RNA Fusions, Tissue Protein, lipids, clinical & surgical pathology, RNA gene expression, computational pathology, DNA CNV, DNA INDEL and DNA SNV). Y axis PPV: Positive Predictive Value, X axis Accuracy. (C) Top 15 multi-omic models for prediction of survival with percent contribution of each individual analyte.

FIG. 4 (panels A-C) show a Biological Relevance of Top Features in Muti-Omic Model and Clustering. (A) Spearman correlation of top multi-omic features with disease survival. Size represents a feature's relative importance to the top multi-omic model; Red color indicates if feature importance pertains to disease survival. (B) Gene ontology network visualization for most informative features from the multi-omic models. Selected functional pathways containing gene sets from multi-omic analytes are displayed as green nodes, with associated genes and measured analyte types represented by a specific shape (based on analyte) and colored according to the strength of a given analyte's correlation to the outcome variable of disease survival. Size of a given analyte node is relative to the frequency with which that analyte was selected for models, with larger analytes more consistently selected and no visible node indicating that the analyte was not selected as important for the DS outcome displayed. (C) UMAP clusters of patients using molecular signatures consisting of all 6363 multi-omic features, colored by survival.

FIG. 5 (panels A-D) show a Performance of Parsimonious Multi-Omic Models and Analyte Contribution for Disease Survival. Parsimonious model of (A) all multi-omic features and full data set. *Parsimonious Model at the inflection point (blue dotted line box). (B), clinical & surgical pathology and computational pathology analytes only, (C) all plasma analytes (lipidomics and protein) only, (D) all clinical & surgical pathology, computational pathology, and plasma analytes (lipidomics and protein) only. Left y-axis—Accuracy and PPV score: multi-omic model performance across feature reduction steps by restricting the maximum selectable features during model training. Right y-axis—Analyte Percent (%) Contribution: each analyte's aggregated absolute feature weight contribution at each feature reduction step.

FIG. 6 shows The Molecular Twin Platform. The Molecular Twin platform, applied to PDAC. Plasma and tissue samples from 74 patients with Stage I/II resectable PDAC were subjected to targeted NGS DNA and whole transcriptome RNA sequencing, tissue proteomics, plasma proteomics, plasma lipidomics and computational pathology to produce individual omic analytes. 6363 features were combined and served as input for 7 different types of MLAs to generate multi-omic biomarker models to predict clinical outcomes, provide patient level clustering data insight into possible therapeutic targets.

FIG. 7 shows the Top Single-omic and Multi-omic Performance for Disease Recurrence and Survival. Asymmetric violin plots showing accuracy and PPV distributions per analyte for predicting survival in decreasing order of accuracy (left to right) for multi-omic and single-omic analytes.

FIG. 8 (panels A and B) shows AI Modeling of Tumor and Stroma. (A) H&E slide with the tumor area and regions of interest (ROIs) marked by pathologist (WT); B) Same area with the cancer cells mask (cyan) predicted by our AI model.

FIG. 9 shows hierarchical co-clustering of 8 features extracted from tumor cell nuclei

FIG. 10 (panels A-C) shows the validation of the Single-omic and Multi-omic and Parsimonious Models on TCGA Validation of RNA gene signatures for disease survival: (A) 39 gene signature of poor survival (HR=2.17, [1.28-3.66], logrank p=0.0031) (B) 40 gene signature of improved survival (HR=0.74 [0.49-1.12], logrank p=0.15) (C) Parsimonious model of clinical, DNA (CNV, INDEL, SNV), RNA gene expression and computational pathology in the original cohort used to select optimal 202 features (peak) for validation in TCGA. Multi-omic model performance across feature reduction steps by restricting the maximum selectable features during model training. Right y-axis—Analyte Percent (%) Contribution: each analyte's aggregated absolute feature weight contribution at each feature reduction step.

FIG. 11 shows an example of a method 900 for prognosticating a subject.

FIG. 12 shows is an example of a method for developing a parsimonious machine learning model.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be including within the scope of this term.

“Treatment” and “treating,” as used herein refer to both therapeutic treatment and prophylactic or preventative measures, wherein the object is to prevent, slow down and/or lessen the disease even if the treatment is ultimately unsuccessful.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant cancers, as well as dormant tumors or micrometastasis. Cancers which migrate from their original location and seed vital organs can eventually lead to the death of the subject through the functional deterioration of the affected organs. As used herein, the term “invasive” refers to the ability to infiltrate and destroy surrounding tissue. In some embodiments, the tumor is a solid tumor.

The term “prognosis,” or “px,” as used herein refers to predicting the likely outcome of a current standing. For example, a prognosis can include the expected duration and course of a disease or disorder, such as progressive decline or expected recovery.

Examples of biological samples include but are not limited to body fluids, whole blood, plasma, serum, stool, intestinal fluids or aspirate, and stomach fluids or aspirate, cerebral spinal fluid (CSF), urine, sweat, saliva, tears, pulmonary secretions, breast aspirate, prostate fluid, seminal fluid, cervical scraping, amniotic fluid, intraocular fluid, mucous, and moisture in breath. In particular embodiments of the method, the biological sample may be whole blood, blood plasma, blood serum, gastrointestinal intestinal fluid or aspirate. In various embodiments, the biological sample may be whole blood. In various embodiments, the biological sample may be serum. In various embodiments, the biological sample may be plasma. Additional examples of biological samples include but are not limited to cell lysates, normal tissue, tumor tissue, hair, skin, buccal scrapings, nails, bone marrow, cartilage, bone powder, ear wax, or even from external or archived sources such as tumor samples (i.e., fresh, frozen or paraffin-embedded).

Described herein, is combining molecular evaluation of the tumor and host with machine learning algorithms (MLA), creating a unique platform that can identify predictors of therapy response including survival and recurrence with the potential to assign therapeutic and also to discover novel therapeutic targets. Several studies in other tumor types have employed MLA methods and various molecular analytes to predict therapy response and refine prognosis. However, most of these investigations, especially those on PDAC, have only focused on a handful of selected biologic variables, such as DNA, combined with MLA to determine whether findings can predict outcomes or accurately prognosticate. Even multi-omic proteogenomic studies in PDAC, which have revealed novel targets, pathways and unique phenotypes of PDAC, have limited ability to predict clinical outcome. In addition, even if effective, the nature of such multi-omic analyses comes with high complexity and cost, as well as significant resource requirements. Thus, an important consideration in the development of novel predictive markers is how to utilize the power of multi-omics to develop parsimonious panels of these, that would be both cost effective and easily deployable in clinical practice.

As further described herein, we use a multi-omic analytic platform that incorporates advanced molecular profiling beyond examination of common analytes, such as proteins, lipids, and DNA. Profiling data was collected from both tumor and host samples, and included computational pathology features, including nuclear morphology on the former. Multiple novel MLAs were developed and then applied to this dataset to test the hypothesis that this approach can provide biomarker panels that accurately predict disease survival (DS) after surgery in patients with resectable PDAC. Through recursive feature/analyte elimination, our approach was able to provide a parsimonious model employing a limited number of features/analytes which maintains a high degree of performance in prediction of DS compared to the full optimal models we developed. Utilizing external samples/data from The Cancer Genome Atlas (TCGA), Johns Hopkins University (JHU), and Massachusetts General Hospital (MGH), we independently validated the power of our full and parsimonious models to predict DS. Through this analysis, we also discovered that among all analytes available in the preoperative setting, serum plasma protein is the most critical biomarker with significant predicative power for survival and superior to CA 19-9. This work is an approach we named the Molecular Twin; a virtual, bioinformatic computational replica of the patient that can be updated and enriched in space and time with additional analytes and types obtained longitudinally. While we utilize PDAC here, this approach is tumor type agnostic, allowing it to potentially impact clinical care and scientific discovery across all cancers.

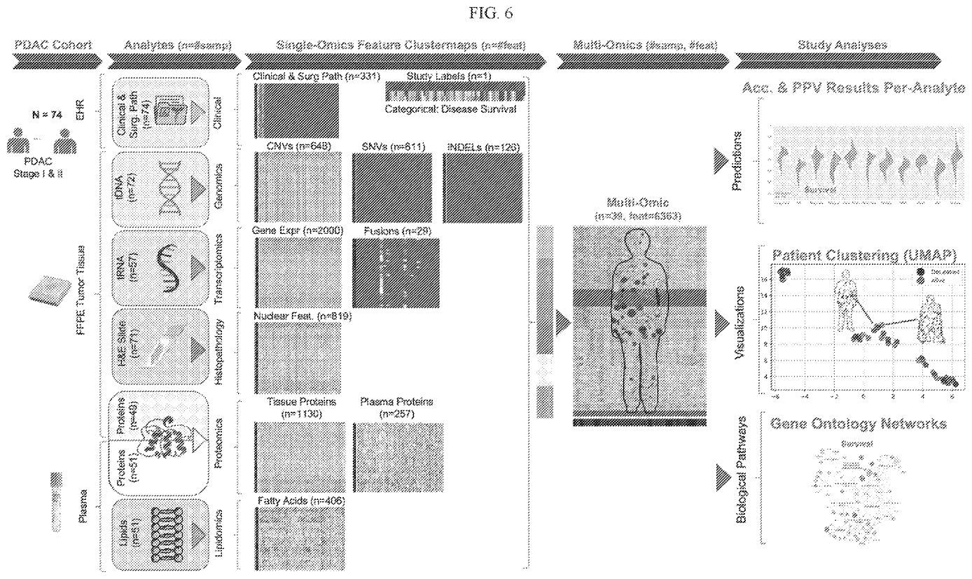

Here we describe an approach that we term the Molecular Twin which incorporates multiple molecular, histopathologic, and clinical features from both host and tumor and a comprehensive machine learning multi-omic analysis to provide novel outcome predictors and possible therapeutic targets for further investigation (FIG. 6). Our Molecular Twin platform has not only allowed us to develop comprehensive multi-omic and highly informative and efficient parsimonious models for clinical outcome prediction, but it has led to the novel discovery that plasma proteins are a highly predictive analyte for DS prediction. Most importantly, testing of the approach on independent four cohorts and datasets have validated its predictive value for DS and revealed its superiority to CA 19-9, currently the most commonly used serum biomarker for this purpose. This approach has the potential to significantly impact how we develop markers in the future and in the case of preoperative markers, may have provided enough rationale to initiate clinical development and large-scale testing to determine its value in surgical decision making. Finally, the approach, by virtue of its ability to generate parsimonious models has laid a foundation for the future democratization of precision oncology and thus reduce national and global disparities in its use.

Our study reveals that the multi-omic analytes incorporating individual single-omic sources is the most accurate clinical predictor of DS and that plasma proteins are the most significant single-omic predictors of DS. We also show that multi-omic models with limited, but highly predictive analytes, perform nearly as well as the top multi-omic models with higher number of individual single-omic analytes. It should be noted that none of the top multi-omic models consisted of all 10 available analytes. This reinforces the concept of complementarity and highlights the overlap of signal across analytes, suggesting that in some embodiments, it may not be necessary to carry out the comprehensive 10-analyte workup to obtain accurate predictions. This is important when considering the implications of analytic capability and cost in resource-poor geographies. A strength of this platform is its resilience, allowing interchangeability and complementarity among analytes. This observation also suggests flexibility in analyte selection to approximate optimal predictive performance, with patient burden, efficiency, ease of testing, time, and cost of analyte acquisition being other notable considerations. Many analytic techniques, especially comprehensive genomics, can be expensive as well as time and labor intensive. However, our study reveals single-omics sources employed in this platform, such as computational pathology-based features or plasma proteins, offer the opportunity to circumvent these challenges using near term practical solutions with clinical implications.

In computational pathology analysis, features of nuclear architecture can predict survival in many cancer types, and our results were consistent with these reports. Although our study focused on quantifying morphological nuclear architecture, a much deeper computational pathology-based profiling of tumor tissue is possible. For instance, MLAs trained on architectural features of tumor nests and stroma can predict metastasis in pancreatic neuroendocrine cancer. To extract features, computational pathology uses only H&E slides prepared to obtain routine pathology reports. Since no special tissue processing or chemical reagents are necessary, the cost of measuring a feature through this platform is low. In addition, digital slides can be sent for computational analysis through the cloud and results sent back to the requester as a multi-omic score generated by combining all other information on the patient electronically.

Studies employing smaller cohorts, for example one study with 14 patients, has shown that certain predefined plasma proteins can predict early recurrence. Our study is larger and more comprehensive with 74 patients and newly identifies many more plasma proteins as significant predictors of DS. Plasma proteins within multi-omic panels also represent a unique opportunity for efficient, informative, and clinically impactful testing since this specific analyte can be obtained quickly and preoperatively in a non-invasive manner. Although preoperative antigen testing, like CA 19-9, continues to be routinely utilized in predicting resectability and survival, our study demonstrated that plasma proteins alone, and even more so when combined with other preoperative analytes such as clinical data is superior to CA 19-9 alone. These results are not surprising since it is well appreciated that preoperative CA 19-9 has limitations which may contribute to its poor performance as a tool predicting DS. For example, between 6% of Caucasians and 22% of African Americans do not generate the CA 19-9 antigen and other conditions involving the hepatobiliary tree and malignancies can lead to elevations of CA 19-9. Unlike CA 19-9, plasma proteins have the potential to inform subsequent therapeutic decisions including the role of perioperative chemotherapy and even appropriate candidacy for complex and surgery with significant morbidity. Our approach provided both novel and known insights into molecular drivers and clinically useful markers of PDAC survival prediction, the latter findings helping to validate the value of our approach. An example of the latter was the plasma protein ANXA1, that we found to be a significant predictor of DS. Published data reported that incorporating ANXA1 into marker panels provides predictive ability in the diagnosis of early-stage PDAC.

Multi-omic analysis across tumor types has been undertaken before but not to this extent. One study employed a smaller number of analytes than in our current analysis, integrating mRNA, microRNA, and DNA for PDAC recurrence and survival prediction. They highlighted hurdles in multi-omic analyses, describing that employing a multi-omic platform, particularly involving genomic signatures in clinical practice, can come with substantial costs. Unlike these prior studies, we sought to address two of the major issues impeding the global use of precision therapy in cancer care, which are cost and technical sophistication. To overcome this challenge, we employed a recursive feature elimination strategy to help identify the minimum number of features across analytes within the multi-omic model with optimal performance in a novel, parsimonious model approach. This approach revealed that not all analytes are needed to achieve high accuracy of clinical outcome prediction. In fact, through our parsimonious model we found that by restricting the maximum selectable features during model training of the multi-omic model performance, only 598 features across 10 analytes are required to achieve an accuracy and positive predictive value of 0.85, similar to the full multi-omic model with 6363 features. As with our full multi-omic model analysis of the MT-Pilot, we found that plasma analytes were the dominant feature type of the parsimonious panel. The parsimonious model uncovers highly informative features while simultaneously minimizing the number of required analytes without compromising predictive performance.

A strength of our study is that we validated our findings in independent datasets of PDAC including the TCGA cohort, two separate cohorts from JHU, and a cohort from MGH. In our validation approach, we recognize that no single multi-omic model contains all 10 single-omic analytes concurrently. This is an inherent shortcoming of our validation datasets as well as many currently available datasets, where none contain complete data of all 10 single-omic sources that our original MT-Pilot cohort provided. Regardless, we externally validated our multi-omic panels with maximal available and complete data within each dataset. For example, we were able to validate our findings that computational pathology and RNA gene expression within our MT-Pilot Cohort and TCGA had similar predictive performance and that it was an informative element within the parsimonious model applied to both to our MT-Pilot Cohort as a training set and TCGA, as a test set. Importantly for the potential democratization aspects of this work, the 202 highly predictive features provided by the optimal parsimonious model found on our original MT-Pilot cohort were applied to the TCGA and led to similar predictive performance. Additionally, single- and multi-omic panels incorporating plasma proteins were validated as a significant predictive tool when our MT-Pilot data was utilized as a training set against two separate prospective test cohorts analyzed separately and employing similar proteomic analysis utilized in our MT-Pilot cohort. Our findings and this validation approach provides evidence to support the development of plasma (or serum or blood) proteins as a potentially clinically usable assay in PDAC.

This externally validated study examined an aggressive malignancy, PDAC, that lacks robust predictive and prognostic biomarkers. The Molecular Twin represents a new way forward for the discovery of promising predictive and clinically meaningful biomarkers, targets for treatment, and ultimately tools to democratize and reduce national and global disparities in the use of precision cancer medicine across all of cancer.

Embodiments of the present invention are based, at least in part, on these findings as described herein.

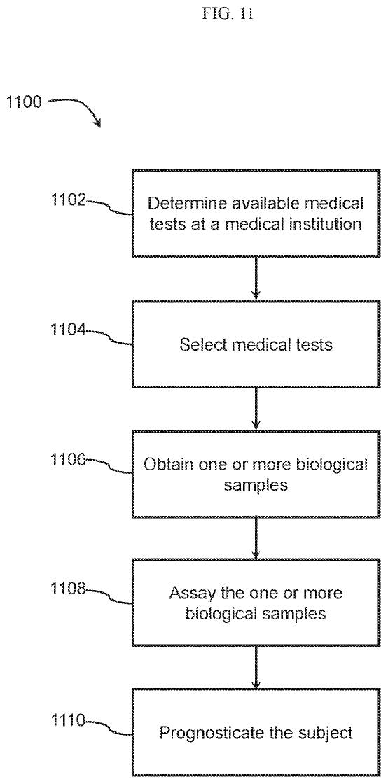

Referring to FIG. 11, disclosed is an example of a method 1100 for prognosticating a subject. At step 1102, available medical tests are determined. The available medical tests are at least a subset of known medical tests that can be performed at various medical institutions. Depending on various limitations, such as the size and location of a medical institution and budget of the medical institution, a subset of medical tests may be available that relate to or are associated with the ability to prognosticate a subject with respect to pancreatic cancer. Accordingly, at step 1102, the available medical tests are determined.

At step 1104, medical tests are selected from the available medical tests based on a trained parsimonious model for pancreatic cancer. The trained parsimonious model determines which of the available medical tests are viable for conducting based on the information used to train the parsimonious model.

At step 1106, one or more biological samples are obtained from a subject for the selected medical tests. The one or more biological samples are determined based on a known relationship between the selected medical tests and the biological samples needed to perform the medical tests. Note, the least invasive sample would be analytes determined from plasma (or from serum or blood).

At step 1108, the one or more biological samples are assayed via the selected medical tests to obtain one or more factors. The one or more factors describe the outcome of the medical tests. The one or more factors can vary depending on the specific medical tests and the specific biological samples.

At step 1110, the subject is prognosticated as having a higher likelihood of survival, as having a higher likelihood of recurrence, or a combination thereof based on the trained parsimonious model and the one or more factors. The trained parsimonious model uses the input of the one or more factors based on the information used to train the parsimonious model to perform the prognostication.

According to some implementations, each factor of the one or more factors can be weighted based on the selected medical tests. For example, Factor A may have a certain weighting when Medical Tests 1, 2, and 3 are selected that generate Factors A, B, and C, respectively. However, when Medical Test 3 is not available at the medical institution, such that Medical Test 3 is not selected and only Medical Tests 1 and 2 are selected, Factor A may have a different weighting. Factor A may be weighted more heavily relative to Factor B when only Factors A and B are present, versus how much Factor A is weighted relative to Factors B and C when Factors A, B, and C are present.

After step 1110, the method 1100 can further include the step of selecting a pancreatic cancer treatment method from among a plurality of pancreatic cancer treatment methods based on the trained parsimonious model and the one or more factors. The method can further include the step of administering the pancreatic cancer treatment method. With the method 1100, the trained parsimonious model provides for efficient prognostication of survival and recurrence likelihoods based on the available medical tests that are the most effective at providing the most accurate prognostication.

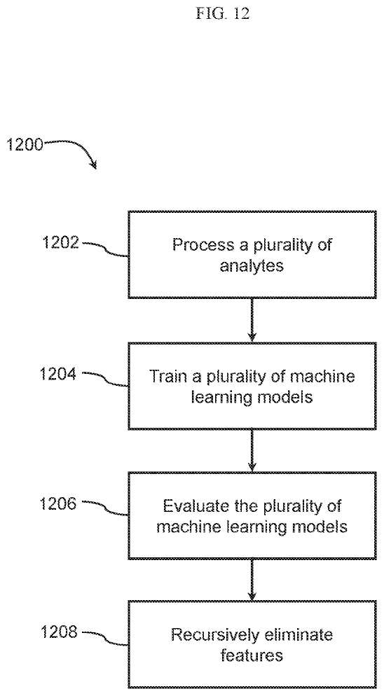

Referring to FIG. 12, disclosed is an example of a method 1200 for developing a parsimonious machine learning model. At step 1202, a plurality of analytes from a plurality of individuals with cancer are processed to obtain a plurality of features. According to some implementations, the plurality of analytes are derived from serum and tissue samples of a subject subjected to targeted NGS DNA sequencing, whole transcriptome RNA sequencing, paired tissue proteomics, unpaired serum proteomics, lipidomics, surgical pathology, and/or computational pathology. However, the plurality of analytes can be derived according to any process, technique, or method disclosed herein. According to some implementations, the plurality of analytes can include plasma (or serum or blood) proteins, RNA fusions, tissue proteins, plasma (or serum) lipids, RNA gene expressions, copy number variations (CNVs), INDELS, SNVs, and tumor nuclei characteristics. In some implications, the plurality of analytes can include clinical & surgical pathology and computational pathology analytes only; all plasma analytes (lipidomics and protein) only; or all clinical & surgical pathology, computational pathology, and plasma analytes (lipidomics and protein) only. However, the plurality of analytes can include any analyte disclosed herein.

At step 1204, a plurality of machine learning models are trained with single-omic and multi-omic combinations of the plurality of features to predict binary survival and disease recurrence outcomes for the plurality of individuals. According to some implementations, the plurality of machine learning models can include one or more of Support Vector Machine (SVM), Principal Component Analysis (PCA)+Logistic Regression, L1-Normalized SVM, L1-Normalized Random Forest, 5-hidden-layer Deep Neural Network, Recursive Feature Elimination (RFE) Logistic Regression and RFE Random Forest. However, the plurality of machine learning models can include any machine learning model disclosed herein.

At step 1206, the plurality of machine learning models are evaluated for positive predictive value and accuracy in predicting the survival and disease recurrence outcomes and feature weights. According to some implementations, the feature weights can be evaluated using a leave-one-subject-out cross-validation strategy.

At step 1208, features are recursively eliminated from the plurality of features based on the evaluating of the plurality of machine learning models to develop a parsimonious machine learning model for predicting survival and disease recurrence outcome. The parsimonious machine learning model can then be used as, for example, the trained parsimonious model in the method 900 disclosed above to provide efficient prognostication of survival and recurrence likelihoods based on available medical tests that are the most effective at providing the most accurate prognostication for a medical institution. Data input is semi-quantitative or quantitative with appropriate quality control use to eliminate data noise and rule out error. Protein and lipid data can be obtained using capture assay (e.g., aptamer or immunoassays) and or mass spectrometry, DNA sequencing can be targeted mutations or from NGS and nuclei staining by HE or other staining methods for nuclei or other methods for differentiating tumor from nontumor areas on tissue slides.

It should also be understood that the disclosure herein can be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

The computing device can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Various embodiments of the present invention provide for a method of prognosticating prostate cancer in a subject, comprising: assaying a plurality of analytes and pathological data to detect the presence of a presence of a plurality of features, wherein the plurality of analytes are derived from serum, plasma, blood and/or tissue samples subjected to targeted NGS DNA sequencing, whole transcriptome RNA sequencing, paired tissue proteomics, unpaired serum proteomics, lipidomics, surgical pathology, computational pathology, or a combination thereof, or wherein the plurality of analytes include plasma (or serum or blood) proteins, RNA fusions, tissue proteins, plasma (or serum) lipids, RNA gene expressions, CNVs, INDELS, SNVs, and tumor nuclei characteristic, or both, and wherein the plurality of features is selected from Tables 4A-4C, Tables 5A-5B, Tables 6A-6B, Tables 7A-7B, Table 8, Table 9, Tables 13A-13B, Table 14 Table 15, Tables 18A-18B or a combination thereof, and prognosticate the subject regarding survival and/or recurrence. In some implications, the plurality of analytes can include clinical & surgical pathology and computational pathology analytes only; all plasma analytes (lipidomics and protein) only; or all clinical & surgical pathology, computational pathology, and plasma analytes (lipidomics and protein) only.

Among Tables 4A-4C, Tables 5A-5B, Tables 6A-6B, Tables 7A-7B, Table 8, Table 9, Tables 13A-13B, Table 14, Table 15, Tables 18A-18B, the ones with the features weights (e.g., highest feature weights), and their spearman rho/p-value provide the following guidance. Feature correlations to study objectives (“Spearman rho” and “Spearman p-value” columns) indicate statistical correlation of the study dataset to the outcomes, where the outcome definition used was label_survival {dead: 0, alive: 1}. Any positive correlation in the “Spearman rho” column, meaning the feature in question correlates positively with survival. “Feature frequency” represents how stable and often selected features are across the training folds (that is, it can be viewed as a corollary to a p-value, where the focus is on highly stable, relevant features with high frequency of selection). “Feature weight” represents relevance and predictive power carried by that specific feature, with positive weight meaning it predicts death. As such, these information contained in these Tables provide the information for prognosticating disease survival and/or recurrence.

In various embodiments for the method of prognosticating prostate cancer in a subject, the plurality of features are selected from Tables 4A-4C. In various embodiments, the plurality of features are the top 10 features from Table 4A. In various embodiments, the plurality of features are all the features from Table 4A. In various embodiments, the plurality of features are 2-5, 6-10, or 11-16 features from Table 4A. In various embodiments, the plurality of features are 2-10, 11-20, 21-30, 31-50, 51-100, 101-150, or 151-161 features from Table 4B. In various embodiments, the plurality of features are 2-50, 51-100, 101-150, 151-200, 201-250, 251-300, 301-350, 351-400, 401-450, or 451-472 features from Table 4C. For example, the Spearman rho, Sperman p-value, feature frequency, and feature weights for these features are used as noted above to prognosticate the subject regarding survival and/or recurrence. Unless otherwise noted, expression levels are normalized using the z-scoring technique which standardizes feature values measured across cases to the distribution which has the mean=0 and standard deviation=1. In this context, moderate to high expression means higher than the average (by 1 to 2 standard deviations) among cases, and low to moderate low means lower than the average (by about 1 to 2 standard deviations) among cases.

In various embodiments for the method of prognosticating prostate cancer in a subject, the plurality of features are selected from Table 5A. In various embodiments, the plurality of features are 2-25 features from Table 5A. In various embodiments, the plurality of features are 26-50 features from Table 5A. In various embodiments, the plurality of features are 50-75 features from Table 5A. In various embodiments, the plurality of features are 76-100 features from Table 5A. In various embodiments, the plurality of features are 101-125 features from Table 5A. In various embodiments, the plurality of features are 126-146 features from Table 5A. In various embodiments, the plurality of feature are all the features from Table 5A. For example, the Spearman rho, Sperman p-value, feature frequency, and feature weights for these features are used as noted above to prognosticate the subject regarding survival and/or recurrence. In various embodiments for the method of prognosticating prostate cancer in a subject, the plurality of features are selected from Table 5B.

In various embodiments for the method of prognosticating prostate cancer in a subject, the plurality of features comprise RAD51, IL6R, FGF20, and SOX2. In these embodiments, the subject is prognosticated regarding the likelihood of disease survival (DS) based on alterations in RAD51, IL6R, FGF20, and SOX2. In various embodiments, the alterations are single nucleotide variations (SNVs). For example, the Spearman rho, Sperman p-value, feature frequency, and feature weights for these features are used as noted above to prognosticate the subject regarding survival and/or recurrence.

In various embodiments an assay system is provided to detect alterations in RAD51, IL6R, FGF20, and SOX2. In various embodiments, the assay system comprises at least two differentially labeled, allele-specific probes and a PCT primer pair to detect RAD51, at least two differentially labeled, allele-specific probes and a PCT primer pair to detect IL6R, at least two differentially labeled, allele-specific probes and a PCT primer pair to detect FGF20, and at least two differentially labeled, allele-specific probes and a PCT primer pair to detect SOX2.

In various embodiments, the plurality of features comprise RIT1. In these embodiments, the subject is prognosticated regarding the likelihood of disease survival (DS) based on an alteration of RIT1. In various embodiments, the alteration is a copy number variation (CNV). For example, the Spearman rho, Sperman p-value, feature frequency, and feature weights for these features are used as noted above to prognosticate the subject regarding survival and/or recurrence.

In various embodiments an assay system is provided to detect an alteration of RIT1. In various embodiments, the assay system comprises a primer that specifically binds to RIT.

In various embodiments, the plurality of features comprises FOXQ1 and KDM5D. In these embodiments, the subject is prognosticated regarding the likelihood of disease survival (DS) based on an alteration of FOXQ1 and KDM5D. In various embodiments, the alterations are copy number variations (CNVs). For example, the Spearman rho, Sperman p-value, feature frequency, and feature weights for these features are used as noted above to prognosticate the subject regarding survival and/or recurrence.

In various embodiments an assay system is provided to detect an alteration of FOXQ1 and KDM5D. In various embodiments, the assay system comprises a primer that specifically binds to FOXQ1 and a primer that specifically binds to KDM5D.

In various embodiments, the plurality of features comprise TP53, CDKN2A and SMAD4. In these embodiments, the subject is prognosticated regarding the likelihood of disease survival (DS) based on alterations of TP53, CDKN2A and SMAD4. In various embodiments, the alterations include gene mutations. For example, the Spearman rho, Sperman p-value, feature frequency, and feature weights for these features are used as noted above to prognosticate the subject regarding survival and/or recurrence.

In various embodiments an assay system is provided to detect an alteration of TP53, CDKN2A and SMAD4. In various embodiments, the assay comprises an allele-specific primer that detects the mutant allele of TP53, a MGB oligonucleotide blocker suppresses the wild type allele of TP53, a locus-specific primer for TP53, and a locus specific dye-labeled MGB probe for TP53; an allele-specific primer that detects the mutant allele of CDKN2A, a MGB oligonucleotide blocker suppresses the wild type allele of CDKN2A, a locus-specific primer for CDKN2A, and a locus specific dye-labeled MGB probe for CDKN2A; and an allele-specific primer that detects the mutant allele of SMAD4, a MGB oligonucleotide blocker suppresses the wild type allele of SMAD4, a locus-specific primer for SMAD4, and a locus specific dye-labeled MGB probe for SMAD4.

In various embodiments, the plurality of features comprise DIS3L2 and CHD4. In these embodiments, the subject is prognosticated regarding the likelihood of disease survival (DS) based on alterations of DIS3L2 and CHD4. In various embodiments, the alterations include gene mutations. For example, the Spearman rho, Sperman p-value, feature frequency, and feature weights for these features are used as noted above to prognosticate the subject regarding survival and/or recurrence.

In various embodiments an assay system is provided to detect an alteration of DIS3L2 and CHD4. In various embodiments, the assay comprises an allele-specific primer that detects the mutant allele of DIS3L2, a MGB oligonucleotide blocker suppresses the wild type allele of DIS3L2, a locus-specific primer for DIS3L2, and a locus specific dye-labeled MGB probe for DIS3L2; and an allele-specific primer that detects the mutant allele of CHD4, a MGB oligonucleotide blocker suppresses the wild type allele of CHD4, a locus-specific primer for CHD4, and a locus specific dye-labeled MGB probe for CHD4.

In various embodiments for the method of prognosticating prostate cancer in a subject, the plurality of features are selected from Table 6A. In various embodiments, the plurality of features are 2-25 features from Table 6A. In various embodiments, the plurality of features are 26-50 features from Table 6A. In various embodiments, the plurality of features are 50-75 features from Table 6A. In various embodiments, the plurality of features are 76-96 features from Table 6A. In various embodiments, the plurality of features are all the features from Table 6A. In various embodiments for the method of prognosticating prostate cancer in a subject, the plurality of features are selected from Table 6B.

In various embodiments, the plurality of features comprise NFE2L2 and LRIG3. In these embodiments, the subject is prognosticated regarding the likelihood of disease survival (DS) based on expression of NFE2L2 and LRIG3. For example, the Spearman rho, Sperman p-value, feature frequency, and feature weights for these features are used as noted above to prognosticate the subject regarding survival and/or recurrence.

In various embodiments an assay system is provided to detect the expression levels of NFE2L2 and LRIG3. In various embodiments, the assays comprise a primer that binds specifically to NFE2L2 and a primer that binds specifically to LRIG3 to detect the expression level of NFE2L2 and LRIG3. In various embodiments, the expression level is mRNA expression level.

In various embodiments, the plurality of features comprise USP22. In these embodiments, the subject is prognosticated regarding the likelihood of disease survival (DS) based on expression of USP22. For example, the Spearman rho, Sperman p-value, feature frequency, and feature weights for these features are used as noted above to prognosticate the subject regarding survival and/or recurrence.

In various embodiments, the plurality of features comprise NFE2L2, LRIG3, and USP22. In these embodiments, the subject is prognosticated regarding the likelihood of disease survival (DS) based on higher expression of NFE2L2, LRIG3, and USP22. For example, the Spearman rho, Sperman p-value, feature frequency, and feature weights for these features are used as noted above to prognosticate the subject regarding survival and/or recurrence.

In various embodiments an assay system is provided to detect the expression levels of NFE2L2, LRIG3, and USP22. In various embodiments, the assays comprise a primer that binds specifically to NFE2L2, a primer that binds specifically to LRIG3, and a primer that binds specifically to USP22 to detect the expression level of NFE2L2, LRIG3, and USP22. In various embodiments, the expression level is mRNA expression level.

In various embodiments for the method of prognosticating prostate cancer in a subject, the plurality of features are selected from Table 7A. In various embodiments, the plurality of features are 2-25 features from Table 7A. In various embodiments, the plurality of features are 26-50 features from Table 7A. In various embodiments, the plurality of features are 50-75 features from Table 7A. In various embodiments, the plurality of features are 76-100 features from Table 7A. In various embodiments, the plurality of features are 101-125 features from Table 7A. In various embodiments, the plurality of features are 126-150 features from Table 7A. In various embodiments, the plurality of features are 151-176 features from Table 7A. In various embodiments, the plurality of features are 176 features from Table 7A. In various embodiments, the plurality of features are all the features from Table 7A. In various embodiments for the method of prognosticating prostate cancer in a subject, the plurality of features are selected from Table 7A. For example, the Spearman rho, Sperman p-value, feature frequency, and feature weights for these features are used as noted above to prognosticate the subject regarding survival and/or recurrence.

In various embodiments, the plurality of features comprise ANXA1. In these embodiments, the subject is prognosticated regarding the likelihood of disease survival (DS) based on plasma (or serum or blood) protein levels of ANXA1. For example, the Spearman rho, Sperman p-value, feature frequency, and feature weights for these features are used as noted above to prognosticate the subject regarding survival and/or recurrence.

In various embodiments an assay system is provided to detect ANXA1. In various embodiments, the assay comprises a binder for ANXA1; for example, an antibody capable of binding to ANXA1.

In various embodiments, the plurality of features comprise diacylglycerols (DAG) and cholesteryl esters (CE). In these embodiments, the subject is prognosticated to regarding the likelihood of disease survival (DS) based on higher plasma (or serum) lipid levels of DAG and CE. For example, the Spearman rho, Sperman p-value, feature frequency, and feature weights for these features are used as noted above to prognosticate the subject regarding survival and/or recurrence.

In various embodiments for the method of prognosticating prostate cancer in a subject, the plurality of features are selected from Table 12. In various embodiments, the plurality of features are 1-4 features in Table 12. In various embodiments, the plurality of features are 5-8 features in Table 12. In various embodiments, the plurality of features are the 8 features in Table 12. For example, the Spearman rho, Sperman p-value, feature frequency, and feature weights for these features are used as noted above to prognosticate the subject regarding survival and/or recurrence.

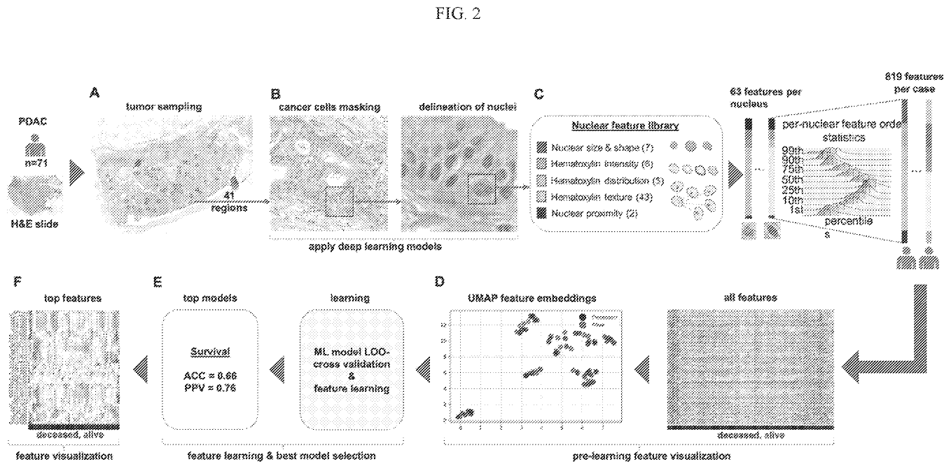

These 8 features in Table 12 quantitate patterns of hematoxylin staining (which reflect chromatin conformation) in cancer cell nuclei. The expression of 1, 2, 3, 4, 5, 6, 7, or 8 of these features is associated with survival status (alive vs. deceased) and separation of subtests in the UMAP plot (FIG. 2D). In these embodiments, disease survival is prognosticated if 1, 2, 3, 4, 5, 6, 7, or 8 of these features are detected. That is, if 1, 2, 3, 4, 5, 6, 7, or 8 of NF40: Large Zone Size Emphasis, NF46: Large Zone/High Gray Emphasis, NF33: Inverse Difference, NF18: Inverse Difference moment, NF32: Maximum Probability, NF31: Cluster Prominence, NF49: Zone Size Percentage, and NF53: Run Percentage are detected. The subject is prognosticated to have a high likelihood of death if high to moderate expression of NF40, NF46, NF33, NF18, NF31 and moderate to low expression of NF49, NF53 are detected. Expression levels are normalized using the z-scoring technique which standardizes feature values measured across cases to the distribution which has the mean=0 and standard deviation=1. In this context, moderate to high expression means higher than the average (by 1 to 2 standard deviations) among cases, and low to moderate low means lower than the average (by about 1 to 2 standard deviations) among cases.

In various embodiments for the method of prognosticating prostate cancer in a subject, the plurality of features are selected from Tables 13A and/or 13B. In various embodiments, the plurality of features are 2-25 features from Tables 13A and/or 13B. In various embodiments, the plurality of features are 26-50 features from Tables 13A and/or 13B. In various embodiments, the plurality of features are 50-79 features from Tables 13A and/or 13B.

In various embodiments for the method of prognosticating prostate cancer in a subject, the plurality of features are selected from Table 15. In various embodiments, the plurality of features are 2-50 features from Table 15. In various embodiments, the plurality of features are 51-100 features from Table 15. In various embodiments, the plurality of features are 101-150 features from Table 15. In various embodiments, the plurality of features are 151-202 features from Table 15. In various embodiments, the plurality of features are all the features from Table 15. For example, the feature weight in Table 15, alone or in combination with the Spearman rho, Sperman p-value, and/or feature frequency (found in other tables for those features), are used as noted above to prognosticate regarding disease survival and/or recurrence.

In various embodiments for the method of prognosticating prostate cancer in a subject, the plurality of features are selected from Table 18A. In various embodiments, the plurality of features are 2-10, 11-20, 21-30, 31-40, 41-50, or 51-56 features from Table 18A. In various embodiments, the plurality of features are the first 56 features from Table 18A. In various embodiments, the plurality of features are 51-75, 76-100, or 100-121 features from Table 18A. In various embodiments for the method of prognosticating prostate cancer in a subject, the plurality of features are selected from Table 18B. For example, the Spearman rho, Sperman p-value, feature frequency, and feature weights for these features are used as noted above to prognosticate the subject regarding survival and/or recurrence.

In various embodiments for the method of prognosticating prostate cancer in a subject, the plurality of features comprises at least about 25 features. In various embodiments, the plurality of features comprises at least about 50 features. In various embodiments, the plurality of features comprises at least about 75 features. In various embodiments, the plurality of features comprises at least about 100 features. In various embodiments, the plurality of features comprises at least about 150 features. In various embodiments, the plurality of features comprises at least about 200 features. In various embodiments, the plurality of features comprises at least about 250 features. For example, the Spearman rho, Sperman p-value, feature frequency, and feature weights for these features are used as noted above to prognosticate the subject regarding survival and/or recurrence.

In various embodiments, the plurality of features comprises a minimum number of features per PPV, such as about 100. In various embodiments, the plurality of features comprises at least 150 features. In various embodiments, the plurality of features comprises at least 200 features. In various embodiments, the plurality of features comprises at least 150 features. In various embodiments, the plurality of features are 202 features. In various embodiments, the plurality of features comprises at least 250 features. In various embodiments, the plurality of features comprises at least 300 features. In various embodiments, the plurality of features comprises at least 400 features. In various embodiments, the plurality of features comprises at least 500 features. In various embodiments, the plurality of features comprises at least 550 features. In various embodiments, the plurality of features comprises at least 600 features. In various embodiments, the plurality of features comprises at least 598 features. In various embodiments, the plurality of features are 598 features. In various embodiments, the plurality of features comprises at least 700 features. In various embodiments, the plurality of feature comprises the top features from Tables 4A, 5A, 6A, 7A, 18A, or a combination thereof. For example, the Spearman rho, Sperman p-value, feature frequency, and feature weights for these features are used as noted above to prognosticate the subject regarding survival and/or recurrence.

In various embodiments, the plurality of analytes comprise at least four analytes. In various embodiments, the at least four analytes comprises proteins (plasma, serum or blood lipids), lipids (plasma or serum lipids), pathology and clinical data. For example, the Spearman rho, Sperman p-value, feature frequency, and feature weights for these features are used as noted above to prognosticate the subject regarding survival and/or recurrence.

In various embodiments, wherein the plurality of analytes comprise at least two analytes and the at least two analytes comprises pathology and clinical, and the plurality of features comprises at least 300 features. In various embodiments, wherein the plurality of analytes comprise at least two analytes and the at least two analytes comprises pathology and clinical, the plurality of features comprises about 265-495 features. For example, the Spearman rho, Sperman p-value, feature frequency, and feature weights for these features are used as noted above to prognosticate the subject regarding survival and/or recurrence.

In various embodiments, wherein the plurality of analytes comprise at least two analytes and the at least two analytes comprises proteins (plasma, serum or blood protein) and lipids (plasma or serum lipids), the plurality of features comprises at least 40 features. In various embodiments, wherein the plurality of analytes comprise at least two analytes and the at least two analytes comprises proteins (plasma, serum or blood protein) and lipids (plasma or serum lipids), the plurality of features comprises about 25-75 features. For example, the Spearman rho, Sperman p-value, feature frequency, and feature weights for these features are used as noted above to prognosticate the subject regarding survival and/or recurrence.

In various embodiments, wherein the plurality of analytes comprise at least four analytes and the at least four analytes comprise proteins (plasma, serum or blood lipids), lipids (plasma or serum lipids), pathology and clinical data, the plurality of features comprises at least 200 features. In various embodiments, wherein the plurality of analytes comprise at least four analytes and the at least four analytes comprise proteins (plasma, serum or blood lipids), lipids (plasma or serum lipids), pathology and clinical data, the plurality of features comprises 202 features. In various embodiments, wherein the plurality of analytes comprise at least four analytes and the at least four analytes comprise proteins (plasma, serum or blood lipids), lipids (plasma or serum lipids), pathology and clinical data, the plurality of features comprises at least 300 features. In various embodiments, wherein the plurality of analytes comprise at least four analytes and the at least four analytes comprise proteins (plasma, serum or blood lipids), lipids (plasma or serum lipids), pathology and clinical data, the plurality of features comprises at least 375 features. In various embodiments, wherein the plurality of analytes comprise at least four analytes and the at least four analytes comprise proteins (plasma, serum or blood lipids), lipids (plasma or serum lipids), pathology and clinical data, the plurality of features comprises about 250-500 features. For example, the Spearman rho, Sperman p-value, feature frequency, and feature weights for these features are used as noted above to prognosticate the subject regarding survival and/or recurrence.

In various embodiments, the method further comprises selecting a pancreatic cancer treatment method from among a plurality of pancreatic cancer treatment methods based on the likelihood of survival, the likelihood of recurrence or both. In various embodiments, the method further comprises administering the pancreatic cancer treatment method.

Examples of pancreatic cancer treatment methods include but are not limited to surgery, radiation therapy, chemotherapy, chemoradiation therapy, and targeted therapy.

Examples of surgeries include but are not limited to whipple procedure, total pancreatectomy (removal of the whole pancreas, part of the stomach, part of the small intestine, the common bile duct, the gallbladder, the spleen, and nearby lymph nodes), distal pancreatectomy, biliary bypass, endoscopic stent placement, and gastric bypass (to: so the patient can continue to eat normally).

Examples of targeted therapy include but are not limited to tyrosine kinase inhibitors (TKIs) (e.g., erlotinib).

Additional example of therapies include but are not limited to Abraxane (Paclitaxel Albumin-stabilized Nanoparticle Formulation), Afinitor (Everolimus), Capecitabine, Erlotinib Hydrochloride, Everolimus, 5-FU (Fluorouracil Injection), Fluorouracil Injection, Gemcitabine Hydrochloride, Gemzar (Gemcitabine Hydrochloride), Infugem (Gemcitabine Hydrochloride), Irinotecan Hydrochloride Liposome, Lynparza (Olaparib), Mitomycin, Olaparib, Onivyde (Irinotecan Hydrochloride Liposome), Paclitaxel Albumin-stabilized Nanoparticle Formulation, Sunitinib Malate, Sutent (Sunitinib Malate), Tarceva (Erlotinib Hydrochloride), and Xeloda (Capecitabine).

Still other therapies include but are not limited to chemotherapy combination containing the drugs leucovorin calcium (folinic acid), fluorouracil, irinotecan hydrochloride, and oxaliplatin, gemcitabine-cisplatin, gemcitabine-oxaliplatin, and chemotherapy combination containing the drugs oxaliplatin, fluorouracil, and leucovorin calcium (folinic acid).

Still other therapies include but are not limited to Afinitor Disperz (Everolimus), Lanreotide Acetate, Lutathera (Lutetium Lu 177-Dotatate), Lutetium Lu 177-Dotatate, and Somatuline Depot (Lanreotide Acetate), Belzutifan, and Welireg (Belzutifan).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1—Methods

### Participants Recruitment, Sample Collection, Processing, and Classification

Patients were selected based on the samples that were available in the Cedars-Sinai Medical Center Biorepository. All patients were consented prior to specimen collection and all specimens were collected as part of standard of care and through protocol IRB STUDY00000806 MT-Pilot Study, Feasibility of Extensive Molecular Profiling of Pancreatic Tumors: Lessons for Molecular Twin. Tissues were procured from surgical specimens as part of the standard of care. Blood samples were collected with routine blood work. The time in which these samples were collected ranged from March 2015 to April 2019. Follow up data were completed based on the standard of care. All cases are pancreatic cancer with the diagnosis of ductal adenocarcinoma. This was chosen based on the availability of formalin fixed paraffin embedded (FFPE), frozen tissue, buffy coat, and plasma. FFPE and frozen tissue were collected following tumor resection and were stored in the biobank for future research use. The process of collection and storage was done on site at Cedars-Sinai Medical Center.

The Cedars-Sinai Medical Center Biobank and Pathology Shared Resource reviewed in-house cases and histologically confirmed PDAC from initially assembled list. Specifically, fresh frozen tissue (tumor and adjacent normal) and FFPE tissues (tumor and adjacent normal) were identified. The Biobank prepared each sample for genomic analysis (10 unstained slides per sample+1 H&E). These slides were de-identified and sent to Tempus Labs (Santa Monica, CA) via overnight shipping for genomic and transcriptomic analyses as well as H&E slide digitization

The following set of samples were shipped to Tempus:
93 FFPE tumor samples (10 unstained slides+1 H&E)
93 FFPE normal samples (10 unstained slides+1 H&E)
93 blood samples (buffy coat at 500 μL aliquots)
Clinical data variables for the cohort

Cedars-Sinai Medical Center Proteomics and Metabolomic Proteomics Core analyzed:
60 Frozen Tissue normal
60 Frozen Tissue Tumor
61 Tumor plasma samples with 81 unpaired normal samples

Stage III and IV patients were excluded. Due to the limited number of samples in this pilot cohort, we trained models in a leave-one-out fashion for every analyte separately. During the train phase, we performed feature selection, missing data imputation, and normalization; the same transformations were then applied to the validation sample (the leave-one-out sample) using the means and variance learned on the train data. For certain analytes, we performed preliminary, analyte-specific transformations and feature selection. We utilized binary endpoints at the time of our analysis, Oct. 21, 2021: disease survival (DS): deceased at time of analysis.

### Clinical Data Analysis

We collected 74 plasma and tissue samples of patients with Clinical stage Ia, Ib, IIa, and IIb, resectable pancreatic adenocarcinoma. We obtained clinical characteristics and longitudinal clinical and surgical pathology information for each patient whose sample was analyzed for our multi-omic analysis (Table 3). Our baseline model for the clinical and surgical pathology analytes included general features such as sex, age, BMI/weight/height, tumor stage/size, histologic grade, pathologic variables, treatment duration and type, family history, and personal history of comorbid conditions including other cancers.

### NGS Targeted Genomics

Bulk tissue samples were processed via NGS Tempus|xT onco-gene panel, specifically v4 xT assay covering 648 genes, spanning ˜3.6 Mb of genomic space at 500× coverage. Industry standard bioinformatics pipeline was run on the NGS data for alignment, quality control, and calling of somatic SNVs, INDELs, and CNVs. SNVs were counted per gene in the target panel, generated via Freebayes snp calling pipeline with matched tumor-normals, resulting in 611 gene-level SNV features. INDELs were counted per gene in the target panel, with INDEL calling via the Pindel pipeline using matched tumor-normals, resulting in 126 gene-level INDEL features. Additionally, called CNVs were counted per gene in the target panel, resulting in 648 CNV features. Upon obtaining gene-level somatic SNVs, INDELs, and CNV features, further feature preprocessing was performed, specifically univariate normalization, pruning of low variance features (with variance threshold <0.05), and dropout of highly correlated features (Spearman correlation coefficient <0.95). Processed genomic features consisted of 337 somatic SNV, 219 CNV, and 72 INDEL gene-level features respectively considered for predictive patient survival outcome models.

### RNA Sequencing

Whole-transcriptome sequencing (RNAseq) was performed on 72 tumor tissue samples. In addition, we used 204 (out of 382 total) RNAseq pancreatic tissues samples from the GTex consortium as controls. The GTex samples were selected using the following criteria: participant did not have a cancer diagnosis and participant's age was matched to the age range of the pilot cohort. We then derived two types of RNAseq features:

Gene-level estimated read counts for a set of genes that we found to be differentially expressed between cancer and non-cancer samples.

Read counts per gene for a set of fusion genes.

We obtained estimated transcript read counts by running Kallisto tool (version 0.46.1) on the fastq files for cancer and non-cancer samples. We aggregated transcript-level read counts to gene-level counts using tximport R package (version 1.14.2, Bioconductor version 3.10); this step reduced the number of features from 169 k transcripts to 30427 genes.

To further reduce the feature space and retain only the most promising features, we ran a differential expression analysis between cancer and non-cancers samples. First, we removed all counts below 2 and then removed any genes (separately for cancer and non-cancer datasets) for which fewer than 25% of samples in the set had non-zero values. This left us with 16470 genes for the cancer set and 10478 genes for the non-cancer set. We then only kept genes in the intersection of non-cancer and cancer gene sets, leaving us with 10185 genes total. We selected 2000 genes with the lowest adjusted p-values using the default analysis in_DESeq2 package (version 1.26.0). Finally, we trained our classifiers using log 10 estimated read counts for these 2000 genes as features.

Fusion gene derivation from RNAseq data was another category of omic features considered in the study to capture translocations, interstitial deletions, or chromosomal inversions of two distant, independent genes. Fusion gene features were derived from RNAseq data using an alignment-free algorithm. Number of reads mapping to each fusion gene were aggregated, then limited to known COSMIC fusion pairs. In total 29 fusion gene features were derived from tumor tissue RNAseq data.

### Proteomics and Lipid Analysis

Proteomics analyses were performed on 58 patients with paired tumor-normal tissue samples, via resection of tumor and normal samples from the same frozen tissue block and on 61 tumor plasma samples with 81 unpaired normal samples (Table 16). Proteomics data was generated using DIA-MS technology, with post-processing bioinformatics pipelines performing QC, peak picking, retention time alignment, scoring and false discovery rate identification, normalization, and quantitation. MS2 peak areas at both protein and peptide levels were computed as proteomics features, using a 3777-protein panel for paired tumor-normal tissue samples and a 1052 protein panel for unpaired plasma samples. Similarly, lipidomics analysis using the Lipidyzer Platform kit with internal lipid class standards for quantification reference was performed on plasma samples to obtain composition and concentrations for lipid species, lipid classes, and fatty acids.

Further pre-processing steps for all proteomics and lipidomics data included filtering out proteins and lipids with more than 25% missing data not meeting quality control criteria, removing proteins with low variance <0.1 threshold, followed by imputation of remaining missing values using MEDIAN/2 value for each column and univariate normalization of each column. Alternate strategies for imputation of missing proteomics values, specifically column mean and kNN (k nearest neighbor) imputation, however both were deemed too sensitive to outliers due to small sample size.

Differential expression analysis was performed on the 58 paired tumor-normal tissue samples. Wilcoxon Rank Sum Test was performed between the dependent tumor—normal proteomics samples, with two-tailed p-value <0.05 threshold applied to further remove tumor tissue protein distributions similar to their respective paired normals.

Differential expression analysis was performed on the 61 tumor plasma samples with unpaired 81 plasma samples. Mann-Whitney U-test was performed between unpaired tumor—normal protein distributions, with two-tailed p-value <0.05 threshold applied to remove plasma tumor protein distributions similar to the unpaired normals. Further details on Plasma Proteomics and Lipidomics is found herein.

### Data Availability

Transcriptomic, genomic and clinical data used in this study is available in NCBI/NIH BioProject: accession BioProject ID: PRJNA889519 and associated SRA database.

Proteomic data used in this study was submitted and is available in proteomics Identification Database (PRIDE) as, Profiling of pancreatic adenocarcinoma using artificial intelligence-based integration of multi-omic and computational pathology features Project accession: PXD037038

Any additional information required to reanalyze the data reported in this work paper is available from the lead contact upon request.

Software resources utilized in this study are included as data in Table 17.

### Computational H&E Slide Analysis

71 cases in our MT-pilot cohort had available formalin fixed paraffin embedded (FFPE) tumors that we used to prepare H&E slides for computational analysis. After slide digitization (Aperio GT450 scanner with 40× magnification objective), the resulting whole slide images (WSIs) (n=71) were loaded up to the slide viewer (Aperio ImageScope ver.12.4.3, Leica Biosystems, Buffalo Grove, IL) for a pathologist to box-outline random regions of interest (ROIs) with cancer cells for the analysis. Our goal was to extract architectural features of cancer cell nuclei and assess their fitness and contribution as an analyte in single- and multi-omic ML-based DS prediction models. The ROIs marked (n=2908) and exported from WSIs, were subsequently analyzed by two neural network models. The first model provided a mask of cancer cells and the second model a mask of all nuclei in the ROI (FIG. 2).

The first model was the DeepLabV3Plus—a semantic convolutional neural network model that we trained and tested for the tumor cell masking task using biobanked digital H&E and IHC slides with PDAC. StarDist—an off the shelf convolutional neural network that predicts cell nucleus instance using star-convex polygons was the second model. Intersection of the masks yielded by these two models was the mask of cancer cell nuclei that we then overlaid onto the ROI images.

Nuclear feature extraction was preceded by color-deconvolution of the ROI image to digitally separate the image of hematoxylin staining from eosin. Subsequently, the cancer cell nuclei mask was overlaid onto the hematoxylin image, and architectural features of morphology (size and shape) and features ofhematoxylin staining were quantitated for each nucleus under the mask by means of the 63-feature library (Table 9) that we assembled from available resources. Nuclear features from tumor cell nuclei across all regions in the case were aggregated by means of order statistics: maximum, minimum, average, standard deviation, and 1^{st}, 5^{th}, 10^{th}, 25^{th}, 50^{th}, 75^{th}, 90^{th}, 95^{th}, and 99^{th} percentiles, thereby yielding 819 (13*63) unique features for each case. Z-scored case-level features were used to develop machine learning models for survival prediction. All features in library are image rotation invariant.

For TCGA validation, 33 diagnostic WSIs with PDAC (1 WSI/case) that closely corresponded to WSI specifications (40× scanning magnification and compression quality=70) of the MT-Pilot WSIs were downloaded. The TCGA WSIs were annotated for cancer areas (624 ROIs total, 20 regions/WSI) and tumor cell nuclei (137,617 total, 4,170 nuclei/WSI) automatically identified and delineated in the ROIs by our pipeline. Subsequently, nuclear features (n=819) were extracted from the tumor cell nuclei in the ROIs, z-scored and classified by the ML models predicting DS that we trained using features extracted from the MT-Pilot WSIs. Prior to feature extraction, the H&E staining coloration in the ROIs was digitally matched to that in the MT-pilot WSIs.

### Validation Cohorts

Four validation Cohorts were utilized in the study. The Cancer Genome Atlas (TCGA), Johns Hopkins University (JHU) Cohort 1 and Cohort 2, and Massachusetts General Hospital (MGH) Cohort. TCGA and JHU are publicly available datasets. JHU Cohort 2 is an independent prospective cohort employing identical proteomic and lipidomic analysis as our MT-Pilot and whose raw data was analyzed utilizing the Molecular Twin MLA algorithm pipeline by the JHU team that we used for ML models validation.

### Development of Machine Learning Models for Outcome Prediction

The goal of our study was to train an ensemble of classification models, ranging from simple linear models (i.e., SVMs) to more sophisticated Random Forests and neural networks, with hyperparameters of each model pre-determined and fixed upfront. The ensemble of pre-determined models' approach was used to assess the level of dependence of multi-omic features and the extent to which subtle, non-linear, cross-feature dependencies would provide additional signal and predictive power for non-linear models. Additionally, the model architecture and model hyperparameters were pre-specified and fixed for the study due to the limited sample size in the study and sample size to feature imbalance. As opposed to a typical inner-loop for hyperparameter selection and optimization, the study instead utilized a fixed, predetermined model architecture and hyper-parameters. This was done to prevent overfitting and over-tuning models on the study dataset, instead showing relative performance across classification techniques and demonstrating directional performance of each approach. The architecture and hyperparameters for each classification model, optimization technique and hyperparameters used in the study were implemented in the Python programming language are listed herein. Depending on the validation scenario (internal MT-pilot cohort or external cohorts), developed models were validated using either the leave-one-out cross validation technique (internal MT-pilot cohort only) or using analyte combinations depending on their availability in the validation cohorts (TCGA, JHU Cohort 1, JHU Cohort 2, and MGH Cohort).

### Plasma Proteomics Methods

### Dual-Workflow Depleted and Undepleted (Native) Plasma Sample Preparation

Depletion of high abundant plasma proteins: To improve proteomic depth, a portion of each set of plasma samples were depleted of 14 highly abundant proteins, albumin, Immunoglobulins A, E, G and M (kappa and lambda light chains), alpha-1-acidglycoprotein, alpha-1-antitrypsin, alpha-2-macroglobulin, apolipoprotein A1, fibrinogen, haptoglobin, and transferrin using the High Select Top 14 Abundant Protein Depletion Camel Antibody Resin (Thermo Fisher Scientific). On the day of depletion, anti-camel antibody-resin, which was stored at 4° C., was equilibrated to room temperature for 30 min mixing at 800 rpm. After equilibration, the anti-camel antibody-resin was vortexed vigorously and 300 μL was aliquoted into the wells of a 96 well plate (Nunc™ 96-Well Polypropylene DeepWell™ Storage Plates). 10 μL of plasma was diluted 1:10 with 100 mM NH4CO3 and added to wells containing depletion resin. To ensure homogenous mixing the plate was mixed at 800 rpm for 1 hour (hr). The unbound fraction was aspirated from the resin with 500 L of 100 mM NH4CO3 and transferred to a filter plate (Nunc™ 96-Well Filter Plates). The depleted fraction was collected by gentle centrifugation (100 rcf for 2 min) into a clean 96 well plate (Beckman Coulter, deep well titer plate polypropylene) and lyophilized.

Trypsin Digestion and Desalting: Proteins from 5 μL of plasma were processed for protein denaturation, reduction, alkylation, and tryptic digestion using the manufacturer protocols for the Protifi S-Trap protein sample preparation workflow. Resulting peptides were quantified by BCA assay and 2 μL of peptide suspension from each sample was pooled to make a master mix used for quality control monitoring purposes and for generation of peptide assay libraries for peptide and protein identification from individual DIA-MS samples (see below).

### High-Throughput DIA LC-MS/MS

Mass spectrometry data were acquired on an Orbitrap Exploris 480 (ThermoFisher, Bremen, Germany) instrument separately for the depleted and undepleted plasma samples. Desalted peptides were separated on an Evosep One system (Odense, Denmark) with a 21-min gradient requiring 25 mins to complete each sample. Peptides were separated on a preformed gradient (ranging from 5-35% organic phase) on a C18 column (8 cm, 3 μm) over the course of 21 mins at a flow rate of 1000 nl/min. Source parameters included spray voltage at 2000 kV, capillary temp of 275° C. and RF funnel level of 40. MS1 resolutions were set to 120,000 and AGC was set to 300% with ion transmission of 45 ms. Mass range of 350-1400 and AGC target value for fragment spectra of 300% were used. Peptide ions were fragmented at a normalized collision energy of 28%. Fragmented ions were detected across 50 DIA windows of 21 Da with an overlap of 1 Da (full precursor mz range 349.5-1400.5). MS 2 resolutions was set to 15,000 with an ion transmission time of 22 ms. All data was acquired in profile mode using positive polarity.

### Informatic Processing to Generate Plasma Protein Quantification Tables

DIA MS raw files were converted to mzML, the raw intensity data for peptide fragments were extracted from DIA files using the OpenSWATH workflow and searched against the Human Twin population plasma peptide assay library as described previously. The final table of identified peptide fragments was filtered to remove outliers and aggregated into quantitative protein abundance estimates using mapDIA software. To generate a single table of quantified plasma proteins from the two parallel sample preparation and MS experiments, we identified the proteins uniquely identified in the ‘depleted plasma’ experiments and appended only these quantified results to the existing identifications from the undepleted plasma experiment. We assumed that increased technical processing during the depletion workflow would be more likely to impact quantitative variability, and thus we prioritized quantitative data from the undepleted workflow for any protein identified in both experiments. Analysis of the pooled digestion QC samples indicated median digestion coefficients of variance of 31%, 17.4%, and 11.3% for the undepleted and 25.5%, 23.5% and 37.3% for the depleted plates of original and two separate validation sets, respectively.

### Plasma Lipidomics Methods

### Sample Processing & Lipid Extraction

Lipids were extracted from plasma using the Bligh-Dyer method. Briefly, 50 μL of plasma was treated with 950 μL of water, 2 mL of methanol and 900 μL of dichloromethane. Internal standards were added at this point according to the manufacturer's protocol and incubated at RT for 30 minutes after which point an additional 1 mL of water and 900 μL of dichloromethane was added to crash out the protein and the samples were quickly vortexed. Samples were centrifuged at 3000 g for 10 min and the dichloromethane layer was removed and dried. The dry lipids were resuspended in 250 μL running buffer (10 mM ammonium acetate, 50:50 methanol: dichloromethane).

### Mass Spectrometry Based Lipid Species Quantification

Extracted lipids were analyzed on a Sciex Lipidyzer™ Platform consisting of a triple quadrupole mass spectrometer (5500 Q-trap) with a SelexION front end with a standardized workflow for the simultaneous analysis of 1153 lipids representing 13 lipid class. Samples were loaded by direct infusion from a Shimadzu LC-30AD LC system equipped with a SIL-30AC auto sampler. Lipid concentrations were determined by the Lipidyzer software using the ratio of the endogenous lipid to internal standard. Data are reported for each individual lipid species, as an aggregated value for lipid classes, and as the relative composition compared to all other measured lipid classes.

### Tissue Proteomics Methods

### Sample Processing & Lysis.

Tumor biopsies as well as biopsies from non-tumor tissue segments were assessed for tumor and stromal cell content by clinical pathologists and a curl of frozen tumor (encompassing the full surface area of pathologist estimated tissue) was collected and submitted for proteomics processing. Tissue sections were then lysed in 8M Urea with 5% SDS and 100 mM glycine and lysed using a handheld motorized homogenizer. Following 5 minutes of sonication to shear DNA, samples were centrifuged at 14,000×G for 10 minutes at 4 degrees to pellet insoluble debris, and the supernatant was transferred to clean, low protein binding tubes and protein concentration determined using Pierce BCA assay (Thermo Fisher Scientific, Waltham, MA, USA). A total of 30 g from each sample were then processed and digested using the S-TRAP micro-elution tips (Protifi, Farmingdale NY) according to manufacturers protocol, and the resulting peptides were dried and stored at −80 C prior to MS acquisition.

### Data Independent Acquisition LC-MS.

Dried peptides were resuspended in 0.1% formic acid with 1:40 dilution of Biognosys iRT reference peptides (Biognosys, Schlieren Switzerland) at a concentration of 1 μg/μL. 5 μL of peptide solution was injected onto a 15 cm Phenomenex Omega Polar C18 3 μm 100A 150×0.3 mm column and separated over a 60 minute gradient transitioning from 0%-45% acetonitrile (buffer B) in 0.1% formic acid (buffer A) at 7 μL/min flow rate. Peptides were ionized by electrospray into a Thermo Fusion Lumos mass spectrometer operating in data independent acquisition mode. The instrument cycled continuously between 1) an intact MS1 scan of all peptides between 400-1600 m/z in the orbitrap detector at resolution 120K, accumulation time of 50 ms and target AGC of 400K and 2) 40 subsequent MS2 scans systematically isolating all ions within 15mz range intervals from 400-1000 m/z and analyzing high energy induced collision (CE 30%) induced fragments between 200-2000 m/z from each window in the orbitrap at 30K resolution, maximum injection time of 54 per scan and target AGC set to 500K. Total cycle time to progress through each MS1 and 40 MS2 scan series was 3 seconds.

### Data Informatics to Generate Tissue Protein Quantification Tables

Data were analyzed using our established workflows as previously described. Briefly, peptides were identified using the openSWATH workflo, searched against the pan human library with decoy sequences appended for false discovery rate calculation using pyprophet algorithm. Peptides with no greater than 5% identification FDR across all samples were compiled into the final experimental results using the TRIC alignment algorithm. Following removal of non-proteotypic peptides (e.g., sequences matching more than one gene product from the Pan Human library), the final aligned results were analyzed using mapDIA software to select only high quality performing fragments for quantification and to compile fragment level data into peptide and protein level abundance estimates.

### Computational Pathology Methods

### Development of Training Dataset and Training of DeepLabV3Plus Neural Network Model

The DeepLabV3Plus neural network model was trained and tested for the tumor cell masking task (FIG. 2) using WSIs of 10 slides sequentially stained with H&E and immunohistochemistry (IHC). Briefly, following our established protocol, the 10 tissue sections were first stained with H&E and digitized, then destained, re-stained with a cocktail of IHC antibodies reactive to cytokeratines (DAB chromogen) and digitized again. By overlaying the WSI of the IHC-stained slide onto the corresponding WSI from the H&E-stained slide, we obtained ground truth delineation of cancer cells in the H&E-stained WSI. The H&E and IHC stained slides were digitized on the same slide scanner (Aperio, 20× magnification) and the 10 tissue sections were from PDAC tumors biobanked at Cedars-Sinai.

Subsequently, matching image regions with tumor cells were in the corresponding H&E and IHC WSIs were extracted and co-registered using affine image registration to obtain accurate alignment. Aligned image regions (n=416) were downsized by the factor of 0.5 and divided into non-overlapping 256×256 pixel tiles (n=2656). To generate ground truth mask for cancer cells in the tiles, the DAB staining was digitally deconvoluted and thresholded, and the resulting cancer cells mask smoothened by mathematical morphology operators. The tiles were then augmented 15 times, and a training set of 39,840 H&E tiles paired with corresponding tumor cell mask tiles was used for the DeepLabV3Plus model training. The model was trained for 75 epochs; the initial learning rate, gamma, L2-regularization, and momentum for stochastic gradient descent optimizer were set to 0.005, 0.9, 0.001 and 0.1 respectively. The learning rate was halved every 5 epochs and reached 3.05e-7 at the end of training. The minibatch size was 12 tiles. After training, the model achieved overall accuracy of 97.5%.

### DeepLabV3Plus Neural Network Model Testing

The trained DeepLabV3Plus model was tested for the tumor cell detection ability on a WSI from a commercial tissue microarray (TMA) (TissueArray, Derwood, MD, TMA #PA483e) comprising 40 PDAC tumor cores (1 subject each) with: 20 duct adenocarcinomas, 13 adenocarcinomas, 1 mucinous adenocarcinoma, 1 papillary adenocarcinoma, and 1 acinar cell carcinoma, and 1 squamous cell carcinoma. The TMA slide was subjected to the same staining/restraining/digitization protocol as the slides used for the DeepLabV3Plus model training. The test WSI provided 80 large image regions with cancer cell ground truth mask that we used to measure the accuracy, mIoU, and F1 scores (tumor and non-tumor) of the DeepLabV3Plus model that was applied to the corresponding 80 H&E regions. Performance metrics are reported herein.

### Architecture and Hyperparameters for Each Classification Model

$$Principal Component Analysis ( PCA ) + Logistic Regression : \{ num\_components = 20 , penalty = 11 , fit\_intercept = true , solver = lbfgs \} ; \\ Support Vector Machine : \{ loss = hinge , penalty = 12 , fit\_intercept = true , alpha = 0.0001 \} ;$$

$$L 1 - norm Support Vector Machine : \{ loss = hinge , penalty = 11 , fit\_intercept = true \} ; \\ L 1 - norm Support Vector Machine + Random Forest : \{ loss = hinge , penalty = 11 , fit\_intercept = true , num\_estimators = 100 , split\_criterion = gini \} ;$$

$$Support Vector Machine + Muti - layer Perceptron : \{ loss = hinge , penalty = 11 , fit\_intercept = true , hidden\_layers = ( 512 , 256 , 128 , 64 , 32 ) , max\_iter = 1000 , activation = relu , solver = adam \} ; \\ Recursive Feature Elimination ( RFE ) + Logistic Regression : \{ penalty = 12 , fit\_intercept = true , solver = lbfgs , alpha = 0.0001 , pct\_feature \_dropout = 0.2 \} ; \\ RFE + Random Forest : \{ num\_estimators = 100 , split\_criterion = gini , pct\_feature \_dropout = 0.2 \}$$

### Example 2—Results

### Patient Baseline Demographics and Specimen Handling

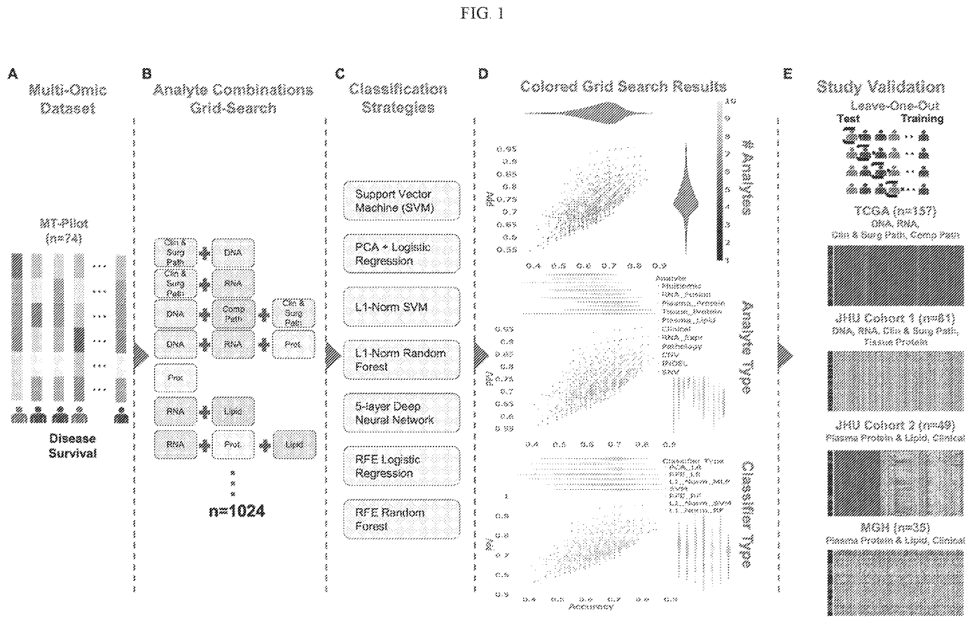

Our Molecular Twin Pilot Cohort (MT-Pilot) included 74 patients with clinical Stage I (n=47) and II (n=27) with surgically resected PDAC between March 2015 and April 2019. Clinical stage III and IV patients were not considered for inclusion. Tumor specimens were collected at the time of surgery and plasma specimens preoperatively. DS for all 74 patients within this cohort was recorded and treated as a binary endpoint at the time of our analysis, Oct. 21, 2021. At this time, 45 (61%) patients were deceased. All demographic and clinical characteristics (Table 3) were included as features for the clinical analyte in our multi-omic analysis. The surgical pathology information was obtained from the pancreas resection. Tumor and plasma specimens were assessed for individual features by molecular profiling including targeted next generation sequencing (NGS) DNA sequencing, full transcriptome RNA sequencing, paired (tumor and normal from same patient) tissue proteomics, unpaired (tumor from patients and normal unrelated controls) plasma proteomics, lipidomics, surgical pathology, and computational pathology. Analyte profiling yielded features that we used to validate single- and multi-omic MLAs for predicting DS; the leave-one-out cross validation approach was applied to the MT-Pilot cohort whereas the 4 independent datasets, TCGA, JHU Cohort 1, JHU Cohort 2 and MGH were used to validate our feature panels generated by applying MLAs on the MT-pilot data (FIG. 1).

### Clinical and Surgical Pathology Features Contribute to Outcome Prediction

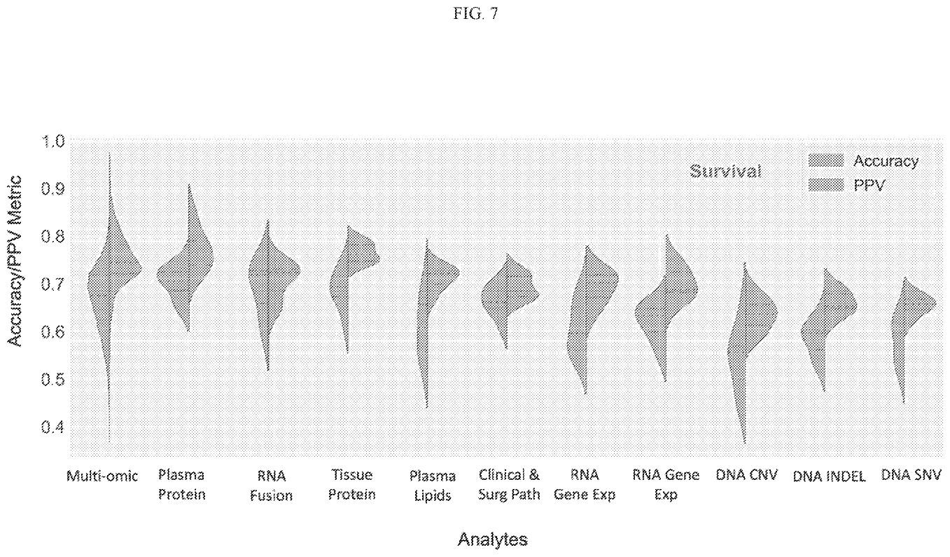

331 clinical features (i.e., tumor stage, age, sex, BMI), surgical pathology features (i.e., margin status, grade, pathologic staging, perineural invasion [PNI], lymphovascular invasion [LVI]), and chemotherapy treatment history (Table 3), as well as comorbidities (Table 4A-4C) were analyzed using multiple MLA models. When trained with these features, the Random Forest was the top performing model in determining DS and achieved accuracy of 0.70 (95% CI 0.60-0.81) and PPV of 0.71 (95% CI 0.60-0.82) (Table 1, FIG. 7). Top features predicting outcome included comorbidities, such as hyperlipidemia, jaundice, and pancreatitis, as well as surgical margin status (Table 4A-4C) which are known in the PDAC field. The model for DS was predominantly driven by comorbid conditions, which accounted for 306 of the 331 total features. The Random Forest model was also trained using the remaining 25 features which included known PDAC predictors such as prior chemotherapy, margin status, PNI, and LVI. This model performed similarly to ones that which included all clinical features (Table 4A-4C). Importantly, the top 10 features of this model included surgical margin status, tumor grade, chemotherapy, and radiation therapy which are known to influence patient outcome.

DNA Analysis Reveals Both Known and Novel Alterations with Prognostic Significance

Point mutations and insertion/deletion polymorphisms (INDELs) are common in the PDAC genome with many oncogenes and tumor suppressor genes harboring mutations. KRAS, TP53, CDKN2A, and SMAD4 are the most prevalent mutated genes in PDAC. Tissue samples were processed for 611 somatic single nucleotide variants (SNVs), 648 CNVs, and 126 INDEL. These features were then used in patient DS prediction models (Table 5A-5B).

Using SNV features, the top performing model to determine DS was Random Forest, with accuracy of 0.64 (95% CI 0.53-0.75) and PPV of 0.66 (95% CI 0.55-0.77) (Table 1, FIG. 7). In models evaluating SNVs, we found alterations in RAD51, IL6R, FGF20, and SOX2 genes as the top features for DS prediction (Table 5A). Their high ranking supports the value of the Random Forest model since RAD51, IL6R, FGF20, and SOX2 and their associated signaling pathways have significant prognostic implications in PDAC. In addition, we found novel genes not previously associated with PDAC prognosis or targetable pathways, such as RIT1, that were top predictive markers identified by our model.

Using CNV features, the top performing model to determine DS was a Random Forest model with accuracy of 0.65 (95% CI 0.57-0.80) and PPV of 0.68 (95% CI 0.57-0.80) (Table 1, FIG. 7). The top CNV features for DS are noted in (Table 5A). Interestingly, we found FOXQ1 and KDM5D were top predictors associated with DS. Both are markers for PDAC prognosis and potential therapeutic targets. In our cohort, the four commonly mutated genes, KRAS, TP53, CDKN2A, and SMAD4, were included among a total of 126 specific INDEL features and were learned by multiple MLA model types. The top performing model for DS was Random Forest with accuracy of 0.64 (95% CI 0.53-0.75) and PPV of 0.70 (95% CI 0.58-0.82) (Table 1, FIG. 7). The top features in the model included mutations of TP53, CDKN2A and SMAD4, which have been shown to correlate with poor prognosis and more aggressive phenotypes of PDAC. Other top feature gene mutations such as DIS3L2 and CHD4 identified by our MLAs have mechanistic data supporting their role in oncogenesis and growth, but their role as predictive markers was limited until our analysis.

RNA Evaluation Found Anti-Tumor Immunity and Drug Resistance Genes with Prognostic Significance

Whole-transcriptome sequencing was performed on 72 of the 74 FFPE tumor tissue samples. To optimize for the most predictive features, we first ran a differential expression analysis between cancer and non-cancers samples from the GTex consortium. Unpaired differential expression was conducted via Mann-Whitney U-test with p-value <0.05, from which the 2000 most differentially expressed RNA gene transcripts were selected for downstream modeling (Table 6A-6B). The top performing model to determine DS was L1-normalized Random Forest which yielded an accuracy of 0.68 (95% CI 0.56-0.80) and PPV of 0.70 (95% CI 0.57-0.83) (Table 1, FIG. 7). In our top model for DS prediction, the NFE2L2 and LRIG3 genes, were the two top features (Table 6A). Recent investigations have shown that the NRF2 pathway through NFE2L2 regulates resistance to drugs and immunotherapy. USP22, previously reported to play a role in anti-tumor immunity in PDAC, was also atop DS predictor. Additionally, a total of 29 RNA fusions were analyzed using multiple model types (Table 6A). The top performing model featuring RNA fusions to determine DS, was Support Vector Machine with accuracy of 0.75 (95% CI 0.64-0.87) and PPV of 0.74 (95% CI 0.62-0.87) (Table 1, FIG. 7).

### Plasma Proteins are a Significant Analyte in Survival Prediction

Proteomics and lipidomics analysis generated 3777 tumor tissue proteomic, 1051 plasma proteomic, and 939 lipidomic features (Table 7A-7B). Redundancy was reduced by elimination of highly correlated features (Spearman correlation, rho <0.95, p-value <0.05) leaving 406 lipidomic features. Tumor tissue proteomic features were pruned to 1130 by eliminating those not expressed at higher levels in tumors compared to normal pancreas (Wilcoxon signed rank test, p-value <0.05). Plasma proteomic features were reduced to 257 via tumor-normal plasma protein differential expression analysis (Mann-Whitney U-test, p-value <0.05).

Using tissue protein features, the top performing model to predict DS was Random Forest model with accuracy of 0.73 (95% CI 0.61-0.86) and PPV of 0.76 (95% CI 0.63-0.89) (Table 1, FIG. 7). For plasma protein features, the top performing model for DS, was the 5-hidden layer Deep Neural Network model with accuracy of 0.75 (95% CI 0.63-0.86) and PPV of 0.80 (95% CI 0.68-0.90) (Table 1, FIG. 7). Among DS predictive plasma proteins, we identified ANXA1, which is an important emerging player in pancreatic carcinogenesis and PDAC drug resistance. Additionally, a plasma proteomics study implicated ANXA1 as an early predictor of PDAC development. The top performing model using plasma lipid features to determine DS was the Random Forest model with accuracy of 0.71 (95% CI 0.58-0.83) and PPV of 0.74 (95% CI 0.61-0.87) (Table 1, FIG. 7). Top plasma lipidomics features for DS were driven by diacylglycerols (DAG) and cholesteryl esters (CE) (Table 7A).

As discussed above, CA 19-9 is routinely utilized in clinical practice at PDAC diagnosis, pre- and post-operatively to assess disease biology, treatment response, and prognosis. CA 19-9 readouts obtained at diagnosis, prior to surgery and postoperatively, were learned by Random Forest model, but the DS prediction had low accuracy (0.59-0.64, 95% CI 0.47-0.76) and PPV (0.52-0.61, 95% CI 0.40-73) across all time points (Table 8).

### Nuclear Morphology Features Assessed by Computational Pathology Predict Outcomes

71 of 74 FFPE, H&E-stained, PDAC tissue whole slide images (WSI) were evaluated by a novel (AI)-based digital pathology pipeline we developed (FIG. 2). Pipeline components included a semantic cancer cell masking model (FIG. 2B) to distinguish tumor cells from other cells for downstream analysis. When tested on images from an independent set of 40 PDAC cases (80 regions in total) from patients not included in our cohort of 71, the model achieved 0.90 global accuracy, 0.784 mean Intersection over Union (mIoU), and mean F1-scores of 0.83 and 0.77 in identifying non-tumor and tumor tissue pixels, respectively. We also built-in a semantic nuclei delineation model into the pipeline (FIG. 2B) and ran the pipeline on 2908 regions (˜41+/−11 regions/case) randomly selected from the 71 digital H&E slides in our cohort. The pipeline automatically isolated 345,038 tumor cell nuclei (˜4,860 nuclei/case). Nuclear morphology and texture were quantitated by a panel of 63 characteristics. Distribution of characteristics in each case was further summarized by 13 order statistics yielding 819 features per case (FIG. 2C, (Table 9). The uniform manifold approximation and projection (UMAP) plot revealed cases with the same outcome clustered together (FIG. 2D) suggesting that some of the features in the panel have the potential to predict outcomes. Using the leave one-patient out (LOO) approach and 819 features per case, we trained and cross-validated 7 classification models for binary DS prediction. The top performing model for predicting DS, was a Random Forest model with accuracy of 0.66 (95% CI 0.55-0.77) and PPV of 0.76 (95% CI 0.63-0.88) (FIG. 2E). Throughout all validation steps, features learned by the top model were ranked based on the impact on determining the outcome label, and the frequency of occurrence of impactful measured features. Impactful features which occurred in at least 10% of validation steps were considered top features. The 17/39 top features to predict survival in FIG. 2F originated from the same 10/63 nuclear characteristics in FIG. 2C.

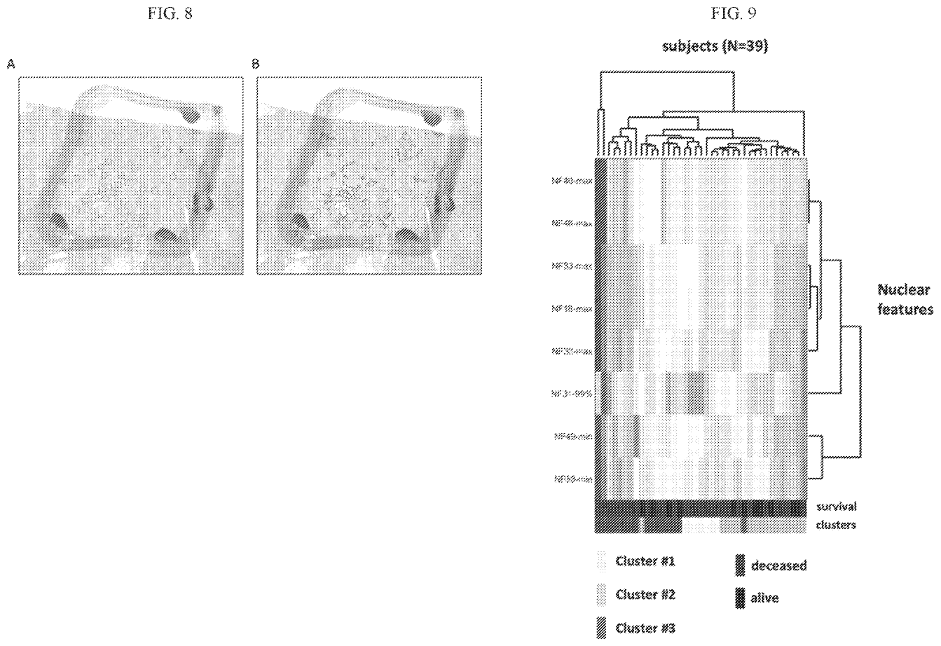

To assess whether the ML-based prediction of DS could benefit from the inclusion of percent of stroma or cancer to stroma ratio in our samples, we applied our A1 pipeline (FIG. 2B) to the cancer region marked by our pathologist (W.T.) and measured the proportion of tumor pixels (pCA), stromal pixels (pST) and the ratio of these two (r=pCA/pST) in the region with cancer (FIG. 8A-B). When this technique was applied, no statistically significant difference in pCA (t-test p-value=0.3) and r (t-test p-value=0.257) was found when tumors associated with poor survival (DS=1, n=28) were compared to those with better survival (DS=0, n=43) As no difference was appreciated, we did not incorporate the above features into the computational pathology analyte. Regardless, we found that the percentage of stroma is significantly larger in tissue after neoadjuvant therapy which can occur following neoadjuvant therapy. Additionally, the percentage of cancer was smaller in tissue after neoadjuvant therapy, which is the intent of neoadjuvant therapy (Table 9). These stromal and tumor findings from our A1 analysis are further supported by in-depth stromal analysis done by others.

### Multi-Omic Analysis Suggests Hierarchical Complementarity Across Analyte Types

6363 individual processed features from each of the single-omic sources were combined and analyzed using 7 independent machine learning (ML) models, trained in a leave-one-patient-out cross validated approach (complete multi-omic feature dataset Table 1). Each single-omic source and multi-omic combinations were evaluated using all ML models. Modeling strategies are shown in FIG. 1C. The hyperparameters of each model were fixed at the initial design of the study to prevent over-optimization and overfitting due to the small cohort size. The top model for prediction of DS was the multi-omic model, which had an accuracy of 0.85 (95% CI 0.73-0.96), and PPV of 0.87 (95% CI 0.75-0.99), followed by single-omic analyte analysis of plasma protein, RNA fusions, tissue protein, plasma lipids, clinical & surgical pathology, RNA gene expression, computational pathology, DNA CNV, DNA INDELS, and DNA SNV in decreasing order of model prediction accuracy (Table 1, FIG. 7).

The accuracy and PPV performance yielded by single-omic models suggest that each single-omic analyte in isolation carries some predictive power and thus potential clinical utility. The best predictors of DS were plasma proteins leading to development of a model with accuracy of 0.75 (95% CI 0.63-0.86) and PPV of 0.80 (95% CI 0.68-0.92). The model learning only pre-surgery CA 19-9 achieved accuracy of 0.59 (95% CI 0.47-0.71) and PPV of 0.53 (95% CI 0.40-0.65), and it was considered the worst among all the single-omic models. As observed in the top two rows of the model performance Table 1, the top multi-omic models outperformed the single-omic ones in accuracy (10%-21%) and PPV (7%-19%) in predicting DS, suggesting complementarity and information gain across analytes when combined under the multi-omic analytical approach. On the other hand, the multi-omic models had a larger dispersion of accuracy and PPV, when compared to the single-omic models (Table 1, FIG. 7) likely resulting from the involvement of a much larger set of features available for multi-omic models training.

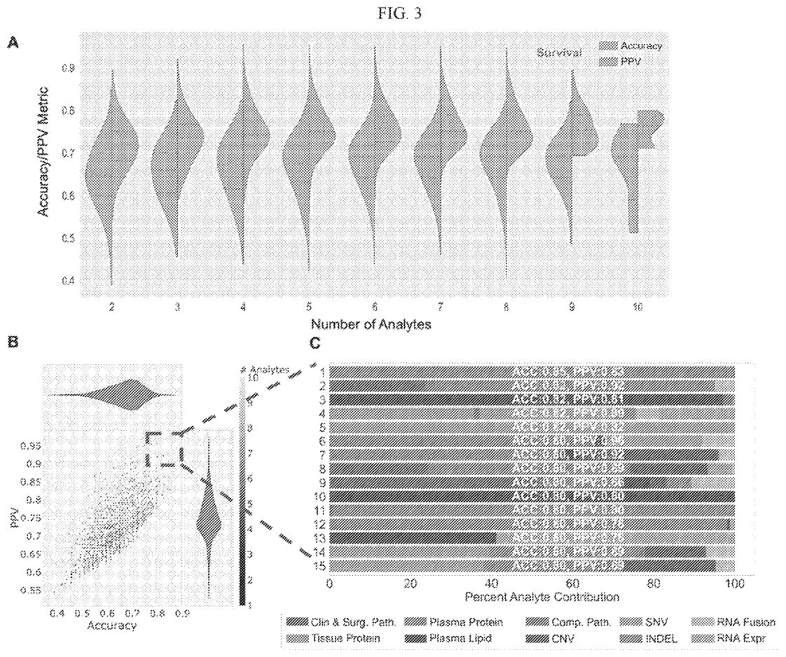

1024 Individual analyte combinations (single and multiple) with all 7 modeling strategies per analyte combination resulted in 7168 grid search runs (FIG. 1). To establish per-analyte importance, the Drop-Column Importance strategy was utilized and adapted, where each analyte's set of features were dropped in their entirety. Using results from the 7168 runs, we evaluated the model's predictive performance, analyte composition, and feature contributions (FIG. 3). Models trained with features from any 2-4 or 9-10 analytes were inferior in accuracy and PPV to the models trained with features from any 4-8 analytes. Interestingly, models trained with 9 or 10 analyte combinations were not among the top performing models (FIG. 3A).

Additionally, with the Drop-Column Importance approach, we were also able to quantify the importance of each analyte category (Table 10). We compared performance when excluding all genomic (SNVs, CNVs, INDELs), all transcriptomic (tissue RNAs, fusions), all proteomics (plasma and tissue), lipidomics (plasma), computational pathology, surgical pathology, and clinical analytes. Furthermore, we assessed several clinically relevant combinations. The results in Table 10 show that exclusion of any one analyte from the study generally reduced but did not significantly alter the performance; the accuracy and PPV for DS prediction were in the range of [0.85-0.83] and [0.84-0.83], respectively.

Next, we focused on the top 15 multi-omic models for DS (FIG. 3B) prediction, which were those with an accuracy >0.80 and PPV >0.78. We plotted proportions of analyte's features learned by each model (FIG. 3C) and observed that the top models had nearly similar accuracies and PPV, however the proportions of contributing features varied across the top 15 models. The predominant feature contribution was from the plasma protein analyte (green bar, FIG. 3C). We also observed a substantial variation in the origin of learned features; the majority of top models learned plasma protein, plasma lipid, or tissue protein features. Features extracted from other analytes were learned to a lesser degree.

Multi-Omic Models Provide Biological Insights into Pancreatic Cancer

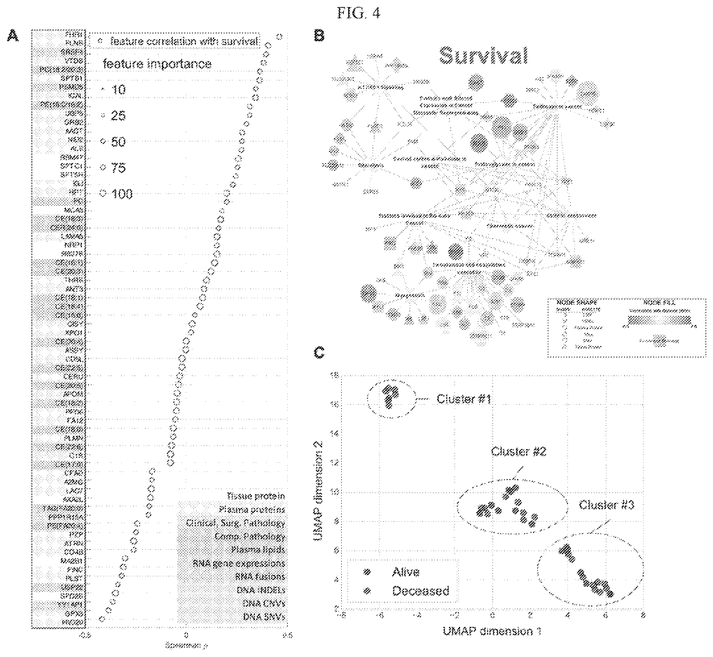

Given the relative paucity of predictive biomarkers and therapeutic advances in PDAC compared to other cancers, an important exploratory objective of our study was to assess if our Molecular Twin platform can identify potential novel pathways and targets of therapy. We began by evaluating unpaired tumor-normal differential expression via Mann-Whitney U-test (p-value <0.05) for plasma proteins and tissue RNA, paired tumor-normal differential expression via Wilcoxon Signed Rank Test (p-value <0.05) for tissue proteins, and Spearman correlation (rho <0.95, p-value <0.05) for plasma lipids. Using a differentially expressed feature set, we were able to ascertain features to study objective Spearman correlation and the importance for all analyte features (FIG. 4A). By evaluating analyte contribution for each model, it was possible to generate ontology visualizations for protein, DNA, and RNA as shown for the top multi-omic models for DS (FIG. 4B). These figures (FIG. 4A-B) enable succinct visual inspection of the models that facilitates interpretation of biological relevance.

mTOR signaling, a known pathway in many tumors including PDAC, was found in the ontology network visualizations of the top multi-omic models (FIG. 4B). mTOR signaling has been targeted in PDAC alone and in combination with other agents with mixed results. Our gene ontology network visualizations also reveal numerous other clinically and biologically relevant pathways in PDAC, including glycolysis, complement, and cellular metabolism.

To examine the relationship of tumor to outcome heterogeneity, all 6363 features across all analytes were used to create patient level clustering based on multi-omic molecular signatures and plotted for binary outcomes of survival, deceased vs. alive (FIG. 4C). Cluster #1 represents patients homogeneous for their clinical outcome (all deceased) and multi-omic features. Cluster #2 represents a heterogeneous population with regards to clinical outcome while cluster #3 represents a more homogenous population compared to cluster #2. Notably, in cluster #3, patients noted to be alive at the time of analysis were strongly predicted to be deceased by the model. Longer follow up will determine if these patients remain well or succumb to their disease. To better understand the association of the heterogenous clusters, (#2 and #3), with other clinical and computational pathology features, we compared the expression of a feature in one cluster to that in the two other clusters combined using t-test or Fisher's test. This analysis revealed proportions of relevant features (p<0.05) in each analyte (Table 11), where except for computational pathology, no other analyte contained features that were present in all three pair-wise comparisons. Subsequently, we used one-way ANOVA which identified 8 differentially expressed features in the computational pathology analyte (Table 12). These 8 features were then analyzed by the Tukey-Kramer test for multiple comparisons where no feature was significantly different between the 3 clusters. Furthermore, hierarchical clustering of the 39 subjects characterized by the 8 computational pathology features (FIG. 9) suggests that they strongly contributed to the formation of cluster #1, #2, and #3. Together, these findings suggest that with more patients and with prospective iterative analysis over time, our approach will result in progressively more accurate predictions especially for patients who fit membership in specific clusters (e.g., cluster #1) and deeper insight into what features are critical to individual patient clusters.

### Development and Evaluation of Parsimonious Multi-Omic Models for Disease Survival

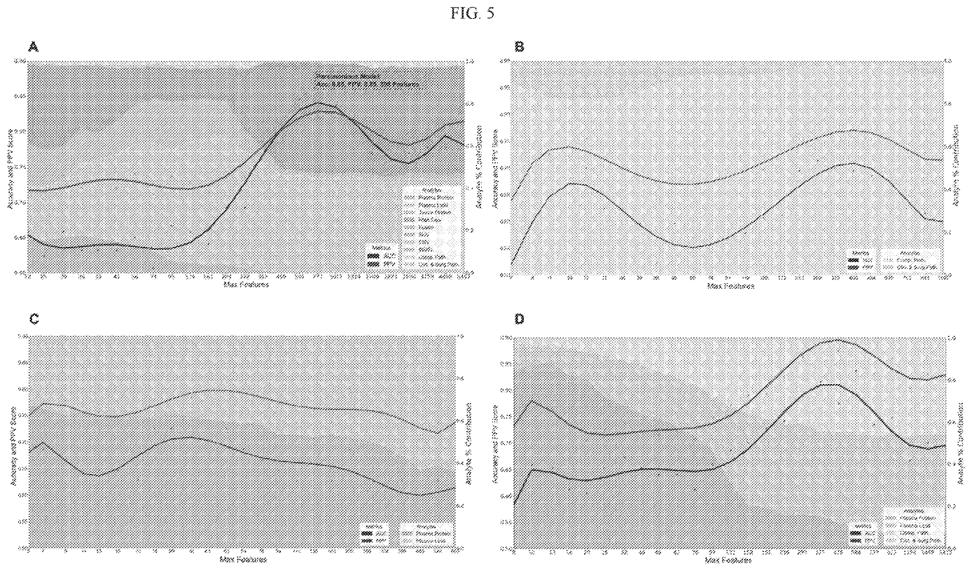

The complementarity of analytes observed in multi-omic models in Table 1, Table 10, and FIG. 3, suggested that a parsimonious multi-omic model offering similar predictive performance to models with larger and more complex analyte compositions could be developed. If true, the global public health and societal impact would be significant as it would potentially begin the process of democratizing precision cancer medicine especially to areas of the world with limited financial and technical healthcare resources. To test this hypothesis, we started with the complete multi-omic feature space of 6363 features, and we trained a Random Forest model for DS utilizing a recursive feature elimination (RFE) strategy such that at each step the least informative features were eliminated from further model iterations (FIG. 5A). This approach established the relationship between model performance and analyte contributions as the number of allowable features was recursively restricted. The curve is comprised of three distinct sections: 1) number of features above 1709 suggesting presence of noise and high feature set dimensionality resulting in sub-optimal performance; 2) features between 459 to 1709 demonstrate peak performance as a majority of noisy features were eliminated; 3) when the number of features were near and before 459 there was an inflection point showing further feature elimination resulted in information loss as evident in drop in accuracy and PPV. Most notably, FIG. 5A highlights the inflection point of the “Parsimonious Model” location on the curve (accuracy of 0.85, PPV of 0.85) learning only 589 multi-omic features. Further, the contribution of respective analytes to the parsimonious model remains mostly stable across iterations after the inflection point, with plasma lipids and RNA being the most relevant. However, note that plasma (proteins or lipids) alone can provide accurate prediction with fewer features. This opens the possibility that a screening of plasma could eventually be used for decision making regarding pancreatic surgery.

Trying to examine the potential of this approach for eventual globalization of precision oncology, we assessed specific limited analyte combinations and feature sets that could be applied to our parsimonious model. These analytes were selected based on criteria of standard availability (pathology specimens or clinical data including surgical pathology) or easily obtained (plasma lipids or proteins) as part of the diagnostic workup. Using this approach, we identified accurate parsimonious models that learned features from clinical, surgical pathology and computational pathology analytes (FIG. 5B), all plasma analytes (lipidomics and protein) (FIG. 5C), and clinical, combined with computational pathology and plasma analytes (FIG. 5D) and which had similar accuracy and PPV to the models that learned features from the entire set of 6363 features in FIG. 5A.

### Validation of RNA Markers as Predictors of Both Improved and Poor Survival on the TCGA PDAC Dataset

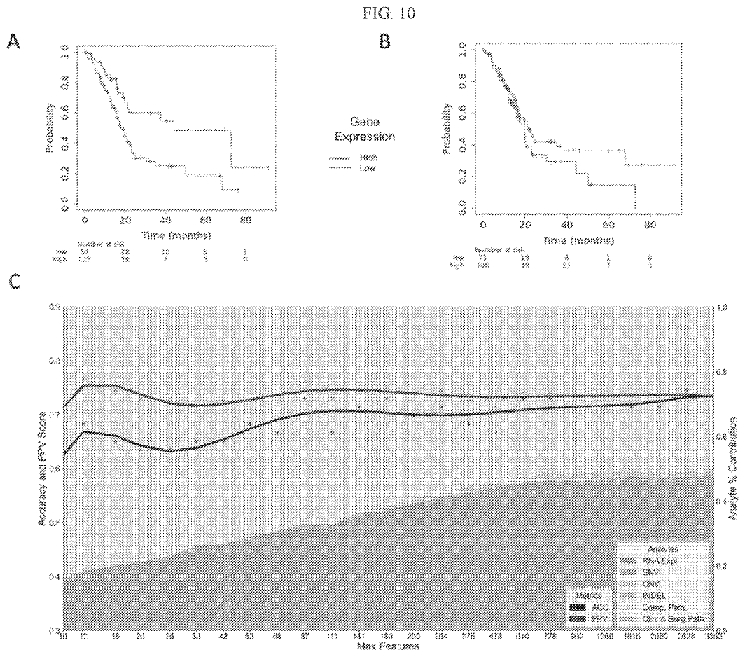

Whole-transcriptome sequencing and analysis as previously described, was performed on 57 samples from our pilot cohort, leading to selection of 2000 differentially expressed RNA gene transcripts for downstream modeling (Tables 6A-6B). Employing L1-normalized Random Forest Modeling, RNA gene transcripts significantly (p≤0.05) predicting survival (n=79) were used to develop two separate gene signatures, one for improved (positive Pearson and Spearman rho for survival, n=40 genes) and the other for poor (negative Pearson and Spearman rho for survival, n=39 genes) survival (Tables 13A-13B). These two signatures were evaluated in an independent dataset of 177 PDAC patients for their ability to stratify DS. High score of the signature composed of genes whose expression was associated with poor prognosis in our data (n=39) was also associated with poor DS in this set (HR=2.17, [1.28-3.66], logrank p=0.0031) (FIG. 10A) while that of genes whose expression was defined as a good prognostic in our data (n=40), had a trend towards improved DS (HR=0.74 [0.49-1.12], logrank p=0.15) (FIG. 10B). We also performed gene enrichment analysis on the RNA transcripts used in the two signatures above (n=79). Enrichr found numerous significant pathways (Table 14) both novel ones and those known to be implicated in PDAC progression and treatment resistance including the interferon signaling pathway, AMP-activated protein kinase (AMPK) and the CXCR4 signaling pathways. These pathways represent mechanisms for tumor metastasis, progression, and immunomodulation, but also novel targets which are actively being investigated for therapeutic targeting in PDAC. Together, these data independently validate the clinical relevance of our RNA expression discoveries.

### Validation of Single and Multi-Omic Analyte Models as Predictors of Disease Survival on Multiple Independent External Sample Cohorts and Datasets

To further validate our single-omic, multi-omic and parsimonious analytes for DS prediction we evaluated their predictive performance on the TCGA dataset, containing 157 evaluable samples that had at least one analyte type (Table 3). Since TCGA has data only on DNA, RNA, WSI (for computational pathology) and clinical analytes, our modeling had a reduced set of 3423 total features compared to 6363 in our original MT-Pilot cohort (Table 1, FIG. 1E). Models trained on features from individual single-omic analytes such as clinical features, computational pathology, DNA and RNA gene expressions in the TCGA cohort had an accuracy and PPV for DS prediction ranging between [0.47-0.96] and [0.56-0.98], respectively (Table 2). The full 3423 analyte model had an accuracy and PPV of 0.94 (95 CI 0.83-1.00) and 0.95 (95% CI 0.84-1.00) (Table 2) for DS prediction. Computational pathology, DNA SNVs, and RNA gene expressions perform strongly in single-omic validation of DS (Table 2).

Next, we examined the validity of our multi-omic parsimonious model on the TCGA dataset. Because this cohort had an overall reduced analyte set, we used an RFE strategy to retrain a Random Forest model for DS on our original cohort (MT-Pilot) and determined that the optimal (top of peak) parsimonious model employed 202 features out 3432 and had accuracy and PPV of 0.74 (0.63-0.85) and 0.77 (0.65-0.89), respectively (FIG. 10C). Importantly, when the model was applied to these same 202 features (Table 15) in the TCGA dataset, it yielded reported an accuracy of 0.88 and PPV of 0.95 for DS prediction. Furthermore, in both our MT-Pilot Cohort and the TCGA validation Cohort, computational pathology and RNA gene expression were found to be primary analytes learned by the DS predicting models on, with CNV and the clinical analyte providing minor additional improvement (FIG. 10C). Signal dominance of RNA is not driven by expression of any single gene, but by a specific set of genes. This is supported by the RNA signature and enrichment analysis results described in the prior section.

Since TCGA lacked tissue proteomic level data, we sought an external dataset with tissue protein data, along with other critical single-omic informative analytes such as DNA, RNA, and clinical. We found an independent publicly available dataset we named JHU Cohort 1 that met these criteria. With DNA, RNA, clinical data, and tissue protein analytes from our MT-Pilot cohort serving as the training set, we trained a L1-normalized Random Forest model and applied it to this validation test set. This model predicted DS with an accuracy and PPV of 0.89 (95% CI 0.83-0.95) and 0.91 (95% CI 0.85-0.98), respectively (Table 2). While a model trained on the tissue protein as a single-omic analyte had an accuracy and PPV of 0.56 (95% CI 0.50-0.63) and 0.53 (95% CI 0.47-0.60) in the JHU Cohort 1 (Table 2), addition of DNA, RNA, and clinical analytes improved predictive performance of the model and validated the multi-omic approach.

### Independent Validation of Plasma Proteins as a Novel Preoperative Biomarker for Treatment Selection

Our multi-omic and parsimonious modeling of the MT-Pilot Cohort, we discovered that plasma protein is an analyte which provides not only significant prediction of DS in PDAC, but does so with fewest features compared to other analytes. As a result of these findings, as well as the poor performance of CA19-9 as a preoperative marker for decision making regarding the benefit of surgery, we next sought to validate our findings solely on analytes that would be available to the clinical practitioner before surgery.

Besides TCGA and JHU Cohort 1, we utilized two more cohorts; JHU Cohort 2 and the MGH Cohort (Table 3). They included similar stage I/II resected PDAC, excluding stage III/IV patients, where clinical and demographic data were collected longitudinally and preoperative plasma samples, including CA 19-9, were obtained and analyzed as described above. Application of the L1-normalized Random Forest model trained on the MT-Pilot data on the two cohorts showed that plasma proteins remained highly predictive of DS in both validation cohorts, with accuracy and PPV of 0.98 (95% CT 0.83-1.00) 0.92 (95% C a 0.79-1.00), respectively in JHU Cohort 2 and 0.89 (95% CI 0.76-1.00) 0.80 (95 C 0.69-0.91), respectively in the MGH Cohort (Table 2). The addition of clinical data to plasma protein improves the multi-omic model for DS prediction. However, the addition of plasma lipidomics to plasma proteins and clinical data did not further improve DS predictions. Overall, preoperative plasma protein was highly predictive of DS among three separate independent datasets and provided a unique preoperative biomarker with significantly better predictive performance than routinely utilized CA 19-9 (Table 2).

### Tables

TABLE 1
Top Single-omic and Multi-omic Performance for Disease Survival

| | # | # | | | | | ACC | PPV | Sensi- | Speci- |
|---|---|---|---|---|---|---|---|---|---|---|
| Analytes | Samples | Features | TP | FP | TN | FN | (95% CI) | (95% CI) | tivity | ficity |
| Multi-omic | 39 | 6363 | 26 | 4 | 7 | 2 | 0.85 | 0.87 | 0.93 | 0.64 |
| | | | | | | | (0.73-0.96) | (0.75-0.99) | | |
| Plasma | 51 | 257 | 32 | 8 | 6 | 5 | 0.75 | 0.80 | 0.86 | 0.43 |
| proteins | | | | | | | (0.63-0.86) | (0.68-0.92) | | |
| RNA | 57 | 29 | 35 | 12 | 8 | 2 | 0.75 | 0.74 | 0.95 | 0.40 |
| fusions | | | | | | | (0.64-0.87) | (0.62-0.87) | | |
| Tissue | 49 | 1130 | 32 | 10 | 4 | 3 | 0.73 | 0.76 | 0.91 | 0.29 |
| proteins | | | | | | | (0.61-0.86) | (0.63-0.89) | | |
| Plasma | 51 | 406 | 34 | 12 | 2 | 3 | 0.71 | 0.74 | 0.92 | 0.14 |
| lipids | | | | | | | (0.58-0.83) | (0.61-0.87) | | |
| Clinical & | 74 | 331 | 47 | 19 | 5 | 3 | 0.70 | 0.71 | 0.94 | 0.21 |
| Surg | | | | | | | (0.60-0.81) | (0.60-0.82) | | |
| pathology | | | | | | | | | | |
| RNA gene | 57 | 2000 | 33 | 14 | 6 | 4 | 0.68 | 0.70 | 0.89 | 0.30 |
| expressions | | | | | | | (0.56-0.80) | (0.57-0.83) | | |
| Comp. | 71 | 819 | 34 | 11 | 13 | 13 | 0.66 | 0.76 | 0.72 | 0.54 |
| pathology | | | | | | | (0.55-0.77) | (0.63-0.88) | | |
| DNA | 72 | 648 | 43 | 20 | 4 | 5 | 0.65 | 0.68 | 0.90 | 0.17 |
| CNVs | | | | | | | (0.54-0.76) | (0.57-0.80) | | |
| DNA | 72 | 126 | 39 | 17 | 7 | 9 | 0.64 | 0.70 | 0.81 | 0.29 |
| INDELs | | | | | | | (0.53-0.75) | (0.58-0.82) | | |
| DNA | 72 | 611 | 45 | 23 | 1 | 3 | 0.64 | 0.66 | 0.94 | 0.04 |
| SNVs | | | | | | | (0.53-0.75) | (0.55-0.77) | | |
| CA 19-9 | 63 | 1 | 17 | 15 | 20 | 11 | 0.59 | 0.53 | 0.61 | 0.57 |
| pre-surgery | | | | | | | (0.47-0.71) | (0.40-0.65) | | |

TABLE 2
Top Single-omic and Multi-omic Performance for Disease Survival:
Study Validation Cohorts (TCGA, JHU 1, JHU 2, MGH) Analytes

| | | | | | | | | | | | |
|---|---|---|---|---|---|---|---|---|---|---|---|
| | | # Vali- | | | | | | | | | |
| | # Train | dation | # | | | | | ACC | PPV | Sensi- | Speci- |
| Analytes | Samples | Samples | Features | TP | FP | TN | FN | (95% CI) | (95% CI) | tivity | ficity |

TCGA

| | | | | | | | | | | | |
|---|---|---|---|---|---|---|---|---|---|---|---|
| Clinical & Surg. | 45 | 109 | 3024 | 63 | 1 | 42 | 3 | 0.96 | 0.98 | 0.95 | 0.98 |
| pathology, DNA | | | | | | | | (0.88-1.00) | (0.90-1.00) | | |
| (SNVs, INDELs, | | | | | | | | | | | |
| CNVs), RNA gene | | | | | | | | | | | |
| expressions, | | | | | | | | | | | |
| Clinical & Surg. | 45 | 33 | 3423 | 20 | 1 | 11 | 1 | 0.94 | 0.95 | 0.95 | 0.92 |
| pathology, DNA | | | | | | | | (0.83-1.00) | (0.84-1.00) | | |
| (SNVs, INDELs, | | | | | | | | | | | |
| CNVs), RNA gene | | | | | | | | | | | |
| expressions, | | | | | | | | | | | |
| Comp. pathology | | | | | | | | | | | |
| DNA SNVs | 72 | 126 | 351 | 73 | 3 | 46 | 4 | 0.94 | 0.96 | 0.95 | 0.94 |
| | | | | | | | | (0.85-1.00) | (0.86-1.00) | | |
| Comp. pathology | 71 | 33 | 819 | 16 | 2 | 10 | 5 | 0.79 | 0.89 | 0.76 | 0.83 |
| | | | | | | | | (0.68-0.89) | (0.78-0.99) | | |
| RNA gene | 57 | 152 | 1974 | 65 | 16 | 51 | 20 | 0.76 | 0.80 | 0.76 | 0.76 |
| expressions | | | | | | | | (0.67-0.85) | (0.71-0.89) | | |
| DNA INDELs | 56 | 120 | 43 | 50 | 11 | 36 | 23 | 0.72 | 0.82 | 0.68 | 0.77 |
| | | | | | | | | (0.60-0.84) | (0.70-0.94) | | |
| Clinical | 74 | 157 | 15 | 61 | 25 | 42 | 29 | 0.66 | 0.71 | 0.68 | 0.63 |
| | | | | | | | | (0.57-0.75) | (0.63-0.80) | | |
| DNA CNVs | 72 | 156 | 645 | 38 | 30 | 36 | 52 | 0.47 | 0.56 | 0.42 | 0.55 |
| | | | | | | | | (0.40-0.54) | (0.49-0.63) | | |

JHU Cohort 1

| | | | | | | | | | | | |
|---|---|---|---|---|---|---|---|---|---|---|---|
| Clinical & Surg. | 39 | 81 | 3270 | 32 | 3 | 40 | 6 | 0.89 | 0.91 | 0.84 | 0.93 |
| pathology, DNA | | | | | | | | (0.83-0.95) | (0.85-0.98) | | |
| (INDELs, CNVs, | | | | | | | | | | | |
| SNVs), RNA gene | | | | | | | | | | | |
| expressions, | | | | | | | | | | | |
| Tissue proteins | | | | | | | | | | | |
| Clinical & Surg. | 40 | 81 | 2480 | 29 | 11 | 32 | 9 | 0.75 | 0.72 | 0.76 | 0.74 |
| pathology, RNA | | | | | | | | (0.69-0.82) | (0.66-0.79) | | |
| gene expressions, | | | | | | | | | | | |
| Tissue proteins | | | | | | | | | | | |
| RNA gene | 46 | 81 | 2466 | 27 | 12 | 31 | 11 | 0.72 | 0.69 | 0.71 | 0.72 |
| expressions, | | | | | | | | (0.66-0.79) | (0.63-0.76) | | |
| Tissue proteins | | | | | | | | | | | |
| RNA gene | 57 | 81 | 1963 | 24 | 12 | 31 | 14 | 0.68 | 0.67 | 0.63 | 0.72 |
| expressions | | | | | | | | (0.62-0.75) | (0.61-0.74) | | |
| Clinical & Surg. | 45 | 81 | 1307 | 24 | 14 | 29 | 14 | 0.65 | 0.63 | 0.63 | 0.67 |
| pathology, DNA | | | | | | | | (0.59-0.72) | (0.57-0.70) | | |
| (INDELs, CNVs, | | | | | | | | | | | |
| SNVs), Tissue | | | | | | | | | | | |
| proteins | | | | | | | | | | | |
| Clinical & Surg. | 45 | 81 | 2767 | 24 | 18 | 25 | 14 | 0.60 | 0.57 | 0.63 | 0.58 |
| pathology, DNA | | | | | | | | (0.54-0.67) | (0.51-0.64) | | |
| (INDELs, CNVs, | | | | | | | | | | | |
| SNVs), RNA gene | | | | | | | | | | | |
| expressions | | | | | | | | | | | |
| Tissue proteins | 49 | 81 | 503 | 20 | 18 | 25 | 18 | 0.56 | 0.53 | 0.53 | 0.58 |
| | | | | | | | | (0.50-0.63) | (0.47-0.60) | | |
| DNA (INDELs, | 56 | 81 | 790 | 17 | 19 | 24 | 21 | 0.51 | 0.47 | 0.45 | 0.56 |
| CNVs, SNVs) | | | | | | | | (0.45-0.58) | (0.41-0.54) | | |
| Clinical | 74 | 81 | 14 | 14 | 26 | 17 | 24 | 0.38 | 0.35 | 0.37 | 0.4 |
| | | | | | | | | (0.32-0.45) | (0.29-0.42) | | |

JHU Cohort 2

| | | | | | | | | | | | |
|---|---|---|---|---|---|---|---|---|---|---|---|
| Clinical, Plasma | 41 | 47 | 255 | 12 | 1 | 34 | 0 | 0.98 | 0.92 | 1.0 | 0.97 |
| proteins | | | | | | | | (0.83-1.00) | (0.79-1.00) | | |
| Plasma proteins | 51 | 47 | 251 | 12 | 1 | 34 | 0 | 0.98 | 0.92 | 1.00 | 0.97 |
| | | | | | | | | (0.83-1.00) | (0.79-1.00) | | |
| Clinical, Plasma | 51 | 47 | 619 | 8 | 6 | 29 | 4 | 0.79 | 0.57 | 0.67 | 0.83 |
| proteins, Plasma | | | | | | | | (0.63-0.94) | (0.44-0.69) | | |
| lipids | | | | | | | | | | | |
| CA 19-9 pre- | 63 | 48 | 1 | 1 | 5 | 32 | 11 | 0.69 | 0.17 | 0.08 | 0.86 |
| surgery | | | | | | | | (0.57-0.81) | (0.04-0.40) | | |
| Plasma proteins, | 51 | 47 | 615 | 7 | 16 | 19 | 5 | 0.55 | 0.30 | 0.58 | 0.54 |
| Plasma lipids | | | | | | | | (0.41-0.69) | (0.16-0.44) | | |
| Clinical | 74 | 49 | 5 | 3 | 19 | 18 | 9 | 0.43 | 0.14 | 0.25 | 0.49 |
| | | | | | | | | (0.29-0.57) | (0.02-0.26) | | |
| Clinical, Plasma | 51 | 47 | 369 | 3 | 23 | 12 | 9 | 0.32 | 0.12 | 0.25 | 0.34 |
| lipids | | | | | | | | (0.20-0.44) | (0.00-0.25) | | |
| Plasma lipids | 51 | 47 | 365 | 5 | 29 | 6 | 7 | 0.23 | 0.15 | 0.42 | 0.17 |
| | | | | | | | | (0.12-0.35) | (0.03-0.27) | | |

MGH Cohort

| | | | | | | | | | | | |
|---|---|---|---|---|---|---|---|---|---|---|---|
| Clinical, Plasma | 51 | 35 | 259 | 16 | 3 | 16 | 0 | 0.91 | 0.84 | 1.00 | 0.84 |
| proteins | | | | | | | | (0.77-1.00) | (0.71-0.97) | | |
| Plasma proteins | 51 | 35 | 250 | 16 | 4 | 15 | 0 | 0.89 | 0.80 | 1.00 | 0.79 |
| | | | | | | | | (0.76-1.00) | (0.69-0.91) | | |
| Plasma proteins, | 51 | 35 | 614 | 13 | 6 | 13 | 3 | 0.74 | 0.68 | 0.81 | 0.68 |
| Plasma lipids | | | | | | | | (0.61-0.87) | (0.54-0.82) | | |
| CA 19-9 pre- | 63 | 32 | 1 | 9 | 6 | 11 | 6 | 0.62 | 0.60 | 0.60 | 0.65 |
| surgery | | | | | | | | (0.51-0.73) | (0.52-0.68) | | |
| Clinical, Plasma | 51 | 35 | 623 | 7 | 8 | 11 | 9 | 0.51 | 0.47 | 0.44 | 0.58 |
| proteins, Plasma | | | | | | | | (0.41-0.62) | (0.33-0.61) | | |
| lipids | | | | | | | | | | | |
| Plasma lipids | 51 | 35 | 365 | 11 | 13 | 6 | 5 | 0.49 | 0.46 | 0.69 | 0.32 |
| | | | | | | | | (0.36-0.62) | (0.30-0.62) | | |
| Clinical | 74 | 35 | 10 | 7 | 12 | 7 | 9 | 0.40 | 0.37 | 0.44 | 0.37 |
| | | | | | | | | (0.29-0.51) | (0.26-0.48) | | |
| Clinical, Plasma | 51 | 35 | 374 | 7 | 13 | 6 | 9 | 0.37 | 0.35 | 0.44 | 0.32 |
| lipids | | | | | | | | (0.22-0.52) | (0.20-0.49) | | |

TABLE 3
Baseline demographics and clinical data of cohorts

| | | MT- | | | | | | | | |
|---|---|---|---|---|---|---|---|---|---|---|
| | | Pilot | | | | | | | | |
| | | Cohort | TCGA | | JHU Cohort 1 | | JHU Cohort 2 | | MGH Cohort | |
| | | % or | % or | | % or | | % or | | % or | |
| | | mean | mean | | mean | | mean | p- | mean | |
| Cohort | type | (std) | (std) | p-value | (std) | p-value | (std) | value | (std) | p-value |
| Sex | F | 47% | 45% | 0.7773 | 48% | 1.0 | 41% | 0.579 | 49% | 1.0 |
| | M | 53% | 55% | | 52% | | 59% | | 51% | |
| Age at | <65 | 27% | — | — | 41% | 0.0903 | 49% | 0.021 | 20% | 0.4843 |
| diagnosis | ≥65 | 73% | — | | 59% | | 51% | | 80% | |
| BMI | numerical | 26.22 | — | — | 25.43 | 0.3557 | — | — | 26.4 | 0.8864 |
| | | (5.63) | | | (5) | | | | (6.77) | |
| Preoperative | naïve | 59% | — | — | — | — | 24% | 1.90E−04 | 46% | 0.2177 |
| therapy | neoadjuvant | 41% | — | | — | | 76% | | 54% | |
| Tobacco | no | 86% | 19% | 3.33E−23 | 1% | 1.49E−31 | — | — | 54% | 5.28E−04 |
| smoking history | yes | 14% | 81% | | 99% | | — | | 46% | |
| Alcohol | no | 55% | 64% | 0.2492 | 16% | 2.86E−07 | — | — | 46% | 0.413 |
| consumption | yes | 45% | 36% | | 84% | | — | | 54% | |
| history | | | | | | | | | | |
| Perineural | absent | 11% | — | — | 16% | 0.36 | 8% | 0.761 | 20% | 0.236 |
| invasion | present | 89% | — | | 84% | | 92% | | 80% | |
| Lymphovascular | absent | 38% | — | — | 44% | 0.4193 | 41% | 0.851 | 46% | 0.5312 |
| Invasion | present | 62% | — | | 56% | | 59% | | 54% | |
| Surgical margin | negative | 86% | — | — | — | — | 86% | 1.000 | — | — |
| summary | positive | 14% | — | | — | | 14% | | — | |
| Clinical | <G3 | 77% | 71% | 0.3443 | — | — | 55% | 0.0169 | 60% | 0.0737 |
| grade | G3 | 23% | 29% | | — | | 45% | | 40% | |
| Clinical TNM | I | 64% | 10% | 3.11E−17 | 28% | 1.27E−05 | 88% | 0.003 | 31% | 0.0021 |
| stage | II | 36% | 90% | | 72% | | 12% | | 69% | |

Clinicopathological characteristics of PDAC cohorts in the study. Differences between cohorts were assessed pairwise for each characteristic using t-test (BMI only) or Fisher's exact test (all other characteristics) with significance level α set to 0.05 for each test.

TABLE 4A
Clinical, Surgical Pathology Top Features

| Analyte | Study Label | Feature | Frequency |
|---|---|---|---|
| Clinical | label_deceased | Age_at_Diagnosis | 1 |
| Clinical | label_deceased | Height | 1 |
| Clinical | label_deceased | Weight | 1 |
| Clinical | label_deceased | BMI | 1 |
| Clinical | label_deceased | TNM_Mixed_Substage | 1 |
| Clinical | label_deceased | Sex_ord | 1 |
| Clinical | label_deceased | Site_-_Primary_ICD-O-3_ord | 1 |
| Clinical | label_deceased | Histology_Behavior_ICD-O-3_ord | 1 |
| Clinical | label_deceased | Grade_Mixed_ord | 1 |
| Clinical | label_deceased | Surgical_Margins_Summary_ord | 1 |
| Clinical | label_deceased | Chemotherapy_Summary_ord | 1 |
| Clinical | label_deceased | Radiation_Summary_ord | 1 |
| Clinical | label_deceased | Perineural_Invasion_ord | 0.98648649 |
| Clinical | label_deceased | TNM_Mixed_Stage_ord | 0.95945946 |
| Clinical | label_deceased | Chemotherapy_Binary | 0.89189189 |
| Clinical | label_deceased | Lymphovascular_Invasion_ord | 0.16216216 |

TABLE 4B
Frequency of Top Clinical Features

| Analyte | Study Label | Feature | Diagnosis | Frequency |
|---|---|---|---|---|
| Clinical | label_deceased | Age_at_Diagnosis | | 1 |
| Clinical | label_deceased | Height | | 1 |
| Clinical | label_deceased | Weight | | 1 |
| Clinical | label_deceased | BMI | | 1 |
| Clinical | label_deceased | TNM_Mixed_Substage | | 1 |
| Clinical | label_deceased | Sex_ord | | 1 |
| Clinical | label_deceased | Site_-_Primary_ICD-O-3_ord | | 1 |
| Clinical | label_deceased | Histology_Behavior_ICD-O- | | 1 |
| | | 3_ord | | |
| Clinical | label_deceased | Grade_Mixed_ord | | 1 |
| Clinical | label_deceased | Surgical_Margins_Summary_{—} | | 1 |
| | | ord | | |
| Clinical | label_deceased | Chemotherapy_Summary_ord | | 1 |
| Clinical | label_deceased | Radiation_Summary_ord | | 1 |
| Clinical | label_deceased | Perineural_Invasion_ord | | 0.98648649 |
| Clinical | label_deceased | TNM_Mixed_Stage_ord | | 0.95945946 |
| Clinical | label_deceased | Chemotherapy_Binary | | 0.89189189 |
| Clinical | label_deceased | Lymphovascular_Invasion_ord | | 0.16216216 |
| Clinical | label_recurred | Histology_Behavior ICD-O- | | 0.1757 |
| | | 3_ord | | |
| Clinical | label_recurred | secondary_diagnosis_onehot_{—} | Gout, unspecified | 0.1351 |
| | | m109 | | |
| Clinical | label_recurred | merged_ethnicity_ord | | 0.1351 |
| Clinical | label_recurred | secondary_diagnosis_onehot_{—} | Urethral stricture, | 0.1216 |
| | | n359 | unspecified | |
| Clinical | label_recurred | secondary_diagnosis_onehot_{—} | Fluid overload, | 0.1216 |
| | | e8770.1 | unspecified | |
| Clinical | label_recurred | secondary_diagnosis_onehot_{—} | Gastro-esophageal reflux | 0.1216 |
| | | k219.4 | disease without | |
| | | | esophagitis | |
| Clinical | label_recurred | secondary_diagnosis_onehot_{—} | Other acute | 0.1216 |
| | | g8918.3 | postprocedural pain | |
| Clinical | label_recurred | secondary_diagnosis_onehot_{—} | Hypoxemia | 0.1216 |
| | | r0902.1 | | |
| Clinical | label_recurred | secondary_diagnosis_onehot_{—} | Moderate protein-calorie | 0.1081 |
| | | e440 | malnutrition | |
| Clinical | label_recurred | secondary_diagnosis_onehot_{—} | Acute embolism and | 0.1081 |
| | | i82890 | thrombosis of other | |
| | | | specified veins | |
| Clinical | label_recurred | secondary_diagnosis_onehot_{—} | Pleural effusion, not | 0.1081 |
| | | j90.1 | elsewhere classified | |
| Clinical | label_recurred | secondary_diagnosis_onehot_{—} | Sleep apnea | 0.1081 |
| | | g4733 | | |
| Clinical | label_recurred | secondary_diagnosis_onehot_{—} | Hypertensive chronic | 0.1081 |
| | | i129 | kidney disease with stage | |
| | | | 1 through stage 4 chronic | |
| | | | kidney disease, or | |
| | | | unspecified chronic | |
| | | | kidney disease | |
| Clinical | label_recurred | secondary_diagnosis_onehot_{—} | Chronic kidney disease, | 0.1081 |
| | | n189 | unspecified | |
| Clinical | label_recurred | secondary_diagnosis_onehot_{—} | Acquired absence of left | 0.1081 |
| | | z9012 | breast and nipple | |
| Clinical | label_recurred | secondary_diagnosis_onehot_{—} | Personal history of | 0.1081 |
| | | z853 | malignant neoplasm of | |
| | | | breast | |
| Clinical | label_recurred | secondary_diagnosis_onehot_{—} | Personal history of other | 0.1081 |
| | | z85828 | malignant neoplasm of | |
| | | | skin | |
| Clinical | label_recurred | secondary_diagnosis_onehot_{—} | Hyperglycemia, | 0.0946 |
| | | r739.1 | unspecified | |
| Clinical | label_recurred | secondary_diagnosis_onehot_{—} | Fluid overload, | 0.0946 |
| | | e8770 | unspecified | |
| Clinical | label_recurred | secondary_diagnosis_onehot_{—} | Bradycardia, unspecified | 0.0946 |
| | | r001 | | |
| Clinical | label_recurred | secondary_diagnosis_onehot_{—} | Chronic kidney disease, | 0.0946 |
| | | n183 | stage 3 (moderate) | |
| Clinical | label_recurred | secondary_diagnosis_onehot_{—} | Hypertensive chronic | 0.0946 |
| | | i129.2 | kidney disease with stage | |
| | | | 1 through stage 4 chronic | |
| | | | kidney disease, or | |
| | | | unspecified chronic | |
| | | | kidney disease | |
| Clinical | label_recurred | secondary_diagnosis_onehot_{—} | Biliary acute pancreatitis | 0.0946 |
| | | k8510 | without necrosis or | |
| | | | infection | |
| Clinical | label_recurred | secondary_diagnosis_onehot_{—} | Klebsiella pneumoniae | 0.0946 |
| | | b961 | [K. pneumoniae] as the | |
| | | | cause of diseases | |
| | | | classified elsewhere | |
| Clinical | label_recurred | secondary_diagnosis_onehot_{—} | Other specified bacterial | 0.0946 |
| | | b9689 | agents as the cause of | |
| | | | diseases classified | |
| | | | elsewhere | |
| Clinical | label_recurred | secondary_diagnosis_onehot_{—} | Enterococcus as the cause | 0.0946 |
| | | b952 | of diseases classified | |
| | | | elsewhere | |
| Clinical | label_recurred | secondary_diagnosis_onehot_{—} | Urinary tract infection, | 0.0946 |
| | | n390 | site not specified | |
| Clinical | label_recurred | secondary_diagnosis_onehot_{—} | Type 2 diabetes mellitus | 0.0946 |
| | | e119.1 | without complications | |
| Clinical | label_recurred | secondary_diagnosis_onehot_{—} | Autoimmune thyroiditis | 0.0946 |
| | | e063 | | |
| Clinical | label_recurred | secondary_diagnosis_onehot_{—} | Type 2 diabetes mellitus | 0.0946 |
| | | e119.4 | without complications | |
| Clinical | label_recurred | secondary_diagnosis_onehot_{—} | Acute embolism and | 0.0811 |
| | | i824z1 | thrombosis of unspecified | |
| | | | deep veins of right distal | |
| | | | lower extremity | |
| Clinical | label_recurred | secondary_diagnosis_onehot_{—} | Unspecified Escherichia | 0.0811 |
| | | b9620 | coli [E. coli] as the cause | |
| | | | of diseases classified | |
| | | | elsewhere | |
| Clinical | label_recurred | secondary_diagnosis_onehot_{—} | Other specified bacterial | 0.0811 |
| | | b9689.1 | agents as the cause of | |
| | | | diseases classified | |
| | | | elsewhere | |
| Clinical | label_recurred | secondary_diagnosis_onehot_{—} | Cyst of pancreas | 0.0811 |
| | | k862.1 | | |
| Clinical | label_recurred | secondary_diagnosis_onehot_{—} | Unspecified protein- | 0.0811 |
| | | e46.2 | calorie malnutrition | |
| Clinical | label_recurred | secondary_diagnosis_onehot_{—} | Type 2 diabetes mellitus | 0.0811 |
| | | e1122 | with diabetic chronic | |
| | | | kidney disease | |
| Clinical | label_recurred | secondary_diagnosis_onehot_{—} | Chronic atrial fibrillation | 0.0811 |
| | | i482 | | |
| Clinical | label_recurred | secondary_diagnosis_onehot_{—} | Acute postprocedural | 0.0811 |
| | | j95821 | respiratory failure | |
| Clinical | label_recurred | secondary_diagnosis_onehot_{—} | Acute hepatitis C without | 0.0811 |
| | | b1710 | hepatic coma | |
| Clinical | label_recurred | secondary_diagnosis_onehot_{—} | Hypothyroidism, | 0.0811 |
| | | e039 | unspecified | |
| Clinical | label_recurred | secondary_diagnosis_onehot_{—} | Unspecified severe | 0.0811 |
| | | e43.1 | protein-calorie | |
| | | | malnutrition | |
| Clinical | label_recurred | secondary_diagnosis_onehot_{—} | Intestinal malabsorption, | 0.0811 |
| | | k909 | unspecified | |
| Clinical | label_recurred | secondary_diagnosis_onehot_{—} | Type 2 diabetes mellitus | 0.0811 |
| | | e1143 | with diabetic autonomic | |
| | | | (poly)neuropathy | |
| Clinical | label_recurred | secondary_diagnosis_onehot_{—} | Unspecified right bundle- | 0.0811 |
| | | i4510 | branch block | |
| Clinical | label_recurred | secondary_diagnosis_onehot_{—} | Gastroparesis | 0.0811 |
| | | k3184 | | |
| Clinical | label_recurred | secondary_diagnosis_onehot_{—} | Acquired partial absence | 0.0811 |
| | | z90411 | of pancreas | |
| Clinical | label_recurred | secondary_diagnosis_onehot_{—} | Body mass index (BMI) | 0.0811 |
| | | z6839 | 30-39, adult | |
| Clinical | label_recurred | secondary_diagnosis_onehot_{—} | Personal history of | 0.0811 |
| | | z87891.3 | nicotine dependence | |
| Clinical | label_recurred | secondary_diagnosis_onehot_{—} | Obstructive sleep apnea | 0.0811 |
| | | g4733.1 | (adult) (pediatric) | |
| Clinical | label_recurred | secondary_diagnosis_onehot_{—} | Body mass index (BMI) | 0.0676 |
| | | z6841 | 40 or greater, adult | |
| Clinical | label_recurred | secondary_diagnosis_onehot_{—} | Supraventricular | 0.0676 |
| | | i471.2 | tachycardia | |
| Clinical | label_recurred | secondary_diagnosis_onehot_{—} | Hyperlipidemia, | 0.0676 |
| | | e785.5 | unspecified | |
| Clinical | label_recurred | secondary_diagnosis_onehot_{—} | Personal history of | 0.0676 |
| | | z8789.1 | nicotine dependence | |
| Clinical | label_recurred | secondary_diagnosis_onehot_{—} | Body mass index (BMI) | 0.0676 |
| | | z6820.1 | 20.0-20.9, adult | |
| Clinical | label_recurred | secondary_diagnosis_onehot_{—} | Other specified diseases | 0.0676 |
| | | k838 | of biliary tract | |
| Clinical | label_recurred | secondary_diagnosis_onehot_{—} | Other functional intestinal | 0.0676 |
| | | k59 | disorders | |
| Clinical | label_recurred | secondary_diagnosis_onehot_{—} | Thoracic aortic ectasia | 0.0676 |
| | | i77810 | | |
| Clinical | label_recurred | secondary_diagnosis_onehot_{—} | Other nonspecific | 0.0676 |
| | | r918 | abnormal finding of lung | |
| | | | field | |
| Clinical | label_recurred | secondary_diagnosis_onehot_{—} | Diverticulosis of intestine, | 0.0676 |
| | | k5790 | part unspecified, without | |
| | | | perforation or abscess | |
| | | | without bleeding | |
| Clinical | label_recurred | secondary_diagnosis_onehot_{—} | Benign prostatic | 0.0676 |
| | | n400 | hyperplasia without lower | |
| | | | urinary tract symptoms | |
| Clinical | label_recurred | secondary_diagnosis_onehot_{—} | Personal history of | 0.0676 |
| | | z87891.1 | nicotine dependence | |
| Clinical | label_recurred | secondary_diagnosis_onehot_{—} | Atherosclerotic heart | 0.0676 |
| | | i2510 | disease of native coronary | |
| | | | artery without angina | |
| | | | pectoris | |
| Clinical | label_recurred | secondary_diagnosis_onehot_{—} | Other postprocedural | 0.0676 |
| | | i9789 | complications and | |
| | | | disorders of the | |
| | | | circulatory system, not | |
| | | | elsewhere classified | |
| Clinical | label_recurred | secondary_diagnosis_onehot_{—} | Unspecified atrial | 0.0676 |
| | | i4891.1 | fibrillation | |
| Clinical | label_recurred | Surgical_Margins_Summary_{—} | | 0.0676 |
| | | ord | | |
| Clinical | label_recurred | secondary_diagnosis_onehot_{—} | Hypokalemia | 0.0676 |
| | | e876 | | |
| Clinical | label_recurred | secondary_diagnosis_onehot_{—} | Other acute | 0.0676 |
| | | g8918.1 | postprocedural pain | |
| Clinical | label_recurred | secondary_diagnosis_onehot_{—} | Type 2 diabetes mellitus | 0.0676 |
| | | e119.3 | without complications | |
| Clinical | label_recurred | secondary_diagnosis_onehot_{—} | Gastro-esophageal reflux | 0.0676 |
| | | k219.3 | disease without | |
| | | | esophagitis | |
| Clinical | label_recurred | secondary_diagnosis_onehot_{—} | Hyperlipidemia, | 0.0676 |
| | | e785.7 | unspecified | |
| Clinical | label_recurred | secondary_diagnosis_onehot_{—} | | 0.0676 |
| | | l299 | | |
| Clinical | label_recurred | secondary_diagnosis_onehot_{—} | Unspecified jaundice | 0.0676 |
| | | r17 | | |
| Clinical | label_recurred | secondary_diagnosis_onehot_{—} | Disease of pancreas, | 0.0676 |
| | | k869.2 | unspecified | |
| Clinical | label_recurred | secondary_diagnosis_onehot_{—} | Acute postprocedural | 0.0676 |
| | | j95821.1 | respiratory failure | |
| Clinical | label_recurred | secondary_diagnosis_onehot_{—} | Cyst of pancreas | 0.0676 |
| | | k862.2 | | |
| Clinical | label_recurred | secondary_diagnosis_onehot_{—} | Unspecified protein- | 0.0676 |
| | | e46.3 | calorie malnutrition | |
| Clinical | label_recurred | secondary_diagnosis_onehot_{—} | Acute embolism and | 0.0676 |
| | | i824z1.1 | thrombosis of unspecified | |
| | | | deep veins of right distal | |
| | | | lower extremity | |
| Clinical | label_recurred | secondary_diagnosis_onehot_{—} | Alzheimer's disease, | 0.0676 |
| | | g309 | unspecified | |
| Clinical | label_recurred | secondary_diagnosis_onehot_{—} | Atherosclerotic heart | 0.0676 |
| | | i2510.4 | disease of native coronary | |
| | | | artery without angina | |
| | | | pectoris | |
| Clinical | label_recurred | secondary_diagnosis_onehot_{—} | Personal history of | 0.0676 |
| | | z923 | irradiation | |
| Clinical | label_recurred | secondary_diagnosis_onehot_{—} | Heartburn | 0.0676 |
| | | r12 | | |
| Clinical | label_recurred | secondary_diagnosis_onehot_{—} | Hyperlipidemia, | 0.0676 |
| | | e785.3 | unspecified | |
| Clinical | label_recurred | secondary_diagnosis_onehot_{—} | Hyperglycemia, | 0.0676 |
| | | r739 | unspecified | |
| Clinical | label_recurred | secondary_diagnosis_onehot_{—} | Body Mass Index 32.0- | 0.0676 |
| | | z6832 | 32.9, adult | |
| Clinical | label_recurred | secondary_diagnosis_onehot_{—} | Presence of left artificial | 0.0676 |
| | | z96642 | hip joint | |
| Clinical | label_recurred | secondary_diagnosis_onehot_{—} | Other acute | 0.0676 |
| | | g8918.4 | postprocedural pain | |
| Clinical | label_recurred | secondary_diagnosis_onehot_{—} | Supraventricular | 0.0676 |
| | | i471.1 | tachycardia | |
| Clinical | label_recurred | secondary_diagnosis_onehot_{—} | Thyrotoxicosis with | 0.0676 |
| | | e0500 | diffuse goiter without | |
| | | | thyrotoxic crisis or storm | |
| Clinical | label_recurred | secondary_diagnosis_onehot_{—} | Other acute | 0.0676 |
| | | g8918.6 | postprocedural pain | |
| Clinical | label_recurred | secondary_diagnosis_onehot_{—} | Nonrheumatic aortic | 0.0676 |
| | | i351 | (valve) insufficiency | |
| Clinical | label_recurred | secondary_diagnosis_onehot_{—} | Nonrheumatic mitral | 0.0676 |
| | | i340 | (valve) insufficiency | |
| Clinical | label_recurred | secondary_diagnosis_onehot_{—} | Atelectasis | 0.0676 |
| | | j9811.3 | | |
| Clinical | label_recurred | secondary_diagnosis_onehot_{—} | Ileus, unspecified | 0.0676 |
| | | k567.2 | | |
| Clinical | label_recurred | secondary_diagnosis_onehot_{—} | Failure in suture and | 0.0676 |
| | | e876.2 | ligature during surgical | |
| | | | operation | |
| Clinical | label_recurred | secondary_diagnosis_onehot_{—} | Other disorders of | 0.0676 |
| | | e8339 | phosphorus metabolism | |
| Clinical | label_recurred | secondary_diagnosis_onehot_{—} | Cachexia | 0.0676 |
| | | r64 | | |
| Clinical | label_recurred | secondary_diagnosis_onehot_{—} | Neoplasm related pain | 0.0676 |
| | | g893 | (acute) (chronic) | |
| Clinical | label_recurred | secondary_diagnosis_onehot_{—} | Personal history of other | 0.0676 |
| | | z8789 | specified conditions | |
| Clinical | label_recurred | secondary_diagnosis_onehot_{—} | Body mass index (BMI) | 0.0676 |
| | | z6825 | 25.0-25.9, adult | |
| Clinical | label_recurred | secondary_diagnosis_onehot_{—} | Unspecified atrial flutter | 0.0541 |
| | | i4892 | | |
| Clinical | label_recurred | secondary_diagnosis_onehot_{—} | Foreign object left in | 0.0541 |
| | | e871.1 | body during infusion or | |
| | | | transfusion | |
| Clinical | label_recurred | Patient_History_of_Cancer_Seq_{—} | | 0.0541 |
| | | 2_ord | | |
| Clinical | label_recurred | secondary_diagnosis_onehot_{—} | Disease of pancreas, | 0.0541 |
| | | k869.1 | unspecified | |
| Clinical | label_recurred | secondary_diagnosis_onehot_{—} | Chronic embolism and | 0.0541 |
| | | i82890.1 | thrombosis of other | |
| | | | specified veins | |
| Clinical | label_recurred | secondary_diagnosis_onehot_{—} | Liver disease, unspecified | 0.0541 |
| | | k769 | | |
| Clinical | label_recurred | secondary_diagnosis_onehot_{—} | Abnormal findings on | 0.0541 |
| | | r932 | diagnostic imaging of | |
| | | | liver and biliary tract | |
| Clinical | label_recurred | secondary_diagnosis_onehot_{—} | Unspecified cirrhosis of | 0.0541 |
| | | k7460.1 | liver | |
| Clinical | label_recurred | secondary_diagnosis_onehot_{—} | Personal history of other | 0.0541 |
| | | z8619 | infectious and parasitic | |
| | | | diseases | |
| Clinical | label_recurred | secondary_diagnosis_onehot_{—} | Family history of | 0.0541 |
| | | z800 | malignant neoplasm of | |
| | | | digestive organs | |
| Clinical | label_recurred | secondary_diagnosis_onehot_{—} | Nonspecific elevation of | 0.0541 |
| | | r740 | levels of transaminase and | |
| | | | lactic acid dehydrogenase | |
| | | | [LDH] | |
| Clinical | label_recurred | secondary_diagnosis_onehot_{—} | Hypoxemia | 0.0541 |
| | | r0902 | | |
| Clinical | label_recurred | secondary_diagnosis_onehot_{—} | Type 2 diabetes mellitus | 0.0541 |
| | | e1142 | with diabetic | |
| | | | polyneuropathy | |
| Clinical | label_recurred | secondary_diagnosis_onehot_{—} | Obesity, unspecified | 0.0541 |
| | | e669.1 | | |
| Clinical | label_recurred | secondary_diagnosis_onehot_{—} | Chronic obstructive | 0.0541 |
| | | j449.1 | pulmonary disease, | |
| | | | unspecified | |
| Clinical | label_recurred | secondary_diagnosis_onehot_{—} | Unspecified asthma, | 0.0541 |
| | | j45909 | uncomplicated | |
| Clinical | label_recurred | secondary_diagnosis_onehot_{—} | Pure hyercholesterolemia | 0.0541 |
| | | e780 | | |
| Clinical | label_recurred | secondary_diagnosis_onehot_{—} | Ventricular tachycardia | 0.0541 |
| | | i472 | | |
| Clinical | label_recurred | secondary_diagnosis_onehot_{—} | Body mass index (BMI) | 0.0541 |
| | | z6820 | 20.0-20.9, adult | |
| Clinical | label_recurred | secondary_diagnosis_onehot_{—} | Gastroparesis | 0.0541 |
| | | k3184.1 | | |
| Clinical | label_recurred | secondary_diagnosis_onehot_{—} | Toxic encephalopathy | 0.0541 |
| | | g92 | | |
| Clinical | label_recurred | secondary_diagnosis_onehot_{—} | Obesity, unspecified | 0.0541 |
| | | e669 | | |
| Clinical | label_recurred | secondary_diagnosis_onehot_{—} | Essential (primary) | 0.0541 |
| | | i10 | hypertension | |
| Clinical | label_recurred | secondary_diagnosis_onehot_{—} | Peripheral vascular | 0.0541 |
| | | i739 | disease, unspecified | |
| Clinical | label_recurred | secondary_diagnosis_onehot_{—} | Personal history of | 0.0541 |
| | | z87442 | urinary calculi | |
| Clinical | label_recurred | secondary_diagnosis_onehot_{—} | Gastro-esophageal reflux | 0.0541 |
| | | k219.2 | disease without | |
| | | | esophagitis | |
| Clinical | label_recurred | secondary_diagnosis_onehot_{—} | Disease of pancreas, | 0.0541 |
| | | k869 | unspecified | |
| Clinical | label_recurred | Family_history_2nd_this_cancer_{—} | | 0.0541 |
| | | ord | | |
| Clinical | label_recurred | secondary_diagnosis_onehot_{—} | Ileus, unspecified | 0.0541 |
| | | k567.1 | | |
| Clinical | label_recurred | secondary_diagnosis_onehot_{—} | Pseudocyst of pancreas | 0.0541 |
| | | k863 | | |
| Clinical | label_recurred | secondary_diagnosis_onehot_{—} | Postprocedural intestinal | 0.0541 |
| | | k913.1 | obstruction | |
| Clinical | label_recurred | secondary_diagnosis_onehot_{—} | Obesity, unspecified | 0.0541 |
| | | e669.2 | | |
| Clinical | label_recurred | secondary_diagnosis_onehot_{—} | Personal history of | 0.0541 |
| | | z859.1 | malignant neoplasm, | |
| | | | unspecified | |
| Clinical | label_recurred | secondary_diagnosis_onehot_{—} | Heart failure, unspecified | 0.0541 |
| | | i509 | | |
| Clinical | label_recurred | secondary_diagnosis_onehot_{—} | Atherosclerotic heart | 0.0541 |
| | | i2510.3 | disease of native coronary | |
| | | | artery without angina | |
| | | | pectoris | |
| Clinical | label_recurred | secondary_diagnosis_onehot_{—} | Type 1 diabetes mellitus | 0.0541 |
| | | e109 | without complications | |
| Clinical | label_recurred | secondary_diagnosis_onehot_{—} | Sleep apnea, unspecified | 0.0541 |
| | | g4730 | | |
| Clinical | label_recurred | secondary_diagnosis_onehot_{—} | Presence of aortocoronary | 0.0541 |
| | | z951 | bypass graft | |
| Clinical | label_recurred | secondary_diagnosis_onehot_{—} | Hypothyroidism, | 0.0541 |
| | | e039.2 | unspecified | |
| Clinical | label_recurred | secondary_diagnosis_onehot_{—} | Alkalosis | 0.0541 |
| | | e873 | | |
| Clinical | label_recurred | secondary_diagnosis_onehot_{—} | Anuria and oliguria | 0.0541 |
| | | r34 | | |
| Clinical | label_recurred | secondary_diagnosis_onehot_{—} | Cholangitis | 0.0541 |
| | | k830.1 | | |
| Clinical | label_recurred | secondary_diagnosis_onehot_{—} | Foreign object left in | 0.0541 |
| | | e871.3 | body during injection or | |
| | | | vaccination | |
| Clinical | label_recurred | secondary_diagnosis_onehot_{—} | Other Chronic | 0.0541 |
| | | k861 | Pancreatitis | |
| Clinical | label_recurred | secondary_diagnosis_onehot_{—} | Paroxysmal atrial | 0.0541 |
| | | i480 | fibrillation | |
| Clinical | label_recurred | secondary_diagnosis_onehot_{—} | Herpesviral | 0.0541 |
| | | b002 | gingivostomatitis and | |
| | | | pharyngotonsillitis | |

TABLE 4C
Clinical, Surgical Pathology Complete Feature Set
Survival

| | Spearman | Spearman | | Spearman | Spearman |
|---|---|---|---|---|---|
| | rho | p-value | | rho | p-value |
| Age_at_Diagnosis | −0.023 | 0.848 | secondary_diagnosis_onehot_i4510 | 0.146 | 0.215 |
| Height | −0.023 | 0.847 | secondary_diagnosis_onehot_i69954 | −0.094 | 0.426 |
| Weight | 0.042 | 0.722 | secondary_diagnosis_onehot_i959.1 | −0.094 | 0.426 |
| Chemotherapy_Binary | 0.102 | 0.387 | secondary_diagnosis_onehot_j449.1 | −0.094 | 0.426 |
| BMI | 0.081 | 0.493 | secondary_diagnosis_onehot_j9811.1 | −0.025 | 0.835 |
| TNM_Mixed_Substage | −0.242 | 0.038 | secondary_diagnosis_onehot_k7689 | −0.094 | 0.426 |
| Sex_ord | 0.071 | 0.547 | secondary_diagnosis_onehot_k769 | −0.094 | 0.426 |
| Site _-_ Primary_ICD-O- | 0.051 | 0.668 | secondary_diagnosis_onehot_k830 | 0.146 | 0.215 |
| 3_ord | | | | | |
| Histology_Behavior_ICD- | 0.080 | 0.500 | secondary_diagnosis_onehot_k912 | −0.094 | 0.426 |
| O-3 ord | | | | | |
| TNM_Mixed_Stage_ord | −0.154 | 0.191 | secondary_diagnosis_onehot_n179.1 | 0.146 | 0.215 |
| Grade_Mixed_ord | −0.328 | 0.004 | secondary_diagnosis_onehot_n390.2 | 0.146 | 0.215 |
| Surgical_Margins_Summary_ord | −0.316 | 0.006 | secondary_diagnosis_onehot_n400.1 | −0.094 | 0.426 |
| Chemotherapy_Summary_ord | 0.124 | 0.294 | secondary_diagnosis_onehot_nan.3 | −0.028 | 0.815 |
| Radiation_Summary_ord | −0.056 | 0.638 | secondary_diagnosis_onehot_r0789 | −0.094 | 0.426 |
| Perineural_Invasion_ord | −0.077 | 0.514 | secondary_diagnosis_onehot_r0902 | −0.094 | 0.426 |
| Lymphovascular_Invasion_ord | −0.197 | 0.093 | secondary_diagnosis_onehot_r17.1 | −0.094 | 0.426 |
| Family_history_1st_any_cancer_ord | −0.003 | 0.977 | secondary_diagnosis_onehot_r34 | −0.094 | 0.426 |
| Family_history_2nd_any_cancer_ord | −0.095 | 0.420 | secondary_diagnosis_onehot_r82994 | 0.146 | 0.215 |
| Family_history_1st_this_cancer_ord | −0.025 | 0.832 | secondary_diagnosis_onehot_z859 | −0.094 | 0.426 |
| Family_history_2nd_this_cancer_ord | −0.236 | 0.043 | secondary_diagnosis_onehot_b952 | −0.094 | 0.426 |
| Patient_History_of_Cancer_Seq_1_ord | 0.036 | 0.763 | secondary_diagnosis_onehot_b9620 | 0.146 | 0.215 |
| Patient_History_of_Cancer_Seq_2_ord | 0.028 | 0.810 | secondary_diagnosis_onehot_e039.2 | −0.094 | 0.426 |
| Patient_History_Alcohol_ord | 0.007 | 0.951 | secondary_diagnosis_onehot_e1165.2 | −0.094 | 0.426 |
| Patient_History_Tobacco_ord | 0.185 | 0.115 | secondary_diagnosis_onehot_e119.3 | 0.146 | 0.215 |
| merged_ethnicity_ord | 0.063 | 0.594 | secondary_diagnosis_onehot_e6601 | 0.146 | 0.215 |
| secondary_diagnosis_onehot_a4159 | −0.094 | 0.426 | secondary_diagnosis_onehot_e785.4 | 0.146 | 0.215 |
| secondary_diagnosis_onehot_b002 | −0.094 | 0.426 | secondary_diagnosis_onehot_g43809 | 0.146 | 0.215 |
| secondary_diagnosis_onehot_b029 | −0.094 | 0.426 | secondary_diagnosis_onehot_g4733 | −0.094 | 0.426 |
| secondary_diagnosis_onehot_e1142 | −0.094 | 0.426 | secondary_diagnosis_onehot_g8918.2 | −0.134 | 0.256 |
| secondary_diagnosis_onehot_e119 | −0.134 | 0.256 | secondary_diagnosis_onehot_i10.4 | 0.037 | 0.755 |
| secondary_diagnosis_onehot_e43 | 0.037 | 0.755 | secondary_diagnosis_onehot_i130 | 0.146 | 0.215 |
| secondary_diagnosis_onehot_e440 | 0.037 | 0.755 | secondary_diagnosis_onehot_i361 | 0.146 | 0.215 |
| secondary_diagnosis_onehot_e46 | −0.094 | 0.426 | secondary_diagnosis_onehot_i482 | 0.146 | 0.215 |
| secondary_diagnosis_onehot_e669 | 0.146 | 0.215 | secondary_diagnosis_onehot_i824z1.1 | 0.146 | 0.215 |
| secondary_diagnosis_onehot_e785 | −0.094 | 0.426 | secondary_diagnosis_onehot_j45909 | −0.094 | 0.426 |
| secondary_diagnosis_onehot_e870 | 0.116 | 0.326 | secondary_diagnosis_onehot_j80 | −0.094 | 0.426 |
| secondary_diagnosis_onehot_e871 | 0.146 | 0.215 | secondary_diagnosis_onehot_k219.2 | −0.025 | 0.835 |
| secondary_diagnosis_onehot_e8809 | 0.146 | 0.215 | secondary_diagnosis_onehot_k3184 | 0.146 | 0.215 |
| secondary_diagnosis_onehot_g92 | 0.146 | 0.215 | secondary_diagnosis_onehot_k5010 | 0.146 | 0.215 |
| secondary_diagnosis_onehot_i10 | −0.094 | 0.426 | secondary_diagnosis_onehot_k830.1 | −0.094 | 0.426 |
| secondary_diagnosis_onehot_i129 | −0.094 | 0.426 | secondary_diagnosis_onehot_k913 | 0.037 | 0.755 |
| secondary_diagnosis_onehot_i471 | −0.094 | 0.426 | secondary_diagnosis_onehot_m1990 | −0.094 | 0.426 |
| secondary_diagnosis_onehot_i4891 | −0.094 | 0.426 | secondary_diagnosis_onehot_n184.1 | −0.094 | 0.426 |
| secondary_diagnosis_onehot_i5023 | −0.094 | 0.426 | secondary_diagnosis_onehot_n390.3 | −0.094 | 0.426 |
| secondary_diagnosis_onehot_i5032 | 0.146 | 0.215 | secondary_diagnosis_onehot_nan.4 | −0.028 | 0.815 |
| secondary_diagnosis_onehot_i519 | −0.094 | 0.426 | secondary_diagnosis_onehot_r739 | −0.094 | 0.426 |
| secondary_diagnosis_onehot_i6521 | −0.094 | 0.426 | secondary_diagnosis_onehot_r932 | −0.094 | 0.426 |
| secondary_diagnosis_onehot_i82890 | 0.146 | 0.215 | secondary_diagnosis_onehot_z8041 | −0.094 | 0.426 |
| secondary_diagnosis_onehot_i9789 | −0.094 | 0.426 | secondary_diagnosis_onehot_z8679.1 | −0.094 | 0.426 |
| secondary_diagnosis_onehot_j189 | 0.146 | 0.215 | secondary_diagnosis_onehot_z8789 | 0.146 | 0.215 |
| secondary_diagnosis_onehot_j95821 | −0.134 | 0.256 | secondary_diagnosis_onehot_z87891.2 | −0.094 | 0.426 |
| secondary_diagnosis_onehot_j9601 | 0.208 | 0.076 | secondary_diagnosis_onehot_z9012 | −0.094 | 0.426 |
| secondary_diagnosis_onehot_k219 | −0.094 | 0.426 | secondary_diagnosis_onehot_b9689.1 | 0.146 | 0.215 |
| secondary_diagnosis_onehot_k567 | 0.146 | 0.215 | secondary_diagnosis_onehot_e1165.3 | 0.175 | 0.135 |
| secondary_diagnosis_onehot_k7460 | −0.094 | 0.426 | secondary_diagnosis_onehot_e119.4 | −0.094 | 0.426 |
| secondary_diagnosis_onehot_k831 | −0.101 | 0.391 | secondary_diagnosis_onehot_e785.5 | −0.094 | 0.426 |
| secondary_diagnosis_onehot_k838 | −0.094 | 0.426 | secondary_diagnosis_onehot_e871.3 | −0.094 | 0.426 |
| secondary_diagnosis_onehot_k8590 | 0.037 | 0.755 | secondary_diagnosis_onehot_g309 | 0.146 | 0.215 |
| secondary_diagnosis_onehot_k862 | 0.146 | 0.215 | secondary_diagnosis_onehot_g8918.3 | −0.094 | 0.426 |
| secondary_diagnosis_onehot_k869 | −0.094 | 0.426 | secondary_diagnosis_onehot_i10.5 | −0.165 | 0.160 |
| secondary_diagnosis_onehot_l299 | 0.146 | 0.215 | secondary_diagnosis_onehot_i2510.1 | 0.146 | 0.215 |
| secondary_diagnosis_onehot_n3090 | −0.094 | 0.426 | secondary_diagnosis_onehot_l6523 | 0.146 | 0.215 |
| secondary_diagnosis_onehot_n359 | 0.146 | 0.215 | secondary_diagnosis_onehot_j9811.2 | 0.037 | 0.755 |
| secondary_diagnosis_onehot_n390 | −0.094 | 0.426 | secondary_diagnosis_onehot_k219.3 | 0.146 | 0.215 |
| secondary_diagnosis_onehot_l011 | −0.094 | 0.426 | secondary_diagnosis_onehot_k660 | 0.146 | 0.215 |
| secondary_diagnosis_onehot_r1011 | 0.146 | 0.215 | secondary_diagnosis_onehot_k7460.1 | −0.094 | 0.426 |
| secondary_diagnosis_onehot_r12 | −0.094 | 0.426 | secondary_diagnosis_onehot_k863 | −0.094 | 0.426 |
| secondary_diagnosis_onehot_r51 | 0.146 | 0.215 | secondary_diagnosis_onehot_n281 | −0.094 | 0.426 |
| secondary_diagnosis_onehot_z6841 | 0.146 | 0.215 | secondary_diagnosis_onehot_n529 | −0.094 | 0.426 |
| secondary_diagnosis_onehot_z8639 | 0.146 | 0.215 | secondary_diagnosis_onehot_nan.5 | −0.059 | 0.615 |
| secondary_diagnosis_onehot_a419 | −0.094 | 0.426 | secondary_diagnosis_onehot_r339 | 0.037 | 0.755 |
| secondary_diagnosis_onehot_b1710 | 0.146 | 0.215 | secondary_diagnosis_onehot_z6825 | 0.146 | 0.215 |
| secondary_diagnosis_onehot_b20 | −0.094 | 0.426 | secondary_diagnosis_onehot_z6841.1 | 0.146 | 0.215 |
| secondary_diagnosis_onehot_e039 | −0.094 | 0.426 | secondary_diagnosis_onehot_z800 | −0.094 | 0.426 |
| secondary_diagnosis_onehot_e1129 | 0.146 | 0.215 | secondary_diagnosis_onehot_z853 | −0.094 | 0.426 |
| secondary_diagnosis_onehot_e1143 | 0.146 | 0.215 | secondary_diagnosis_onehot_z859.1 | −0.094 | 0.426 |
| secondary_diagnosis_onehot_e1165 | 0.037 | 0.755 | secondary_diagnosis_onehot_z8739 | −0.094 | 0.426 |
| secondary_diagnosis_onehot_e119.1 | −0.094 | 0.426 | secondary_diagnosis_onehot_z90411 | 0.146 | 0.215 |
| secondary_diagnosis_onehot_e43.1 | −0.094 | 0.426 | secondary_diagnosis_onehot_z9221 | −0.094 | 0.426 |
| secondary_diagnosis_onehot_e46.1 | −0.094 | 0.426 | secondary_diagnosis_onehot_e6601.1 | 0.208 | 0.076 |
| secondary_diagnosis_onehot_e785.1 | −0.134 | 0.256 | secondary_diagnosis_onehot_e785.6 | 0.116 | 0.326 |
| secondary_diagnosis_onehot_e873 | −0.094 | 0.426 | secondary_diagnosis_onehot_e8770 | 0.146 | 0.215 |
| secondary_diagnosis_onehot_i10.1 | −0.070 | 0.552 | secondary_diagnosis_onehot_g629 | −0.094 | 0.426 |
| secondary_diagnosis_onehot_i119 | −0.094 | 0.426 | secondary_diagnosis_onehot_i10.6 | 0.208 | 0.076 |
| secondary_diagnosis_onehot_i252 | 0.146 | 0.215 | secondary_diagnosis_onehot_i2510.2 | −0.094 | 0.426 |
| secondary_diagnosis_onehot_i471.1 | 0.146 | 0.215 | secondary_diagnosis_onehot_i351 | −0.094 | 0.426 |
| secondary_diagnosis_onehot_i472 | −0.094 | 0.426 | secondary_diagnosis_onehot_i509 | −0.094 | 0.426 |
| secondary_diagnosis_onehot_i480 | 0.208 | 0.076 | secondary_diagnosis_onehot_i69351 | 0.146 | 0.215 |
| secondary_diagnosis_onehot_i4891.1 | −0.094 | 0.426 | secondary_diagnosis_onehot_j90.1 | −0.094 | 0.426 |
| secondary_diagnosis_onehot_i4892 | −0.094 | 0.426 | secondary_diagnosis_onehot_j9811.3 | −0.094 | 0.426 |
| secondary_diagnosis_onehot_i5033 | 0.146 | 0.215 | secondary_diagnosis_onehot_k5900.1 | 0.146 | 0.215 |
| secondary_diagnosis_onehot_i517 | −0.094 | 0.426 | secondary_diagnosis_onehot_k8020 | 0.146 | 0.215 |
| secondary_diagnosis_onehot_i714 | −0.094 | 0.426 | secondary_diagnosis_onehot_k913.1 | −0.094 | 0.426 |
| secondary_diagnosis_onehot_i739 | −0.094 | 0.426 | secondary_diagnosis_onehot_n189.1 | 0.146 | 0.215 |
| secondary_diagnosis_onehot_j918 | −0.094 | 0.426 | secondary_diagnosis_onehot_n400.2 | −0.094 | 0.426 |
| secondary_diagnosis_onehot_j95821.1 | 0.146 | 0.215 | secondary_diagnosis_onehot_n8110 | 0.146 | 0.215 |
| secondary_diagnosis_onehot_j9811 | 0.146 | 0.215 | secondary_diagnosis_onehot_nan.6 | −0.116 | 0.326 |
| secondary_diagnosis_onehot_k567.1 | 0.146 | 0.215 | secondary_diagnosis_onehot_r0902.1 | −0.094 | 0.426 |
| secondary_diagnosis_onehot_k59 | −0.094 | 0.426 | secondary_diagnosis_onehot_r740 | −0.094 | 0.426 |
| secondary_diagnosis_onehot_k5900 | −0.094 | 0.426 | secondary_diagnosis_onehot_z6820 | −0.094 | 0.426 |
| secondary_diagnosis_onehot_k8012 | −0.094 | 0.426 | secondary_diagnosis_onehot_z8619 | −0.094 | 0.426 |
| secondary_diagnosis_onehot_k831.1 | −0.134 | 0.256 | secondary_diagnosis_onehot_z8673 | 0.146 | 0.215 |
| secondary_diagnosis_onehot_k8500 | 0.146 | 0.215 | secondary_diagnosis_onehot_z8789.1 | −0.094 | 0.426 |
| secondary_diagnosis_onehot_k8510 | 0.146 | 0.215 | secondary_diagnosis_onehot_z96642 | −0.094 | 0.426 |
| secondary_diagnosis_onehot_k861 | −0.094 | 0.426 | secondary_diagnosis_onehot_e0500 | −0.094 | 0.426 |
| secondary_diagnosis_onehot_k862.1 | 0.146 | 0.215 | secondary_diagnosis_onehot_e1122.1 | 0.146 | 0.215 |
| secondary_diagnosis_onehot_k869.1 | −0.094 | 0.426 | secondary_diagnosis_onehot_e785.7 | 0.146 | 0.215 |
| secondary_diagnosis_onehot_n189 | −0.094 | 0.426 | secondary_diagnosis_onehot_e876.1 | 0.116 | 0.326 |
| secondary_diagnosis_onehot_n390.1 | 0.037 | 0.755 | secondary_diagnosis_onehot_e8770.1 | −0.094 | 0.426 |
| secondary_diagnosis_onehot_n400 | −0.094 | 0.426 | secondary_diagnosis_onehot_g8918.4 | −0.094 | 0.426 |
| secondary_diagnosis_onehot_nan.1 | 0.022 | 0.851 | secondary_diagnosis_onehot_h409 | 0.146 | 0.215 |
| secondary_diagnosis_onehot_r17 | 0.146 | 0.215 | secondary_diagnosis_onehot_i2510.3 | −0.094 | 0.426 |
| secondary_diagnosis_onehot_r197 | 0.146 | 0.215 | secondary_diagnosis_onehot_i340 | −0.094 | 0.426 |
| secondary_diagnosis_onehot_r64 | 0.146 | 0.215 | secondary_diagnosis_onehot_i4510.1 | 0.146 | 0.215 |
| secondary_diagnosis_onehot_z681 | 0.146 | 0.215 | secondary_diagnosis_onehot_i77810 | −0.094 | 0.426 |
| secondary_diagnosis_onehot_z8679 | −0.094 | 0.426 | secondary_diagnosis_onehot_k3184.1 | −0.094 | 0.426 |
| secondary_diagnosis_onehot_z87891 | −0.094 | 0.426 | secondary_diagnosis_onehot_k567.2 | −0.094 | 0.426 |
| secondary_diagnosis_onehot_b961 | −0.094 | 0.426 | secondary_diagnosis_onehot_k8064 | 0.146 | 0.215 |
| secondary_diagnosis_onehot_e1165.1 | 0.208 | 0.076 | secondary_diagnosis_onehot_k861.1 | 0.146 | 0.215 |
| secondary_diagnosis_onehot_e230 | −0.094 | 0.426 | secondary_diagnosis_onehot_n319 | −0.094 | 0.426 |
| secondary_diagnosis_onehot_e441 | 0.146 | 0.215 | secondary_diagnosis_onehot_nan.7 | −0.187 | 0.110 |
| secondary_diagnosis_onehot_e46.2 | 0.146 | 0.215 | secondary_diagnosis_onehot_r001 | 0.146 | 0.215 |
| secondary_diagnosis_onehot_e669.1 | −0.094 | 0.426 | secondary_diagnosis_onehot_r739.1 | 0.146 | 0.215 |
| secondary_diagnosis_onehot_e785.2 | 0.256 | 0.028 | secondary_diagnosis_onehot_z6820.1 | −0.094 | 0.426 |
| secondary_diagnosis_onehot_e871.1 | −0.094 | 0.426 | secondary_diagnosis_onehot_z6839 | 0.146 | 0.215 |
| secondary_diagnosis_onehot_e876 | 0.146 | 0.215 | secondary_diagnosis_onehot_z800.1 | −0.094 | 0.426 |
| secondary_diagnosis_onehot_g8918 | −0.094 | 0.426 | secondary_diagnosis_onehot_z87442 | 0.146 | 0.215 |
| secondary_diagnosis_onehot_i10.2 | −0.069 | 0.556 | secondary_diagnosis_onehot_z951 | 0.146 | 0.215 |
| secondary_diagnosis_onehot_i110 | 0.146 | 0.215 | secondary_diagnosis_onehot_e109 | −0.094 | 0.426 |
| secondary_diagnosis_onehot_i255 | −0.094 | 0.426 | secondary_diagnosis_onehot_e876.2 | −0.094 | 0.426 |
| secondary_diagnosis_onehot_i471.2 | −0.094 | 0.426 | secondary_diagnosis_onehot_g8918.5 | −0.025 | 0.835 |
| secondary_diagnosis_onehot_i739.1 | −0.094 | 0.426 | secondary_diagnosis_onehot_i10.7 | −0.134 | 0.256 |
| secondary_diagnosis_onehot_i824z1 | 0.146 | 0.215 | secondary_diagnosis_onehot_i129.1 | −0.094 | 0.426 |
| secondary_diagnosis_onehot_i82612 | 0.146 | 0.215 | secondary_diagnosis_onehot_i2510.4 | 0.146 | 0.215 |
| secondary_diagnosis_onehot_i82890.1 | −0.094 | 0.426 | secondary_diagnosis_onehot_i493 | 0.146 | 0.215 |
| secondary_diagnosis_onehot_i959 | −0.094 | 0.426 | secondary_diagnosis_onehot_k219.4 | −0.094 | 0.426 |
| secondary_diagnosis_onehot_j449 | 0.146 | 0.215 | secondary_diagnosis_onehot_k5900.2 | 0.146 | 0.215 |
| secondary_diagnosis_onehot_j90 | −0.134 | 0.256 | secondary_diagnosis_onehot_k760 | 0.146 | 0.215 |
| secondary_diagnosis_onehot_k219.1 | −0.134 | 0.256 | secondary_diagnosis_onehot_m170 | 0.146 | 0.215 |
| secondary_diagnosis_onehot_k311 | −0.094 | 0.426 | secondary_diagnosis_onehot_n183 | 0.146 | 0.215 |
| secondary_diagnosis_onehot_k560 | 0.146 | 0.215 | secondary_diagnosis_onehot_n2581 | 0.146 | 0.215 |
| secondary_diagnosis_onehot_k8309 | −0.094 | 0.426 | secondary_diagnosis_onehot_nan.8 | −0.162 | 0.168 |
| secondary_diagnosis_onehot_k831.2 | −0.094 | 0.426 | secondary_diagnosis_onehot_r918 | −0.094 | 0.426 |
| secondary_diagnosis_onehot_k862.2 | 0.146 | 0.215 | secondary_diagnosis_onehot_z87891.3 | 0.146 | 0.215 |
| secondary_diagnosis_onehot_k869.2 | 0.146 | 0.215 | secondary_diagnosis_onehot_z923 | 0.146 | 0.215 |
| secondary_diagnosis_onehot_k909 | −0.094 | 0.426 | secondary_diagnosis_onehot_b952.1 | 0.146 | 0.215 |
| secondary_diagnosis_onehot_n179 | −0.094 | 0.426 | secondary_diagnosis_onehot_e669.3 | −0.094 | 0.426 |
| secondary_diagnosis_onehot_n184 | 0.146 | 0.215 | secondary_diagnosis_onehot_e780 | −0.134 | 0.256 |
| secondary_diagnosis_onehot_nan.2 | 0.014 | 0.908 | secondary_diagnosis_onehot_e8339 | −0.094 | 0.426 |
| secondary_diagnosis_onehot_r160 | −0.094 | 0.426 | secondary_diagnosis_onehot_g4730 | −0.094 | 0.426 |
| secondary_diagnosis_onehot_r635 | −0.094 | 0.426 | secondary_diagnosis_onehot_g4733.1 | 0.146 | 0.215 |
| secondary_diagnosis_onehot_z6832 | −0.094 | 0.426 | secondary_diagnosis_onehot_g8918.6 | −0.094 | 0.426 |
| secondary_diagnosis_onehot_z8719 | −0.094 | 0.426 | secondary_diagnosis_onehot_i129.2 | 0.146 | 0.215 |
| secondary_diagnosis_onehot_z87891.1 | −0.094 | 0.426 | secondary_diagnosis_onehot_j9811.4 | 0.146 | 0.215 |
| secondary_diagnosis_onehot_b9689 | −0.094 | 0.426 | secondary_diagnosis_onehot_k5790 | −0.094 | 0.426 |
| secondary_diagnosis_onehot_e039.1 | 0.146 | 0.215 | secondary_diagnosis_onehot_m109 | −0.094 | 0.426 |
| secondary_diagnosis_onehot_e063 | −0.094 | 0.426 | secondary_diagnosis_onehot_m8580 | 0.146 | 0.215 |
| secondary_diagnosis_onehot_e1122 | 0.146 | 0.215 | secondary_diagnosis_onehot_n183.1 | 0.146 | 0.215 |
| secondary_diagnosis_onehot_e118 | 0.146 | 0.215 | secondary_diagnosis_onehot_nan.9 | −0.049 | 0.678 |
| secondary_diagnosis_onehot_e119.2 | 0.146 | 0.215 | secondary_diagnosis_onehot_z85828 | −0.094 | 0.426 |
| secondary_diagnosis_onehot_e46.3 | 0.146 | 0.215 | secondary_diagnosis_onehot_z87442.1 | 0.146 | 0.215 |
| secondary_diagnosis_onehot_e669.2 | −0.134 | 0.256 | | | |
| secondary_diagnosis_onehot_e785.3 | −0.094 | 0.426 | | | |
| secondary_diagnosis_onehot_e871.2 | 0.146 | 0.215 | | | |
| secondary_diagnosis_onehot_g8918.1 | 0.146 | 0.215 | | | |
| secondary_diagnosis_onehot_g893 | 0.146 | 0.215 | | | |
| secondary_diagnosis_onehot_i10.3 | −0.025 | 0.835 | | | |
| secondary_diagnosis_onehot_i2510 | −0.094 | 0.426 | | | |

TABLE 5A
DNA Top Features

| Analyte | Study Label | Feature | Frequency | Analyte | Study Label | Feature | Frequency |
|---|---|---|---|---|---|---|---|
| CNV | label_deceased | KDM5D | 0.875 | SNV | label_deceased | UBA52 | 0.52777778 |
| CNV | label_deceased | TSC2 | 0.86111111 | SNV | label_deceased | RHBDD3 | 0.51388889 |
| CNV | label_deceased | HLA-A | 0.66666667 | SNV | label_deceased | MYBBP1A | 0.5 |
| CNV | label_deceased | NUP98 | 0.625 | SNV | label_deceased | HOXB13 | 0.5 |
| CNV | label_deceased | DDR2 | 0.59722222 | SNV | label_deceased | CASKIN1 | 0.43055556 |
| CNV | label_deceased | ABL2 | 0.55555556 | SNV | label_deceased | IDH2 | 0.40277778 |
| CNV | label_deceased | CD274 | 0.51388889 | SNV | label_deceased | CCT7 | 0.36111111 |
| CNV | label_deceased | HRAS | 0.47222222 | SNV | label_deceased | TGFBR1 | 0.33333333 |
| CNV | label_deceased | FOXQ1 | 0.43055556 | SNV | label_deceased | GNA11 | 0.25 |
| CNV | label_deceased | KEL | 0.41666667 | SNV | label_deceased | BRCA1 | 0.23611111 |
| CNV | label_deceased | RAC1 | 0.38888889 | SNV | label_deceased | HES4 | 0.20833333 |
| CNV | label_deceased | H3F3A | 0.375 | SNV | label_deceased | QKI | 0.18055556 |
| CNV | label_deceased | HLA-DOA | 0.33333333 | SNV | label_deceased | ATM | 0.18055556 |
| CNV | label_deceased | SMC3 | 0.33333333 | SNV | label_deceased | KEL | 0.13888889 |
| CNV | label_deceased | FANCL | 0.33333333 | SNV | label_deceased | NTRK1 | 0.125 |
| CNV | label_deceased | C8orf34 | 0.31944444 | SNV | label_deceased | CSDE1 | 0.125 |
| CNV | label_deceased | IRF4 | 0.29166667 | SNV | label_deceased | TP63 | 0.11111111 |
| CNV | label_deceased | EGLN1 | 0.29166667 | SNV | label_deceased | CEP57 | 0.11111111 |
| CNV | label_deceased | RIT1 | 0.29166667 | SNV | label_deceased | STAG2 | 0.11111111 |
| CNV | label_deceased | PTCH1 | 0.29166667 | SNV | label_deceased | HOXA10-HOXA9 | 0.11111111 |
| CNV | label_deceased | TFEB | 0.27777778 | SNV | label_deceased | MKI67 | 0.09722222 |
| CNV | label_deceased | MN1 | 0.26388889 | SNV | label_deceased | ARID1A | 0.09722222 |
| CNV | label_deceased | HLA-DPB1 | 0.26388889 | SNV | label_deceased | VEGFA | 0.08333333 |
| CNV | label_deceased | BCR | 0.26388889 | SNV | label_deceased | SMAD3 | 0.08333333 |
| CNV | label_deceased | SMARCB1 | 0.22222222 | SNV | label_deceased | MTRR | 0.08333333 |
| CNV | label_deceased | HDAC4 | 0.22222222 | SNV | label_deceased | KIT | 0.08333333 |
| CNV | label_deceased | SHH | 0.22222222 | SNV | label_deceased | SHC2 | 0.06944444 |
| CNV | label_deceased | VEGFB | 0.19444444 | SNV | label_deceased | KDM6A | 0.05555556 |
| CNV | label_deceased | EWSR1 | 0.19444444 | SNV | label_deceased | EPHA7 | 0.05555556 |
| CNV | label_deceased | TPMT | 0.19444444 | SNV | label_deceased | CBFB | 0.05555556 |
| CNV | label_deceased | ARID5B | 0.18055556 | INDEL | label_deceased | L2HGDH | 1 |
| CNV | label_deceased | PAK1 | 0.15277778 | INDEL | label_deceased | HOTS | 0.94444444 |
| CNV | label_deceased | GRIN2A | 0.15277778 | INDEL | label_deceased | APC | 0.84722222 |
| CNV | label_deceased | MYB | 0.13888889 | INDEL | label_deceased | CDKN2A | 0.80555556 |
| CNV | label_deceased | MEN1 | 0.13888889 | INDEL | label_deceased | FGF20 | 0.76388889 |
| CNV | label_deceased | PPARA | 0.13888889 | INDEL | label_deceased | BRAF | 0.73611111 |
| CNV | label_deceased | MGMT | 0.13888889 | INDEL | label_deceased | CREBBP | 0.70833333 |
| CNV | label_deceased | SDHAF2 | 0.125 | INDEL | label_deceased | DIS3L2 | 0.66666667 |
| CNV | label_deceased | DYNC2H1 | 0.125 | INDEL | label_deceased | SMAD4 | 0.66666667 |
| CNV | label_deceased | XRCC2 | 0.09722222 | INDEL | label_deceased | CHD4 | 0.65277778 |
| CNV | label_deceased | HLA-DRB6 | 0.09722222 | INDEL | label_deceased | RB1 | 0.625 |
| CNV | label_deceased | FLT1 | 0.09722222 | INDEL | label_deceased | TP53 | 0.58333333 |
| CNV | label_deceased | FANCI | 0.09722222 | INDEL | label_deceased | G6PD | 0.55555556 |
| CNV | label_deceased | XRCC3 | 0.09722222 | INDEL | label_deceased | ARHGAP35 | 0.48611111 |
| CNV | label_deceased | ACVR1B | 0.09722222 | INDEL | label_deceased | MTAP | 0.41666667 |
| CNV | label_deceased | CDKN1B | 0.09722222 | INDEL | label_deceased | TOP2A | 0.375 |
| CNV | label_deceased | LAG3 | 0.09722222 | INDEL | label_deceased | MET | 0.33333333 |
| CNV | label_deceased | NF2 | 0.08333333 | INDEL | label_deceased | RUNX1 | 0.33333333 |
| CNV | label_deceased | RANBP2 | 0.08333333 | INDEL | label_deceased | PHLPP1 | 0.30555556 |
| CNV | label_deceased | GPC3 | 0.08333333 | INDEL | label_deceased | BIRC3 | 0.30555556 |
| CNV | label_deceased | SOX2 | 0.08333333 | INDEL | label_deceased | SH2B3 | 0.30555556 |
| CNV | label_deceased | HLA-DRB5 | 0.06944444 | INDEL | label_deceased | ETV1 | 0.25 |
| CNV | label_deceased | CRKL | 0.06944444 | INDEL | label_deceased | RUSC1 | 0.25 |
| CNV | label_deceased | CHEK2 | 0.06944444 | INDEL | label_deceased | XRCC1 | 0.23611111 |
| CNV | label_deceased | ZNRF3 | 0.06944444 | INDEL | label_deceased | VEGFA | 0.18055556 |
| CNV | label_deceased | DIS3 | 0.05555556 | INDEL | label_deceased | INPP4B | 0.13888889 |
| CNV | label_deceased | NSD2 | 0.05555556 | INDEL | label_deceased | NOTCH3 | 0.125 |
| CNV | label_deceased | TSC1 | 0.05555556 | INDEL | label_deceased | STK11 | 0.125 |
| CNV | label_deceased | FGFR3 | 0.05555556 | INDEL | label_deceased | MRPL2 | 0.11111111 |
| SNV | label_deceased | SYNE1 | 1 | INDEL | label_deceased | BCOR | 0.11111111 |
| SNV | label_deceased | BIRC8 | 0.95833333 | INDEL | label_deceased | CIITA | 0.09722222 |
| SNV | label_deceased | SOX2 | 0.94444444 | INDEL | label_deceased | FANCA | 0.09722222 |
| SNV | label_deceased | RAD51D | 0.94444444 | INDEL | label_deceased | ZBTB1 | 0.09722222 |
| SNV | label_deceased | FGF20 | 0.83333333 | INDEL | label_deceased | ATR | 0.08333333 |
| SNV | label_deceased | IL6R | 0.73611111 | INDEL | label_deceased | AXIN2 | 0.06944444 |
| SNV | label_deceased | DOT1L | 0.66666667 | INDEL | label_deceased | CBL | 0.06944444 |
| SNV | label_deceased | RIT1 | 0.63888889 | INDEL | label_deceased | PPP2R2A | 0.06944444 |
| SNV | label_deceased | MAPK11 | 0.63888889 | INDEL | label_deceased | TLR6 | 0.06944444 |
| SNV | label_deceased | PPP1R3E | 0.54166667 | INDEL | label_deceased | NPM1 | 0.05555556 |
| SNV | label_deceased | CRLF2 | 0.54166667 | INDEL | label_deceased | ANKRD20A11P-LIPI | 0.05555556 |

TABLE 5B
All DNA Features to Endpoints
Survival

| | Spearman | Spearman | | Spearman | Spearman | | Spearman | Spearman |
|---|---|---|---|---|---|---|---|---|
| | rho | p-value | | rho | p-value | | rho | p-value |
| SNV_HES4 | | | SNV_RPTOR | 0.145 | 0.226 | CNV_MSH2 | −0.296 | 0.012 |
| SNV_PPP1R3E | −0.097 | 0.415 | SNV_XRCC3 | −0.097 | 0.415 | CNV_PDCD1LG2 | 0.086 | 0.475 |
| SNV_UBA52 | −0.097 | 0.415 | SNV_FAM175A | −0.139 | 0.245 | CNV_MTHFR | −0.277 | 0.018 |
| SNV_FGF20 | 0.034 | 0.780 | SNV_ERCC2 | −0.097 | 0.415 | CNV_BCORL1 | −0.129 | 0.280 |
| SNV_RIT1 | −0.097 | 0.415 | SNV_POU2F2 | −0.097 | 0.415 | CNV_RPL5 | −0.041 | 0.732 |
| SNV_IL6R | −0.139 | 0.245 | SNV_FBXW7 | −0.097 | 0.415 | CNV_MAFB | −0.028 | 0.813 |
| SNV_SOX2 | −0.002 | 0.990 | SNV_EIF5A | −0.097 | 0.415 | CNV_RASA1 | 0.052 | 0.666 |
| SNV_SYNE1 | 0.079 | 0.507 | SNV_VHL | −0.097 | 0.415 | CNV_ZNRF3 | 0.119 | 0.321 |
| SNV_DOT1L | −0.139 | 0.245 | SNV_WRN | −0.139 | 0.245 | CNV_KEAP1 | −0.003 | 0.978 |
| SNV_RAD51D | 0.034 | 0.780 | SNV_NCOR2 | −0.139 | 0.245 | CNV_BUB1B | −0.019 | 0.873 |
| SNV_RHBDD3 | −0.097 | 0.415 | SNV_GATA4 | 0.145 | 0.226 | CNV_MLH3 | −0.064 | 0.592 |
| SNV_CRLF2 | −0.097 | 0.415 | SNV_MNX1-AS1 | −0.097 | 0.415 | CNV_ERG | −0.100 | 0.405 |
| SNV_BIRC8 | 0.145 | 0.226 | SNV_POLE | −0.097 | 0.415 | CNV_EP300 | 0.096 | 0.421 |
| SNV_MYBBP1A | | | SNV_SETD2 | −0.031 | 0.793 | CNV_TFE3 | −0.011 | 0.926 |
| SNV_MAPK11 | −0.097 | 0.415 | SNV_TRIM5 | −0.097 | 0.415 | CNV_BCL7A | 0.045 | 0.710 |
| SNV_CCT7 | −0.097 | 0.415 | SNV_KEAP1 | 0.036 | 0.765 | CNV_POU2F2 | 0.136 | 0.255 |
| SNV_HOXA10- | −0.097 | 0.415 | SNV_ADRB1 | | | CNV_SMAD4 | 0.022 | 0.855 |
| HOXA9 | | | | | | | | |
| SNV_CSDE1 | −0.097 | 0.415 | SNV_PRKDC | −0.076 | 0.528 | CNV_GSTP1 | −0.140 | 0.240 |
| SNV_IDH2 | 0.145 | 0.226 | SNV_FLT3 | −0.139 | 0.245 | CNV_GATA1 | −0.011 | 0.926 |
| SNV_EPHA7 | −0.097 | 0.415 | SNV_AXL | 0.145 | 0.226 | CNV_SMARCA1 | −0.129 | 0.280 |
| SNV_GNA11 | −0.139 | 0.245 | SNV_ZFHX3 | −0.057 | 0.634 | CNV_TPM1 | −0.067 | 0.577 |
| SNV_PLCG2 | −0.097 | 0.415 | SNV_CTC1 | −0.097 | 0.415 | CNV_STAT4 | 0.073 | 0.543 |
| SNV_HOXB13 | 0.145 | 0.226 | SNV_APC | −0.005 | 0.969 | CNV_MLH1 | 0.110 | 0.357 |
| SNV_SHC2 | −0.097 | 0.415 | SNV_FOXG1-AS1 | 0.145 | 0.226 | CNV_CUL1 | −0.017 | 0.888 |
| SNV_KIT | −0.097 | 0.415 | SNV_KMT2C | −0.043 | 0.722 | CNV_LZTR1 | −0.027 | 0.819 |
| SNV_CASKIN1 | 0.145 | 0.226 | SNV_SLC22A18AS | | | CNV_SMAD2 | 0.004 | 0.976 |
| SNV_NTRK1 | −0.139 | 0.245 | SNV_CTRC | −0.097 | 0.415 | CNV_MAD2L2 | −0.277 | 0.018 |
| SNV_BRCA1 | −0.171 | 0.150 | SNV_AASDH | | | CNV_FCGR2A | −0.054 | 0.651 |
| SNV_WHSC1 | −0.097 | 0.415 | SNV_CD79A | 0.034 | 0.780 | CNV_KRAS | 0.051 | 0.671 |
| SNV_TGFBR1 | 0.172 | 0.149 | SNV_TSHZ3 | −0.097 | 0.415 | CNV_CXCR4 | 0.073 | 0.543 |
| SNV_ELF3 | −0.097 | 0.415 | SNV_NKX2-2 | −0.097 | 0.415 | CNV_MAP2K1 | −0.146 | 0.221 |
| SNV_CCBL2 | −0.097 | 0.415 | SNV_CIITA | −0.030 | 0.806 | CNV_GNAS | −0.028 | 0.813 |
| SNV_SMAD3 | 0.112 | 0.348 | SNV_B9D1 | | | CNV_TOP1 | −0.028 | 0.813 |
| SNV_STAG2 | −0.030 | 0.806 | SNV_SLC26A3 | −0.097 | 0.415 | CNV_KIT | −0.108 | 0.368 |
| SNV_MNX1 | −0.097 | 0.415 | SNV_MIR6769B | −0.097 | 0.415 | CNV_ELF3 | −0.079 | 0.507 |
| SNV_TFEB | −0.139 | 0.245 | SNV_IFNAR2 | −0.097 | 0.415 | CNV_CCND3 | −0.075 | 0.530 |
| SNV_QKI | 0.145 | 0.226 | SNV_RXRA | 0.034 | 0.780 | CNV_POT1 | 0.007 | 0.955 |
| SNV_CXCR4 | −0.097 | 0.415 | SNV_PRIM2 | 0.034 | 0.780 | CNV_JAK1 | −0.045 | 0.708 |
| SNV_CARD11 | −0.097 | 0.415 | SNV_ZNRF3 | −0.076 | 0.528 | CNV_TMEM173 | −0.003 | 0.977 |
| SNV_SETBP1 | −0.097 | 0.415 | SNV_TMPRSS2 | −0.139 | 0.245 | CNV_PIK3CA | −0.088 | 0.464 |
| SNV_CXorf65 | −0.097 | 0.415 | SNV_HIST1H1E | 0.034 | 0.780 | CNV_POLE | 0.054 | 0.653 |
| SNV_MTRR | 0.145 | 0.226 | SNV_HLA-DQA2 | −0.030 | 0.806 | CNV_CHD4 | −0.040 | 0.737 |
| SNV_{—} | −0.097 | 0.415 | SNV_EPM2AIP1 | −0.097 | 0.415 | CNV_BCL2L11 | 0.021 | 0.860 |
| SNV_KEL | 0.112 | 0.348 | SNV_KLLN | 0.034 | 0.780 | CNV_STK11 | −0.015 | 0.899 |
| SNV_ATM | 0.034 | 0.780 | SNV_GRM3 | | | CNV_HIST1H1E | −0.082 | 0.496 |
| SNV_VEGFA | −0.030 | 0.806 | SNV_PTEN | 0.031 | 0.795 | CNV_AURKA | −0.028 | 0.813 |
| SNV_MIR6515 | 0.145 | 0.226 | SNV_MYH11 | −0.139 | 0.245 | CNV_PIK3C2B | −0.078 | 0.517 |
| SNV_ACTN4 | −0.097 | 0.415 | SNV_FGF14 | −0.097 | 0.415 | CNV_RAD21 | −0.007 | 0.955 |
| SNV_GNAS | −0.171 | 0.150 | SNV_HAPLN1 | −0.097 | 0.415 | CNV_TOP2A | −0.150 | 0.210 |
| SNV_NRG1 | −0.076 | 0.528 | SNV_CD70 | 0.034 | 0.780 | CNV_H19 | −0.137 | 0.250 |
| SNV_IKBKE | −0.097 | 0.415 | SNV_MIR4539- | 0.031 | 0.795 | CNV_CYP1B1 | −0.296 | 0.012 |
| | | | KIAA0125 | | | | | |
| SNV_CBFB | 0.145 | 0.226 | SNV_FANCE | −0.097 | 0.415 | CNV_FANCE | −0.030 | 0.804 |
| SNV_TP63 | 0.112 | 0.348 | SNV_FGFR1 | 0.034 | 0.780 | CNV_SETBP1 | 0.004 | 0.976 |
| SNV_NKX2-1 | 0.112 | 0.348 | SNV_MYL1 | 0.145 | 0.226 | CNV_SOX10 | 0.096 | 0.421 |
| SNV_BCL10 | −0.097 | 0.415 | SNV_DYNC2H1 | −0.171 | 0.150 | CNV_RICTOR | 0.052 | 0.666 |
| SNV_DCUN1D2 | −0.097 | 0.415 | SNV_NF1 | 0.036 | 0.765 | CNV_ATM | −0.085 | 0.477 |
| SNV_MTAP | 0.045 | 0.710 | SNV_LINC01060- | −0.199 | 0.093 | CNV_NKX2-1 | −0.064 | 0.592 |
| | | | LINC01262 | | | | | |
| SNV_FANCB | 0.034 | 0.780 | SNV_ASXL1 | −0.139 | 0.245 | CNV_EPHB2 | −0.207 | 0.081 |
| SNV_THAP7-AS1 | −0.097 | 0.415 | SNV_PATZ1 | −0.097 | 0.415 | CNV_ABL1 | 0.008 | 0.945 |
| SNV_PIM1 | 0.034 | 0.780 | SNV_CREBBP | 0.172 | 0.149 | CNV_NOTCH4 | 0.021 | 0.864 |
| SNV_WNT4 | −0.097 | 0.415 | SNV_SF3B1 | 0.034 | 0.780 | CNV_C11orf65 | −0.085 | 0.477 |
| SNV_IRF1 | −0.097 | 0.415 | SNV_ZBTB16 | | | CNV_CTNNA1 | −0.003 | 0.977 |
| SNV_MKI67 | 0.048 | 0.688 | SNV_HLA-DRB1 | −0.005 | 0.969 | CNV_FANCG | 0.075 | 0.532 |
| SNV_ARID2 | −0.171 | 0.150 | SNV_PREX2 | −0.030 | 0.806 | CNV_FANCC | 0.186 | 0.117 |
| SNV_ECT2L | −0.097 | 0.415 | SNV_KMT2D | 0.048 | 0.688 | CNV_SMARCB1 | 0.133 | 0.266 |
| SNV_CCND2 | −0.097 | 0.415 | SNV_MYC | 0.145 | 0.226 | CNV_HIST1H3B | −0.082 | 0.496 |
| SNV_LRRC14 | −0.097 | 0.415 | SNV_BCORL1 | 0.031 | 0.795 | CNV_ATIC | 0.073 | 0.543 |
| SNV_MAB21L3 | −0.097 | 0.415 | SNV_FOXG1 | 0.034 | 0.780 | CNV_MTRR | 0.052 | 0.666 |
| SNV_CEP57 | 0.145 | 0.226 | SNV_ASPSCR1 | −0.139 | 0.245 | CNV_BMPR1A | −0.276 | 0.019 |
| SNV_ARID1A | 0.032 | 0.789 | SNV_UGT1A9 | −0.097 | 0.415 | CNV_FNTB | −0.064 | 0.592 |
| SNV_KDM6A | −0.024 | 0.843 | SNV_HRNR | −0.097 | 0.415 | CNV_FANCM | −0.064 | 0.592 |
| SNV_APOB | 0.162 | 0.173 | SNV_C16orf58 | −0.097 | 0.415 | CNV_CCND2 | −0.040 | 0.737 |
| SNV_ASCL2 | | | SNV_IRS2 | 0.113 | 0.345 | CNV_IL2RA | −0.218 | 0.066 |
| SNV_UBC | −0.097 | 0.415 | SNV_PAX8 | 0.112 | 0.348 | CNV_SDHD | −0.085 | 0.477 |
| SNV_AXIN2 | −0.097 | 0.415 | SNV_RPS16 | −0.097 | 0.415 | CNV_KDR | −0.108 | 0.368 |
| SNV_JAK1 | −0.097 | 0.415 | SNV_PSMB6 | 0.145 | 0.226 | CNV_FGF14 | 0.087 | 0.468 |
| SNV_SPRY3 | −0.097 | 0.415 | SNV_PIK3CG | 0.031 | 0.795 | CNV_MAP2K2 | −0.003 | 0.978 |
| SNV_WNT9A | −0.139 | 0.245 | SNV_ARHGAP26 | −0.097 | 0.415 | CNV_ARAF | −0.011 | 0.926 |
| SNV_ARAF | | | SNV_NTHL1 | 0.206 | 0.083 | CNV_RNF43 | −0.018 | 0.882 |
| SNV_U2AF1 | −0.097 | 0.415 | SNV_MED12 | 0.017 | 0.886 | CNV_MSH6 | −0.296 | 0.012 |
| SNV_CBX5 | 0.145 | 0.226 | SNV_KRAS | 0.159 | 0.182 | CNV_CTNNB1 | 0.137 | 0.251 |
| SNV_MIR6891.7 | −0.139 | 0.245 | SNV_ANKRD20A11P- | −0.097 | 0.415 | CNV_FHIT | −0.025 | 0.835 |
| | | | LIPI | | | | | |
| SNV_RAC2 | −0.097 | 0.415 | SNV_PSME2 | −0.097 | 0.415 | CNV_MAP3K1 | 0.052 | 0.666 |
| SNV_TCL1A | −0.031 | 0.793 | SNV_SYK | −0.139 | 0.245 | CNV_HLA-G | −0.034 | 0.774 |
| SNV_HLA-DRB6 | 0.034 | 0.780 | SNV_HSPA1B | 0.145 | 0.226 | CNV_PPP2R1A | 0.062 | 0.605 |
| SNV_GAPDH | −0.097 | 0.415 | SNV_GPC3 | −0.097 | 0.415 | CNV_MALT1 | 0.043 | 0.719 |
| SNV_EZH2 | −0.139 | 0.245 | SNV_TFEC-TES | 0.145 | 0.226 | CNV_SPRED1 | −0.019 | 0.873 |
| SNV_PDCD1LG2 | −0.097 | 0.415 | SNV_PRCC | 0.145 | 0.226 | CNV_FGFR1 | −0.165 | 0.167 |
| SNV_HSPH1 | −0.097 | 0.415 | SNV_EGFR | −0.139 | 0.245 | CNV_XPC | 0.110 | 0.357 |
| SNV_PRKAR1A | | | SNV_PTCH1 | −0.199 | 0.093 | CNV_PLCG1 | −0.028 | 0.813 |
| SNV_KAT6A | 0.145 | 0.226 | SNV_RHEB | 0.145 | 0.226 | CNV_HLA-DMB | −0.022 | 0.852 |
| SNV_TARP | −0.097 | 0.415 | SNV_RECQL4 | −0.171 | 0.150 | CNV_DIS3L2 | 0.129 | 0.279 |
| SNV_CYP2D6 | 0.145 | 0.226 | SNV_UGDH | −0.097 | 0.415 | CNV_MDM4 | −0.078 | 0.517 |
| SNV_EP300 | −0.097 | 0.415 | SNV_KDM8 | −0.097 | 0.415 | CNV_TBX3 | 0.045 | 0.710 |
| SNV_LEF1 | | | SNV_TRAF7 | 0.145 | 0.226 | CNV_CBR3 | −0.100 | 0.405 |
| SNV_HLF | −0.097 | 0.415 | SNV_BCR | −0.224 | 0.058 | CNV_HLA-DPB2 | −0.008 | 0.947 |
| SNV_BCL2L11 | −0.097 | 0.415 | SNV_SPNS2 | −0.097 | 0.415 | CNV_YEATS4 | 0.012 | 0.917 |
| SNV_PDCD1 | −0.097 | 0.415 | SNV_BCL11B | −0.002 | 0.990 | CNV_FANCA | 0.138 | 0.247 |
| SNV_TCF3 | 0.034 | 0.780 | SNV_ZNF781 | −0.097 | 0.415 | CNV_ARHGAP35 | 0.136 | 0.255 |
| SNV_SRC | 0.145 | 0.226 | SNV_RARA | −0.139 | 0.245 | CNV_ERCC2 | 0.136 | 0.255 |
| SNV_PTPRJ | 0.145 | 0.226 | SNV_MIR4673 | −0.097 | 0.415 | CNV_RAD51B | −0.064 | 0.592 |
| SNV_HLA-DPB2- | −0.097 | 0.415 | SNV_TMEM173 | 0.145 | 0.226 | CNV_WRN | −0.143 | 0.230 |
| COL11A2.2 | | | | | | | | |
| SNV_SOX17-RP1 | −0.097 | 0.415 | SNV_SMAD4 | 0.070 | 0.559 | CNV_SMARCE1 | −0.110 | 0.358 |
| SNV_TAOK3 | −0.097 | 0.415 | SNV_MSH2 | 0.031 | 0.795 | CNV_HSD11B2 | 0.088 | 0.461 |
| SNV_TLR8-AS1 | −0.097 | 0.415 | SNV_GLI2 | −0.030 | 0.806 | CNV_PCBP1 | −0.200 | 0.091 |
| SNV_FANCD2 | | | SNV_NPM1 | −0.097 | 0.415 | CNV_SPINK1 | −0.003 | 0.977 |
| SNV_CDK6 | −0.139 | 0.245 | SNV_CYP3A5 | −0.139 | 0.245 | CNV_TP63 | −0.128 | 0.284 |
| SNV_PAX7 | −0.097 | 0.415 | SNV_ZMYM3 | 0.145 | 0.226 | CNV_CBL | −0.110 | 0.357 |
| SNV_MLLT11 | −0.097 | 0.415 | SNV_SPIDR | −0.097 | 0.415 | CNV_IL15 | −0.108 | 0.368 |
| SNV_DAXX | | | SNV_MRPS15 | | | CNV_GNA13 | 0.040 | 0.739 |
| SNV_HLA-DQB1.3 | | | SNV_BCLAF1 | 0.145 | 0.226 | CNV_EBF1 | −0.070 | 0.561 |
| SNV_FGF22 | −0.097 | 0.415 | SNV_CACNA1S | −0.097 | 0.415 | CNV_AMER1 | −0.070 | 0.559 |
| SNV_AHSA1 | −0.097 | 0.415 | SNV_HLA-DPA1.2 | 0.145 | 0.226 | CNV_CD79A | 0.136 | 0.255 |
| SNV_RNF43 | 0.110 | 0.358 | SNV_RUSC1 | −0.139 | 0.245 | CNV_MYC | −0.007 | 0.955 |
| SNV_ACVR1 | | | SNV_MITF | −0.097 | 0.415 | CNV_HLA-C | −0.008 | 0.947 |
| SNV_HLA-F.6 | −0.097 | 0.415 | SNV_CBX4 | | | CNV_NTRK1 | 0.034 | 0.779 |
| SNV_CPNE8 | −0.139 | 0.245 | SNV_ARID1B | −0.114 | 0.338 | CNV_MED12 | −0.070 | 0.559 |
| SNV_PIK3CB | 0.145 | 0.226 | SNV_IDO1 | 0.145 | 0.226 | CNV_FLT3 | −0.031 | 0.797 |
| SNV_HSD3B2 | 0.145 | 0.226 | SNV_HLA-F-AS1 | −0.097 | 0.415 | CNV_HLA-DRA | 0.021 | 0.864 |
| SNV_CYP1B1 | −0.139 | 0.245 | SNV_JUN | −0.097 | 0.415 | CNV_ITPKB | −0.127 | 0.286 |
| SNV_TSC1 | −0.139 | 0.245 | SNV_RB1 | −0.076 | 0.528 | CNV_SRSF2 | −0.044 | 0.716 |
| SNV_RSPO3 | −0.097 | 0.415 | SNV_RET | −0.139 | 0.245 | CNV_RECQL4 | 0.062 | 0.605 |
| SNV_FGF18 | −0.097 | 0.415 | SNV_KDM5C | 0.172 | 0.149 | CNV_HLA-F | −0.034 | 0.774 |
| SNV_TNFAIP6 | −0.097 | 0.415 | SNV_TNFRSF17 | −0.097 | 0.415 | CNV_HLA-B | 0.021 | 0.864 |
| SNV_SLIT2 | 0.048 | 0.688 | SNV_RAC1 | −0.097 | 0.415 | CNV_AKT1 | 0.088 | 0.461 |
| SNV_ATR | 0.034 | 0.780 | SNV_NTRK2 | 0.112 | 0.348 | CNV_GREM1 | −0.047 | 0.696 |
| SNV_FGFR3 | −0.030 | 0.806 | SNV_C10orf54 | −0.097 | 0.415 | CNV_HNF1A | 0.045 | 0.710 |
| SNV_XPOT | | | SNV_LMNA | −0.031 | 0.793 | CNV_ATRX | −0.070 | 0.559 |
| SNV_MLH3 | −0.097 | 0.415 | SNV_PDGFRB | 0.145 | 0.226 | CNV_IRS2 | 0.090 | 0.451 |
| SNV_ABL1 | | | SNV_AGO1 | −0.139 | 0.245 | CNV_U2AF1 | −0.046 | 0.699 |
| SNV_IRF4 | −0.097 | 0.415 | SNV_GATA3 | −0.139 | 0.245 | CNV_PDCD1 | −0.080 | 0.506 |
| SNV_PDGFA | −0.097 | 0.415 | SNV_EPCAM | 0.145 | 0.226 | CNV_IFNAR2 | −0.076 | 0.525 |
| SNV_XPA | −0.097 | 0.415 | SNV_ERBB3 | −0.097 | 0.415 | CNV_MTAP | 0.119 | 0.317 |
| SNV_HLA-DRB5 | −0.097 | 0.415 | SNV_BAGE3 | 0.145 | 0.226 | CNV_BRCA1 | −0.084 | 0.481 |
| SNV_C9orf129 | 0.034 | 0.780 | SNV_FOXQ1 | −0.097 | 0.415 | CNV_CD19 | 0.176 | 0.140 |
| SNV_SH2B3 | 0.034 | 0.780 | SNV_ETV6 | −0.097 | 0.415 | CNV_RNF139 | −0.005 | 0.968 |
| SNV_BCL2 | 0.145 | 0.226 | SNV_TGFBR2 | −0.145 | 0.224 | CNV_CHD7 | 0.066 | 0.582 |
| SNV_NOTCH3 | −0.002 | 0.990 | SNV_TBX3 | −0.171 | 0.150 | CNV_ATR | −0.118 | 0.324 |
| SNV_CDK8 | −0.097 | 0.415 | SNV_MAP3K1 | 0.034 | 0.780 | CNV_HLA-DOB | −0.032 | 0.789 |
| SNV_JAG2 | 0.145 | 0.226 | SNV_MIR650-MIR5571 | −0.097 | 0.415 | CNV_IL6R | −0.020 | 0.869 |
| SNV_GRIN2A | 0.048 | 0.688 | SNV_MAGEB1- | −0.097 | 0.415 | CNV_PPARD | −0.030 | 0.804 |
| | | | NR0B1 | | | | | |
| SNV_SNRNP70 | | | SNV_TAP2.2 | −0.097 | 0.415 | CNV_ATP7B | 0.060 | 0.616 |
| SNV_SHB | −0.097 | 0.415 | SNV_SDHD | 0.145 | 0.226 | CNV_FCGR3A | −0.049 | 0.681 |
| SNV_HEATR1 | −0.097 | 0.415 | SNV_PMS2 | 0.034 | 0.780 | CNV_FGF4 | 0.039 | 0.742 |
| SNV_PALB2 | | | SNV_PTPRT | −0.097 | 0.415 | CNV_PAX3 | 0.040 | 0.740 |
| SNV_TET2 | 0.145 | 0.226 | SNV_HEY2 | 0.031 | 0.795 | CNV_TAF1 | −0.070 | 0.559 |
| SNV_AMER1 | 0.145 | 0.226 | SNV_SOX9 | 0.034 | 0.780 | CNV_CRLF2 | 0.087 | 0.467 |
| SNV_DCD | 0.034 | 0.780 | SNV_RICTOR | 0.034 | 0.780 | CNV_ING1 | 0.108 | 0.367 |
| SNV_PROS1 | −0.097 | 0.415 | CNV_HLA-DPB1 | −0.002 | 0.989 | CNV_FBXO11 | −0.296 | 0.012 |
| SNV_KCNMA1 | 0.145 | 0.226 | CNV_GPC3 | −0.129 | 0.280 | CNV_TNFRSF14 | −0.277 | 0.018 |
| SNV_MAP2K2 | 0.034 | 0.780 | CNV_SOX2 | −0.128 | 0.284 | CNV_PAX5 | 0.121 | 0.311 |
| SNV_LYN | 0.145 | 0.226 | CNV_XRCC3 | −0.001 | 0.993 | CNV_CBLB | −0.056 | 0.639 |
| SNV_TPM1 | 0.145 | 0.226 | CNV_PAK1 | −0.036 | 0.767 | CNV_FGFR2 | −0.245 | 0.038 |
| SNV_FOXO1 | 0.034 | 0.780 | CNV_ACVR1B | 0.012 | 0.917 | CNV_HNF1B | −0.215 | 0.069 |
| SNV_EGLN1 | 0.034 | 0.780 | CNV_DDR2 | −0.043 | 0.722 | CNV_PHF6 | −0.129 | 0.280 |
| SNV_ATP7B | −0.139 | 0.245 | CNV_FANCL | −0.296 | 0.012 | CNV_ABCC3 | −0.162 | 0.175 |
| SNV_FLT4 | −0.113 | 0.345 | CNV_CDKN1B | −0.046 | 0.700 | CNV_ESR1 | 0.090 | 0.453 |
| SNV_MAP2K4 | 0.034 | 0.780 | CNV_DYNC2H1 | −0.110 | 0.357 | CNV_EGFR | 0.083 | 0.486 |
| SNV_PHLPP1 | −0.078 | 0.513 | CNV_SHH | 0.009 | 0.943 | CNV_ZNF471 | 0.062 | 0.605 |
| SNV_MAPK12 | 0.295 | 0.012 | CNV_SDHAF2 | −0.139 | 0.246 | CNV_TFEC | 0.007 | 0.955 |
| SNV_HSD11B2 | −0.030 | 0.806 | CNV_GRIN2A | 0.176 | 0.140 | CNV_HOXB13 | −0.139 | 0.246 |
| SNV_MSH2- | −0.097 | 0.415 | CNV_LAG3 | −0.040 | 0.737 | CNV_PIK3R2 | −0.001 | 0.993 |
| KCNK12 | | | | | | | | |
| SNV_PBX4 | −0.097 | 0.415 | CNV_MGMT | −0.256 | 0.030 | CNV_CIC | 0.136 | 0.255 |
| SNV_TSC2 | 0.114 | 0.340 | CNV_ABL2 | −0.061 | 0.611 | CNV_PRKAR1A | 0.011 | 0.926 |
| SNV_RAD51C | −0.097 | 0.415 | CNV_PTCH1 | 0.142 | 0.235 | CNV_TCF7L2 | −0.282 | 0.016 |
| SNV_SRGAP2B | −0.097 | 0.415 | CNV_DDX3X | −0.011 | 0.926 | CNV_PIK3CG | 0.007 | 0.955 |
| SNV_CTCF | 0.145 | 0.226 | CNV_H3F3A | −0.078 | 0.517 | CNV_CYP2D6 | 0.096 | 0.421 |
| SNV_XBP1 | −0.097 | 0.415 | CNV_MIB1 | 0.012 | 0.923 | CNV_ERBB3 | 0.012 | 0.917 |
| SNV_MLLT3 | 0.034 | 0.780 | CNV_NUP98 | −0.161 | 0.178 | CNV_SDHC | −0.035 | 0.772 |
| SNV_AR | −0.076 | 0.528 | CNV_PDGFRB | −0.070 | 0.561 | CNV_TGFBR1 | 0.063 | 0.598 |
| SNV_RAD50 | −0.097 | 0.415 | CNV_FAT1 | −0.151 | 0.205 | CNV_VHL | 0.110 | 0.357 |
| SNV_LMO1 | −0.097 | 0.415 | CNV_NHP2 | −0.070 | 0.561 | CNV_DDB2 | −0.114 | 0.339 |
| SNV_FGF2 | | | CNV_ABCB1 | 0.083 | 0.486 | CNV_ACTA2 | −0.276 | 0.019 |
| SNV_POLD1 | −0.097 | 0.415 | CNV_HLA-A | −0.038 | 0.754 | CNV_LATS1 | 0.090 | 0.453 |
| SNV_OCIAD2- | 0.145 | 0.226 | CNV_HLA-DOA | −0.022 | 0.852 | CNV_APC | 0.039 | 0.747 |
| CWH43 | | | | | | | | |
| SNV_CBL | −0.097 | 0.415 | CNV_C8orf34 | 0.060 | 0.617 | CNV_POLQ | −0.077 | 0.518 |
| SNV_PALLD | −0.076 | 0.528 | CNV_NSD1 | −0.070 | 0.561 | CNV_SDHA | 0.052 | 0.666 |
| SNV_NFKBIA | | | CNV_HDAC4 | 0.153 | 0.198 | CNV_DAXX | −0.008 | 0.947 |
| SNV_MAP2K3 | | | CNV_TSC2 | 0.116 | 0.330 | CNV_BRAF | 0.040 | 0.737 |
| SNV_NFKBIE | −0.097 | 0.415 | CNV_KEL | −0.081 | 0.496 | CNV_CTLA4 | 0.073 | 0.543 |
| SNV_PPP2R1A | −0.139 | 0.245 | CNV_BTG1 | 0.045 | 0.710 | CNV_MAX | −0.064 | 0.592 |
| SNV_BIRC3 | −0.097 | 0.415 | CNV_VEGFB | −0.173 | 0.146 | CNV_EPCAM | −0.296 | 0.012 |
| SNV_PIK3R2 | −0.070 | 0.557 | CNV_RPS6KB1 | −0.016 | 0.895 | CNV_AXIN1 | 0.116 | 0.330 |
| SNV_ABCC3 | −0.199 | 0.094 | CNV_HRAS | −0.180 | 0.131 | CNV_FLT4 | −0.024 | 0.840 |
| SNV_FGF4 | −0.097 | 0.415 | CNV_PTCH2 | −0.080 | 0.506 | CNV_HAS3 | 0.088 | 0.461 |
| SNV_RIMBP2 | −0.097 | 0.415 | CNV_STAG2 | −0.129 | 0.280 | CNV_BIRC3 | −0.092 | 0.440 |
| SNV_RUNX1 | 0.034 | 0.780 | CNV_KDM5D | 0.109 | 0.363 | CNV_SLC26A3 | 0.007 | 0.955 |
| SNV_RBM15 | −0.097 | 0.415 | CNV_FLT1 | −0.031 | 0.797 | CNV_TANC1 | 0.073 | 0.543 |
| SNV_MDM4 | 0.145 | 0.226 | CNV_MEN1 | −0.173 | 0.146 | CNV_HLA-E | −0.008 | 0.947 |
| SNV_DICER1 | −0.139 | 0.245 | CNV_WEE1 | −0.116 | 0.330 | CNV_APOB | −0.245 | 0.038 |
| SNV_NGF | 0.145 | 0.226 | CNV_ERCC3 | 0.073 | 0.543 | CNV_NCOR1 | −0.031 | 0.793 |
| SNV_ZBTB22 | 0.145 | 0.226 | CNV_ARID2 | 0.053 | 0.656 | CNV_PTEN | −0.276 | 0.019 |
| SNV_PDPK1 | 0.114 | 0.340 | CNV_ERCC4 | 0.176 | 0.140 | CNV_MTHFD2 | −0.302 | 0.010 |
| SNV_HLA-DOA.6 | 0.145 | 0.226 | CNV_SMC3 | −0.329 | 0.005 | CNV_STAT6 | 0.012 | 0.917 |
| SNV_SCARNA11 | −0.139 | 0.245 | CNV_PPP6C | 0.014 | 0.909 | CNV_SH2B3 | 0.045 | 0.710 |
| SNV_MIR3135B | −0.097 | 0.415 | CNV_MEF2B | −0.014 | 0.906 | CNV_PPM1D | 0.041 | 0.730 |
| SNV_CUL4A | 0.145 | 0.226 | CNV_RIT1 | −0.020 | 0.869 | CNV_MAP3K7 | 0.002 | 0.985 |
| SNV_HLA-B | −0.076 | 0.528 | CNV_BCOR | −0.011 | 0.926 | CNV_NFE2L2 | 0.073 | 0.543 |
| SNV_MN1 | 0.173 | 0.145 | CNV_UMPS | −0.077 | 0.518 | CNV_TRAF3 | −0.064 | 0.592 |
| SNV_CD274 | 0.031 | 0.795 | CNV_ARHGAP26 | −0.003 | 0.977 | CNV_AXIN2 | 0.011 | 0.926 |
| SNV_CIC | −0.199 | 0.093 | CNV_ARID1A | −0.187 | 0.116 | CNV_FAS | −0.276 | 0.019 |
| SNV_FAT1 | 0.034 | 0.780 | CNV_KIF1B | −0.277 | 0.018 | CNV_FANCB | 0.036 | 0.767 |
| SNV_DDB2 | −0.097 | 0.415 | CNV_EGLN1 | −0.076 | 0.526 | CNV_HLA-DPA1 | −0.002 | 0.989 |
| SNV_KDR | 0.036 | 0.765 | CNV_IFNL3 | 0.002 | 0.986 | CNV_NT5C2 | −0.329 | 0.005 |
| SNV_INPP4B | 0.145 | 0.226 | CNV_RANBP2 | 0.053 | 0.656 | CNV_ERCC6 | −0.233 | 0.049 |
| SNV_DLST | | | CNV_RAC1 | −0.047 | 0.692 | CNV_PRSS1 | −0.053 | 0.658 |
| SNV_PTPN13 | 0.145 | 0.226 | CNV_XRCC2 | 0.009 | 0.943 | CNV_AJUBA | −0.172 | 0.148 |
| SNV_WNT6 | −0.097 | 0.415 | CNV_CD274 | 0.086 | 0.475 | CNV_MITF | −0.063 | 0.600 |
| SNV_TYMS | −0.097 | 0.415 | CNV_ARID5B | −0.304 | 0.009 | CNV_MAP2K4 | −0.154 | 0.197 |
| SNV_MIR4436A- | −0.097 | 0.415 | CNV_TFEB | −0.075 | 0.530 | CNV_BCL2 | 0.043 | 0.719 |
| LOC654342 | | | | | | | | |
| SNV_LCK | −0.139 | 0.245 | CNV_ERBB2 | −0.256 | 0.030 | CNV_UGT1A9 | 0.127 | 0.288 |
| SNV_CBLC | 0.145 | 0.226 | CNV_SUZ12 | −0.150 | 0.210 | CNV_LRP1B | 0.073 | 0.543 |
| SNV_LRP1B | 0.129 | 0.282 | CNV_CTC1 | −0.154 | 0.197 | CNV_PML | −0.209 | 0.078 |
| SNV_GAB3 | −0.097 | 0.415 | CNV_CREBBP | 0.176 | 0.140 | CNV_KLF4 | 0.037 | 0.755 |
| SNV_LINC01219 | 0.034 | 0.780 | CNV_ERBB4 | 0.073 | 0.543 | CNV_TBL1XR1 | −0.087 | 0.470 |
| SNV_RANBP2 | −0.139 | 0.245 | CNV_PALB2 | 0.176 | 0.140 | CNV_WT1 | −0.116 | 0.330 |
| SNV_TP53 | −0.180 | 0.131 | CNV_DIRC2 | −0.077 | 0.518 | CNV_RARA | −0.195 | 0.100 |
| SNV_MSH3 | 0.145 | 0.226 | CNV_FANCI | −0.169 | 0.157 | CNV_PTPRD | 0.093 | 0.435 |
| SNV_NDE1 | −0.097 | 0.415 | CNV_BCL10 | −0.020 | 0.868 | CNV_MPL | −0.129 | 0.282 |
| SNV_SMO | −0.076 | 0.528 | CNV_NOTCH3 | −0.046 | 0.698 | CNV_ZNF217 | −0.028 | 0.813 |
| SNV_EPHA2 | −0.097 | 0.415 | CNV_TSC1 | 0.008 | 0.945 | CNV_SDHB | −0.261 | 0.027 |
| SNV_NXN | −0.139 | 0.245 | CNV_DIS3 | 0.060 | 0.616 | CNV_SPOP | −0.177 | 0.136 |
| SNV_SUZ12 | −0.097 | 0.415 | CNV_KLHL6 | −0.128 | 0.284 | CNV_FGF10 | 0.052 | 0.666 |
| SNV_PBRM1 | 0.206 | 0.083 | CNV_MYCL | −0.129 | 0.282 | CNV_L2HGDH | −0.064 | 0.592 |
| SNV_PHOX2B | −0.139 | 0.245 | CNV_EWSR1 | 0.119 | 0.321 | CNV_PBRM1 | 0.073 | 0.543 |
| SNV_SLC35B2 | −0.097 | 0.415 | CNV_PPARA | 0.096 | 0.421 | CNV_BLM | −0.169 | 0.157 |
| SNV_CASR | 0.145 | 0.226 | CNV_CEBPA | 0.185 | 0.120 | CNV_FGF5 | −0.108 | 0.368 |
| SNV_MCL1 | 0.034 | 0.780 | CNV_BRD4 | −0.046 | 0.698 | CNV_OLIG2 | −0.076 | 0.525 |
| SNV_PAX3 | −0.097 | 0.415 | CNV_SLC47A2 | −0.031 | 0.793 | CNV_CSF3R | −0.240 | 0.042 |
| SNV_ELK3 | −0.097 | 0.415 | CNV_RAD50 | −0.003 | 0.977 | CNV_NRG1 | −0.175 | 0.142 |
| SNV_RSF1 | 0.145 | 0.226 | CNV_MCL1 | −0.030 | 0.804 | CNV_ACVR1 | 0.073 | 0.543 |
| SNV_MIR5196 | −0.097 | 0.415 | CNV_FGF8 | −0.297 | 0.011 | CNV_RINT1 | 0.034 | 0.777 |
| SNV_DDX3X | −0.097 | 0.415 | CNV_PLCG2 | 0.138 | 0.247 | CNV_IRF1 | −0.003 | 0.977 |
| SNV_SOX1 | −0.097 | 0.415 | CNV_DICER1 | −0.064 | 0.592 | CNV_FBXW7 | −0.108 | 0.368 |
| SNV_MAF | 0.145 | 0.226 | CNV_GATA6 | −0.017 | 0.889 | CNV_TERT | 0.052 | 0.666 |
| SNV_IL7R | −0.139 | 0.245 | CNV_HLA-DRB6 | 0.071 | 0.556 | CNV_ZMYM3 | −0.070 | 0.559 |
| SNV_CYLD | 0.145 | 0.226 | CNV_PMS2 | −0.047 | 0.692 | CNV_PRCC | −0.029 | 0.811 |
| SNV_CD79B | 0.034 | 0.780 | CNV_AKT3 | −0.076 | 0.526 | CNV_GEN1 | −0.308 | 0.008 |
| SNV_FANCA | 0.051 | 0.667 | CNV_IFNGR2 | −0.076 | 0.525 | CNV_RAD51 | −0.019 | 0.873 |
| SNV_TFDP1 | −0.097 | 0.415 | CNV_PPP1R15A | 0.174 | 0.144 | CNV_KAT6A | −0.025 | 0.832 |
| SNV_HOTS | 0.145 | 0.226 | CNV_CD22 | 0.185 | 0.120 | CNV_FGF6 | −0.040 | 0.737 |
| SNV_FUT1 | −0.097 | 0.415 | CNV_FOXO3 | 0.040 | 0.737 | CNV_ETS1 | −0.089 | 0.458 |
| SNV_CMPK1 | −0.097 | 0.415 | CNV_HLA-DQA2 | 0.044 | 0.716 | CNV_ETV5 | −0.128 | 0.284 |
| SNV_ZNF750 | −0.097 | 0.415 | CNV_TCF3 | −0.015 | 0.899 | CNV_NQO1 | 0.088 | 0.461 |
| SNV_TBCD | −0.097 | 0.415 | CNV_FGFR4 | −0.070 | 0.561 | CNV_MLLT3 | 0.121 | 0.311 |
| SNV_WNK1 | −0.139 | 0.245 | CNV_JUN | −0.026 | 0.827 | CNV_CDKN1C | −0.161 | 0.178 |
| SNV_NOTCH2 | | | CNV_HLA-DQA1 | 0.017 | 0.886 | CNV_PREX2 | 0.060 | 0.617 |
| SNV_FBXO11 | −0.097 | 0.415 | CNV_KMT2C | 0.009 | 0.943 | CNV_CDKN2B | 0.119 | 0.317 |
| SNV_CYSLTR2 | −0.097 | 0.415 | CNV_SMO | 0.007 | 0.955 | CNV_HDAC1 | −0.277 | 0.018 |
| SNV_CDKN2B | −0.097 | 0.415 | CNV_FAM46C | 0.014 | 0.909 | CNV_CARD11 | −0.043 | 0.720 |
| SNV_PPP2R2A | −0.097 | 0.415 | CNV_CYP3A5 | 0.078 | 0.513 | CNV_ETS2 | −0.100 | 0.405 |
| SNV_HMGA2 | −0.097 | 0.415 | CNV_FGF1 | −0.003 | 0.977 | CNV_ZFHX3 | 0.138 | 0.247 |
| SNV_BTK | −0.097 | 0.415 | CNV_RAD54L | −0.080 | 0.506 | CNV_LDLR | −0.046 | 0.698 |
| SNV_CBX8 | −0.097 | 0.415 | CNV_EPHA7 | 0.002 | 0.985 | CNV_B2M | −0.019 | 0.873 |
| SNV_EBF1 | −0.097 | 0.415 | CNV_MYH11 | 0.176 | 0.140 | CNV_LYN | 0.066 | 0.582 |
| SNV_SMARCA4 | −0.113 | 0.345 | CNV_GATA4 | −0.135 | 0.259 | CNV_KDM5C | −0.011 | 0.926 |
| SNV_EWSR1 | −0.097 | 0.415 | CNV_TP53 | −0.154 | 0.197 | CNV_CKS1B | −0.020 | 0.869 |
| SNV_MAGI2 | −0.077 | 0.519 | CNV_CDK6 | 0.083 | 0.486 | CNV_IDH1 | 0.073 | 0.543 |
| SNV_CHD2 | −0.097 | 0.415 | CNV_FDPS | −0.020 | 0.869 | CNV_RAF1 | 0.110 | 0.357 |
| SNV_IL10RA | −0.097 | 0.415 | CNV_KDM5A | −0.040 | 0.737 | CNV_MAF | 0.138 | 0.247 |
| SNV_CDKN1A | −0.097 | 0.415 | CNV_CFTR | 0.007 | 0.955 | CNV_SF3B1 | 0.073 | 0.543 |
| SNV_CWH43- | −0.097 | 0.415 | CNV_NFKBIA | −0.032 | 0.791 | CNV_GNA11 | −0.062 | 0.605 |
| DCUN1D4 | | | | | | | | |
| SNV_HSF5 | 0.145 | 0.226 | CNV_CTCF | 0.088 | 0.461 | CNV_UGT1A1 | 0.157 | 0.187 |
| SNV_AKT2 | | | CNV_RUNX1 | −0.076 | 0.525 | CNV_FOXA1 | −0.064 | 0.592 |
| SNV_SFRP1 | −0.097 | 0.415 | CNV_CUL3 | 0.040 | 0.740 | CNV_FH | −0.076 | 0.526 |
| SNV_FADD | | | CNV_PPARG | 0.110 | 0.357 | CNV_SOX9 | 0.011 | 0.926 |
| SNV_CSF1R | | | CNV_STAT3 | −0.110 | 0.358 | CNV_ARID1B | 0.090 | 0.453 |
| SNV_KSR1 | 0.145 | 0.226 | CNV_SRC | −0.028 | 0.813 | CNV_PAX7 | −0.261 | 0.027 |
| SNV_MET | 0.112 | 0.348 | CNV_KDM6A | −0.011 | 0.926 | CNV_RAD51D | −0.150 | 0.210 |
| SNV_BRCA2 | 0.034 | 0.780 | CNV_CBLC | 0.174 | 0.143 | CNV_TSHR | −0.064 | 0.592 |
| SNV_BMPR1A | 0.145 | 0.226 | CNV_AGO1 | −0.240 | 0.042 | CNV_EMSY | −0.036 | 0.767 |
| SNV_CDH26 | −0.139 | 0.245 | CNV_BTK | −0.070 | 0.559 | CNV_IKZF1 | 0.083 | 0.486 |
| SNV_ENG | −0.097 | 0.415 | CNV_SOCS1 | 0.176 | 0.140 | CNV_INPP4B | −0.108 | 0.368 |
| SNV_GLI1 | −0.097 | 0.415 | CNV_APLNR | −0.139 | 0.246 | CNV_FGF2 | −0.108 | 0.368 |
| SNV_KMT2B | −0.139 | 0.245 | CNV_IRF2 | −0.151 | 0.205 | CNV_MS4A1 | −0.139 | 0.246 |
| SNV_CUL3 | −0.030 | 0.806 | CNV_BCL6 | −0.128 | 0.284 | CNV_PRDM1 | 0.040 | 0.737 |
| SNV_FAM110C | −0.097 | 0.415 | CNV_GALNT12 | 0.063 | 0.598 | CNV_UBE2T | −0.078 | 0.517 |
| SNV_ERCC6 | | | CNV_HIF1A | −0.064 | 0.592 | CNV_TAP1 | −0.022 | 0.852 |
| SNV_BARD1 | 0.034 | 0.780 | CNV_PHOX2B | −0.151 | 0.205 | CNV_XRCC1 | 0.101 | 0.398 |
| SNV_FAM227B | 0.145 | 0.226 | CNV_NRAS | 0.014 | 0.909 | CNV_CTRC | −0.277 | 0.018 |
| SNV_FUBP1 | 0.034 | 0.780 | CNV_CARM1 | −0.046 | 0.698 | CNV_SOD2 | 0.090 | 0.453 |
| SNV_EPHA3-PROS1 | −0.097 | 0.415 | CNV_SUFU | −0.329 | 0.005 | CNV_CCDC6 | −0.201 | 0.091 |
| SNV_CUL1 | −0.030 | 0.806 | CNV_MDM2 | 0.012 | 0.917 | CNV_MC1R | 0.084 | 0.483 |
| SNV_CDH1 | 0.145 | 0.226 | CNV_ETV4 | −0.088 | 0.460 | CNV_SLIT2 | −0.151 | 0.205 |
| SNV_PHGDH | 0.145 | 0.226 | CNV_MET | 0.007 | 0.955 | CNV_HSPH1 | −0.055 | 0.645 |
| SNV_CCDC178 | 0.145 | 0.226 | CNV_DPYD | −0.038 | 0.749 | CNV_TGFBR2 | 0.110 | 0.357 |
| SNV_ESR1 | 0.034 | 0.780 | CNV_ERRFI1 | −0.277 | 0.018 | CNV_BRIP1 | 0.041 | 0.730 |
| SNV_IFNGR1 | −0.097 | 0.415 | CNV_KMT2B | 0.185 | 0.120 | CNV_CDKN2A | 0.119 | 0.317 |
| SNV_HOXC13 | −0.097 | 0.415 | CNV_IKBKE | −0.078 | 0.517 | CNV_NPM1 | −0.070 | 0.561 |
| SNV_STAT6 | −0.097 | 0.415 | CNV_GPS2 | −0.154 | 0.197 | CNV_GABRA6 | −0.070 | 0.561 |
| SNV_CRK | −0.097 | 0.415 | CNV_FGF23 | −0.040 | 0.737 | CNV_GATA3 | −0.218 | 0.066 |
| SNV_ARHGAP35 | −0.097 | 0.415 | CNV_EPOR | −0.046 | 0.698 | CNV_DNMT3A | −0.247 | 0.036 |
| SNV_GATA6 | 0.048 | 0.688 | CNV_STAT5B | −0.110 | 0.358 | CNV_LMNA | −0.029 | 0.811 |
| SNV_JAZF1 | −0.097 | 0.415 | CNV_KLLN | −0.276 | 0.019 | CNV_SMARCA4 | −0.046 | 0.698 |
| SNV_ETV1 | 0.034 | 0.780 | CNV_IFIT3 | −0.294 | 0.012 | CNV_RHEB | 0.009 | 0.943 |
| SNV_TLX1 | −0.097 | 0.415 | CNV_NOTCH1 | 0.008 | 0.945 | CNV_CSF1R | −0.070 | 0.561 |
| SNV_RBM10 | 0.034 | 0.780 | CNV_C3orf70 | −0.128 | 0.284 | CNV_IFNGR1 | 0.081 | 0.497 |
| SNV_TBC1D12 | −0.097 | 0.415 | CNV_ELOC | 0.060 | 0.617 | CNV_GNAQ | 0.054 | 0.655 |
| SNV_IFNAR1 | 0.145 | 0.226 | CNV_NOP10 | −0.019 | 0.873 | CNV_RPS15 | −0.015 | 0.899 |
| SNV_PTPRD | 0.145 | 0.226 | CNV_SGK1 | 0.057 | 0.635 | CNV_HIST1H4E | −0.082 | 0.496 |
| SNV_BRAF | −0.097 | 0.415 | CNV_FGF3 | 0.039 | 0.742 | CNV_MRE11 | 0.003 | 0.981 |
| SNV_CDKN1C | 0.034 | 0.780 | CNV_HAVCR2 | −0.070 | 0.561 | CNV_CEP57 | −0.048 | 0.690 |
| SNV_BRD4 | −0.097 | 0.415 | CNV_CASP8 | 0.073 | 0.543 | CNV_ZRSR2 | −0.011 | 0.926 |
| SNV_G6PD | 0.206 | 0.083 | CNV_BCL2L1 | −0.028 | 0.813 | CNV_DOT1L | −0.070 | 0.559 |
| SNV_ZBTB1 | −0.097 | 0.415 | CNV_EPHA2 | −0.277 | 0.018 | CNV_FUBP1 | 0.021 | 0.859 |
| SNV_RAD23B | −0.097 | 0.415 | CNV_LEF1 | −0.108 | 0.368 | CNV_BCLAF1 | 0.108 | 0.368 |
| SNV_LDLR | 0.034 | 0.780 | CNV_CDKN2C | −0.026 | 0.827 | CNV_HDAC2 | 0.040 | 0.737 |
| SNV_CFTR | 0.048 | 0.688 | CNV_HOXA11 | −0.062 | 0.605 | CNV_AR | −0.034 | 0.780 |
| SNV_SLX4 | −0.097 | 0.415 | CNV_TNFAIP3 | 0.081 | 0.497 | CNV_SYK | 0.186 | 0.117 |
| SNV_TRAF3 | −0.097 | 0.415 | CNV_SYNE1 | 0.090 | 0.453 | CNV_NTRK3 | −0.167 | 0.162 |
| SNV_MLH1 | −0.139 | 0.245 | CNV_TMEM127 | 0.022 | 0.858 | CNV_NUDT15 | −0.011 | 0.929 |
| SNV_MPL | −0.097 | 0.415 | CNV_CHEK1 | −0.110 | 0.357 | CNV_RSF1 | −0.036 | 0.767 |
| SNV_NRTN | −0.097 | 0.415 | CNV_RUNX1T1 | 0.014 | 0.909 | CNV_P2RY8 | 0.087 | 0.467 |
| SNV_IKZF1 | 0.034 | 0.780 | CNV_CIITA | 0.176 | 0.140 | CNV_IFIT1 | −0.294 | 0.012 |
| SNV_SOX10 | −0.097 | 0.415 | CNV_IRF4 | −0.061 | 0.613 | CNV_IL10RA | −0.085 | 0.477 |
| SNV_HNF1B | −0.171 | 0.150 | CNV_ERCC1 | 0.136 | 0.255 | CNV_CDK4 | 0.012 | 0.917 |
| SNV_RRBP1 | −0.097 | 0.415 | CNV_NF2 | 0.117 | 0.327 | CNV_FRS2 | 0.012 | 0.917 |
| SNV_MIR3147- | 0.034 | 0.780 | CNV_SLC9A3R1 | 0.011 | 0.926 | CNV_BCR | 0.087 | 0.466 |
| ZNF716 | | | | | | | | |
| SNV_TOP2A | −0.139 | 0.245 | CNV_PIK3R1 | 0.052 | 0.666 | CNV_PTPRT | −0.001 | 0.993 |
| SNV_MSH6 | −0.139 | 0.245 | CNV_VEGFA | −0.060 | 0.614 | CNV_CDK12 | −0.236 | 0.046 |
| SNV_SNORD96A | | | CNV_NTRK2 | 0.133 | 0.266 | CNV_MSH3 | 0.052 | 0.666 |
| SNV_CASC11 | 0.145 | 0.226 | CNV_NF1 | −0.113 | 0.346 | CNV_CCND1 | 0.008 | 0.945 |
| SNV_WEE1 | −0.139 | 0.245 | CNV_PTPN13 | −0.108 | 0.368 | CNV_JAK2 | 0.109 | 0.362 |
| SNV_BIRC5 | −0.097 | 0.415 | CNV_FOXO1 | −0.057 | 0.636 | CNV_NBN | 0.012 | 0.921 |
| SNV_BORA | −0.097 | 0.415 | CNV_MAPK1 | 0.016 | 0.895 | CNV_HLA-DQB2 | −0.030 | 0.799 |
| SNV_FOXL2 | −0.030 | 0.806 | CNV_ROS1 | 0.040 | 0.737 | CNV_BRCA2 | −0.079 | 0.511 |
| SNV_KMT2A | 0.254 | 0.031 | CNV_XPO1 | −0.219 | 0.065 | CNV_RAD51C | −0.016 | 0.895 |
| SNV_ASNS | | | CNV_HLA-DRB5 | 0.071 | 0.556 | CNV_NOTCH2 | −0.008 | 0.949 |
| SNV_GPX4 | −0.097 | 0.415 | CNV_CD79B | 0.011 | 0.926 | CNV_DNM2 | −0.003 | 0.978 |
| SNV_PCDH17 | 0.206 | 0.083 | CNV_TMPRSS2 | −0.091 | 0.446 | CNV_TNFRSF9 | −0.277 | 0.018 |
| SNV_SMC1A | | | CNV_FOXP1 | −0.063 | 0.600 | CNV_FGF9 | −0.031 | 0.797 |
| SNV_MIR4733 | 0.034 | 0.780 | CNV_MYB | 0.097 | 0.419 | CNV_ECT2L | 0.083 | 0.489 |
| SNV_P2RY8 | 0.145 | 0.226 | CNV_ZNF750 | −0.017 | 0.889 | CNV_CDKN1A | −0.030 | 0.804 |
| SNV_DIS3L2 | 0.108 | 0.365 | CNV_NTHL1 | 0.116 | 0.330 | INDEL_STK11 | −0.097 | 0.415 |
| SNV_TAX1BP1 | −0.097 | 0.415 | CNV_CHEK2 | 0.119 | 0.321 | INDEL_XRCC1 | −0.139 | 0.245 |
| SNV_ZBTB33 | −0.139 | 0.245 | CNV_IFIT2 | −0.276 | 0.019 | INDEL_L2HGDH | 0.070 | 0.559 |
| SNV_FGF19 | −0.097 | 0.415 | CNV_BARD1 | 0.073 | 0.543 | INDEL_ETV1 | −0.097 | 0.415 |
| SNV_ASCL1 | −0.097 | 0.415 | CNV_WNK1 | −0.040 | 0.737 | INDEL_APC | 0.112 | 0.348 |
| SNV_C3orf70 | 0.145 | 0.226 | CNV_TET2 | −0.108 | 0.368 | INDEL_BIRC3 | −0.097 | 0.415 |
| SNV_PIAS4 | 0.145 | 0.226 | CNV_CD40 | −0.028 | 0.813 | INDEL_KMT2D | | |
| SNV_CHTF8 | 0.145 | 0.226 | CNV_GRM3 | 0.083 | 0.486 | INDEL_NPM1 | −0.097 | 0.415 |
| SNV_BCL6 | | | CNV_EZH2 | 0.009 | 0.943 | INDEL_VEGFA | −0.097 | 0.415 |
| SNV_KLHL6 | −0.097 | 0.415 | CNV_ASNS | 0.081 | 0.501 | INDEL_CSF1R | | |
| SNV_CACNA1B | 0.145 | 0.226 | CNV_VSIR | −0.284 | 0.016 | INDEL_MRPL2 | −0.097 | 0.415 |
| SNV_RHOA | −0.139 | 0.245 | CNV_ERCC5 | 0.084 | 0.485 | INDEL_ARHGAP35 | 0.145 | 0.226 |
| SNV_FGF6 | −0.097 | 0.415 | CNV_PMS1 | 0.073 | 0.543 | INDEL_RUSC1 | −0.097 | 0.415 |
| SNV_NTRK3 | 0.110 | 0.358 | CNV_SPEN | −0.277 | 0.018 | INDEL_MDM4 | | |
| SNV_SNAP47 | −0.097 | 0.415 | CNV_FOXL2 | −0.118 | 0.324 | INDEL_MTAP | 0.045 | 0.710 |
| SNV_CRKL | −0.097 | 0.415 | CNV_POLH | −0.075 | 0.530 | INDEL_BCOR | −0.097 | 0.415 |
| SNV_POLQ | −0.097 | 0.415 | CNV_PHLPP1 | 0.043 | 0.719 | INDEL_HOTS | 0.145 | 0.226 |
| SNV_FANCM | −0.097 | 0.415 | CNV_ASPSCR1 | −0.017 | 0.889 | INDEL_ZBTB1 | −0.097 | 0.415 |
| SNV_PTPN11 | −0.097 | 0.415 | CNV_PRKN | 0.090 | 0.453 | INDEL_NOTCH3 | −0.097 | 0.415 |
| SNV_JAK2 | −0.031 | 0.793 | CNV_TUSC3 | −0.115 | 0.337 | INDEL_PPP2R2A | −0.097 | 0.415 |
| SNV_SUMO1P1 | | | CNV_MKI67 | −0.256 | 0.030 | INDEL_FANCA | −0.097 | 0.415 |
| SNV_BCOR | −0.070 | 0.557 | CNV_RET | −0.233 | 0.049 | INDEL_SPNS2 | −0.097 | 0.415 |
| SNV_AJUBA | 0.145 | 0.226 | CNV_KMT2A | −0.110 | 0.357 | INDEL_ERBB4 | −0.097 | 0.415 |
| SNV_L2HGDH | 0.166 | 0.164 | CNV_FGFR3 | 0.008 | 0.949 | INDEL_FBXO11 | | |
| SNV_SPI1 | −0.097 | 0.415 | CNV_RHOA | 0.073 | 0.543 | INDEL_CDKN2B | −0.097 | 0.415 |
| SNV_MYCL | 0.145 | 0.226 | CNV_ZNF620 | 0.110 | 0.357 | INDEL_AXIN2 | −0.097 | 0.415 |
| SNV_CEBPA | −0.076 | 0.528 | CNV_TAP2 | −0.022 | 0.852 | INDEL_ZFHX3 | −0.097 | 0.415 |
| SNV_MED29 | −0.097 | 0.415 | CNV_FANCD2 | 0.110 | 0.357 | INDEL_DCD | −0.097 | 0.415 |
| SNV_BTG1 | −0.097 | 0.415 | CNV_EGF | −0.108 | 0.368 | INDEL_NTRK1 | −0.097 | 0.415 |
| SNV_EPHB1 | 0.206 | 0.083 | CNV_PTPN11 | 0.045 | 0.710 | INDEL_MED12 | −0.097 | 0.415 |
| SNV_EIF1AX | −0.097 | 0.415 | CNV_CDC73 | −0.061 | 0.611 | INDEL_UGDH | −0.097 | 0.415 |
| SNV_PIK3CD | −0.097 | 0.415 | CNV_PHLPP2 | 0.138 | 0.247 | INDEL_MET | −0.030 | 0.806 |
| SNV_PINK1 | 0.145 | 0.226 | CNV_RPTOR | 0.021 | 0.859 | INDEL_BRAF | −0.199 | 0.093 |
| SNV_STAT3 | −0.097 | 0.415 | CNV_GATA2 | −0.039 | 0.744 | INDEL_SETD2 | | |
| SNV_HOXC8 | −0.097 | 0.415 | CNV_CD70 | −0.003 | 0.978 | INDEL_HSF5 | 0.145 | 0.226 |
| SNV_CDKN1B | −0.139 | 0.245 | CNV_CDH1 | 0.088 | 0.461 | INDEL_SH2B3 | 0.145 | 0.226 |
| SNV_MTRNR2L7- | 0.145 | 0.226 | CNV_PTPN22 | 0.014 | 0.909 | INDEL_CREBBP | 0.145 | 0.226 |
| ZNF248 | | | | | | | | |
| SNV_HOXC13-AS | −0.097 | 0.415 | CNV_CHD2 | −0.177 | 0.138 | INDEL_RUNX1 | 0.145 | 0.226 |
| SNV_DNMT3A | −0.171 | 0.150 | CNV_CASR | −0.077 | 0.518 | INDEL_BRD4 | −0.097 | 0.415 |
| SNV_SMARCB1 | | | CNV_KMT2D | 0.012 | 0.917 | INDEL_DYNC2H1 | −0.097 | 0.415 |
| SNV_PDK1 | −0.097 | 0.415 | CNV_CYLD | 0.138 | 0.247 | INDEL_CDKN2A | 0.113 | 0.345 |
| SNV_TUSC3 | 0.034 | 0.780 | CNV_TARBP2 | 0.012 | 0.917 | INDEL_FGF20 | 0.145 | 0.226 |
| SNV_IGF2 | −0.097 | 0.415 | CNV_PHGDH | −0.001 | 0.992 | INDEL_PRKDC | −0.097 | 0.415 |
| SNV_RAD51B | −0.097 | 0.415 | CNV_MAGI2 | 0.083 | 0.486 | INDEL_ACVR1B | −0.139 | 0.245 |
| SNV_KDM5D | −0.097 | 0.415 | CNV_ABRAXAS1 | −0.108 | 0.368 | INDEL_ATR | 0.145 | 0.226 |
| SNV_ARHGAP39 | −0.097 | 0.415 | CNV_RBM10 | −0.011 | 0.926 | INDEL_FAT1 | | |
| SNV_FGF8 | −0.030 | 0.806 | CNV_PIM1 | −0.030 | 0.804 | INDEL_CBL | −0.097 | 0.415 |
| SNV_PCBP1 | 0.145 | 0.226 | CNV_AURKB | −0.154 | 0.197 | INDEL_FUS | −0.097 | 0.415 |
| SNV_MTHFR | −0.097 | 0.415 | CNV_FGF7 | −0.019 | 0.873 | INDEL_CSDE1 | −0.097 | 0.415 |
| SNV_MYB | 0.145 | 0.226 | CNV_TCL1A | −0.001 | 0.993 | INDEL_G6PD | 0.145 | 0.226 |
| SNV_ERBB4 | −0.139 | 0.245 | CNV_HLA-DRB1 | 0.033 | 0.784 | INDEL_DIS3L2 | 0.145 | 0.226 |
| SNV_FGF1 | −0.097 | 0.415 | CNV_HSD3B2 | 0.014 | 0.909 | INDEL_ANKRD20A11P- | −0.097 | 0.415 |
| | | | | | | LIPI | | |
| SNV_RAD21 | −0.139 | 0.245 | CNV_PRKDC | 0.066 | 0.582 | INDEL_SMC3 | −0.139 | 0.245 |
| SNV_CEBPE | −0.171 | 0.150 | CNV_ASXL1 | −0.028 | 0.813 | INDEL_CBFB | | |
| SNV_LPAR6 | −0.097 | 0.415 | CNV_PIK3CB | −0.118 | 0.324 | INDEL_SPIDR | −0.097 | 0.415 |
| SNV_OLIG2 | −0.097 | 0.415 | CNV_CALR | −0.001 | 0.993 | INDEL_EGFR | 0.034 | 0.780 |
| SNV_BAP1 | 0.145 | 0.226 | CNV_FANCF | −0.116 | 0.330 | INDEL_TLR6 | −0.097 | 0.415 |
| SNV_CLSTN1 | −0.097 | 0.415 | CNV_RB1 | 0.023 | 0.850 | INDEL_GRIN2A | −0.139 | 0.245 |
| SNV_CWH43 | 0.145 | 0.226 | CNV_CDK8 | −0.031 | 0.797 | INDEL_GAB3 | −0.097 | 0.415 |
| SNV_CD22 | 0.048 | 0.688 | CNV_NCOR2 | 0.045 | 0.710 | INDEL_SMAD4 | 0.048 | 0.688 |
| SNV_CTNNB1 | −0.097 | 0.415 | CNV_AKT2 | 0.034 | 0.778 | INDEL_CHD4 | 0.145 | 0.226 |
| SNV_HLA-C | −0.097 | 0.415 | CNV_PDGFRA | −0.108 | 0.368 | INDEL_RB1 | 0.145 | 0.226 |
| SNV_KIAA0125- | −0.097 | 0.415 | CNV_JAK3 | −0.046 | 0.698 | INDEL_SMO | 0.145 | 0.226 |
| ADAM6 | | | | | | | | |
| SNV_ING1 | −0.097 | 0.415 | CNV_HSD3B1 | −0.054 | 0.654 | INDEL_BRCA1 | −0.097 | 0.415 |
| SNV_FUS | −0.097 | 0.415 | CNV_HGF | 0.083 | 0.486 | INDEL_CUX1 | −0.097 | 0.415 |
| SNV_MAP3K12 | −0.030 | 0.806 | CNV_TIGIT | −0.118 | 0.324 | INDEL_TOP2A | −0.097 | 0.415 |
| SNV_XRCC1 | −0.139 | 0.245 | CNV_SEMA3C | 0.083 | 0.486 | INDEL_PHLPP1 | 0.034 | 0.780 |
| SNV_FCGR3A | 0.145 | 0.226 | CNV_IFNAR1 | −0.076 | 0.525 | INDEL_ERCC2 | −0.097 | 0.415 |
| SNV_STK11 | 0.113 | 0.345 | CNV_TPMT | −0.061 | 0.613 | INDEL_{—} | −0.097 | 0.415 |
| SNV_NUP98 | −0.097 | 0.415 | CNV_ETV6 | −0.040 | 0.737 | INDEL_TRIM5 | −0.097 | 0.415 |
| SNV_BUB3 | −0.097 | 0.415 | CNV_MTOR | −0.277 | 0.018 | INDEL_VHL | −0.097 | 0.415 |
| SNV_BAGE4 | 0.173 | 0.145 | CNV_GLI2 | 0.073 | 0.543 | INDEL_ASXL1 | −0.097 | 0.415 |
| SNV_PARK2 | −0.139 | 0.245 | CNV_CBFB | 0.088 | 0.461 | INDEL_DHH | −0.097 | 0.415 |
| SNV_ACVR1B | −0.026 | 0.831 | CNV_ENG | 0.014 | 0.909 | INDEL_KMT2C | −0.097 | 0.415 |
| SNV_ERG | 0.206 | 0.083 | CNV_SMC1A | −0.011 | 0.926 | INDEL_SCARNA11 | −0.097 | 0.415 |
| SNV_WT1 | −0.076 | 0.528 | CNV_EIF1AX | −0.011 | 0.926 | INDEL_CIITA | −0.030 | 0.806 |
| SNV_NF2 | −0.074 | 0.538 | CNV_SETD2 | 0.103 | 0.390 | INDEL_WT1 | −0.097 | 0.415 |
| SNV_ALK | | | CNV_CUX1 | 0.039 | 0.748 | INDEL_INPP4B | 0.145 | 0.226 |
| SNV_ERBB2 | −0.097 | 0.415 | CNV_CRKL | −0.015 | 0.900 | INDEL_RASA1 | | |
| SNV_CUX1 | −0.171 | 0.150 | CNV_TNFRSF17 | 0.176 | 0.140 | INDEL_PNPT1 | −0.097 | 0.415 |
| SNV_NOTCH1 | −0.005 | 0.969 | CNV_ETV1 | −0.037 | 0.757 | INDEL_LRP1B | 0.206 | 0.083 |
| SNV_PRDM1 | −0.097 | 0.415 | CNV_EPHB1 | −0.039 | 0.744 | INDEL_TAOK3 | −0.097 | 0.415 |
| SNV_WNK2 | −0.063 | 0.597 | CNV_TYMS | 0.150 | 0.209 | INDEL_NF1 | 0.145 | 0.226 |
| SNV_TLR6 | −0.097 | 0.415 | CNV_ALK | −0.296 | 0.012 | INDEL_EPM2AIP1 | −0.097 | 0.415 |
| SNV_PCSK6 | −0.097 | 0.415 | CNV_PPP2R2A | −0.143 | 0.230 | INDEL_TP53 | 0.165 | 0.167 |
| SNV_SPEN | 0.112 | 0.348 | CNV_MUTYH | −0.080 | 0.506 | INDEL_ARID1A | 0.048 | 0.688 |
| SNV_TLX1NB | −0.097 | 0.415 | CNV_PDK1 | 0.073 | 0.543 | INDEL_MSH6 | −0.139 | 0.245 |
| SNV_PIK3CA | 0.034 | 0.780 | CNV_RRM1 | −0.156 | 0.190 | INDEL_FGFR3 | −0.139 | 0.245 |
| SNV_HLA-G | 0.145 | 0.226 | CNV_CUL4A | 0.077 | 0.523 | INDEL_HLA-B | −0.097 | 0.415 |
| SNV_CBLB | −0.139 | 0.245 | CNV_FOXQ1 | −0.061 | 0.613 | INDEL_BMPR1A | 0.145 | 0.226 |
| SNV_TLX3 | −0.097 | 0.415 | CNV_STAT5A | −0.110 | 0.358 | INDEL_ATIC | −0.097 | 0.415 |
| SNV_TERT | −0.081 | 0.499 | CNV_DKC1 | −0.119 | 0.320 | INDEL_KEAP1 | 0.145 | 0.226 |
| SNV_CUL4B | 0.145 | 0.226 | CNV_WNK2 | 0.186 | 0.117 | INDEL_MIR4733 | 0.034 | 0.780 |
| SNV_NOTCH4 | −0.139 | 0.245 | CNV_SMAD3 | −0.189 | 0.112 | INDEL_ATM | −0.097 | 0.415 |
| SNV_TGFB1I1 | −0.097 | 0.415 | CNV_PALLD | −0.108 | 0.368 | INDEL_TGFBR1 | 0.034 | 0.780 |
| SNV_EPHB2 | 0.145 | 0.226 | CNV_MYD88 | 0.110 | 0.357 | INDEL_HOXC8 | 0.034 | 0.780 |
| SNV_FGF3 | −0.139 | 0.245 | CNV_MYCN | −0.245 | 0.038 | INDEL_RBM10 | 0.145 | 0.226 |
| SNV_DHH | −0.097 | 0.415 | CNV_LMO1 | −0.156 | 0.190 | INDEL_KDM6A | −0.031 | 0.793 |
| SNV_PHLPP2 | −0.097 | 0.415 | CNV_IDO1 | −0.111 | 0.354 | INDEL_IRF1 | −0.097 | 0.415 |
| SNV_MIB1 | 0.145 | 0.226 | CNV_G6PD | −0.129 | 0.280 | INDEL_SYNE1 | 0.112 | 0.348 |
| SNV_CASP5 | 0.145 | 0.226 | CNV_HLA-DMA | −0.022 | 0.852 | INDEL_CXCR4 | −0.097 | 0.415 |
| SNV_MDM2 | −0.097 | 0.415 | CNV_AXL | 0.134 | 0.263 | INDEL_ERBB3 | −0.097 | 0.415 |
| SNV_FLT1 | 0.172 | 0.149 | CNV_SCG5 | −0.047 | 0.696 | INDEL_GABRG2 | −0.097 | 0.415 |
| SNV_NBN | | | CNV_PIAS4 | −0.003 | 0.978 | INDEL_TNFAIP6 | −0.097 | 0.415 |
| SNV_PPP1R15A | −0.097 | 0.415 | CNV_SEC23B | −0.001 | 0.993 | INDEL_POLQ | −0.097 | 0.415 |
| SNV_NKX2-8 | −0.139 | 0.245 | CNV_XPA | 0.063 | 0.598 | INDEL_NOTCH1 | −0.097 | 0.415 |
| SNV_FGF23 | 0.145 | 0.226 | CNV_FLCN | −0.031 | 0.793 | INDEL_KMT2A | 0.145 | 0.226 |
| SNV_ROS1 | −0.097 | 0.415 | CNV_IDH2 | −0.169 | 0.157 | INDEL_KRAS | 0.206 | 0.083 |
| SNV_TLR5 | −0.097 | 0.415 | CNV_QKI | 0.090 | 0.453 | INDEL_RHEB | 0.145 | 0.226 |
| SNV_JAK3 | 0.145 | 0.226 | CNV_PAX8 | 0.073 | 0.543 | INDEL_IL3 | −0.097 | 0.415 |
| SNV_MIR6765 | | | CNV_BAP1 | 0.073 | 0.543 | INDEL_RAD23B | −0.097 | 0.415 |
| SNV_RHOB | −0.097 | 0.415 | CNV_CUL4B | −0.129 | 0.280 | INDEL_KEL | 0.034 | 0.780 |
| SNV_HLA-A | −0.145 | 0.224 | CNV_TRAF7 | 0.114 | 0.339 | INDEL_CEBPA | | |
| SNV_NTF3 | −0.097 | 0.415 | CNV_FUS | 0.176 | 0.140 | INDEL_TERT | −0.171 | 0.150 |
| SNV_LAG3 | 0.034 | 0.780 | CNV_PIK3CD | −0.277 | 0.018 | INDEL_AJUBA | 0.145 | 0.226 |
| SNV_HNF1A | 0.145 | 0.226 | CNV_RXRA | 0.008 | 0.945 | INDEL_DNM2 | 0.145 | 0.226 |
| SNV_FCGR2A | 0.145 | 0.226 | CNV_SLX4 | 0.176 | 0.140 | INDEL_TAX1BP1 | −0.097 | 0.415 |
| SNV_ATIC | −0.097 | 0.415 | CNV_TBC1D12 | −0.343 | 0.003 | INDEL_RIMBP2 | −0.097 | 0.415 |
| SNV_LZTR1 | 0.145 | 0.226 | CNV_BCL11B | −0.001 | 0.993 | INDEL_NOTCH4 | −0.097 | 0.415 |
| SNV_ARHGEF1 | 0.112 | 0.348 | CNV_IL7R | 0.052 | 0.666 | INDEL_WHSC1 | −0.097 | 0.415 |
| SNV_CHD7 | −0.097 | 0.415 | CNV_TNF | 0.021 | 0.864 | INDEL_STAG2 | −0.097 | 0.415 |
| SNV_CTNNA1 | −0.097 | 0.415 | CNV_POLD1 | 0.062 | 0.605 | INDEL_HEATR1 | −0.097 | 0.415 |
| SNV_MEN1 | −0.097 | 0.415 | CNV_LCK | −0.212 | 0.073 | INDEL_EWSR1 | −0.097 | 0.415 |
| SNV_DNM2 | 0.206 | 0.083 | CNV_CCNE1 | 0.016 | 0.892 | INDEL_ARID2 | −0.097 | 0.415 |
| SNV_PAF1 | −0.097 | 0.415 | CNV_HLA-DQB1 | 0.017 | 0.886 | INDEL_RNF43 | 0.034 | 0.780 |
| SNV_SYNDIG1 | 0.145 | 0.226 | CNV_NSD2 | −0.010 | 0.935 | INDEL_EPCAM | 0.145 | 0.226 |
| SNV_CDKN2A | 0.187 | 0.116 | CNV_CYSLTR2 | 0.057 | 0.636 | | | |
| SNV_SOCS1 | 0.031 | 0.795 | CNV_GLI1 | 0.012 | 0.917 | | | |
| SNV_AXIN1 | −0.097 | 0.415 | CNV_HSP90AA1 | −0.064 | 0.592 | | | |
| SNV_IGFLR1 | −0.097 | 0.415 | CNV_MN1 | 0.093 | 0.435 | | | |

TABLE 6A
RNA Top Features

| Analyte | Study Label | Feature | Frequency | Analyte | Study Label | Feature | Frequency |
|---|---|---|---|---|---|---|---|
| RNA_Fusion | label_deceased | KMT2A/SORBS2 | 1 | RNA_Expr | label_deceased | MSANTD3 | 0.2105 |
| RNA_Fusion | label_deceased | KMT2A/EPS15 | 1 | RNA_Expr | label_deceased | CPD | 0.2105 |
| RNA_Fusion | label_deceased | ARFIP1/FHDC1 | 1 | RNA_Expr | label_deceased | AP2B1 | 0.2105 |
| RNA_Fusion | label_deceased | SET/NUP214 | 0.9825 | RNA_Expr | label_deceased | BMS1 | 0.193 |
| RNA_Fusion | label_deceased | DHH/RHEBL1 | 0.9825 | RNA_Expr | label_deceased | ANKMY1 | 0.193 |
| RNA_Fusion | label_deceased | EZR/ROS1 | 0.9825 | RNA_Expr | label_deceased | RAB8A | 0.193 |
| RNA_Fusion | label_deceased | KMT2A/ARHGAP26 | 0.9825 | RNA_Expr | label_deceased | SMC4 | 0.193 |
| RNA_Fusion | label_deceased | INTS4/GAB2 | 0.9825 | RNA_Expr | label_deceased | DKK3 | 0.1754 |
| RNA_Fusion | label_deceased | COL1A1/PDGFB | 0.9474 | RNA_Expr | label_deceased | SORL1 | 0.1579 |
| RNA_Fusion | label_deceased | PLXND1/TMCC1 | 0.9474 | RNA_Expr | label_deceased | GNE | 0.1579 |
| RNA_Fusion | label_deceased | KMT2A/ARHGEF12 | 0.9123 | RNA_Expr | label_deceased | SKIL | 0.1404 |
| RNA_Fusion | label_deceased | PLA2R1/RBMS1 | 0.8947 | RNA_Expr | label_deceased | CLTC | 0.1228 |
| RNA_Fusion | label_deceased | STRN/ALK | 0.7018 | RNA_Expr | label_deceased | DDI2 | 0.1228 |
| RNA_Fusion | label_deceased | KMT2A/AFF4 | 0.5263 | RNA_Expr | label_deceased | CDS2 | 0.1228 |
| RNA_Fusion | label_deceased | SLC45A3/ELK4 | 0.4737 | RNA_Expr | label_deceased | TRIM25 | 0.1053 |
| RNA_Fusion | label_deceased | KMT2A/ELL | 0.386 | RNA_Expr | label_deceased | FARP2 | 0.1053 |
| RNA_Fusion | label_deceased | LSM14A/BRAF | 0.3333 | RNA_Expr | label_deceased | ICAM1 | 0.0877 |
| RNA_Fusion | label_deceased | KMT2A/PRRC1 | 0.2982 | RNA_Expr | label_deceased | NEDD9 | 0.0877 |
| RNA_Fusion | label_deceased | ETV6/ITPR2 | 0.2982 | RNA_Expr | label_deceased | YY1AP1 | 0.0877 |
| RNA_Fusion | label_deceased | NAB2/STAT6 | 0.0877 | RNA_Expr | label_deceased | GNAQ | 0.0877 |
| RNA_Fusion | label_deceased | PCM1/JAK2 | 0.0702 | RNA_Expr | label_deceased | ACTN4 | 0.0877 |
| RNA_Fusion | label_deceased | SND1/BRAF | 0.0526 | RNA_Expr | label_deceased | AGAP1 | 0.0877 |
| RNA_Fusion | label_deceased | KMT2A/MLLT6 | 0.0526 | RNA_Expr | label_deceased | TASP1 | 0.0877 |
| RNA_Fusion | label_deceased | KMT2A/GMPS | 0.0526 | RNA_Expr | label_deceased | TIPARP | 0.0877 |
| RNA_Expr | label_deceased | NFE2L2 | 0.8246 | RNA_Expr | label_deceased | SKAP2 | 0.0702 |
| RNA_Expr | label_deceased | LRIG3 | 0.5439 | RNA_Expr | label_deceased | STAT2 | 0.0702 |
| RNA_Expr | label_deceased | SRSF4 | 0.5439 | RNA_Expr | label_deceased | DEK | 0.0702 |
| RNA_Expr | label_deceased | TOMM7 | 0.5263 | RNA_Expr | label_deceased | WWC2 | 0.0702 |
| RNA_Expr | label_deceased | PRKX | 0.4561 | RNA_Expr | label_deceased | WDR60 | 0.0702 |
| RNA_Expr | label_deceased | ABHD2 | 0.4386 | RNA_Expr | label_deceased | VAMP3 | 0.0702 |
| RNA_Expr | label_deceased | SLC25A13 | 0.4211 | RNA_Expr | label_deceased | TMEM248 | 0.0702 |
| RNA_Expr | label_deceased | PSMD5 | 0.4035 | RNA_Expr | label_deceased | TOM1L2 | 0.0702 |
| RNA_Expr | label_deceased | BAZ2A | 0.3684 | RNA_Expr | label_deceased | SMC3 | 0.0702 |
| RNA_Expr | label_deceased | USP22 | 0.3684 | RNA_Expr | label_deceased | KCNK1 | 0.0702 |
| RNA_Expr | label_deceased | CACNA1D | 0.3158 | RNA_Expr | label_deceased | GADD45G | 0.0526 |
| RNA_Expr | label_deceased | DLG1 | 0.2982 | RNA_Expr | label_deceased | TTC33 | 0.0526 |
| RNA_Expr | label_deceased | VPS41 | 0.2982 | RNA_Expr | label_deceased | AKAP13 | 0.0526 |
| RNA_Expr | label_deceased | NIPAL2 | 0.2982 | RNA_Expr | label_deceased | UBE3C | 0.0526 |
| RNA_Expr | label_deceased | RGS5 | 0.2982 | RNA_Expr | label_deceased | SMC6 | 0.0526 |
| RNA_Expr | label_deceased | SLC40A1 | 0.2807 | RNA_Expr | label_deceased | GRAMD2B | 0.0526 |
| RNA_Expr | label_deceased | APPBP2 | 0.2807 | RNA_Expr | label_deceased | PRMT3 | 0.0526 |
| RNA_Expr | label_deceased | SERINC5 | 0.2807 | RNA_Expr | label_deceased | MYL6 | 0.0526 |
| RNA_Expr | label_deceased | ZNF704 | 0.2456 | RNA_Expr | label_deceased | IGFBP5 | 0.0526 |
| RNA_Expr | label_deceased | BCL9L | 0.2456 | RNA_Expr | label_deceased | ATAD3A | 0.0526 |
| RNA_Expr | label_deceased | RNA5SP389 | 0.2281 | RNA_Expr | label_deceased | UBE2Z | 0.0526 |
| RNA_Expr | label_deceased | ANKRD13A | 0.2281 | RNA_Expr | label_deceased | ZDHHC7 | 0.0526 |
| RNA_Expr | label_deceased | NBPF26 | 0.2105 | RNA_Expr | label_deceased | NLRC5 | 0.0526 |

TABLE 6B
All RNA Features to Endpoints
Survival

| | Spearman | Spearman | | Spearman | Spearman | | Spearman | Spearman |
|---|---|---|---|---|---|---|---|---|
| | rho | p-value | | rho | p-value | | rho | p-value |
| AF4_SND1/BRAF | −0.114 | 0.399 | RNA_CSNK1G3 | −0.110 | 0.415 | RNA_RAC1 | 0.095 | 0.482 |
| AF4_KMT2A/ELL | 0.157 | 0.244 | RNA_KANSL1 | 0.019 | 0.886 | RNA_LRRK2 | 0.123 | 0.362 |
| AF4_INTS4/GAB2 | 0.157 | 0.244 | RNA_NIPBL | −0.045 | 0.738 | RNA_CPLANE1 | −0.026 | 0.848 |
| AF4_KMT2A/EPS15 | 0.224 | 0.095 | RNA_CNOT1 | −0.063 | 0.643 | RNA_ISLR | 0.060 | 0.655 |
| AF4_SET/NUP214 | 0.157 | 0.244 | RNA_PARP4 | 0.037 | 0.786 | RNA_SERP1 | −0.168 | 0.210 |
| AF4_ETV6/ITPR2 | 0.157 | 0.244 | RNA_DPY19L4 | 0.186 | 0.167 | RNA_TAF15 | −0.121 | 0.370 |
| AF4_KMT2A/MLLT6 | 0.157 | 0.244 | RNA_ZMYND8 | −0.084 | 0.533 | RNA_MPP5 | −0.106 | 0.433 |
| AF4_TECTA/TBCEL | 0.030 | 0.827 | RNA_WASH2P | −0.162 | 0.229 | RNA_VGLL4 | 0.089 | 0.512 |
| AF4_KMT2A/LPP | 0.114 | 0.397 | RNA_XRRA1 | −0.054 | 0.690 | RNA_TICAM1 | 0.076 | 0.576 |
| AF4_VTI1A/TCF7L2 | −0.048 | 0.721 | RNA_BHLHA15 | −0.063 | 0.643 | RNA_WASH9P | 0.112 | 0.406 |
| AF4_PCM1/JAK2 | 0.157 | 0.244 | RNA_MFSD14C | 0.186 | 0.167 | RNA_PRKX | 0.421 | 0.001 |
| AF4_DHH/RHEBL1 | 0.157 | 0.244 | RNA_PPP1R12A | −0.082 | 0.544 | RNA_S100A11 | −0.067 | 0.621 |
| AF4_KMT2A/GMPS | 0.157 | 0.244 | RNA_MFAP3 | −0.186 | 0.167 | RNA_CIITA | 0.011 | 0.936 |
| AF4_KMT2A/SORBS2 | 0.224 | 0.095 | RNA_MICU1 | 0.102 | 0.452 | RNA_ATR | −0.071 | 0.598 |
| AF4_SLC45A3/ELK4 | 0.212 | 0.114 | RNA_DTX3L | −0.400 | 0.002 | RNA_MYO1D | 0.110 | 0.415 |
| AF4_KMT2A/SEPT2 | | | RNA_CC2D2A | 0.041 | 0.762 | RNA_PSMD1 | −0.158 | 0.241 |
| AF4_KMT2A/EP300 | | | RNA_RAP1B | −0.104 | 0.443 | RNA_ATAD1 | 0.099 | 0.462 |
| AF4_NAB2/STAT6 | −0.114 | 0.399 | RNA_CSNK1A1 | −0.093 | 0.492 | RNA_NOTCH2NLA | 0.017 | 0.898 |
| AF4_PLA2R1/RBMS1 | 0.034 | 0.802 | RNA_NFIC | 0.052 | 0.702 | RNA_CD58 | 0.125 | 0.353 |
| AF4_LSM14A/BRAF | 0.157 | 0.244 | RNA_ACVR1 | 0.216 | 0.107 | RNA_DENND4C | 0.177 | 0.188 |
| AF4_ARFIP1/FHDC1 | 0.322 | 0.015 | RNA_NIN | −0.004 | 0.975 | RNA_ILF3 | −0.175 | 0.193 |
| AF4_EZR/ROS1 | 0.157 | 0.244 | RNA_OPHN1 | 0.348 | 0.008 | RNA_TCEA1 | −0.166 | 0.216 |
| AF4_KMT2A/PRRC1 | 0.030 | 0.822 | RNA_BPTF | 0.102 | 0.452 | RNA_RSKR | −0.099 | 0.462 |
| AF4_KMT2A/AFF4 | −0.114 | 0.399 | RNA_HOOK3 | 0.130 | 0.337 | RNA_ARHGAP29 | −0.091 | 0.502 |
| AF4_KMT2A/ARHGAP26 | 0.157 | 0.244 | RNA_SNRK | 0.166 | 0.216 | RNA_GNAQ | 0.337 | 0.010 |
| AF4_STRN/ALK | | | RNA_TRIM2 | 0.235 | 0.078 | RNA_RMND5A | 0.106 | 0.433 |
| AF4_KMT2A/ARHGEF12 | −0.114 | 0.399 | RNA_ADAM9 | −0.130 | 0.337 | RNA_FMNL1 | −0.114 | 0.397 |
| AF4_COL1A1/PDGFB | 0.157 | 0.244 | RNA_UVRAG | 0.102 | 0.452 | RNA_SORBS1 | 0.149 | 0.269 |
| AF4_PLXND1/TMCC1 | | | RNA_CEL | 0.108 | 0.424 | RNA_IST1 | 0.052 | 0.702 |
| RNA_IL6ST | 0.050 | 0.714 | RNA_ASB3 | −0.104 | 0.443 | RNA_ERAP2 | −0.123 | 0.362 |
| RNA_LPP | −0.069 | 0.609 | RNA_DNM2 | −0.002 | 0.987 | RNA_EFEMP1 | 0.017 | 0.898 |
| RNA_REG1A | 0.067 | 0.621 | RNA_BCL10 | −0.024 | 0.861 | RNA_SLC41A1 | 0.045 | 0.738 |
| RNA_WASF2 | 0.156 | 0.248 | RNA_ZSWIM6 | 0.035 | 0.799 | RNA_CAPRIN1 | −0.106 | 0.433 |
| RNA_UBXN7 | −0.119 | 0.379 | RNA_ANO6 | −0.110 | 0.415 | RNA_BTN2A1 | −0.162 | 0.229 |
| RNA_NFAT5 | −0.132 | 0.329 | RNA_MET | −0.261 | 0.050 | RNA_TTYH3 | 0.019 | 0.886 |
| RNA_MT-ND4 | 0.093 | 0.492 | RNA_PRKCI | 0.078 | 0.565 | RNA_ST3GAL6 | −0.212 | 0.114 |
| RNA_NOTCH2 | −0.015 | 0.911 | RNA_SAMD4A | −0.078 | 0.565 | RNA_ZCCHC7 | −0.019 | 0.886 |
| RNA_ARID1A | −0.201 | 0.134 | RNA_ZNF124 | 0.089 | 0.512 | RNA_MGP | 0.162 | 0.229 |
| RNA_PLEC | 0.069 | 0.609 | RNA_NUP50 | −0.019 | 0.886 | RNA_PBX3 | −0.080 | 0.555 |
| RNA_TLK1 | −0.086 | 0.523 | RNA_EPB41L4A | −0.015 | 0.911 | RNA_SPRED2 | 0.244 | 0.067 |
| RNA_LIMS1 | −0.175 | 0.193 | RNA_UPF2 | −0.184 | 0.172 | RNA_SEC61B | 0.002 | 0.987 |
| RNA_OSBPL8 | 0.060 | 0.655 | RNA_MYLK | −0.024 | 0.861 | RNA_GMIP | 0.032 | 0.811 |
| RNA_PUM1 | −0.145 | 0.283 | RNA_SLMAP | −0.045 | 0.738 | RNA_NFKB1 | 0.037 | 0.786 |
| RNA_MTATP6P1 | 0.022 | 0.873 | RNA_PNLIP | 0.184 | 0.172 | RNA_PDLIM7 | −0.104 | 0.443 |
| RNA_EVI5 | 0.151 | 0.262 | RNA_FAM222B | −0.112 | 0.406 | RNA_EPC2 | 0.106 | 0.433 |
| RNA_PHC3 | 0.138 | 0.305 | RNA_RPL29 | 0.194 | 0.147 | RNA_GPATCH2 | −0.067 | 0.621 |
| RNA_RABGAP1L | 0.136 | 0.313 | RNA_YME1L1 | −0.069 | 0.609 | RNA_ACTN1 | −0.063 | 0.643 |
| RNA_RANBP2 | −0.086 | 0.523 | RNA_CYTH3 | 0.222 | 0.096 | RNA_MS4A7 | 0.104 | 0.443 |
| RNA_VPS13B | −0.039 | 0.774 | RNA_ZNF791 | −0.002 | 0.987 | RNA_EHF | −0.002 | 0.987 |
| RNA_ZBTB20 | 0.233 | 0.081 | RNA_PTPRA | −0.026 | 0.848 | RNA_TRIM35 | −0.041 | 0.762 |
| RNA_RGPD6 | 0.054 | 0.690 | RNA_ATXN2 | −0.089 | 0.512 | RNA_CPNE3 | 0.330 | 0.012 |
| RNA_NCOA2 | 0.274 | 0.039 | RNA_PAK2 | −0.039 | 0.774 | RNA_CCDC88A | 0.015 | 0.911 |
| RNA_ACTB | −0.177 | 0.188 | RNA_TNS3 | −0.011 | 0.936 | RNA_WDR45B | 0.080 | 0.555 |
| RNA_MAP4K4 | −0.076 | 0.576 | RNA_MIGA1 | 0.069 | 0.609 | RNA_MOB1A | 0.017 | 0.898 |
| RNA_MT-CO3 | 0.073 | 0.587 | RNA_UBC | 0.019 | 0.886 | RNA_BMP1 | −0.026 | 0.848 |
| RNA_RPS3A | 0.153 | 0.255 | RNA_FBN1 | 0.179 | 0.182 | RNA_PDE12 | 0.035 | 0.799 |
| RNA_ELK4 | 0.168 | 0.210 | RNA_DDX60L | −0.406 | 0.002 | RNA_WWP1 | 0.140 | 0.298 |
| RNA_MT-CYB | 0.158 | 0.241 | RNA_TAF1 | 0.024 | 0.861 | RNA_TCIRG1 | −0.121 | 0.370 |
| RNA_AEBP1 | 0.134 | 0.321 | RNA_SECISBP2L | −0.019 | 0.886 | RNA_HIVEP1 | −0.190 | 0.157 |
| RNA_AC008755.1 | 0.004 | 0.975 | RNA_DHX29 | 0.050 | 0.714 | RNA_SMAD2 | −0.060 | 0.655 |
| RNA_SFT2D2 | 0.022 | 0.873 | RNA_CSDE1 | −0.041 | 0.762 | RNA_WDR3 | −0.207 | 0.122 |
| RNA_FCHO2 | 0.143 | 0.290 | RNA_STAT3 | 0.013 | 0.924 | RNA_HK1 | 0.032 | 0.811 |
| RNA_BAZ2B | 0.050 | 0.714 | RNA_SYTL2 | 0.210 | 0.118 | RNA_PSAT1 | −0.125 | 0.353 |
| RNA_MT-CO1 | 0.119 | 0.379 | RNA_MAP3K20 | 0.192 | 0.152 | RNA_HDGFL3 | −0.024 | 0.861 |
| RNA_CLASP1 | 0.121 | 0.370 | RNA_LTBP2 | 0.114 | 0.397 | RNA_NUDT4P2 | 0.037 | 0.786 |
| RNA_MT-ND2 | 0.119 | 0.379 | RNA_CELA3B | 0.092 | 0.497 | RNA_ARHGEF3 | 0.166 | 0.216 |
| RNA_AGO3 | 0.000 | 1.000 | RNA_CD55 | 0.017 | 0.898 | RNA_STXBP3 | 0.151 | 0.262 |
| RNA_RC3H2 | −0.220 | 0.100 | RNA_FMNL2 | 0.134 | 0.321 | RNA_IGF2BP2 | −0.058 | 0.667 |
| RNA_MT-ATP6 | 0.022 | 0.873 | RNA_SPOCK1 | −0.119 | 0.379 | RNA_SCTR | 0.086 | 0.523 |
| RNA_RBMS2 | −0.089 | 0.512 | RNA_ZNF142 | 0.099 | 0.462 | RNA_CRTC3 | 0.218 | 0.103 |
| RNA_FNDC3B | −0.073 | 0.587 | RNA_AC241952.1 | 0.050 | 0.714 | RNA_F13A1 | 0.099 | 0.462 |
| RNA_MT-ND1 | 0.060 | 0.655 | RNA_PRSS1 | 0.125 | 0.353 | RNA_TCP11L2 | 0.024 | 0.861 |
| RNA_SNX27 | 0.214 | 0.110 | RNA_SEPT10 | −0.071 | 0.598 | RNA_APPL2 | 0.002 | 0.987 |
| RNA_ITGA1 | −0.013 | 0.924 | RNA_PPP2R5E | −0.132 | 0.329 | RNA_DAAM1 | −0.011 | 0.936 |
| RNA_AFF4 | −0.043 | 0.750 | RNA_TNFRSF1B | −0.009 | 0.949 | RNA_PSMD5 | −0.287 | 0.030 |
| RNA_ALS2 | −0.091 | 0.502 | RNA_UBE4B | −0.156 | 0.248 | RNA_PAG1 | 0.136 | 0.313 |
| RNA_COL1A1 | 0.043 | 0.750 | RNA_PIK3CA | 0.125 | 0.353 | RNA_NAP1L1 | 0.123 | 0.362 |
| RNA_DOCK5 | 0.160 | 0.235 | RNA_KLHL24 | 0.110 | 0.415 | RNA_ZBTB1 | −0.054 | 0.690 |
| RNA_RC3H1 | 0.333 | 0.011 | RNA_PRKAA1 | −0.006 | 0.962 | RNA_CBFB | 0.039 | 0.774 |
| RNA_EIF4G3 | −0.240 | 0.072 | RNA_UEVLD | −0.048 | 0.726 | RNA_FBXO2 | −0.317 | 0.016 |
| RNA_CDYL | −0.102 | 0.452 | RNA_YBX1 | −0.294 | 0.027 | RNA_ETF1 | −0.067 | 0.621 |
| RNA_SMG1P1 | −0.017 | 0.898 | RNA_CAPN2 | −0.013 | 0.924 | RNA_MST1R | 0.043 | 0.750 |
| RNA_POM121C | 0.019 | 0.886 | RNA_NPLOC4 | −0.056 | 0.678 | RNA_MTOR | −0.276 | 0.037 |
| RNA_AAK1 | −0.032 | 0.811 | RNA_PHC2 | −0.017 | 0.898 | RNA_STAG2 | −0.175 | 0.193 |
| RNA_TCF4 | 0.071 | 0.598 | RNA_EML4 | −0.082 | 0.544 | RNA_ITGA6 | 0.004 | 0.975 |
| RNA_AFF1 | 0.210 | 0.118 | RNA_DIS3 | −0.022 | 0.873 | RNA_DEPTOR | 0.112 | 0.406 |
| RNA_DCP2 | −0.156 | 0.248 | RNA_MAP2K4 | 0.156 | 0.248 | RNA_MSN | −0.048 | 0.726 |
| RNA_CDC42BPA | 0.004 | 0.975 | RNA_CBWD2 | −0.089 | 0.512 | RNA_DSG2 | −0.013 | 0.924 |
| RNA_MBNL1 | 0.032 | 0.811 | RNA_KRT19 | −0.201 | 0.134 | RNA_MEF2A | 0.130 | 0.337 |
| RNA_MAP3K2 | −0.009 | 0.949 | RNA_CLTC | 0.184 | 0.172 | RNA_ELF1 | 0.097 | 0.472 |
| RNA_NSD1 | −0.117 | 0.388 | RNA_POGK | 0.160 | 0.235 | RNA_SFMBT1 | 0.296 | 0.025 |
| RNA_DNAJB14 | 0.110 | 0.415 | RNA_QTRT2 | −0.151 | 0.262 | RNA_FKBP10 | −0.004 | 0.975 |
| RNA_MACF1 | −0.097 | 0.472 | RNA_FCHSD2 | 0.052 | 0.702 | RNA_FNTB | −0.015 | 0.911 |
| RNA_COL3A1 | −0.013 | 0.924 | RNA_PGD | −0.063 | 0.643 | RNA_OSBPL1A | 0.052 | 0.702 |
| RNA_MT-CO2 | 0.084 | 0.533 | RNA_SS18 | 0.130 | 0.337 | RNA_COLGALT1 | −0.136 | 0.313 |
| RNA_AGO2 | 0.069 | 0.609 | RNA_FTO | 0.173 | 0.199 | RNA_CCNI | 0.058 | 0.667 |
| RNA_HELZ | 0.130 | 0.337 | RNA_RBAK | 0.076 | 0.576 | RNA_NSUN5P1 | −0.058 | 0.667 |
| RNA_BBX | 0.065 | 0.632 | RNA_EGFR | 0.089 | 0.512 | RNA_MST1L | 0.102 | 0.452 |
| RNA_PRR14L | −0.002 | 0.987 | RNA_TEP1 | −0.011 | 0.936 | RNA_G3BP1 | 0.013 | 0.924 |
| RNA_ATF7IP | −0.233 | 0.081 | RNA_TAOK3 | −0.080 | 0.555 | RNA_PRNP | −0.063 | 0.643 |
| RNA_FAM114A1 | 0.013 | 0.924 | RNA_ACBD5 | 0.041 | 0.762 | RNA_INSR | 0.216 | 0.107 |
| RNA_CEP170 | −0.199 | 0.138 | RNA_UBR3 | 0.151 | 0.262 | RNA_CARNMT1 | 0.026 | 0.848 |
| RNA_REST | −0.205 | 0.126 | RNA_DNMT3A | −0.108 | 0.424 | RNA_PLAU | −0.184 | 0.172 |
| RNA_PTPN11 | −0.197 | 0.143 | RNA_DUSP16 | 0.082 | 0.544 | RNA_MGAT5 | 0.017 | 0.898 |
| RNA_AGAP4 | −0.009 | 0.949 | RNA_CAP1 | −0.089 | 0.512 | RNA_PTPN23 | −0.037 | 0.786 |
| RNA_RGPD5 | 0.037 | 0.786 | RNA_USP4 | 0.028 | 0.836 | RNA_TFPI | 0.013 | 0.924 |
| RNA_SMG1 | −0.006 | 0.962 | RNA_FNBP1 | −0.024 | 0.861 | RNA_SP2 | 0.067 | 0.621 |
| RNA_ARHGAP26 | 0.164 | 0.222 | RNA_CELA2B | 0.110 | 0.415 | RNA_VPS4B | −0.104 | 0.443 |
| RNA_TPM4 | −0.259 | 0.052 | RNA_WWTR1 | 0.158 | 0.241 | RNA_ALDH1A3 | −0.127 | 0.345 |
| RNA_SKAP2 | 0.233 | 0.081 | RNA_CTSK | −0.002 | 0.987 | RNA_BROX | 0.106 | 0.433 |
| RNA_FGD4 | 0.242 | 0.070 | RNA_CDK14 | 0.022 | 0.873 | RNA_DST | 0.048 | 0.726 |
| RNA_PRDM2 | 0.030 | 0.823 | RNA_RNF168 | −0.037 | 0.786 | RNA_SUCLG2 | 0.073 | 0.587 |
| RNA_SORL1 | 0.171 | 0.204 | RNA_PRUNE1 | 0.112 | 0.406 | RNA_CSGALNACT2 | −0.043 | 0.750 |
| RNA_QKI | 0.030 | 0.823 | RNA_ADAMTS9 | 0.028 | 0.836 | RNA_TRMT1L | −0.093 | 0.492 |
| RNA_DDI2 | −0.235 | 0.078 | RNA_NPIPB2 | 0.060 | 0.655 | RNA_CDC42EP3 | 0.315 | 0.017 |
| RNA_TRAK1 | 0.147 | 0.276 | RNA_RPS5 | 0.192 | 0.152 | RNA_FRMD4B | 0.097 | 0.472 |
| RNA_ANKRD12 | 0.013 | 0.924 | RNA_TNFAIP3 | −0.022 | 0.873 | RNA_MTRR | −0.173 | 0.199 |
| RNA_PLEKHA2 | −0.099 | 0.462 | RNA_CUZD1 | 0.054 | 0.690 | RNA_NAPG | −0.039 | 0.774 |
| RNA_CBLB | 0.006 | 0.962 | RNA_MYH10 | −0.039 | 0.774 | RNA_XPO1 | −0.028 | 0.836 |
| RNA_POM121 | 0.082 | 0.544 | RNA_DYNC1LI2 | 0.013 | 0.924 | RNA_DKK3 | 0.397 | 0.002 |
| RNA_LCOR | −0.173 | 0.199 | RNA_MMP2 | −0.084 | 0.533 | RNA_CARMIL1 | 0.168 | 0.210 |
| RNA_SP3 | 0.037 | 0.786 | RNA_ZNF431 | −0.058 | 0.667 | RNA_ANGEL2 | 0.063 | 0.643 |
| RNA_ANKRD36C | −0.024 | 0.861 | RNA_CAMK2D | 0.156 | 0.248 | RNA_BGN | 0.251 | 0.060 |
| RNA_SPECC1 | 0.032 | 0.811 | RNA_AC242843.1 | 0.261 | 0.050 | RNA_MTPAP | 0.147 | 0.276 |
| RNA_ARHGEF12 | 0.045 | 0.738 | RNA_RBM33 | −0.102 | 0.452 | RNA_ACLY | −0.058 | 0.667 |
| RNA_CHD2 | −0.199 | 0.138 | RNA_NUP58 | 0.026 | 0.848 | RNA_PKP4 | 0.037 | 0.786 |
| RNA_ASAP2 | −0.060 | 0.655 | RNA_EPB41 | −0.117 | 0.388 | RNA_ITSN1 | 0.158 | 0.241 |
| RNA_GON4L | 0.356 | 0.007 | RNA_SMC5 | 0.173 | 0.199 | RNA_AKR7A3 | 0.000 | 1.000 |
| RNA_PTBP3 | −0.050 | 0.714 | RNA_SMG1P5 | 0.102 | 0.452 | RNA_YTHDC1 | 0.022 | 0.873 |
| RNA_ZC3H13 | 0.076 | 0.576 | RNA_TRRAP | 0.063 | 0.643 | RNA_MXRA8 | 0.099 | 0.462 |
| RNA_MT-ND4L | 0.108 | 0.424 | RNA_BABAM2 | −0.039 | 0.774 | RNA_CLIP1 | −0.173 | 0.199 |
| RNA_PRKAR1A | 0.045 | 0.738 | RNA_YAP1 | −0.093 | 0.492 | RNA_GJB1 | 0.192 | 0.152 |
| RNA_SPINK1 | 0.015 | 0.911 | RNA_ARHGEF7 | 0.151 | 0.262 | RNA_STX7 | 0.037 | 0.786 |
| RNA_STRN3 | −0.175 | 0.193 | RNA_KLF5 | 0.013 | 0.924 | RNA_TLE3 | −0.311 | 0.019 |
| RNA_COL1A2 | 0.015 | 0.911 | RNA_STOM | −0.054 | 0.690 | RNA_ABCC1 | 0.108 | 0.424 |
| RNA_MT-ATP8 | 0.073 | 0.587 | RNA_NPEPPS | −0.095 | 0.482 | RNA_NABP1 | −0.138 | 0.305 |
| RNA_ATP13A3 | −0.028 | 0.836 | RNA_MTR | 0.190 | 0.157 | RNA_ANKRD28 | −0.056 | 0.678 |
| RNA_SPARC | 0.093 | 0.492 | RNA_MYL6 | −0.341 | 0.009 | RNA_NR3C2 | 0.218 | 0.103 |
| RNA_AHR | 0.175 | 0.193 | RNA_PRICKLE2 | 0.138 | 0.305 | RNA_DCAF5 | 0.024 | 0.861 |
| RNA_CFLAR | −0.177 | 0.188 | RNA_SYNJ1 | −0.082 | 0.544 | RNA_MYO18A | −0.205 | 0.126 |
| RNA_ZNF609 | −0.147 | 0.276 | RNA_SMC4 | −0.313 | 0.018 | RNA_HNRNPR | −0.235 | 0.078 |
| RNA_MED13 | 0.050 | 0.714 | RNA_MDC1 | −0.022 | 0.873 | RNA_LOXL2 | −0.112 | 0.406 |
| RNA_KIF1B | −0.028 | 0.836 | RNA_P4HB | −0.067 | 0.621 | RNA_APOE | −0.039 | 0.774 |
| RNA_VCL | −0.112 | 0.406 | RNA_LUM | 0.263 | 0.048 | RNA_UBE4A | 0.017 | 0.898 |
| RNA_APBB2 | 0.121 | 0.370 | RNA_C1GALT1 | 0.238 | 0.075 | RNA_WWC2 | −0.246 | 0.065 |
| RNA_LRRFIP1 | −0.032 | 0.811 | RNA_TRIM25 | −0.272 | 0.041 | RNA_DCUN1D1 | 0.000 | 1.000 |
| RNA_PRSS2 | 0.177 | 0.188 | RNA_BTRC | −0.162 | 0.229 | RNA_MORF4L1 | 0.000 | 1.000 |
| RNA_EPB41L2 | −0.058 | 0.667 | RNA_SYNRG | −0.149 | 0.269 | RNA_CEP89 | −0.035 | 0.799 |
| RNA_NAV1 | −0.181 | 0.177 | RNA_SYNJ2 | −0.097 | 0.472 | RNA_RPS6KB1 | −0.156 | 0.248 |
| RNA_SERINC5 | 0.352 | 0.007 | RNA_WDR82 | 0.089 | 0.512 | RNA_CKLF | 0.006 | 0.962 |
| RNA_ARNT | 0.261 | 0.050 | RNA_PTPN12 | 0.050 | 0.714 | RNA_UBE2K | −0.214 | 0.110 |
| RNA_PIKFYVE | 0.114 | 0.397 | RNA_NCOA7 | 0.002 | 0.987 | RNA_DCN | 0.348 | 0.008 |
| RNA_MICAL2 | 0.011 | 0.936 | RNA_TP53BP2 | 0.017 | 0.898 | RNA_NUP43 | 0.052 | 0.702 |
| RNA_FNIP1 | 0.009 | 0.949 | RNA_FSTL1 | 0.084 | 0.533 | RNA_TAF2 | −0.114 | 0.397 |
| RNA_RASA1 | −0.058 | 0.667 | RNA_TMSB10 | −0.171 | 0.204 | RNA_ZC3H18 | −0.024 | 0.861 |
| RNA_PUM2 | −0.073 | 0.587 | RNA_KCTD10 | −0.086 | 0.523 | RNA_RAB3GAP1 | 0.156 | 0.248 |
| RNA_ANKRD36 | 0.032 | 0.811 | RNA_TIMM23B | −0.073 | 0.587 | RNA_CLPTM1L | −0.160 | 0.235 |
| RNA_ZNF148 | −0.063 | 0.643 | RNA_CNN2 | −0.048 | 0.726 | RNA_EPM2AIP1 | −0.065 | 0.632 |
| RNA_FOXP1 | 0.140 | 0.298 | RNA_ADGRE5 | −0.071 | 0.598 | RNA_ELAVL1 | −0.188 | 0.162 |
| RNA_BIRC6 | −0.084 | 0.533 | RNA_PKD2 | 0.244 | 0.067 | RNA_INVS | 0.028 | 0.836 |
| RNA_PPP1CB | 0.069 | 0.609 | RNA_AC139256.1 | −0.013 | 0.924 | RNA_PARG | −0.179 | 0.182 |
| RNA_ZC3H11A | 0.173 | 0.199 | RNA_DOCK9 | 0.156 | 0.248 | RNA_KAT7 | 0.104 | 0.443 |
| RNA_ADAR | −0.026 | 0.848 | RNA_PKM | −0.186 | 0.167 | RNA_MAP3K4 | −0.136 | 0.313 |
| RNA_XRN1 | −0.235 | 0.078 | RNA_TMEM248 | 0.238 | 0.075 | RNA_ZNF346 | 0.164 | 0.222 |
| RNA_MT-ND6 | 0.235 | 0.078 | RNA_TRAF3IP1 | −0.006 | 0.962 | RNA_PLXDC1 | 0.095 | 0.482 |
| RNA_FRYL | −0.048 | 0.726 | RNA_ZCCHC4 | −0.043 | 0.750 | RNA_EID1 | 0.013 | 0.924 |
| RNA_TNS1 | 0.173 | 0.199 | RNA_KIAA2026 | −0.041 | 0.762 | RNA_SDCCAG8 | 0.030 | 0.823 |
| RNA_UBAP2L | 0.136 | 0.313 | RNA_SNX9 | −0.006 | 0.962 | RNA_CFAP97 | −0.121 | 0.370 |
| RNA_WIPF1 | 0.024 | 0.861 | RNA_KIF16B | 0.233 | 0.081 | RNA_SF3B3 | −0.192 | 0.152 |
| RNA_TBC1D14 | 0.067 | 0.621 | RNA_EPHA2 | −0.153 | 0.255 | RNA_MAL2 | −0.093 | 0.492 |
| RNA_MARCH7 | 0.058 | 0.667 | RNA_FOXN3 | 0.151 | 0.262 | RNA_USP34 | −0.082 | 0.544 |
| RNA_TRIO | −0.065 | 0.632 | RNA_RNF217 | 0.002 | 0.987 | RNA_GNS | 0.121 | 0.370 |
| RNA_PBRM1 | 0.076 | 0.576 | RNA_NEK7 | −0.019 | 0.886 | RNA_ZNF117 | −0.022 | 0.873 |
| RNA_NPIPB4 | 0.041 | 0.762 | RNA_DPY19L1 | 0.143 | 0.290 | RNA_NCOR2 | 0.119 | 0.379 |
| RNA_ZMYM4 | −0.069 | 0.609 | RNA_SNED1 | 0.175 | 0.193 | RNA_LIPH | 0.026 | 0.848 |
| RNA_COL16A1 | 0.065 | 0.632 | RNA_OSMR | −0.257 | 0.054 | RNA_M6PR | −0.121 | 0.370 |
| RNA_AP2B1 | −0.285 | 0.032 | RNA_WDR37 | −0.080 | 0.555 | RNA_AIDA | −0.095 | 0.482 |
| RNA_S100A6 | 0.112 | 0.406 | RNA_ESF1 | −0.032 | 0.811 | RNA_ZNF638 | 0.028 | 0.836 |
| RNA_TYW1 | 0.171 | 0.204 | RNA_TUBA1C | −0.253 | 0.058 | RNA_USP45 | −0.032 | 0.811 |
| RNA_ELK3 | −0.194 | 0.147 | RNA_CTRB2 | 0.108 | 0.424 | RNA_PGGT1B | −0.052 | 0.702 |
| RNA_ZNF827 | 0.123 | 0.362 | RNA_ATF7 | 0.233 | 0.081 | RNA_LSM14A | −0.132 | 0.329 |
| RNA_ABL2 | 0.058 | 0.667 | RNA_AKAP12 | 0.104 | 0.443 | RNA_RNF212 | 0.071 | 0.598 |
| RNA_TRIP12 | −0.084 | 0.533 | RNA_FBXO32 | 0.073 | 0.587 | RNA_GAPVD1 | −0.004 | 0.975 |
| RNA_CLPS | 0.186 | 0.166 | RNA_TPST1 | −0.028 | 0.836 | RNA_ARPC5 | −0.006 | 0.962 |
| RNA_THBS2 | 0.045 | 0.738 | RNA_CRLF3 | −0.043 | 0.750 | RNA_MAPKAPK2 | −0.028 | 0.836 |
| RNA_ATP6V1A | −0.166 | 0.216 | RNA_FEM1B | 0.043 | 0.750 | RNA_SULF2 | 0.041 | 0.762 |
| RNA_ZFYVE16 | −0.093 | 0.492 | RNA_TGOLN2 | 0.199 | 0.138 | RNA_WASH3P | 0.026 | 0.848 |
| RNA_ARAP2 | −0.190 | 0.157 | RNA_NFE2L1 | −0.071 | 0.598 | RNA_ZNF841 | −0.017 | 0.898 |
| RNA_MAP4 | 0.179 | 0.182 | RNA_CTDSPL2 | −0.039 | 0.774 | RNA_FANCC | 0.257 | 0.054 |
| RNA_AGAP1 | 0.240 | 0.072 | RNA_GOLGA8N | 0.123 | 0.362 | RNA_ZNF782 | 0.017 | 0.898 |
| RNA_HIPK1 | −0.017 | 0.898 | RNA_ITGB5 | 0.117 | 0.388 | RNA_ACTR3C | 0.002 | 0.987 |
| RNA_SPTBN1 | 0.248 | 0.062 | RNA_SLC43A1 | 0.175 | 0.193 | RNA_PPP1R15A | −0.019 | 0.886 |
| RNA_ATAD2B | 0.015 | 0.911 | RNA_RNF19A | −0.082 | 0.544 | RNA_XPC | 0.104 | 0.443 |
| RNA_ABHD2 | 0.367 | 0.005 | RNA_SELENOI | −0.147 | 0.276 | RNA_USP53 | 0.162 | 0.229 |
| RNA_SCAF8 | −0.011 | 0.936 | RNA_ATP2B4 | −0.082 | 0.544 | RNA_ATP6V0A2 | 0.104 | 0.443 |
| RNA_LATS1 | 0.054 | 0.690 | RNA_ST6GAL1 | 0.093 | 0.492 | RNA_BRD1 | 0.186 | 0.167 |
| RNA_RO60 | −0.030 | 0.823 | RNA_TMEM87B | −0.035 | 0.799 | RNA_NET1 | −0.093 | 0.492 |
| RNA_SLC35E2A | 0.022 | 0.873 | RNA_DYNC1I2 | 0.093 | 0.492 | RNA_YWHAE | −0.050 | 0.714 |
| RNA_MED13L | 0.041 | 0.762 | RNA_ZCCHC2 | −0.117 | 0.388 | RNA_SSX2IP | 0.045 | 0.738 |
| RNA_PPP3CA | −0.019 | 0.886 | RNA_IL22RA1 | −0.136 | 0.313 | RNA_SMAP2 | 0.156 | 0.248 |
| RNA_MAP4K3 | 0.024 | 0.861 | RNA_YLPM1 | −0.082 | 0.544 | RNA_ATE1 | 0.045 | 0.738 |
| RNA_SPOPL | −0.091 | 0.502 | RNA_NDRG1 | 0.060 | 0.655 | RNA_MNT | 0.132 | 0.329 |
| RNA_ATXN7 | 0.028 | 0.836 | RNA_CAST | −0.024 | 0.861 | RNA_MDM2 | 0.039 | 0.774 |
| RNA_ASAP1 | −0.084 | 0.533 | RNA_NMNAT3 | 0.168 | 0.210 | RNA_DENND4B | 0.093 | 0.492 |
| RNA_PPP1R12B | 0.201 | 0.134 | RNA_KMT2E | 0.082 | 0.544 | RNA_LYN | −0.076 | 0.576 |
| RNA_SLC39A5 | 0.039 | 0.774 | RNA_CPB1 | 0.117 | 0.388 | RNA_WASHC4 | −0.056 | 0.678 |
| RNA_MT-ND5 | 0.145 | 0.283 | RNA_CNST | 0.076 | 0.576 | RNA_FYCO1 | 0.233 | 0.081 |
| RNA_WASH5P | −0.091 | 0.502 | RNA_FAM185A | 0.099 | 0.462 | RNA_SETD5 | −0.002 | 0.987 |
| RNA_DCP1A | 0.043 | 0.750 | RNA_HIST2H2AA3 | −0.184 | 0.172 | RNA_DDX19A | 0.045 | 0.738 |
| RNA_EHMT1 | −0.132 | 0.329 | RNA_SLC2A3 | −0.030 | 0.823 | RNA_NOTCH3 | −0.026 | 0.848 |
| RNA_ARID1B | 0.065 | 0.632 | RNA_LIMCH1 | −0.037 | 0.786 | RNA_PLD1 | 0.125 | 0.353 |
| RNA_HERC3 | −0.089 | 0.512 | RNA_SEC24B | 0.123 | 0.362 | RNA_SLC39A10 | −0.060 | 0.655 |
| RNA_DIP2C | −0.045 | 0.738 | RNA_ADAMTSL4 | 0.028 | 0.836 | RNA_SMARCA2 | 0.119 | 0.379 |
| RNA_RBMS1 | −0.045 | 0.738 | RNA_HERC4 | −0.121 | 0.370 | RNA_S100A10 | −0.089 | 0.512 |
| RNA_PALLD | 0.151 | 0.262 | RNA_NPAT | −0.108 | 0.424 | RNA_TINAGL1 | 0.024 | 0.861 |
| RNA_ENAH | 0.043 | 0.750 | RNA_CDK6 | 0.017 | 0.898 | RNA_SENP2 | −0.140 | 0.298 |
| RNA_PANK3 | 0.024 | 0.861 | RNA_RCOR1 | −0.166 | 0.216 | RNA_MTF2 | −0.220 | 0.100 |
| RNA_ABLIM1 | 0.175 | 0.193 | RNA_NSRP1 | 0.058 | 0.667 | RNA_RCAN3 | −0.054 | 0.690 |
| RNA_DPYSL2 | 0.166 | 0.216 | RNA_RNA5SP389 | −0.402 | 0.002 | RNA_SETD2 | −0.030 | 0.823 |
| RNA_NSD2 | −0.361 | 0.006 | RNA_DNM1L | 0.002 | 0.987 | RNA_GUCY1A1 | 0.203 | 0.130 |
| RNA_RPRD2 | 0.203 | 0.130 | RNA_MFSD6 | 0.071 | 0.598 | RNA_RNF38 | 0.203 | 0.130 |
| RNA_CCNT1 | 0.056 | 0.678 | RNA_FBXW2 | −0.149 | 0.269 | RNA_CDC27 | 0.002 | 0.987 |
| RNA_FN1 | −0.019 | 0.886 | RNA_PTPN4 | 0.043 | 0.750 | RNA_KIRREL2 | 0.095 | 0.482 |
| RNA_STAG1 | 0.117 | 0.388 | RNA_CNOT2 | −0.127 | 0.345 | RNA_PDS5B | 0.073 | 0.587 |
| RNA_CHD6 | 0.009 | 0.949 | RNA_WAPL | −0.073 | 0.587 | RNA_DTX4 | −0.104 | 0.443 |
| RNA_ARID4B | −0.032 | 0.811 | RNA_ARSB | −0.045 | 0.738 | RNA_LINC01145 | −0.261 | 0.050 |
| RNA_PSD3 | 0.177 | 0.188 | RNA_USP15 | −0.099 | 0.462 | RNA_ARHGAP1 | 0.041 | 0.762 |
| RNA_ASH1L | 0.162 | 0.229 | RNA_SHOC2 | −0.071 | 0.598 | RNA_BRD3 | −0.041 | 0.762 |
| RNA_NFIA | 0.315 | 0.017 | RNA_SOS1 | 0.041 | 0.762 | RNA_ATP2B1 | −0.056 | 0.678 |
| RNA_AC124319.1 | −0.240 | 0.072 | RNA_TMEM135 | −0.060 | 0.655 | RNA_CCND1 | −0.022 | 0.873 |
| RNA_ROCK2 | 0.173 | 0.199 | RNA_ARPC2 | −0.058 | 0.667 | RNA_PDP1 | 0.110 | 0.415 |
| RNA_WDFY3 | 0.060 | 0.655 | RNA_ZBTB43 | −0.158 | 0.241 | RNA_EZR | −0.153 | 0.255 |
| RNA_WDFY1 | −0.076 | 0.576 | RNA_DYRK1A | 0.050 | 0.714 | RNA_SLC25A30 | 0.084 | 0.533 |
| RNA_NDUFS1 | 0.065 | 0.632 | RNA_ARHGAP32 | −0.045 | 0.738 | RNA_GABRP | −0.071 | 0.598 |
| RNA_WASHC2C | −0.058 | 0.667 | RNA_ACTR3 | −0.231 | 0.084 | RNA_CBWD1 | −0.060 | 0.655 |
| RNA_BRD4 | −0.145 | 0.283 | RNA_FAM49B | −0.298 | 0.024 | RNA_UBE3C | −0.201 | 0.134 |
| RNA_DENND1A | −0.080 | 0.555 | RNA_RGS5 | 0.406 | 0.002 | RNA_METTL2A | 0.045 | 0.738 |
| RNA_ATP11B | 0.153 | 0.255 | RNA_CLCN6 | 0.091 | 0.502 | RNA_RPL34 | 0.123 | 0.362 |
| RNA_RUNX1 | −0.041 | 0.762 | RNA_PIK3C2A | 0.089 | 0.512 | RNA_SDCBP | 0.231 | 0.084 |
| RNA_MYSM1 | 0.069 | 0.609 | RNA_MAPK1IP1L | −0.171 | 0.204 | RNA_SLC25A51 | 0.065 | 0.632 |
| RNA_ANKRD36B | 0.091 | 0.502 | RNA_POLK | 0.130 | 0.337 | RNA_YAF2 | −0.060 | 0.655 |
| RNA_NAA15 | 0.011 | 0.936 | RNA_PPP4R1 | −0.009 | 0.949 | RNA_TOMM7 | 0.272 | 0.041 |
| RNA_WDR26 | −0.104 | 0.443 | RNA_ZNF83 | 0.048 | 0.726 | RNA_CLIP4 | −0.136 | 0.313 |
| RNA_HEG1 | −0.052 | 0.702 | RNA_PRRC2B | 0.069 | 0.609 | RNA_PPARA | 0.199 | 0.138 |
| RNA_ERCC6 | −0.156 | 0.248 | RNA_SETD7 | 0.132 | 0.329 | RNA_FOXK2 | 0.179 | 0.182 |
| RNA_EPS15 | 0.138 | 0.305 | RNA_SLC7A6 | 0.004 | 0.975 | RNA_DDX42 | 0.043 | 0.750 |
| RNA_DENND6A | 0.024 | 0.861 | RNA_SMCHD1 | 0.093 | 0.492 | RNA_PHGDH | 0.013 | 0.924 |
| RNA_TCF7L2 | −0.093 | 0.492 | RNA_DNAJB6 | −0.048 | 0.726 | RNA_SLC38A1 | −0.009 | 0.949 |
| RNA_ANKRD11 | −0.108 | 0.424 | RNA_ADAM10 | −0.147 | 0.276 | RNA_SGK3 | −0.004 | 0.975 |
| RNA_ALB | −0.065 | 0.632 | RNA_CTRB1 | 0.123 | 0.362 | RNA_STARD4 | 0.028 | 0.836 |
| RNA_PARP8 | −0.071 | 0.598 | RNA_C1orf198 | 0.173 | 0.199 | RNA_VEZF1 | 0.006 | 0.962 |
| RNA_BMP2K | 0.104 | 0.443 | RNA_SEC61A2 | −0.112 | 0.406 | RNA_ATAD3B | −0.160 | 0.235 |
| RNA_FAM172A | 0.058 | 0.667 | RNA_EIF4G2 | −0.218 | 0.103 | RNA_APLP2 | 0.089 | 0.512 |
| RNA_OSBPL3 | 0.035 | 0.799 | RNA_ATP11A | 0.011 | 0.936 | RNA_DCUN1D4 | 0.050 | 0.714 |
| RNA_ZNF532 | −0.151 | 0.262 | RNA_RALGAPA2 | 0.145 | 0.283 | RNA_AC026470.1 | −0.216 | 0.107 |
| RNA_SMARCC1 | −0.019 | 0.886 | RNA_ADAM17 | −0.013 | 0.924 | RNA_TNFRSF21 | −0.056 | 0.678 |
| RNA_SMG7 | −0.004 | 0.975 | RNA_DNMT1 | −0.231 | 0.084 | RNA_C6orf106 | −0.263 | 0.048 |
| RNA_CBS | 0.084 | 0.533 | RNA_EPS8 | 0.171 | 0.204 | RNA_LRRC8B | −0.080 | 0.555 |
| RNA_ZNF621 | 0.071 | 0.598 | RNA_FKBP9 | 0.238 | 0.075 | RNA_KLHL5 | −0.233 | 0.081 |
| RNA_WDR36 | −0.017 | 0.898 | RNA_CFH | 0.229 | 0.087 | RNA_ATP1B1 | 0.084 | 0.533 |
| RNA_ITPR2 | −0.013 | 0.924 | RNA_AGAP9 | −0.106 | 0.433 | RNA_GM2A | 0.002 | 0.987 |
| RNA_APPBP2 | 0.274 | 0.039 | RNA_CCDC93 | 0.052 | 0.702 | RNA_INPP5B | 0.045 | 0.738 |
| RNA_KAT6A | 0.063 | 0.643 | RNA_ETNK1 | 0.073 | 0.587 | RNA_CTSC | 0.041 | 0.762 |
| RNA_ANO1 | −0.089 | 0.512 | RNA_RAB10 | −0.162 | 0.229 | RNA_MAP4K5 | 0.037 | 0.786 |
| RNA_TEAD1 | −0.205 | 0.126 | RNA_IQGAP1 | −0.045 | 0.738 | RNA_CXCR4 | 0.153 | 0.255 |
| RNA_SNIP1 | −0.086 | 0.523 | RNA_ERCC4 | 0.099 | 0.462 | RNA_FUT10 | −0.019 | 0.886 |
| RNA_MIER3 | 0.022 | 0.873 | RNA_TRAPPC11 | −0.067 | 0.621 | RNA_ARHGAP35 | 0.024 | 0.861 |
| RNA_PRKAR2A | −0.097 | 0.472 | RNA_MAGI1 | 0.153 | 0.255 | RNA_ZNF608 | −0.048 | 0.726 |
| RNA_TACC1 | 0.201 | 0.134 | RNA_KMT2D | −0.091 | 0.502 | RNA_PAFAH1B2 | −0.151 | 0.262 |
| RNA_LNPEP | 0.138 | 0.305 | RNA_CTIF | 0.235 | 0.078 | RNA_NAMPT | −0.065 | 0.632 |
| RNA_AHNAK | 0.043 | 0.750 | RNA_KCTD9 | 0.145 | 0.283 | RNA_PRDX4 | 0.000 | 1.000 |
| RNA_TUBA1B | −0.102 | 0.452 | RNA_ZNF586 | 0.000 | 1.000 | RNA_EXOSC10 | −0.017 | 0.898 |
| RNA_KPNA6 | 0.006 | 0.962 | RNA_XAF1 | −0.361 | 0.006 | RNA_ZNF518A | −0.084 | 0.533 |
| RNA_ZKSCAN1 | 0.225 | 0.093 | RNA_TIA1 | −0.006 | 0.962 | RNA_MEX3C | −0.117 | 0.388 |
| RNA_CNOT6 | −0.177 | 0.188 | RNA_CASP2 | −0.156 | 0.248 | RNA_HIST4H4 | −0.052 | 0.702 |
| RNA_RAI14 | −0.235 | 0.078 | RNA_RBFOX2 | −0.117 | 0.388 | RNA_RACK1 | −0.006 | 0.962 |
| RNA_NPIPB13 | −0.153 | 0.255 | RNA_ANKRD10 | −0.022 | 0.873 | RNA_SP110 | −0.186 | 0.167 |
| RNA_EYA3 | −0.205 | 0.126 | RNA_KIF2A | −0.143 | 0.290 | RNA_BBS9 | 0.121 | 0.370 |
| RNA_GOLGA6L5P | −0.125 | 0.353 | RNA_SRPK2 | 0.028 | 0.836 | RNA_CLIC4 | −0.030 | 0.823 |
| RNA_FAM120A | 0.179 | 0.182 | RNA_GATAD2A | −0.110 | 0.415 | RNA_GNPTAB | 0.210 | 0.118 |
| RNA_EFCAB14 | 0.244 | 0.067 | RNA_STAM2 | 0.104 | 0.443 | RNA_MCM9 | −0.052 | 0.702 |
| RNA_NPIPB5 | 0.056 | 0.678 | RNA_DDX46 | −0.156 | 0.248 | RNA_ZNF516 | 0.069 | 0.609 |
| RNA_SEC14L1 | −0.175 | 0.193 | RNA_UGGT1 | −0.024 | 0.861 | RNA_SPTAN1 | 0.110 | 0.415 |
| RNA_RAB31 | 0.071 | 0.598 | RNA_TMEM123 | −0.028 | 0.836 | RNA_WDR48 | 0.004 | 0.975 |
| RNA_DCBLD2 | −0.179 | 0.182 | RNA_SLC10A7 | −0.177 | 0.188 | RNA_TAB2 | −0.190 | 0.157 |
| RNA_NFIB | 0.136 | 0.313 | RNA_YTHDF2 | −0.168 | 0.210 | RNA_ISG20L2 | 0.119 | 0.379 |
| RNA_YY1AP1 | 0.382 | 0.003 | RNA_EPB41L3 | 0.013 | 0.924 | RNA_TIAM2 | 0.089 | 0.512 |
| RNA_SH3D19 | 0.300 | 0.023 | RNA_NEMF | −0.039 | 0.774 | RNA_ZC3H12A | 0.035 | 0.799 |
| RNA_ARHGEF2 | −0.048 | 0.726 | RNA_GLIS3 | −0.022 | 0.873 | RNA_RPAP2 | −0.002 | 0.987 |
| RNA_COL6A3 | −0.022 | 0.873 | RNA_ZKSCAN8 | 0.004 | 0.975 | RNA_PTF1A | 0.026 | 0.847 |
| RNA_CELA3A | 0.158 | 0.241 | RNA_ANKHD1 | −0.065 | 0.632 | RNA_CDC73 | 0.197 | 0.143 |
| RNA_WNK1 | −0.026 | 0.848 | RNA_RBSN | 0.069 | 0.609 | RNA_NBN | −0.058 | 0.667 |
| RNA_AQP12B | 0.066 | 0.626 | RNA_DNAJC13 | −0.188 | 0.162 | RNA_RIT1 | 0.108 | 0.424 |
| RNA_NAA25 | −0.114 | 0.397 | RNA_GOLGA2P7 | −0.130 | 0.337 | RNA_HERC6 | −0.233 | 0.081 |
| RNA_SHPRH | −0.032 | 0.811 | RNA_STX12 | 0.071 | 0.598 | RNA_CCDC80 | 0.095 | 0.482 |
| RNA_PDLIM5 | 0.140 | 0.298 | RNA_ARHGEF11 | 0.330 | 0.012 | RNA_USP10 | −0.266 | 0.046 |
| RNA_RAB3GAP2 | −0.153 | 0.255 | RNA_TRIP11 | 0.043 | 0.750 | RNA_SIK3 | −0.158 | 0.241 |
| RNA_FBXO28 | −0.028 | 0.836 | RNA_ZFAND4 | 0.037 | 0.786 | RNA_CDC5L | 0.035 | 0.799 |
| RNA_BZW1 | −0.184 | 0.172 | RNA_KDM1B | −0.266 | 0.046 | RNA_FAR1 | 0.184 | 0.172 |
| RNA_FAM120B | −0.002 | 0.987 | RNA_PAN3 | 0.043 | 0.750 | RNA_NCOA4 | 0.117 | 0.388 |
| RNA_BCLAF1 | −0.054 | 0.690 | RNA_RND3 | −0.015 | 0.911 | RNA_DCAF6 | 0.251 | 0.060 |
| RNA_TMEM125 | −0.117 | 0.388 | RNA_RNF19B | −0.218 | 0.103 | RNA_LSP1 | −0.056 | 0.678 |
| RNA_COPA | 0.240 | 0.072 | RNA_CDK2AP2 | −0.181 | 0.177 | RNA_ZFR | 0.041 | 0.762 |
| RNA_DIXDC1 | 0.006 | 0.962 | RNA_PDE5A | 0.156 | 0.248 | RNA_CBR4 | −0.017 | 0.898 |
| RNA_RPS12 | −0.039 | 0.774 | RNA_NMT1 | −0.086 | 0.523 | RNA_ARF3 | −0.097 | 0.472 |
| RNA_ASPH | −0.065 | 0.632 | RNA_CAMSAP1 | 0.177 | 0.188 | RNA_OXR1 | 0.233 | 0.081 |
| RNA_SESTD1 | 0.242 | 0.070 | RNA_PSIP1 | −0.065 | 0.632 | RNA_TTC33 | −0.164 | 0.222 |
| RNA_RBPJL | 0.082 | 0.544 | RNA_ANKH | 0.080 | 0.555 | RNA_NBPF19 | 0.110 | 0.415 |
| RNA_LTBP1 | 0.108 | 0.424 | RNA_PWWP2A | −0.084 | 0.533 | RNA_ZMYM2 | −0.011 | 0.936 |
| RNA_ARID2 | −0.143 | 0.290 | RNA_ATP9B | −0.097 | 0.472 | RNA_MALT1 | 0.019 | 0.886 |
| RNA_PATJ | 0.048 | 0.726 | RNA_TUBA1A | −0.002 | 0.987 | RNA_DBF4 | −0.119 | 0.379 |
| RNA_KIF13B | 0.216 | 0.107 | RNA_ROCK1 | 0.233 | 0.081 | RNA_AHI1 | −0.006 | 0.962 |
| RNA_AKAP2 | −0.225 | 0.093 | RNA_COX7C | 0.197 | 0.143 | RNA_PLCD3 | 0.017 | 0.898 |
| RNA_SEPT11 | 0.227 | 0.090 | RNA_EXOC5 | −0.052 | 0.702 | RNA_KALRN | 0.197 | 0.143 |
| RNA_ZEB2 | −0.110 | 0.415 | RNA_MXD1 | −0.117 | 0.388 | RNA_CCDC186 | −0.130 | 0.337 |
| RNA_MYO10 | 0.110 | 0.415 | RNA_NCBP1 | 0.050 | 0.714 | RNA_MX2 | −0.210 | 0.118 |
| RNA_BACH1 | −0.285 | 0.032 | RNA_PAFAH1B1 | 0.134 | 0.321 | RNA_MYCBP2 | −0.002 | 0.987 |
| RNA_PCNX2 | 0.069 | 0.609 | RNA_JMJD1C | 0.119 | 0.379 | RNA_NPIPB14P | 0.145 | 0.283 |
| RNA_ERBIN | −0.080 | 0.555 | RNA_VPS8 | −0.009 | 0.949 | RNA_GNB1 | −0.078 | 0.565 |
| RNA_EIF2AK2 | −0.276 | 0.037 | RNA_ERAP1 | −0.054 | 0.690 | RNA_ANXA2 | −0.039 | 0.774 |
| RNA_MT-ND3 | 0.067 | 0.621 | RNA_FAM126A | −0.032 | 0.811 | RNA_RAD50 | −0.106 | 0.433 |
| RNA_MGA | −0.084 | 0.533 | RNA_EVC | 0.127 | 0.345 | RNA_SRM | −0.056 | 0.678 |
| RNA_RAPGEF2 | −0.220 | 0.100 | RNA_SMC6 | −0.268 | 0.044 | RNA_ITPR1 | 0.110 | 0.415 |
| RNA_CBWD3 | −0.147 | 0.276 | RNA_AFDN | 0.009 | 0.949 | RNA_MAD1L1 | −0.248 | 0.062 |
| RNA_MYOF | −0.231 | 0.084 | RNA_DMXL1 | 0.011 | 0.936 | RNA_ZDHHC20 | −0.037 | 0.786 |
| RNA_CPA2 | 0.134 | 0.321 | RNA_PJA2 | −0.039 | 0.774 | RNA_CLEC2D | 0.076 | 0.576 |
| RNA_ATF2 | 0.048 | 0.726 | RNA_FAM91A1 | −0.017 | 0.898 | RNA_LYPLA1 | −0.192 | 0.152 |
| RNA_PEAK1 | 0.006 | 0.962 | RNA_EPB41L4B | −0.076 | 0.576 | RNA_CASK | −0.112 | 0.406 |
| RNA_YTHDF3 | 0.073 | 0.587 | RNA_DAB2IP | −0.140 | 0.298 | RNA_PLS1 | 0.138 | 0.305 |
| RNA_MTDH | −0.106 | 0.433 | RNA_SLC25A12 | 0.056 | 0.678 | RNA_UBE2Z | −0.285 | 0.032 |
| RNA_WAC | −0.048 | 0.726 | RNA_BCAR3 | −0.205 | 0.126 | RNA_SMARCC2 | 0.127 | 0.345 |
| RNA_DOCK7 | 0.158 | 0.241 | RNA_PLIN5 | −0.156 | 0.248 | RNA_PARD6B | −0.043 | 0.750 |
| RNA_TLK2 | −0.071 | 0.598 | RNA_CDK12 | −0.125 | 0.353 | RNA_SHLD2 | −0.043 | 0.750 |
| RNA_ZFYVE26 | −0.099 | 0.462 | RNA_TAF1B | −0.106 | 0.433 | RNA_KLHL2 | −0.024 | 0.861 |
| RNA_SEC23A | −0.011 | 0.936 | RNA_RAB8A | −0.324 | 0.014 | RNA_CYP20A1 | 0.274 | 0.039 |
| RNA_SEPT9 | 0.076 | 0.576 | RNA_TBC1D9 | −0.080 | 0.555 | RNA_CELF1 | −0.188 | 0.162 |
| RNA_USP46 | 0.080 | 0.555 | RNA_RAB28 | −0.099 | 0.462 | RNA_SRPK1 | −0.166 | 0.216 |
| RNA_ATXN1 | −0.024 | 0.861 | RNA_PPMIB | 0.086 | 0.523 | RNA_PPIP5K1 | 0.011 | 0.936 |
| RNA_ACTR2 | 0.024 | 0.861 | RNA_VPS45 | 0.259 | 0.052 | RNA_TAGLN | −0.125 | 0.353 |
| RNA_ZBTB38 | −0.123 | 0.362 | RNA_JAG1 | 0.229 | 0.087 | RNA_CLCN3 | 0.002 | 0.987 |
| RNA_COQ8A | 0.015 | 0.911 | RNA_NFATC3 | −0.011 | 0.936 | RNA_MLKL | −0.222 | 0.096 |
| RNA_ZNF587 | −0.108 | 0.424 | RNA_CALD1 | −0.030 | 0.823 | RNA_TGM2 | 0.043 | 0.750 |
| RNA_WDR1 | −0.266 | 0.046 | RNA_EIF4EBP1 | −0.166 | 0.216 | RNA_SRCAP | 0.028 | 0.836 |
| RNA_PRSS3 | 0.110 | 0.415 | RNA_NUFIP2 | −0.026 | 0.848 | RNA_ZNF226 | 0.004 | 0.975 |
| RNA_DGKD | 0.160 | 0.235 | RNA_ANKRD13C | −0.179 | 0.182 | RNA_RFFL | −0.140 | 0.298 |
| RNA_AEBP2 | −0.069 | 0.609 | RNA_FAM193A | 0.121 | 0.370 | RNA_YEATS2 | −0.287 | 0.030 |
| RNA_SH3RF1 | 0.093 | 0.492 | RNA_RUFY2 | 0.127 | 0.345 | RNA_DESI2 | 0.063 | 0.643 |
| RNA_MSI2 | 0.019 | 0.886 | RNA_CELP | 0.117 | 0.388 | RNA_NIPAL2 | 0.404 | 0.002 |
| RNA_MLLT10 | 0.093 | 0.492 | RNA_ACIN1 | −0.104 | 0.443 | RNA_TFCP2 | 0.112 | 0.406 |
| RNA_NCOR1 | 0.166 | 0.216 | RNA_MIS18BP1 | −0.037 | 0.786 | RNA_GBP3 | −0.104 | 0.443 |
| RNA_TRIM37 | −0.121 | 0.370 | RNA_TMPO | −0.127 | 0.345 | RNA_XIAP | −0.114 | 0.397 |
| RNA_STAT1 | −0.339 | 0.010 | RNA_PIP4K2A | 0.052 | 0.702 | RNA_ENSA | 0.102 | 0.452 |
| RNA_LAMC1 | 0.181 | 0.177 | RNA_STX17 | 0.030 | 0.823 | RNA_UHMK1 | 0.041 | 0.762 |
| RNA_SPATA6 | 0.190 | 0.157 | RNA_DIS3L2 | 0.093 | 0.492 | RNA_TRIM33 | 0.082 | 0.544 |
| RNA_SPATS2L | −0.035 | 0.799 | RNA_SYCN | 0.168 | 0.213 | RNA_TMED7 | 0.037 | 0.786 |
| RNA_LRCH3 | −0.181 | 0.177 | RNA_SLC30A2 | 0.021 | 0.880 | RNA_ZZEF1 | 0.002 | 0.987 |
| RNA_KIAA1217 | 0.184 | 0.172 | RNA_PRKACB | 0.212 | 0.114 | RNA_PRDM4 | −0.130 | 0.337 |
| RNA_TNIK | −0.106 | 0.433 | RNA_ANKFY1 | 0.093 | 0.492 | RNA_ATAD3A | −0.292 | 0.028 |
| RNA_THRAP3 | 0.000 | 1.000 | RNA_ANKRD52 | −0.153 | 0.255 | RNA_DCUN1D2 | 0.024 | 0.861 |
| RNA_DLG1 | −0.214 | 0.110 | RNA_DCAF17 | 0.117 | 0.388 | RNA_PXN | −0.145 | 0.283 |
| RNA_TNRC18 | 0.164 | 0.222 | RNA_TTC28 | 0.164 | 0.222 | RNA_SNX25 | −0.045 | 0.738 |
| RNA_IL1R1 | −0.006 | 0.962 | RNA_OXSR1 | 0.022 | 0.873 | RNA_TCF25 | −0.069 | 0.609 |
| RNA_RREB1 | 0.050 | 0.714 | RNA_ERP27 | 0.125 | 0.353 | RNA_BICDL2 | 0.119 | 0.379 |
| RNA_VDAC1 | −0.050 | 0.714 | RNA_SLC23A2 | 0.147 | 0.276 | RNA_LRRK1 | 0.190 | 0.157 |
| RNA_WASHC2A | 0.019 | 0.886 | RNA_FNBP1L | 0.104 | 0.443 | RNA_HIST2H2BE | −0.181 | 0.177 |
| RNA_SCAF11 | −0.149 | 0.269 | RNA_CYLD | −0.153 | 0.255 | RNA_WDR70 | 0.054 | 0.690 |
| RNA_VPS13D | −0.127 | 0.345 | RNA_TMOD3 | −0.240 | 0.072 | RNA_AP000347.1 | 0.153 | 0.255 |
| RNA_CYBRD1 | 0.307 | 0.020 | RNA_SLC30A6 | −0.030 | 0.823 | RNA_GOLGA4 | −0.004 | 0.975 |
| RNA_TNPO1 | −0.009 | 0.949 | RNA_PHF20 | 0.281 | 0.034 | RNA_A2M | 0.121 | 0.370 |
| RNA_ZMIZ1 | 0.099 | 0.462 | RNA_SLFN11 | −0.188 | 0.162 | RNA_CPEB4 | −0.104 | 0.443 |
| RNA_AKAP13 | 0.214 | 0.110 | RNA_RAB14 | −0.004 | 0.975 | RNA_TUBB6 | 0.026 | 0.848 |
| RNA_KDM4A | −0.011 | 0.936 | RNA_GRAMD2B | 0.145 | 0.283 | RNA_ZNF141 | −0.073 | 0.587 |
| RNA_STK4 | 0.000 | 1.000 | RNA_PHACTR4 | −0.145 | 0.283 | RNA_SH3TC1 | 0.056 | 0.678 |
| RNA_PNLIPRP1 | 0.094 | 0.487 | RNA_ZNF704 | 0.408 | 0.002 | RNA_CHD7 | −0.060 | 0.655 |
| RNA_VTI1A | −0.166 | 0.216 | RNA_DCLRE1C | −0.082 | 0.544 | RNA_HUS1 | 0.110 | 0.415 |
| RNA_CTRC | 0.134 | 0.321 | RNA_VIM | 0.045 | 0.738 | RNA_AK2 | −0.119 | 0.379 |
| RNA_RBM12 | −0.210 | 0.118 | RNA_EHD1 | −0.261 | 0.050 | RNA_KIAA1109 | 0.071 | 0.598 |
| RNA_CCSER2 | 0.060 | 0.655 | RNA_GOLGA3 | 0.082 | 0.544 | RNA_USP8 | −0.091 | 0.502 |
| RNA_CHD9 | 0.175 | 0.193 | RNA_TANC1 | 0.130 | 0.337 | RNA_SLC25A13 | 0.292 | 0.028 |
| RNA_ZNF512 | −0.069 | 0.609 | RNA_MAPRE2 | 0.186 | 0.167 | RNA_BTN3A1 | −0.197 | 0.143 |
| RNA_RIF1 | −0.041 | 0.762 | RNA_USP25 | −0.102 | 0.452 | RNA_CACNA1D | 0.322 | 0.015 |
| RNA_EDEM3 | 0.181 | 0.177 | RNA_RPS7 | −0.045 | 0.738 | RNA_MCU | 0.043 | 0.750 |
| RNA_ITGAV | 0.117 | 0.388 | RNA_SH3PXD2B | 0.179 | 0.182 | RNA_LPCAT1 | −0.156 | 0.248 |
| RNA_QSER1 | −0.045 | 0.738 | RNA_PTPRF | −0.130 | 0.337 | RNA_EML1 | −0.032 | 0.811 |
| RNA_CELA2A | 0.138 | 0.305 | RNA_EFTUD2 | −0.108 | 0.424 | RNA_PDLIM3 | −0.097 | 0.472 |
| RNA_GLS | 0.017 | 0.898 | RNA_PPFIBP1 | −0.168 | 0.210 | RNA_ATP11C | 0.009 | 0.949 |
| RNA_FCGR2A | 0.026 | 0.848 | RNA_CPM | 0.248 | 0.062 | RNA_CACNB3 | −0.136 | 0.313 |
| RNA_DEK | −0.225 | 0.093 | RNA_GPATCH8 | 0.168 | 0.210 | RNA_SMC3 | −0.212 | 0.114 |
| RNA_VMP1 | −0.004 | 0.975 | RNA_IMPA2 | 0.199 | 0.138 | RNA_SPARCL1 | 0.261 | 0.050 |
| RNA_BCL9L | −0.266 | 0.046 | RNA_TMEM245 | 0.166 | 0.216 | RNA_AL732372.3 | −0.064 | 0.637 |
| RNA_AZGP1 | 0.134 | 0.321 | RNA_USP33 | 0.002 | 0.987 | RNA_LZIC | −0.186 | 0.167 |
| RNA_DEPDC5 | 0.024 | 0.861 | RNA_DDX60 | −0.259 | 0.052 | RNA_AP3S2 | −0.006 | 0.962 |
| RNA_N4BP2L2 | 0.015 | 0.911 | RNA_ATP6V0A1 | 0.060 | 0.655 | RNA_JUP | −0.134 | 0.321 |
| RNA_DYNC1H1 | −0.006 | 0.962 | RNA_GATM | 0.162 | 0.229 | RNA_PPP1R9B | −0.097 | 0.472 |
| RNA_ERCC6L2 | 0.130 | 0.337 | RNA_ADAM28 | 0.052 | 0.702 | RNA_PIK3AP1 | −0.175 | 0.193 |
| RNA_PIK3R1 | 0.313 | 0.018 | RNA_NAV3 | 0.080 | 0.555 | RNA_USF3 | −0.009 | 0.949 |
| RNA_LIMA1 | 0.177 | 0.188 | RNA_MEF2D | 0.110 | 0.415 | RNA_ZMYM5 | 0.058 | 0.667 |
| RNA_PPP4R2 | 0.117 | 0.388 | RNA_ZHX2 | −0.112 | 0.406 | RNA_DOCK1 | 0.164 | 0.222 |
| RNA_NPIPB12 | −0.112 | 0.406 | RNA_VEZT | −0.060 | 0.655 | RNA_TCAIM | 0.011 | 0.936 |
| RNA_SREK1 | −0.026 | 0.848 | RNA_PPIG | 0.084 | 0.533 | RNA_FAM210B | 0.173 | 0.199 |
| RNA_RPSA | −0.039 | 0.774 | RNA_RAB5A | −0.011 | 0.936 | RNA_PANK2 | −0.151 | 0.262 |
| RNA_LMO7 | −0.086 | 0.523 | RNA_C3orf38 | −0.063 | 0.643 | RNA_GADD45G | 0.222 | 0.096 |
| RNA_SYNE1 | 0.026 | 0.848 | RNA_RPL35A | 0.158 | 0.241 | RNA_SUZ12 | −0.151 | 0.262 |
| RNA_ATP2A2 | −0.173 | 0.199 | RNA_SLC25A32 | 0.041 | 0.762 | RNA_BAIAP2 | 0.093 | 0.492 |
| RNA_CPA1 | 0.123 | 0.362 | RNA_RPS6KC1 | 0.006 | 0.962 | RNA_ACSL3 | 0.199 | 0.138 |
| RNA_HSPG2 | 0.188 | 0.162 | RNA_SRSF4 | −0.384 | 0.003 | RNA_MAP3K13 | −0.143 | 0.290 |
| RNA_ZNF680 | 0.024 | 0.861 | RNA_ZNF800 | 0.022 | 0.873 | RNA_UBE2D3 | −0.140 | 0.298 |
| RNA_VOPP1 | −0.108 | 0.424 | RNA_ZNF292 | −0.041 | 0.762 | RNA_UBE2E1 | −0.216 | 0.107 |
| RNA_LYST | −0.060 | 0.655 | RNA_COMTD1 | −0.119 | 0.379 | RNA_AREL1 | −0.082 | 0.544 |
| RNA_COL4A1 | 0.091 | 0.502 | RNA_TXNRD1 | −0.222 | 0.096 | RNA_RALA | −0.190 | 0.157 |
| RNA_PTAR1 | 0.227 | 0.090 | RNA_ELMSAN1 | 0.043 | 0.750 | RNA_NFKBIZ | −0.285 | 0.032 |
| RNA_CDK17 | −0.045 | 0.738 | RNA_IGFBP3 | 0.225 | 0.093 | RNA_TRIM50 | 0.069 | 0.609 |
| RNA_BAZ1B | −0.162 | 0.229 | RNA_WWP2 | 0.043 | 0.750 | RNA_TFDP2 | 0.181 | 0.177 |
| RNA_ITGB1 | −0.190 | 0.157 | RNA_NUP153 | 0.108 | 0.424 | RNA_PPP1R8 | −0.244 | 0.067 |
| RNA_NCOA1 | 0.095 | 0.482 | RNA_U2SURP | 0.078 | 0.565 | RNA_LAT2 | −0.121 | 0.370 |
| RNA_SKIL | 0.240 | 0.072 | RNA_CAB39 | −0.067 | 0.621 | RNA_ERO1A | −0.140 | 0.298 |
| RNA_GASK1B | 0.158 | 0.241 | RNA_ZNF592 | −0.028 | 0.836 | RNA_RBPJ | −0.132 | 0.329 |
| RNA_ZNF106 | −0.097 | 0.472 | RNA_SLC38A5 | −0.106 | 0.433 | RNA_LARP4B | −0.015 | 0.911 |
| RNA_ACTN4 | −0.287 | 0.030 | RNA_PFKP | −0.238 | 0.075 | RNA_ARHGDIB | 0.009 | 0.949 |
| RNA_ACAP2 | −0.073 | 0.587 | RNA_FBXO34 | −0.022 | 0.873 | RNA_GRB2 | −0.117 | 0.388 |
| RNA_NEDD4 | 0.028 | 0.836 | RNA_ZDHHC7 | 0.253 | 0.058 | RNA_CSPP1 | 0.147 | 0.276 |
| RNA_UBR1 | 0.035 | 0.799 | RNA_SLC2A1 | −0.050 | 0.714 | RNA_LYZ | 0.205 | 0.126 |
| RNA_ZNF33A | 0.093 | 0.492 | RNA_NT5C2 | −0.199 | 0.138 | RNA_UTP23 | −0.060 | 0.655 |
| RNA_TBC1D5 | 0.045 | 0.738 | RNA_RBBP4 | −0.220 | 0.100 | RNA_PSMD12 | −0.067 | 0.621 |
| RNA_RASAL2 | 0.097 | 0.472 | RNA_PNLIPRP2 | 0.087 | 0.518 | RNA_TEX10 | −0.071 | 0.598 |
| RNA_BAZ2A | −0.279 | 0.036 | RNA_LCP1 | 0.097 | 0.472 | RNA_LDAH | −0.248 | 0.062 |
| RNA_ABI2 | −0.032 | 0.811 | RNA_ZNF644 | −0.069 | 0.609 | RNA_EGF | 0.093 | 0.492 |
| RNA_ANAPC1 | 0.000 | 1.000 | RNA_CPSF6 | 0.045 | 0.738 | RNA_ORC2 | 0.002 | 0.987 |
| RNA_KLK1 | 0.004 | 0.975 | RNA_SRRM1 | −0.119 | 0.379 | RNA_CWC27 | −0.043 | 0.750 |
| RNA_KDM5B | 0.028 | 0.836 | RNA_SP140L | −0.058 | 0.667 | RNA_VRK1 | −0.108 | 0.424 |
| RNA_BTAF1 | −0.078 | 0.565 | RNA_PMS2 | 0.002 | 0.987 | RNA_RNASE1 | 0.227 | 0.090 |
| RNA_HDAC4 | 0.229 | 0.087 | RNA_GALNT10 | 0.013 | 0.924 | RNA_TLE4 | 0.108 | 0.424 |
| RNA_NF1 | 0.009 | 0.949 | RNA_TAF5L | 0.063 | 0.643 | RNA_TBC1D22A | 0.078 | 0.565 |
| RNA_MGLL | 0.089 | 0.512 | RNA_URI1 | −0.093 | 0.492 | RNA_RAD51D | −0.080 | 0.555 |
| RNA_ACTA2 | −0.071 | 0.598 | RNA_STK10 | −0.024 | 0.861 | RNA_TRPM7 | 0.052 | 0.702 |
| RNA_SVIL | 0.086 | 0.523 | RNA_BNIP3L | 0.102 | 0.452 | RNA_NR1D2 | 0.030 | 0.823 |
| RNA_ICE1 | −0.121 | 0.370 | RNA_TARDBP | −0.233 | 0.081 | RNA_EIF4E2 | 0.013 | 0.924 |
| RNA_PDIA2 | 0.253 | 0.058 | RNA_ZFX | 0.041 | 0.762 | RNA_RPL31 | −0.026 | 0.848 |
| RNA_TBL1XR1 | −0.060 | 0.655 | RNA_AKAP10 | 0.184 | 0.172 | RNA_FARP2 | 0.235 | 0.078 |
| RNA_APOL1 | −0.011 | 0.936 | RNA_TSC22D1 | 0.231 | 0.084 | RNA_ABCC5 | 0.210 | 0.118 |
| RNA_APOL2 | −0.091 | 0.502 | RNA_SKI | 0.134 | 0.321 | RNA_ITGA3 | −0.138 | 0.305 |
| RNA_BMS1 | −0.274 | 0.039 | RNA_BICC1 | −0.069 | 0.609 | RNA_GNE | 0.143 | 0.290 |
| RNA_SRGAP2 | 0.153 | 0.255 | RNA_CD68 | 0.076 | 0.576 | RNA_CASP8 | −0.134 | 0.321 |
| RNA_PXDN | −0.147 | 0.276 | RNA_SMYD4 | −0.009 | 0.949 | RNA_MSRB1 | −0.073 | 0.587 |
| RNA_CRIM1 | 0.106 | 0.433 | RNA_GTDC1 | 0.037 | 0.786 | RNA_LGALS9 | −0.071 | 0.598 |
| RNA_HIPK3 | −0.035 | 0.799 | RNA_STEAP2 | −0.041 | 0.762 | RNA_THEMIS2 | −0.037 | 0.786 |
| RNA_FNIP2 | 0.009 | 0.949 | RNA_ARFIP1 | 0.089 | 0.512 | RNA_ANKAR | 0.201 | 0.134 |
| RNA_SHROOM3 | 0.173 | 0.199 | RNA_RESF1 | −0.006 | 0.962 | RNA_AGTPBP1 | 0.028 | 0.836 |
| RNA_SMURF2 | 0.006 | 0.962 | RNA_GPR137B | 0.276 | 0.037 | RNA_PDGFRB | 0.192 | 0.152 |
| RNA_SORT1 | 0.132 | 0.329 | RNA_ANKRD17 | −0.030 | 0.823 | RNA_PRSS23 | 0.056 | 0.678 |
| RNA_SEPT8 | 0.153 | 0.255 | RNA_GAB1 | 0.114 | 0.397 | RNA_AP1S2 | 0.108 | 0.424 |
| RNA_RPL5 | 0.132 | 0.329 | RNA_UBR5 | −0.190 | 0.157 | RNA_ARIH1 | −0.067 | 0.621 |
| RNA_CEMIP2 | 0.138 | 0.305 | RNA_RHBDD1 | 0.181 | 0.177 | RNA_TMEM134 | −0.138 | 0.305 |
| RNA_SOS2 | 0.056 | 0.678 | RNA_USP9X | 0.063 | 0.643 | RNA_CNTRL | 0.015 | 0.911 |
| RNA_R3HDM1 | −0.121 | 0.370 | RNA_UBAP2 | 0.050 | 0.714 | RNA_OGDH | 0.041 | 0.762 |
| RNA_ALG11 | 0.095 | 0.482 | RNA_TVP23B | 0.032 | 0.811 | RNA_BCAR1 | −0.134 | 0.321 |
| RNA_HIST2H2BF | −0.186 | 0.167 | RNA_SLK | 0.022 | 0.873 | RNA_SUSD1 | 0.082 | 0.544 |
| RNA_SERPINI2 | 0.138 | 0.305 | RNA_OPA1 | −0.011 | 0.936 | RNA_EHBP1L1 | −0.251 | 0.060 |
| RNA_MDM4 | 0.104 | 0.443 | RNA_DZIP1L | 0.166 | 0.216 | RNA_SRBD1 | −0.089 | 0.512 |
| RNA_DDX5 | −0.130 | 0.337 | RNA_SP100 | −0.134 | 0.321 | RNA_RALB | −0.335 | 0.011 |
| RNA_TUT7 | 0.017 | 0.898 | RNA_MBTD1 | 0.156 | 0.248 | RNA_MANIA2 | −0.022 | 0.873 |
| RNA_MEF2C | −0.004 | 0.975 | RNA_ITGB4 | −0.048 | 0.726 | RNA_RASA4 | 0.162 | 0.229 |
| RNA_TPM3 | −0.054 | 0.690 | RNA_TM9SF3 | 0.011 | 0.936 | RNA_LBR | 0.125 | 0.353 |
| RNA_CTNNA1 | −0.024 | 0.861 | RNA_APP | −0.056 | 0.678 | RNA_RPTOR | 0.112 | 0.406 |
| RNA_ABI3BP | 0.125 | 0.353 | RNA_WDPCP | 0.028 | 0.836 | RNA_UBR2 | −0.058 | 0.667 |
| RNA_SLAIN2 | 0.073 | 0.587 | RNA_MAP3K8 | −0.039 | 0.774 | RNA_FAM168A | 0.041 | 0.762 |
| RNA_POGZ | 0.276 | 0.037 | RNA_TBCEL | −0.030 | 0.823 | RNA_OSGEPL1 | 0.112 | 0.406 |
| RNA_ETS1 | −0.151 | 0.262 | RNA_AUTS2 | 0.281 | 0.034 | RNA_TCERG1 | −0.063 | 0.643 |
| RNA_UBXN2B | 0.017 | 0.898 | RNA_SFPQ | 0.032 | 0.811 | RNA_SAP130 | 0.117 | 0.388 |
| RNA_TTLL5 | −0.060 | 0.655 | RNA_MTSS1L | −0.028 | 0.836 | RNA_HNRNPA1 | −0.197 | 0.143 |
| RNA_PHLDB2 | −0.121 | 0.370 | RNA_GAMT | 0.279 | 0.036 | RNA_SYNGAP1 | 0.024 | 0.861 |
| RNA_ZNF611 | 0.011 | 0.936 | RNA_RAB35 | −0.009 | 0.949 | RNA_DAP3 | −0.015 | 0.911 |
| RNA_DAB2 | −0.004 | 0.975 | RNA_MTMR2 | −0.073 | 0.587 | RNA_COG5 | 0.071 | 0.598 |
| RNA_HPN | 0.041 | 0.762 | RNA_SPECC1L | 0.028 | 0.836 | RNA_BTG2 | 0.097 | 0.472 |
| RNA_ZNF91 | −0.030 | 0.823 | RNA_HP1BP3 | −0.073 | 0.587 | RNA_STXBP5 | 0.054 | 0.690 |
| RNA_RICTOR | −0.050 | 0.714 | RNA_USP24 | 0.069 | 0.609 | RNA_SPIRE1 | 0.147 | 0.276 |
| RNA_PDS5A | 0.032 | 0.811 | RNA_AMPD3 | −0.257 | 0.054 | RNA_ATM | 0.006 | 0.962 |
| RNA_EP300 | 0.173 | 0.199 | RNA_MMP240S | 0.192 | 0.152 | RNA_SUN1 | −0.112 | 0.406 |
| RNA_AKT3 | 0.017 | 0.898 | RNA_TNKS | 0.024 | 0.861 | RNA_PCMTD1 | 0.082 | 0.544 |
| RNA_THBS1 | −0.037 | 0.786 | RNA_NPC1 | −0.091 | 0.502 | RNA_ARHGAP22 | −0.117 | 0.388 |
| RNA_PRKD3 | 0.084 | 0.533 | RNA_ZFC3H1 | −0.162 | 0.229 | RNA_IREB2 | −0.153 | 0.255 |
| RNA_ACACA | 0.132 | 0.329 | RNA_STARD13 | 0.199 | 0.138 | RNA_PCDH1 | 0.060 | 0.655 |
| RNA_SP1 | −0.110 | 0.415 | RNA_PLEKHG2 | −0.231 | 0.084 | RNA_FAS | −0.076 | 0.576 |
| RNA_ARHGAP21 | 0.030 | 0.823 | RNA_LRIG3 | 0.317 | 0.016 | RNA_KDELC2 | 0.119 | 0.379 |
| RNA_STK38L | 0.095 | 0.482 | RNA_KPNA4 | −0.011 | 0.936 | RNA_MYL12A | 0.108 | 0.424 |
| RNA_PLA2G1B | 0.108 | 0.424 | RNA_LRRC37A3 | −0.024 | 0.861 | RNA_EPAS1 | 0.106 | 0.433 |
| RNA_COL5A1 | 0.013 | 0.924 | RNA_CAND1 | −0.287 | 0.030 | RNA_ABLIM2 | 0.000 | 1.000 |
| RNA_TPST2 | −0.089 | 0.512 | RNA_TRAK2 | 0.197 | 0.143 | RNA_RNF111 | −0.045 | 0.738 |
| RNA_ACBD3 | 0.082 | 0.544 | RNA_TWF1 | −0.158 | 0.241 | RNA_RPLP1 | 0.043 | 0.750 |
| RNA_PDE4DIP | 0.147 | 0.276 | RNA_ZMYND11 | 0.225 | 0.093 | RNA_CTNND1 | −0.065 | 0.632 |
| RNA_DDX6 | 0.006 | 0.962 | RNA_OXCT1 | 0.158 | 0.241 | RNA_PCNX1 | 0.013 | 0.924 |
| RNA_GOLIM4 | 0.104 | 0.443 | RNA_DDX3X | 0.017 | 0.898 | RNA_CHN1 | −0.071 | 0.598 |
| RNA_USP22 | 0.449 | 0.000 | RNA_IFI16 | −0.102 | 0.452 | RNA_PIP4K2B | 0.179 | 0.182 |
| RNA_RALGPS2 | 0.030 | 0.823 | RNA_TAF8 | −0.121 | 0.370 | RNA_PDE8A | 0.039 | 0.774 |
| RNA_RHOA | −0.028 | 0.836 | RNA_LAMA4 | −0.136 | 0.313 | RNA_APIM1 | 0.067 | 0.621 |
| RNA_ZNFX1 | −0.298 | 0.024 | RNA_APAF1 | 0.048 | 0.726 | RNA_PXYLP1 | 0.106 | 0.433 |
| RNA_WSB1 | −0.181 | 0.177 | RNA_SMAD3 | −0.194 | 0.147 | RNA_TUBGCP3 | 0.078 | 0.565 |
| RNA_SLC38A2 | 0.065 | 0.632 | RNA_ITCH | 0.073 | 0.587 | RNA_CREBBP | 0.296 | 0.025 |
| RNA_EPG5 | −0.071 | 0.598 | RNA_TOM1L2 | 0.315 | 0.017 | RNA_ANKMY1 | 0.233 | 0.081 |
| RNA_ANKIB1 | −0.158 | 0.241 | RNA_RABEP1 | 0.136 | 0.313 | RNA_ZNF264 | 0.043 | 0.750 |
| RNA_MYO1F | −0.089 | 0.512 | RNA_SBF2 | −0.162 | 0.229 | RNA_MRE11 | −0.058 | 0.667 |
| RNA_YTHDC2 | 0.037 | 0.786 | RNA_PLAT | −0.002 | 0.987 | RNA_NAB2 | −0.030 | 0.823 |
| RNA_IGFBP5 | 0.127 | 0.345 | RNA_ADD3 | 0.069 | 0.609 | RNA_USP28 | −0.097 | 0.472 |
| RNA_NBL1 | −0.017 | 0.898 | RNA_FOXO1 | 0.160 | 0.235 | RNA_MEIS1 | 0.229 | 0.087 |
| RNA_COL4A2 | 0.043 | 0.750 | RNA_PFKFB3 | −0.015 | 0.911 | RNA_ST3GAL1 | −0.035 | 0.799 |
| RNA_AGPS | 0.065 | 0.632 | RNA_SGMS2 | −0.233 | 0.081 | RNA_TCAF1 | 0.112 | 0.406 |
| RNA_TGFBR1 | 0.212 | 0.114 | RNA_TBC1D23 | 0.048 | 0.726 | RNA_NOL4L | −0.119 | 0.379 |
| RNA_KIF13A | 0.181 | 0.177 | RNA_DNAJC11 | −0.140 | 0.298 | RNA_PPT1 | 0.056 | 0.678 |
| RNA_HIST1H2BK | −0.201 | 0.134 | RNA_TRIP10 | −0.099 | 0.462 | RNA_PSPC1 | −0.084 | 0.533 |
| RNA_VCAN | 0.017 | 0.898 | RNA_CCNT2 | 0.091 | 0.502 | RNA_RCOR3 | 0.216 | 0.107 |
| RNA_LRP1 | 0.205 | 0.126 | RNA_EIF4B | −0.082 | 0.544 | RNA_MUC1 | 0.175 | 0.193 |
| RNA_TPM2 | −0.173 | 0.199 | RNA_LMBR1 | −0.004 | 0.975 | RNA_DLG5 | −0.184 | 0.172 |
| RNA_PTPRK | 0.076 | 0.576 | RNA_H2AFY | −0.026 | 0.848 | RNA_ICAM1 | −0.320 | 0.015 |
| RNA_ITGB3BP | −0.011 | 0.936 | RNA_STK24 | −0.004 | 0.975 | RNA_DPP9 | −0.052 | 0.702 |
| RNA_CTRL | 0.108 | 0.424 | RNA_UBA6 | −0.106 | 0.433 | RNA_UBE2S | −0.333 | 0.011 |
| RNA_CNOT6L | 0.104 | 0.443 | RNA_ARIH2 | 0.030 | 0.823 | RNA_TANK | −0.030 | 0.823 |
| RNA_NRP1 | 0.013 | 0.924 | RNA_TAX1BP1 | −0.006 | 0.962 | RNA_ACVR2A | 0.292 | 0.028 |
| RNA_CBLL1 | −0.097 | 0.472 | RNA_SHQ1 | −0.102 | 0.452 | RNA_GCAT | 0.158 | 0.241 |
| RNA_BTN3A2 | −0.106 | 0.433 | RNA_NEK6 | −0.002 | 0.987 | RNA_POLR1A | 0.093 | 0.492 |
| RNA_EEF2K | 0.296 | 0.025 | RNA_RSPRY1 | 0.095 | 0.482 | RNA_MECOM | 0.112 | 0.406 |
| RNA_PARP14 | −0.430 | 0.001 | RNA_SCFD2 | −0.076 | 0.576 | RNA_UBALD2 | −0.058 | 0.667 |
| RNA_MAST2 | 0.231 | 0.084 | RNA_PPP3R1 | −0.071 | 0.598 | RNA_NFASC | 0.106 | 0.433 |
| RNA_MYO1E | −0.082 | 0.544 | RNA_NFIX | 0.231 | 0.084 | RNA_RALGAPB | −0.015 | 0.911 |
| RNA_SNX13 | 0.143 | 0.290 | RNA_ARID4A | −0.102 | 0.452 | RNA_PIGR | 0.192 | 0.152 |
| RNA_NR3C1 | 0.000 | 1.000 | RNA_ABCC9 | 0.056 | 0.678 | RNA_TEFM | 0.032 | 0.811 |
| RNA_SZT2 | −0.065 | 0.632 | RNA_ZBTB25 | −0.125 | 0.353 | RNA_TSC22D2 | 0.035 | 0.799 |
| RNA_KHDRBS1 | −0.238 | 0.075 | RNA_WDR59 | 0.125 | 0.353 | RNA_STEAP1 | −0.050 | 0.714 |
| RNA_RABGAP1 | 0.082 | 0.544 | RNA_CUL4A | 0.132 | 0.329 | RNA_PRKAB1 | 0.086 | 0.523 |
| RNA_PEA15 | 0.179 | 0.182 | RNA_XRCC5 | 0.086 | 0.523 | RNA_TRNT1 | −0.086 | 0.523 |
| RNA_SMG1P3 | −0.019 | 0.886 | RNA_SLC12A2 | 0.132 | 0.329 | RNA_BAG5 | −0.112 | 0.406 |
| RNA_CORO1C | −0.160 | 0.235 | RNA_ZFP64 | 0.026 | 0.848 | RNA_SPICE1 | 0.011 | 0.936 |
| RNA_CREB1 | 0.082 | 0.544 | RNA_PRKCD | 0.093 | 0.492 | RNA_PER2 | 0.134 | 0.321 |
| RNA_SMAD5 | 0.147 | 0.276 | RNA_AC090114.3 | 0.037 | 0.786 | RNA_ASXL1 | −0.019 | 0.886 |
| RNA_WDR60 | −0.233 | 0.081 | RNA_RUBCN | −0.168 | 0.210 | RNA_PRR12 | −0.006 | 0.962 |
| RNA_COL4A3BP | −0.048 | 0.726 | RNA_IGF1R | 0.106 | 0.433 | RNA_SPEN | −0.140 | 0.298 |
| RNA_RPS27A | 0.132 | 0.329 | RNA_KRAS | −0.019 | 0.886 | RNA_ARHGEF9 | −0.073 | 0.587 |
| RNA_PIP5K1A | 0.054 | 0.690 | RNA_XPNPEP3 | 0.130 | 0.337 | RNA_TOX4 | −0.162 | 0.229 |
| RNA_ESYT2 | 0.099 | 0.462 | RNA_DAZAP2 | 0.004 | 0.975 | RNA_AMBRA1 | −0.041 | 0.762 |
| RNA_RPL41 | −0.175 | 0.193 | RNA_MCL1 | 0.171 | 0.204 | RNA_KHSRP | −0.125 | 0.353 |
| RNA_RALGAPA1 | 0.268 | 0.044 | RNA_IFI44 | −0.194 | 0.147 | RNA_G3BP2 | 0.030 | 0.823 |
| RNA_ARMC9 | −0.235 | 0.078 | RNA_DPY19L3 | 0.095 | 0.482 | RNA_RPS2 | −0.143 | 0.290 |
| RNA_CTNNB1 | 0.210 | 0.118 | RNA_STAU2 | 0.067 | 0.621 | RNA_BTBD3 | 0.242 | 0.070 |
| RNA_LCLAT1 | −0.076 | 0.576 | RNA_ARHGDIG | 0.015 | 0.911 | RNA_CTSB | −0.024 | 0.861 |
| RNA_TBK1 | −0.194 | 0.147 | RNA_ITPKB | −0.067 | 0.621 | RNA_WBP11 | −0.119 | 0.379 |
| RNA_MPDZ | −0.067 | 0.621 | RNA_ATG16L1 | −0.026 | 0.848 | RNA_UBE2W | 0.030 | 0.823 |
| RNA_DPYD | −0.009 | 0.949 | RNA_CTSS | 0.216 | 0.107 | RNA_ADGRA2 | 0.117 | 0.388 |
| RNA_PTK2 | −0.045 | 0.738 | RNA_PIK3C2B | −0.082 | 0.544 | RNA_HDGF | −0.095 | 0.482 |
| RNA_KIAA0232 | 0.076 | 0.576 | RNA_R3HDM2 | 0.056 | 0.678 | RNA_CD74 | 0.171 | 0.204 |
| RNA_LARP1 | −0.197 | 0.143 | RNA_F11R | 0.114 | 0.397 | RNA_KIDINS220 | 0.147 | 0.276 |
| RNA_TRIM16 | −0.043 | 0.750 | RNA_ZNF160 | −0.095 | 0.482 | RNA_DHX8 | 0.125 | 0.353 |
| RNA_AQP8 | 0.110 | 0.415 | RNA_RAB5B | −0.032 | 0.811 | RNA_ACACB | 0.203 | 0.130 |
| RNA_GP2 | 0.132 | 0.329 | RNA_MTHFD1L | −0.093 | 0.492 | RNA_KRT7 | −0.181 | 0.177 |
| RNA_PRRC2C | 0.140 | 0.298 | RNA_MATR3 | −0.026 | 0.848 | RNA_SULT1A3 | −0.095 | 0.482 |
| RNA_YWHAZ | −0.004 | 0.975 | RNA_WSB2 | −0.011 | 0.936 | RNA_CCDC66 | 0.095 | 0.482 |
| RNA_AC080038.1 | 0.052 | 0.702 | RNA_HEATR5A | −0.015 | 0.911 | RNA_ERO1B | 0.151 | 0.262 |
| RNA_GCC2 | 0.006 | 0.962 | RNA_MAX | −0.099 | 0.462 | RNA_TPRN | −0.028 | 0.836 |
| RNA_ELF2 | 0.019 | 0.886 | RNA_LPIN2 | 0.153 | 0.255 | RNA_MPZL1 | 0.242 | 0.070 |
| RNA_ATP8B1 | 0.050 | 0.714 | RNA_SEC61A1 | −0.013 | 0.924 | RNA_PPP2CA | −0.121 | 0.370 |
| RNA_CEP350 | 0.259 | 0.052 | RNA_GTF2IRD2B | 0.326 | 0.013 | RNA_ZNHIT6 | −0.086 | 0.523 |
| RNA_NFE2L2 | 0.425 | 0.001 | RNA_LASP1 | −0.123 | 0.362 | RNA_ENC1 | −0.259 | 0.052 |
| RNA_BCAS3 | 0.166 | 0.216 | RNA_RIN2 | −0.078 | 0.565 | RNA_UBA3 | −0.017 | 0.898 |
| RNA_HIF1A | −0.104 | 0.443 | RNA_TMEM182 | 0.000 | 1.000 | RNA_SPG11 | 0.035 | 0.799 |
| RNA_MIER1 | 0.112 | 0.406 | RNA_PSEN1 | −0.019 | 0.886 | RNA_MECP2 | −0.030 | 0.823 |
| RNA_TSHZ2 | 0.192 | 0.152 | RNA_FILIP1L | −0.052 | 0.702 | RNA_QRICH1 | 0.134 | 0.321 |
| RNA_DENND3 | −0.058 | 0.667 | RNA_AKNA | 0.058 | 0.667 | RNA_VPS37B | −0.015 | 0.911 |
| RNA_RPE | 0.127 | 0.345 | RNA_KDM3B | 0.143 | 0.290 | RNA_CMTM3 | 0.060 | 0.655 |
| RNA_CRISPLD2 | 0.315 | 0.017 | RNA_INPP4A | −0.069 | 0.609 | RNA_GAB2 | 0.002 | 0.987 |
| RNA_FBXL17 | 0.305 | 0.021 | RNA_NAB1 | −0.194 | 0.147 | RNA_DYNC2H1 | 0.173 | 0.199 |
| RNA_PRMT3 | −0.317 | 0.016 | RNA_NEDD9 | 0.339 | 0.010 | RNA_AMOTL1 | 0.069 | 0.609 |
| RNA_RAB6A | −0.177 | 0.188 | RNA_ETV6 | −0.112 | 0.406 | RNA_CMTM6 | 0.097 | 0.472 |
| RNA_SMARCAD1 | 0.091 | 0.502 | RNA_ZCCHC24 | 0.313 | 0.018 | RNA_RB1 | 0.089 | 0.512 |
| RNA_UGCG | −0.104 | 0.443 | RNA_FYN | 0.048 | 0.726 | RNA_MPP6 | 0.136 | 0.313 |
| RNA_LARP4 | −0.130 | 0.337 | RNA_NDEL1 | −0.030 | 0.823 | RNA_RECQL | −0.080 | 0.555 |
| RNA_REG1B | 0.017 | 0.898 | RNA_CDK13 | 0.091 | 0.502 | RNA_ARHGAP9 | −0.099 | 0.462 |
| RNA_USP32 | −0.039 | 0.774 | RNA_ABCA9 | 0.121 | 0.370 | RNA_GDAP2 | 0.114 | 0.397 |
| RNA_TRIM22 | −0.086 | 0.523 | RNA_NUP155 | −0.091 | 0.502 | RNA_ZNF493 | 0.082 | 0.544 |
| RNA_PTCH1 | 0.179 | 0.182 | RNA_MYO9B | −0.156 | 0.248 | RNA_MICAL3 | −0.043 | 0.750 |
| RNA_STAT2 | −0.339 | 0.010 | RNA_MBD5 | 0.162 | 0.229 | RNA_THY1 | 0.134 | 0.321 |
| RNA_SLC44A1 | 0.149 | 0.269 | RNA_PHF20L1 | −0.011 | 0.936 | RNA_BCL2L1 | −0.179 | 0.182 |
| RNA_MYH9 | 0.052 | 0.702 | RNA_PLEKHA5 | 0.138 | 0.305 | RNA_GMNN | 0.080 | 0.555 |
| RNA_AHCYL1 | 0.184 | 0.172 | RNA_STK35 | −0.138 | 0.305 | RNA_C19orf48 | −0.121 | 0.370 |
| RNA_CAPZA1 | −0.067 | 0.621 | RNA_SEMA4D | 0.009 | 0.949 | RNA_NEXN | 0.106 | 0.433 |
| RNA_LBH | −0.019 | 0.886 | RNA_AC125232.1 | −0.086 | 0.523 | RNA_JOSD1 | 0.069 | 0.609 |
| RNA_SYTL1 | −0.220 | 0.100 | RNA_ZDHHC3 | 0.099 | 0.462 | RNA_PPP2R2A | −0.143 | 0.290 |
| RNA_CPD | 0.279 | 0.036 | RNA_PIAS1 | 0.132 | 0.329 | RNA_DCAF7 | −0.093 | 0.492 |
| RNA_NPIPB3 | −0.231 | 0.084 | RNA_RPL22 | −0.119 | 0.379 | RNA_RBMXL1 | 0.177 | 0.188 |
| RNA_DOCK4 | −0.015 | 0.911 | RNA_VPS41 | 0.207 | 0.122 | RNA_VPS26A | −0.138 | 0.305 |
| RNA_DFFA | −0.402 | 0.002 | RNA_HMGN2 | 0.058 | 0.667 | RNA_ATF1 | 0.058 | 0.667 |
| RNA_GOLGA8B | 0.037 | 0.786 | RNA_PCDHGC3 | −0.104 | 0.443 | RNA_RPL24 | 0.015 | 0.911 |
| RNA_TCF12 | 0.149 | 0.269 | RNA_TMCC1 | −0.035 | 0.799 | RNA_ZNF383 | 0.121 | 0.370 |
| RNA_LMNA | 0.108 | 0.424 | RNA_ZNF362 | 0.006 | 0.962 | RNA_CMIP | −0.153 | 0.255 |
| RNA_FBXW7 | −0.127 | 0.345 | RNA_VASP | −0.266 | 0.046 | RNA_ADSS | −0.043 | 0.750 |
| RNA_ZNF506 | 0.002 | 0.987 | RNA_WWOX | 0.106 | 0.433 | RNA_SLC40A1 | 0.145 | 0.283 |
| RNA_UACA | −0.017 | 0.898 | RNA_SUPT6H | 0.004 | 0.975 | RNA_SCYL2 | −0.089 | 0.512 |
| RNA_JADE2 | 0.071 | 0.598 | RNA_ACAD9 | −0.056 | 0.678 | RNA_RBM5 | 0.095 | 0.482 |
| RNA_NKTR | 0.127 | 0.345 | RNA_VPS13A | 0.069 | 0.609 | RNA_SNX6 | −0.210 | 0.118 |
| RNA_TENT2 | 0.069 | 0.609 | RNA_AP3M2 | 0.048 | 0.726 | RNA_EXT1 | −0.162 | 0.229 |
| RNA_ZNF207 | 0.050 | 0.714 | RNA_NR2C2 | 0.076 | 0.576 | RNA_IGLC1 | 0.043 | 0.750 |
| RNA_AGAP5 | −0.058 | 0.667 | RNA_MSANTD3 | −0.320 | 0.015 | RNA_B4GAT1 | 0.233 | 0.081 |
| RNA_ARHGAP5 | −0.117 | 0.388 | RNA_PPP4R3B | 0.041 | 0.762 | RNA_ATP6V1B2 | −0.013 | 0.924 |
| RNA_RAPGEF5 | 0.248 | 0.062 | RNA_SAFB | −0.214 | 0.110 | RNA_NUCKS1 | 0.037 | 0.786 |
| RNA_ZCCHC14 | 0.043 | 0.750 | RNA_KLHL20 | 0.205 | 0.126 | RNA_ZC4H2 | 0.175 | 0.193 |
| RNA_UBE3B | −0.186 | 0.167 | RNA_FOSL2 | 0.138 | 0.305 | RNA_UBE3A | 0.039 | 0.774 |
| RNA_ATRX | −0.032 | 0.811 | RNA_PTPRS | 0.199 | 0.138 | RNA_LRRFIP2 | 0.106 | 0.433 |
| RNA_HIVEP2 | −0.242 | 0.070 | RNA_ELMO1 | 0.082 | 0.544 | RNA_TMF1 | −0.069 | 0.609 |
| RNA_FOXJ3 | 0.030 | 0.823 | RNA_NLRC5 | −0.285 | 0.032 | RNA_CDS2 | 0.212 | 0.114 |
| RNA_AGAP6 | −0.121 | 0.370 | RNA_ANAPC16 | 0.136 | 0.313 | RNA_USP31 | 0.041 | 0.762 |
| RNA_CASC4 | −0.011 | 0.936 | RNA_ERGIC1 | 0.102 | 0.452 | RNA_TMEM67 | 0.037 | 0.786 |
| RNA_SPTLC2 | −0.032 | 0.811 | RNA_SLC41A2 | −0.028 | 0.836 | RNA_TENT4A | −0.022 | 0.873 |
| RNA_TASOR2 | −0.102 | 0.452 | RNA_ZFAND5 | 0.315 | 0.017 | RNA_CHD1L | 0.251 | 0.060 |
| RNA_BCL2L11 | 0.028 | 0.836 | RNA_TM7SF2 | −0.106 | 0.433 | RNA_HSPA4 | −0.082 | 0.544 |
| RNA_AP003419.1 | −0.001 | 0.994 | RNA_NF2 | 0.175 | 0.193 | RNA_DCUN1D3 | 0.218 | 0.103 |
| RNA_PLEKHF2 | 0.037 | 0.786 | RNA_ZDHHC17 | 0.114 | 0.397 | RNA_STX2 | 0.078 | 0.565 |
| RNA_MMP14 | −0.069 | 0.609 | RNA_FAT1 | −0.080 | 0.555 | RNA_HIST1H2AC | −0.162 | 0.229 |
| RNA_CSNK2A1 | −0.166 | 0.216 | RNA_PDGFRA | 0.190 | 0.157 | RNA_GOLGA6L4 | −0.041 | 0.762 |
| RNA_GNMT | 0.026 | 0.848 | RNA_SFXN1 | −0.052 | 0.702 | RNA_REV3L | 0.127 | 0.345 |
| RNA_SPIN1 | 0.119 | 0.379 | RNA_NREP | 0.106 | 0.433 | RNA_MACROD1 | −0.091 | 0.502 |
| RNA_STAT5B | 0.056 | 0.678 | RNA_TULP3 | −0.147 | 0.276 | RNA_PPWD1 | −0.013 | 0.924 |
| RNA_CEP120 | 0.227 | 0.090 | RNA_SETD1B | −0.037 | 0.786 | RNA_GSTA2 | 0.081 | 0.549 |
| RNA_SPAST | −0.076 | 0.576 | RNA_SYNPO | 0.048 | 0.726 | RNA_RBM41 | −0.147 | 0.276 |
| RNA_FGL1 | 0.082 | 0.544 | RNA_PSMD6 | 0.006 | 0.962 | RNA_ERCC5 | 0.017 | 0.898 |
| RNA_HEATR1 | 0.143 | 0.290 | RNA_PABPC4 | 0.035 | 0.799 | RNA_C12orf4 | 0.039 | 0.774 |
| RNA_ZNF417 | −0.022 | 0.873 | RNA_GIGYF2 | −0.082 | 0.544 | RNA_JAGN1 | 0.080 | 0.555 |
| RNA_TTC3 | 0.056 | 0.678 | RNA_POMZP3 | 0.002 | 0.987 | RNA_DHX9 | 0.037 | 0.786 |
| RNA_WIPF2 | −0.026 | 0.848 | RNA_LIMD1 | 0.017 | 0.898 | RNA_ZNF282 | −0.039 | 0.774 |
| RNA_SH3PXD2A | −0.125 | 0.353 | RNA_MAP7D1 | −0.179 | 0.182 | RNA_TSPAN1 | −0.024 | 0.861 |
| RNA_PHTF2 | −0.050 | 0.714 | RNA_TIPARP | 0.309 | 0.019 | RNA_RAP1A | −0.073 | 0.587 |
| RNA_TASP1 | 0.253 | 0.058 | RNA_AQP12A | 0.001 | 0.994 | RNA_BIRC3 | −0.268 | 0.044 |
| RNA_WDR33 | 0.160 | 0.235 | RNA_SWAP70 | 0.121 | 0.370 | RNA_RAB21 | −0.009 | 0.949 |
| RNA_KMT2C | −0.091 | 0.502 | RNA_PLEKHA7 | 0.028 | 0.836 | RNA_PARP9 | −0.365 | 0.005 |
| RNA_SIPA1L3 | −0.080 | 0.555 | RNA_KDM4B | 0.145 | 0.283 | RNA_SLTM | 0.004 | 0.975 |
| RNA_KHDC4 | 0.106 | 0.433 | RNA_BCLAF3 | 0.080 | 0.555 | RNA_AC138969.1 | 0.171 | 0.204 |
| RNA_WDR43 | −0.039 | 0.774 | RNA_ABCG1 | 0.019 | 0.886 | RNA_DHDDS | −0.175 | 0.193 |
| RNA_RSF1 | −0.082 | 0.544 | RNA_ABR | 0.041 | 0.762 | RNA_SLC25A46 | −0.151 | 0.262 |
| RNA_OTUD4 | −0.121 | 0.370 | RNA_HIST1H2BD | −0.192 | 0.152 | RNA_EMP1 | 0.050 | 0.714 |
| RNA_TGFBRAP1 | 0.127 | 0.345 | RNA_ZNF736 | 0.028 | 0.836 | RNA_CRK | −0.097 | 0.472 |
| RNA_TVP23C | −0.006 | 0.962 | RNA_MPRIP | 0.099 | 0.462 | RNA_RPS8 | −0.043 | 0.750 |
| RNA_VAMP3 | −0.298 | 0.024 | RNA_EFL1 | −0.060 | 0.655 | RNA_METTL25 | 0.004 | 0.975 |
| RNA_BRWD1 | −0.063 | 0.643 | RNA_SHC1 | −0.045 | 0.738 | RNA_LRBA | 0.002 | 0.987 |
| RNA_CBX5 | 0.000 | 1.000 | RNA_LMCD1 | 0.078 | 0.565 | RNA_DICER1 | 0.156 | 0.248 |
| RNA_EXOC2 | −0.073 | 0.587 | RNA_CYTH1 | 0.117 | 0.388 | RNA_TP53 | −0.110 | 0.415 |
| RNA_PRKCH | 0.106 | 0.433 | RNA_NUBPL | −0.084 | 0.533 | RNA_S100A14 | 0.099 | 0.462 |
| RNA_JAZF1 | 0.117 | 0.388 | RNA_RASSF3 | −0.050 | 0.714 | RNA_UBE2H | −0.227 | 0.090 |
| RNA_SGPL1 | 0.084 | 0.533 | RNA_SPAG9 | 0.151 | 0.262 | RNA_ANKRD13A | −0.387 | 0.003 |
| RNA_CASP10 | −0.227 | 0.090 | RNA_PRKD1 | 0.158 | 0.241 | RNA_LIMK1 | −0.093 | 0.492 |
| RNA_FAM3B | −0.043 | 0.750 | RNA_SEC24A | −0.125 | 0.353 | RNA_TUFT1 | 0.015 | 0.911 |
| RNA_POLR2J3 | −0.097 | 0.472 | RNA_ZNF814 | −0.043 | 0.750 | RNA_MSL2 | 0.199 | 0.138 |
| RNA_FAM13B | −0.164 | 0.222 | RNA_NBR1 | 0.121 | 0.370 | RNA_UBTF | 0.097 | 0.472 |
| RNA_ATL2 | −0.004 | 0.975 | RNA_MPHOSPH9 | −0.194 | 0.147 | RNA_PPIP5K2 | 0.145 | 0.283 |
| RNA_VPS13C | 0.050 | 0.714 | RNA_ANXA1 | 0.086 | 0.523 | RNA_C5orf24 | −0.043 | 0.750 |
| RNA_STAM | −0.177 | 0.188 | RNA_PLEKHH2 | 0.140 | 0.298 | RNA_SENP5 | −0.011 | 0.936 |
| RNA_HMGXB4 | 0.160 | 0.235 | RNA_SERPINA3 | −0.203 | 0.130 | RNA_TPP2 | 0.067 | 0.621 |
| RNA_BMPR1A | 0.181 | 0.177 | RNA_NBPF26 | −0.443 | 0.001 | RNA_CARD8 | −0.097 | 0.472 |
| RNA_EFR3A | 0.058 | 0.667 | RNA_PHF3 | 0.071 | 0.598 | RNA_DOCK6 | 0.099 | 0.462 |
| RNA_USP48 | −0.138 | 0.305 | RNA_REG3G | 0.060 | 0.660 | RNA_PACRGL | 0.089 | 0.512 |
| RNA_RBM12B | −0.091 | 0.502 | RNA_C5orf15 | 0.048 | 0.726 | RNA_ZNF528 | 0.060 | 0.655 |
| RNA_AVL9 | 0.160 | 0.235 | RNA_BNIP3 | 0.037 | 0.786 | RNA_CHMP3 | 0.093 | 0.492 |
| RNA_STK3 | −0.071 | 0.598 | RNA_TPR | 0.108 | 0.424 | RNA_STT3B | 0.004 | 0.975 |
| RNA_PICALM | 0.006 | 0.962 | RNA_RALBP1 | 0.028 | 0.836 | RNA_PYGL | −0.210 | 0.118 |
| RNA_RAB11FIP2 | 0.026 | 0.848 | RNA_ATPSCKMT | −0.015 | 0.911 | RNA_USP40 | 0.060 | 0.655 |
| RNA_METTL2B | 0.009 | 0.949 | RNA_ARL5A | −0.130 | 0.337 | RNA_SSH2 | 0.160 | 0.235 |
| RNA_LPGAT1 | 0.147 | 0.276 | RNA_FAP | −0.071 | 0.598 | RNA_KANSL1L | 0.255 | 0.056 |
| RNA_GSK3B | −0.121 | 0.370 | RNA_COL6A2 | −0.052 | 0.702 | RNA_SUMO2 | −0.097 | 0.472 |
| RNA_ACSL4 | −0.194 | 0.147 | RNA_ZNF451 | 0.013 | 0.924 | RNA_PPHLN1 | −0.231 | 0.084 |
| RNA_KDM5A | −0.052 | 0.702 | RNA_AP1G1 | 0.032 | 0.811 | RNA_BTN2A2 | −0.177 | 0.188 |
| RNA_UTRN | 0.060 | 0.655 | RNA_RSRC1 | 0.030 | 0.823 | RNA_NISCH | 0.268 | 0.044 |
| RNA_ABI1 | 0.091 | 0.502 | RNA_EIF4E | −0.078 | 0.565 | RNA_MFN2 | −0.328 | 0.013 |
| RNA_WDR75 | 0.110 | 0.415 | RNA_GALNT1 | 0.002 | 0.987 | RNA_RNF103 | 0.307 | 0.020 |
| RNA_TFRC | 0.106 | 0.433 | RNA_CHD3 | 0.063 | 0.643 | RNA_KCNK1 | 0.298 | 0.024 |
| RNA_LARGE1 | 0.164 | 0.222 | RNA_BTBD1 | −0.076 | 0.576 | RNA_VASH1 | −0.024 | 0.861 |
| RNA_CBWD5 | −0.009 | 0.949 | RNA_RPRD1B | −0.127 | 0.345 | RNA_TBC1D4 | 0.203 | 0.130 |
| RNA_NSD3 | −0.104 | 0.443 | RNA_DUSP6 | −0.086 | 0.523 | RNA_EPS15L1 | −0.019 | 0.886 |
| RNA_NBPF14 | 0.043 | 0.750 | RNA_UBA2 | −0.039 | 0.774 | RNA_MANBA | 0.019 | 0.886 |
| RNA_OTULIN | −0.084 | 0.533 | RNA_ANTXR2 | 0.086 | 0.523 | RNA_ZNF561 | 0.004 | 0.975 |
| RNA_PTEN | 0.151 | 0.262 | RNA_TJP2 | 0.199 | 0.138 | RNA_CHD8 | −0.231 | 0.084 |
| RNA_RAD21 | −0.052 | 0.702 | RNA_ATP10D | 0.192 | 0.152 | | | |
| RNA_METTL8 | 0.050 | 0.714 | RNA_MLXIP | −0.158 | 0.241 | | | |

TABLE 7A
Protein and Lipid Top Features

| Analyte | Study Label | Feature | Frequency |
|---|---|---|---|
| Plasma_Protein | label_deceased | ANXA1 | 0.8824 |
| Plasma_Protein | label_deceased | CO6 | 0.8627 |
| Plasma_Protein | label_deceased | GPC1 | 0.8431 |
| Plasma_Protein | label_deceased | D19L1 | 0.7843 |
| Plasma_Protein | label_deceased | ALS | 0.7255 |
| Plasma_Protein | label_deceased | HV320 | 0.6078 |
| Plasma_Protein | label_deceased | SPTB1 | 0.5882 |
| Plasma_Protein | label_deceased | VTDB | 0.5294 |
| Plasma_Protein | label_deceased | FIBA | 0.4902 |
| Plasma_Protein | label_deceased | ICAL | 0.451 |
| Plasma_Protein | label_deceased | B2L13 | 0.4314 |
| Plasma_Protein | label_deceased | RET4 | 0.4314 |
| Plasma_Protein | label_deceased | HSP74 | 0.3922 |
| Plasma_Protein | label_deceased | IGJ | 0.3333 |
| Plasma_Protein | label_deceased | FHR1 | 0.3333 |
| Plasma_Protein | label_deceased | PZP | 0.3137 |
| Plasma_Protein | label_deceased | MUC19 | 0.2941 |
| Plasma_Protein | label_deceased | PROZ | 0.2941 |
| Plasma_Protein | label_deceased | GRB2 | 0.2745 |
| Plasma_Protein | label_deceased | AACT | 0.2549 |
| Plasma_Protein | label_deceased | GPX3 | 0.2353 |
| Plasma_Protein | label_deceased | PRG4 | 0.2157 |
| Plasma_Protein | label_deceased | FINC | 0.2157 |
| Plasma_Protein | label_deceased | UBP5 | 0.2157 |
| Plasma_Protein | label_deceased | FRPD1 | 0.1961 |
| Plasma_Protein | label_deceased | UBP14 | 0.1961 |
| Plasma_Protein | label_deceased | THY1 | 0.1765 |
| Plasma_Protein | label_deceased | APOC3 | 0.1569 |
| Plasma_Protein | label_deceased | LBP | 0.1569 |
| Plasma_Protein | label_deceased | CD14 | 0.1569 |
| Plasma_Protein | label_deceased | A2AP | 0.1373 |
| Plasma_Protein | label_deceased | PSMD1 | 0.1373 |
| Plasma_Protein | label_deceased | TRFE | 0.1176 |
| Plasma_Protein | label_deceased | MBL2 | 0.1176 |
| Plasma_Protein | label_deceased | RNAS6 | 0.1176 |
| Plasma_Protein | label_deceased | KV203 | 0.1176 |
| Plasma_Protein | label_deceased | CNDP1 | 0.1176 |
| Plasma_Protein | label_deceased | IGHA2 | 0.1176 |
| Plasma_Protein | label_deceased | IGHD | 0.1176 |
| Plasma_Protein | label_deceased | CH60 | 0.1176 |
| Plasma_Protein | label_deceased | HABP2 | 0.098 |
| Plasma_Protein | label_deceased | LUZP1 | 0.098 |
| Plasma_Protein | label_deceased | STK10 | 0.098 |
| Plasma_Protein | label_deceased | ENPL | 0.098 |
| Plasma_Protein | label_deceased | STAT3 | 0.0784 |
| Plasma_Protein | label_deceased | APOC1 | 0.0784 |
| Plasma_Protein | label_deceased | LDHB | 0.0784 |
| Plasma_Protein | label_deceased | CAD13 | 0.0588 |
| Plasma_Protein | label_deceased | FA12 | 0.0588 |
| Plasma_Protein | label_deceased | ILEU | 0.0588 |
| Plasma_Protein | label_deceased | MED30 | 0.0588 |
| Plasma_Protein | label_deceased | FLNB | 0.0588 |
| Plasma_Protein | label_deceased | NAMPT | 0.0588 |
| Tissue_Protein | label_deceased | COBA1 | 0.8776 |
| Tissue_Protein | label_deceased | CO8A1 | 0.7551 |
| Tissue_Protein | label_deceased | RLA0 | 0.7143 |
| Tissue_Protein | label_deceased | COLA1 | 0.6122 |
| Tissue_Protein | label_deceased | WASC5 | 0.6122 |
| Tissue_Protein | label_deceased | MLP3A | 0.5714 |
| Tissue_Protein | label_deceased | FABP4 | 0.5714 |
| Tissue_Protein | label_deceased | ALDOA | 0.5306 |
| Tissue_Protein | label_deceased | CD82 | 0.5306 |
| Tissue_Protein | label_deceased | BUD31 | 0.4286 |
| Tissue_Protein | label_deceased | KCRB | 0.4286 |
| Tissue_Protein | label_deceased | BCAM | 0.3878 |
| Tissue_Protein | label_deceased | GPC1 | 0.3469 |
| Tissue_Protein | label_deceased | COAA1 | 0.3265 |
| Tissue_Protein | label_deceased | LONM | 0.2653 |
| Tissue_Protein | label_deceased | LCAP | 0.2245 |
| Tissue_Protein | label_deceased | VAMP3 | 0.2245 |
| Tissue_Protein | label_deceased | FGD4 | 0.2041 |
| Tissue_Protein | label_deceased | SGTA | 0.1837 |
| Tissue_Protein | label_deceased | FUMH | 0.1837 |
| Tissue_Protein | label_deceased | CO5A1 | 0.1633 |
| Tissue_Protein | label_deceased | COMP | 0.1633 |
| Tissue_Protein | label_deceased | PRDX2 | 0.1429 |
| Tissue_Protein | label_deceased | CO1A1 | 0.1429 |
| Tissue_Protein | label_deceased | ATP5H | 0.1429 |
| Tissue_Protein | label_deceased | TBD2B | 0.1429 |
| Tissue_Protein | label_deceased | LMO7 | 0.1429 |
| Tissue_Protein | label_deceased | ENOA | 0.1429 |
| Tissue_Protein | label_deceased | IDH3A | 0.1429 |
| Tissue_Protein | label_deceased | GGYF2 | 0.1224 |
| Tissue_Protein | label_deceased | AP3M1 | 0.1224 |
| Tissue_Protein | label_deceased | ARSA | 0.102 |
| Tissue_Protein | label_deceased | MTX2 | 0.102 |
| Tissue_Protein | label_deceased | AT5F1 | 0.102 |
| Tissue_Protein | label_deceased | MYADM | 0.102 |
| Tissue_Protein | label_deceased | SPD2B | 0.102 |
| Tissue_Protein | label_deceased | ITA3 | 0.102 |
| Tissue_Protein | label_deceased | GOSR1 | 0.102 |
| Tissue_Protein | label_deceased | 5NTC | 0.102 |
| Tissue_Protein | label_deceased | ADDA | 0.102 |
| Tissue_Protein | label_deceased | MYG | 0.0816 |
| Tissue_Protein | label_deceased | TENA | 0.0816 |
| Tissue_Protein | label_deceased | STS | 0.0816 |
| Tissue_Protein | label_deceased | CHD8 | 0.0816 |
| Tissue_Protein | label_deceased | MOXD1 | 0.0816 |
| Tissue_Protein | label_deceased | STMN1 | 0.0816 |
| Tissue_Protein | label_deceased | CBG | 0.0816 |
| Tissue_Protein | label_deceased | CO8A2 | 0.0816 |
| Tissue_Protein | label_deceased | THSD4 | 0.0612 |
| Tissue_Protein | label_deceased | RAB23 | 0.0612 |
| Tissue_Protein | label_deceased | MON2 | 0.0612 |
| Tissue_Protein | label_deceased | AP2A1 | 0.0612 |
| Tissue_Protein | label_deceased | CO3A1 | 0.0612 |
| Tissue_Protein | label_deceased | C1QT5 | 0.0612 |
| Tissue_Protein | label_deceased | RM13 | 0.0612 |
| Tissue_Protein | label_deceased | TGM2 | 0.0612 |
| Tissue_Protein | label_deceased | ODPB | 0.0612 |
| Tissue_Protein | label_deceased | OSBP1 | 0.0612 |
| Tissue_Protein | label_deceased | ITB4 | 0.0612 |
| Tissue_Protein | label_deceased | TIMP2 | 0.0612 |
| Plasma_Lipid | label_deceased | species_conc_DAG(18:1/18:1) | 0.66666667 |
| Plasma_Lipid | label_deceased | species_conc_DAG(18:1/18:2) | 0.62745098 |
| Plasma_Lipid | label_deceased | species_conc_DAG(16:1/18:1) | 0.58823529 |
| Plasma_Lipid | label_deceased | species_conc_DAG(16:0/18:1) | 0.52941177 |
| Plasma_Lipid | label_deceased | species_conc_CE(18:0) | 0.49019608 |
| Plasma_Lipid | label_deceased | species_conc_CE(18:1) | 0.49019608 |
| Plasma_Lipid | label_deceased | species_conc_CE(20:5) | 0.49019608 |
| Plasma_Lipid | label_deceased | species_conc_CE(18:4) | 0.47058824 |
| Plasma_Lipid | label_deceased | species_conc_DAG(18:0/18:1) | 0.47058824 |
| Plasma_Lipid | label_deceased | species_conc_CER(24:0) | 0.43137255 |
| Plasma_Lipid | label_deceased | species_conc_CE(18:2) | 0.43137255 |
| Plasma_Lipid | label_deceased | species_conc_CE(16:1) | 0.39215686 |
| Plasma_Lipid | label_deceased | species_conc_CE(20:3) | 0.39215686 |
| Plasma_Lipid | label_deceased | species_conc_CE(18:3) | 0.39215686 |
| Plasma_Lipid | label_deceased | species_conc_CE(14:1) | 0.37254902 |
| Plasma_Lipid | label_deceased | species_conc_CE(17:0) | 0.35294118 |
| Plasma_Lipid | label_deceased | species_conc_CE(15:0) | 0.33333333 |
| Plasma_Lipid | label_deceased | species_conc_CE(16:0) | 0.33333333 |
| Plasma_Lipid | label_deceased | species_conc_DAG(16:0/18:0) | 0.31372549 |
| Plasma_Lipid | label_deceased | species_conc_DAG(16:0/18:2) | 0.31372549 |
| Plasma_Lipid | label_deceased | species_conc_CE(20:4) | 0.31372549 |
| Plasma_Lipid | label_deceased | species_conc_CE(22:5) | 0.31372549 |
| Plasma_Lipid | label_deceased | species_conc_LPC(18:2) | 0.2745098 |
| Plasma_Lipid | label_deceased | species_conc_FFA(20:2) | 0.2745098 |
| Plasma_Lipid | label_deceased | species_conc_LCER(16:0) | 0.23529412 |
| Plasma_Lipid | label_deceased | species_conc_PC(16:0/18:1) | 0.21568628 |
| Plasma_Lipid | label_deceased | species_conc_DAG(16:0/16:0) | 0.19607843 |
| Plasma_Lipid | label_deceased | species_conc_CE(14:0) | 0.19607843 |
| Plasma_Lipid | label_deceased | species_conc_LPC(20:3) | 0.17647059 |
| Plasma_Lipid | label_deceased | species_conc_FFA(16:0) | 0.15686275 |
| Plasma_Lipid | label_deceased | species_conc_PC(16:0/20:3) | 0.15686275 |
| Plasma_Lipid | label_deceased | species_conc_LPC(18:0) | 0.15686275 |
| Plasma_Lipid | label_deceased | species_conc_LPC(18:1) | 0.1372549 |
| Plasma_Lipid | label_deceased | species_conc_FFA(14:0) | 0.1372549 |
| Plasma_Lipid | label_deceased | species_conc_CE(22:6) | 0.1372549 |
| Plasma_Lipid | label_deceased | species_conc_FFA(22:6) | 0.1372549 |
| Plasma_Lipid | label_deceased | species_conc_FFA(22:5) | 0.1372549 |
| Plasma_Lipid | label_deceased | species_conc_FFA(12:0) | 0.11764706 |
| Plasma_Lipid | label_deceased | species_conc_FFA(14:1) | 0.11764706 |
| Plasma_Lipid | label_deceased | species_conc_FFA(17:0) | 0.11764706 |
| Plasma_Lipid | label_deceased | species_conc_FFA(18:0) | 0.11764706 |
| Plasma_Lipid | label_deceased | species_conc_FFA(24:1) | 0.11764706 |
| Plasma_Lipid | label_deceased | species_conc_LPC(20:4) | 0.11764706 |
| Plasma_Lipid | label_deceased | species_conc_FFA(22:4) | 0.09803922 |
| Plasma_Lipid | label_deceased | species_conc_PC(16:0/22:4) | 0.09803922 |
| Plasma_Lipid | label_deceased | species_conc_LPE(18:1) | 0.07843137 |
| Plasma_Lipid | label_deceased | species_conc_PC(16:0/18:2) | 0.07843137 |
| Plasma_Lipid | label_deceased | species_conc_LPC(16:1) | 0.07843137 |
| Plasma_Lipid | label_deceased | species_conc_LPC(17:0) | 0.07843137 |
| Plasma_Lipid | label_deceased | species_conc_FFA(16:1) | 0.07843137 |
| Plasma_Lipid | label_deceased | species_conc_FFA(20:3) | 0.07843137 |
| Plasma_Lipid | label_deceased | species_conc_PC(18:0/18:1) | 0.07843137 |
| Plasma_Lipid | label_deceased | species_conc_PC(16:0/20:5) | 0.07843137 |
| Plasma_Lipid | label_deceased | species_conc_FFA(18:1) | 0.07843137 |
| Plasma_Lipid | label_deceased | species_conc_PC(18:1/20:5) | 0.07843137 |
| Plasma_Lipid | label_deceased | species_conc_LPC(16:0) | 0.05882353 |
| Plasma_Lipid | label_deceased | species_conc_FFA(18:2) | 0.05882353 |
| Plasma_Lipid | label_deceased | species_conc_FFA(20:4) | 0.05882353 |
| Plasma_Lipid | label_deceased | species_conc_PC(18:0/20:5) | 0.05882353 |
| Plasma_Lipid | label_deceased | species_conc_PC(18:0/22:6) | 0.05882353 |
| Plasma_Lipid | label_deceased | species_conc_PC(16:0/14:0) | 0.05882353 |
| Plasma_Lipid | label_deceased | species_conc_PC(18:1/16:1) | 0.05882353 |
| Plasma_Lipid | label_deceased | species_conc_PC(14:0/18:2) | 0.05882353 |

TABLE 7B
Protein Lipid Features to Endpoints
Survival

| | | Spearman |
|---|---|---|
| | Spearman | p- |
| | rho | value |
| plasma_protein_{—} | 0.116 | 0.416 |
| A0M8Q6_LAC7 | | |
| plasma_protein_{—} | −0.052 | 0.719 |
| B9A064_IGLL5 | | |
| plasma_protein_{—} | 0.164 | 0.251 |
| O14791_APOL1 | | |
| plasma_protein_{—} | 0.029 | 0.841 |
| O14980_XPO1 | | |
| plasma_protein_{—} | 0.117 | 0.412 |
| O15212_PFD6 | | |
| plasma_protein_{—} | −0.148 | 0.300 |
| O15230_LAMA5 | | |
| plasma_protein_{—} | −0.006 | 0.968 |
| O43866_CD5L | | |
| plasma_protein_{—} | −0.345 | 0.013 |
| O75369_FLNB | | |
| plasma_protein_{—} | −0.120 | 0.400 |
| O75390_CISY | | |
| plasma_protein_{—} | 0.307 | 0.028 |
| O75882_ATRN | | |
| plasma_protein_{—} | −0.003 | 0.984 |
| O95445_APOM | | |
| plasma_protein_{—} | 0.020 | 0.889 |
| P00450_CERU | | |
| plasma_protein_{—} | −0.070 | 0.624 |
| P00734_THRB | | |
| plasma_protein_{—} | 0.086 | 0.548 |
| P00736_C1R | | |
| plasma_protein_{—} | −0.175 | 0.219 |
| P00738_HPT | | |
| plasma_protein_{—} | 0.144 | 0.312 |
| P00746_CFAD | | |
| plasma_protein_{—} | 0.052 | 0.719 |
| P00747_PLMN | | |
| plasma_protein_{—} | 0.032 | 0.826 |
| P00748_FA12 | | |
| plasma_protein_{—} | 0.016 | 0.912 |
| P00751_CFAB | | |
| plasma_protein_{—} | −0.054 | 0.705 |
| P00966_ASSY | | |
| plasma_protein_{—} | −0.122 | 0.394 |
| P01008_ANT3 | | |
| plasma_protein_{—} | −0.168 | 0.239 |
| P01009_A1AT | | |
| plasma_protein_{—} | −0.228 | 0.107 |
| P01011_AACT | | |
| plasma_protein_{—} | 0.126 | 0.377 |
| P01019_ANGT | | |
| plasma_protein_{—} | 0.195 | 0.170 |
| P01023_A2MG | | |
| plasma_protein_{—} | 0.129 | 0.366 |
| P01024_CO3 | | |
| plasma_protein_{—} | 0.126 | 0.377 |
| P01031_CO5 | | |
| plasma_protein_{—} | −0.027 | 0.849 |
| P01042_KNG1 | | |
| plasma_protein_{—} | −0.270 | 0.055 |
| P01591_IGJ | | |
| plasma_protein_{—} | 0.224 | 0.114 |
| P01593_KV101 | | |
| plasma_protein_{—} | −0.069 | 0.631 |
| P01601_KV109 | | |
| plasma_protein_{—} | 0.080 | 0.575 |
| P01608_KV116 | | |
| plasma_protein_{—} | −0.031 | 0.830 |
| P01616_KV203 | | |
| plasma_protein_{—} | 0.014 | 0.920 |
| P01714_LV301 | | |
| plasma_protein_{—} | −0.126 | 0.377 |
| P01717_LV403 | | |
| plasma_protein_{—} | 0.293 | 0.037 |
| P01764_HV303 | | |
| plasma_protein_{—} | 0.275 | 0.051 |
| P01767_HV306 | | |
| plasma_protein_{—} | 0.127 | 0.373 |
| P01780_HV319 | | |
| plasma_protein_{—} | 0.411 | 0.003 |
| P01781_HV320 | | |
| plasma_protein_{—} | −0.020 | 0.889 |
| P01859_IGHG2 | | |
| plasma_protein_{—} | −0.006 | 0.968 |
| P01860_IGHG3 | | |
| plasma_protein_{—} | 0.037 | 0.795 |
| P01861_IGHG4 | | |
| plasma_protein_{—} | −0.037 | 0.795 |
| P01871_IGHM | | |
| plasma_protein_{—} | 0.001 | 0.992 |
| P01876_IGHA1 | | |
| plasma_protein_{—} | −0.142 | 0.320 |
| P01877_IGHA2 | | |
| plasma_protein_{—} | 0.174 | 0.223 |
| P01880_IGHD | | |
| plasma_protein_{—} | −0.256 | 0.070 |
| P02042_HBD | | |
| plasma_protein_{—} | −0.266 | 0.060 |
| P02649_APOE | | |
| plasma_protein_{—} | −0.132 | 0.356 |
| P02652_APOA2 | | |
| plasma_protein_{—} | −0.060 | 0.674 |
| P02654_APOC1 | | |
| plasma_protein_{—} | −0.131 | 0.361 |
| P02656_APOC3 | | |
| plasma_protein_{—} | −0.168 | 0.239 |
| P02671_FIBA | | |
| plasma_protein_{—} | −0.264 | 0.061 |
| P02743_SAMP | | |
| plasma_protein_{—} | −0.079 | 0.582 |
| P02745_C1QA | | |
| plasma_protein_{—} | 0.238 | 0.092 |
| P02746_C1QB | | |
| plasma_protein_{—} | −0.126 | 0.377 |
| P02747_C1QC | | |
| plasma_protein_{—} | −0.052 | 0.719 |
| P02748_CO9 | | |
| plasma_protein_{—} | −0.342 | 0.014 |
| P02749_APOH | | |
| plasma_protein_{—} | 0.362 | 0.009 |
| P02751_FINC | | |
| plasma_protein_{—} | −0.138 | 0.335 |
| P02753_RET4 | | |
| plasma_protein_{—} | 0.134 | 0.350 |
| P02760_AMBP | | |
| plasma_protein_{—} | −0.063 | 0.660 |
| P02763_A1AG1 | | |
| plasma_protein_{—} | −0.228 | 0.107 |
| P02766_TTHY | | |
| plasma_protein_{—} | −0.362 | 0.009 |
| P02774_VTDB | | |
| plasma_protein_{—} | 0.113 | 0.429 |
| P02775_CXCL7 | | |
| plasma_protein_{—} | 0.244 | 0.084 |
| P02787_TRFE | | |
| plasma_protein_{—} | −0.006 | 0.968 |
| P02790_HEMO | | |
| plasma_protein_{—} | 0.129 | 0.366 |
| P03952_KLKB1 | | |
| plasma_protein_{—} | 0.042 | 0.772 |
| P04003_C4BPA | | |
| plasma_protein_{—} | 0.151 | 0.291 |
| P04004_VTNC | | |
| plasma_protein_{—} | −0.114 | 0.424 |
| P04075_ALDOA | | |
| plasma_protein_{—} | −0.024 | 0.865 |
| P04114_APOB | | |
| plasma_protein_{—} | 0.052 | 0.719 |
| P04180_LCAT | | |
| plasma_protein_{—} | 0.039 | 0.787 |
| P04196_HRG | | |
| plasma_protein_{—} | −0.062 | 0.667 |
| P04217_A1BG | | |
| plasma_protein_{—} | −0.175 | 0.219 |
| P04275_VWF | | |
| plasma_protein_{—} | 0.293 | 0.037 |
| P04278_SHBG | | |
| plasma_protein_{—} | 0.000 | 1.000 |
| P04433_KV309 | | |
| plasma_protein_{—} | −0.098 | 0.496 |
| P04434_KV310 | | |
| plasma_protein_{—} | −0.080 | 0.575 |
| P04632_CPNS1 | | |
| plasma_protein_{—} | 0.034 | 0.810 |
| P05090_APOD | | |
| plasma_protein_{—} | 0.122 | 0.392 |
| P05154_IPSP | | |
| plasma_protein_{—} | −0.080 | 0.575 |
| P05155_IC1 | | |
| plasma_protein_{—} | −0.014 | 0.920 |
| P05156_CFAI | | |
| plasma_protein_{—} | 0.151 | 0.290 |
| P05452_TETN | | |
| plasma_protein_{—} | 0.037 | 0.795 |
| P05546_HEP2 | | |
| plasma_protein_{—} | 0.198 | 0.163 |
| P06310_KV206 | | |
| plasma_protein_{—} | 0.086 | 0.548 |
| P06396_GELS | | |
| plasma_protein_{—} | −0.106 | 0.458 |
| P06681_CO2 | | |
| plasma_protein_{—} | −0.231 | 0.103 |
| P06737_PYGL | | |
| plasma_protein_{—} | −0.148 | 0.300 |
| P06865_HEXA | | |
| plasma_protein_{—} | 0.075 | 0.602 |
| P07195_LDHB | | |
| plasma_protein_{—} | −0.095 | 0.508 |
| P07225_PROS | | |
| plasma_protein_{—} | 0.195 | 0.170 |
| P07357_CO8A | | |
| plasma_protein_{—} | 0.234 | 0.098 |
| P07358_CO8B | | |
| plasma_protein_{—} | −0.152 | 0.286 |
| P07384_CAN1 | | |
| plasma_protein_{—} | 0.032 | 0.825 |
| P07602_SAP | | |
| plasma_protein_{—} | −0.252 | 0.075 |
| P08133_ANXA6 | | |
| plasma_protein_{—} | 0.149 | 0.296 |
| P08185_CBG | | |
| plasma_protein_{—} | 0.011 | 0.936 |
| P08519_APOA | | |
| plasma_protein_{—} | 0.085 | 0.552 |
| P08571_CD14 | | |
| plasma_protein_{—} | 0.032 | 0.826 |
| P08603_CFAH | | |
| plasma_protein_{—} | 0.049 | 0.733 |
| P08670_VIME | | |
| plasma_protein_{—} | 0.032 | 0.826 |
| P08697_A2AP | | |
| plasma_protein_{—} | 0.072 | 0.617 |
| P09871_C1S | | |
| plasma_protein_{—} | 0.324 | 0.020 |
| POCOL5_CO4B | | |
| plasma_protein_{—} | −0.075 | 0.600 |
| PODJI8_SAA1 | | |
| plasma_protein_{—} | 0.235 | 0.096 |
| P10643_CO7 | | |
| plasma_protein_{—} | −0.268 | 0.057 |
| P10909_CLUS | | |
| plasma_protein_{—} | −0.222 | 0.118 |
| P11226_MBL2 | | |
| plasma_protein_{—} | −0.353 | 0.011 |
| P11277_SPTB1 | | |
| plasma_protein_{—} | −0.168 | 0.238 |
| P11413_G6PD | | |
| plasma_protein_{—} | 0.166 | 0.245 |
| P12109_CO6A1 | | |
| plasma_protein_{—} | −0.109 | 0.448 |
| P12429_ANXA3 | | |
| plasma_protein_{—} | −0.147 | 0.305 |
| P13671_CO6 | | |
| plasma_protein_{—} | −0.140 | 0.329 |
| P15169_CBPN | | |
| plasma_protein_{—} | 0.029 | 0.839 |
| P17936_IBP3 | | |
| plasma_protein_{—} | 0.271 | 0.055 |
| P18206_VINC | | |
| plasma_protein_{—} | 0.109 | 0.446 |
| P18428_LBP | | |
| plasma_protein_{—} | −0.079 | 0.582 |
| P19652_A1AG2 | | |
| plasma_protein_{—} | 0.030 | 0.834 |
| P19823_ITIH2 | | |
| plasma_protein_{—} | 0.121 | 0.399 |
| P19827_ITIH1 | | |
| plasma_protein_{—} | 0.285 | 0.043 |
| P20742_PZP | | |
| plasma_protein_{—} | −0.246 | 0.082 |
| P20810_ICAL | | |
| plasma_protein_{—} | −0.109 | 0.446 |
| P21333_FLNA | | |
| plasma_protein_{—} | 0.350 | 0.012 |
| P22352_GPX3 | | |
| plasma_protein_{—} | −0.118 | 0.411 |
| P22792_CPN2 | | |
| plasma_protein_{—} | −0.236 | 0.095 |
| P22891_PROZ | | |
| plasma_protein_{—} | −0.040 | 0.779 |
| P23083_HV103 | | |
| plasma_protein_{—} | 0.063 | 0.661 |
| P25815_S100P | | |
| plasma_protein_{—} | −0.187 | 0.189 |
| P26640_SYVC | | |
| plasma_protein_{—} | 0.184 | 0.195 |
| P27797_CALR | | |
| plasma_protein_{—} | −0.060 | 0.674 |
| P27918_PROP | | |
| plasma_protein_{—} | 0.195 | 0.170 |
| P29622_KAIN | | |
| plasma_protein_{—} | −0.047 | 0.741 |
| P30520_PURA2 | | |
| plasma_protein_{—} | 0.131 | 0.361 |
| P30740_ILEU | | |
| plasma_protein_{—} | 0.152 | 0.286 |
| P32754_HPPD | | |
| plasma_protein_{—} | 0.163 | 0.254 |
| P34932_HSP74 | | |
| plasma_protein_{—} | −0.121 | 0.399 |
| P35542_SAA4 | | |
| plasma_protein_{—} | −0.014 | 0.920 |
| P35579_MYH9 | | |
| plasma_protein_{—} | −0.187 | 0.188 |
| P35858_ALS | | |
| plasma_protein_{—} | 0.253 | 0.073 |
| P40763_STAT3 | | |
| plasma_protein_{—} | −0.139 | 0.329 |
| P42785_PCP | | |
| plasma_protein_{—} | −0.104 | 0.466 |
| P43490_NAMPT | | |
| plasma_protein_{—} | −0.118 | 0.411 |
| P46939_UTRO | | |
| plasma_protein_{—} | −0.135 | 0.345 |
| P49908_SEPP1 | | |
| plasma_protein_{—} | 0.178 | 0.211 |
| P50213_IDH3A | | |
| plasma_protein_{—} | −0.138 | 0.335 |
| P51884_LUM | | |
| plasma_protein_{—} | −0.014 | 0.924 |
| P53396_ACLY | | |
| plasma_protein_{—} | 0.251 | 0.076 |
| P54136_SYRC | | |
| plasma_protein_{—} | 0.207 | 0.145 |
| P54578_UBP14 | | |
| plasma_protein_{—} | −0.052 | 0.715 |
| P58107_EPIPL | | |
| plasma_protein_{—} | −0.134 | 0.349 |
| P62277_RS13 | | |
| plasma_protein_{—} | −0.175 | 0.219 |
| P68871_HBB | | |
| plasma_protein_{—} | 0.021 | 0.886 |
| P78347_GTF2I | | |
| plasma_protein_{—} | −0.009 | 0.952 |
| P80748_LV302 | | |
| plasma_protein_{—} | 0.051 | 0.721 |
| Q01082_SPTB2 | | |
| plasma_protein_{—} | −0.453 | 0.001 |
| Q03591_FHR1 | | |
| plasma_protein_{—} | 0.142 | 0.320 |
| Q05707_COEA1 | | |
| plasma_protein_{—} | −0.066 | 0.645 |
| Q08380_LG3BP | | |
| plasma_protein_{—} | −0.053 | 0.710 |
| Q09666_AHNK | | |
| plasma_protein_{—} | −0.187 | 0.190 |
| Q13451_FKBP5 | | |
| plasma_protein_{—} | −0.066 | 0.645 |
| Q13790_APOF | | |
| plasma_protein_{—} | −0.118 | 0.409 |
| Q14520_HABP2 | | |
| plasma_protein_{—} | 0.063 | 0.660 |
| Q14624_ITIH4 | | |
| plasma_protein_{—} | −0.237 | 0.094 |
| Q15149_PLEC | | |
| plasma_protein_{—} | 0.055 | 0.704 |
| Q15166_PON3 | | |
| plasma_protein_{—} | 0.100 | 0.485 |
| Q15363_TMED2 | | |
| plasma_protein_{—} | 0.063 | 0.659 |
| Q15833_STXB2 | | |
| plasma_protein_{—} | 0.175 | 0.219 |
| Q16610_ECM1 | | |
| plasma_protein_{—} | 0.092 | 0.519 |
| Q16719_KYNU | | |
| plasma_protein_{—} | 0.220 | 0.121 |
| Q2PZI1_D19L1 | | |
| plasma_protein_{—} | 0.065 | 0.648 |
| Q5SYB0_FRPD1 | | |
| plasma_protein_{—} | −0.171 | 0.230 |
| Q6YN16_HSDL2 | | |
| plasma_protein_{—} | −0.175 | 0.219 |
| Q7Z5P9_MUC19 | | |
| plasma_protein_{—} | −0.201 | 0.158 |
| Q86V48_LUZP1 | | |
| plasma_protein_{—} | −0.020 | 0.888 |
| Q86VP6_CAND1 | | |
| plasma_protein_{—} | −0.034 | 0.810 |
| Q8IWV7_UBR1 | | |
| plasma_protein_{—} | −0.220 | 0.121 |
| Q92496_FHR4 | | |
| plasma_protein_{—} | −0.067 | 0.639 |
| Q92835_SHIP1 | | |
| plasma_protein_{—} | −0.176 | 0.218 |
| Q92945_FUBP2 | | |
| plasma_protein_{—} | −0.276 | 0.050 |
| Q92954_PRG4 | | |
| plasma_protein_{—} | 0.078 | 0.589 |
| Q96HR3_MED30 | | |
| plasma_protein_{—} | −0.187 | 0.190 |
| Q96JQ2_CLMN | | |
| plasma_protein_{—} | −0.034 | 0.810 |
| Q96PD5_PGRP2 | | |
| plasma_protein_{—} | 0.003 | 0.983 |
| Q96Q06_PLIN4 | | |
| plasma_protein_{—} | −0.003 | 0.983 |
| Q99459_CDC5L | | |
| plasma_protein_{—} | −0.230 | 0.105 |
| Q99460_PSMD1 | | |
| plasma_protein_{—} | −0.123 | 0.388 |
| Q9BRA2_TXD17 | | |
| plasma_protein_{—} | 0.129 | 0.368 |
| Q9BXK5_B2L13 | | |
| plasma_protein_{—} | 0.002 | 0.992 |
| Q9HDC9_APMAP | | |
| plasma_protein_{—} | −0.102 | 0.477 |
| Q9NPR2_SEM4B | | |
| plasma_protein_{—} | −0.102 | 0.478 |
| Q9NTK5_OLA1 | | |
| plasma_protein_{—} | 0.106 | 0.458 |
| Q9NZ08_ERAP1 | | |
| plasma_protein_{—} | 0.051 | 0.723 |
| Q9NZP8_C1RL | | |
| plasma_protein_{—} | 0.017 | 0.904 |
| Q9P2E9_RRBP1 | | |
| plasma_protein_{—} | −0.153 | 0.284 |
| Q9P2T1_GMPR2 | | |
| plasma_protein_{—} | 0.126 | 0.377 |
| Q9UGM5_FETUB | | |
| plasma_protein_{—} | −0.122 | 0.394 |
| Q9Y2G3_AT11B | | |
| plasma_protein_{—} | 0.032 | 0.825 |
| O15067_PUR4 | | |
| plasma_protein_{—} | −0.068 | 0.633 |
| O43707_ACTN4 | | |
| plasma_protein_{—} | 0.165 | 0.248 |
| O94804_STK10 | | |
| plasma_protein_{—} | −0.235 | 0.097 |
| P00367_DHE3 | | |
| plasma_protein_{—} | 0.029 | 0.839 |
| P00558_PGK1 | | |
| plasma_protein_{—} | −0.003 | 0.982 |
| P00740_FA9 | | |
| plasma_protein_{—} | 0.127 | 0.374 |
| P02008_HBAZ | | |
| plasma_protein_{—} | −0.212 | 0.134 |
| P02545_LMNA | | |
| plasma_protein_{—} | −0.169 | 0.237 |
| P02788_TRFL | | |
| plasma_protein_{—} | −0.346 | 0.013 |
| P04083_ANXA1 | | |
| plasma_protein_{—} | 0.022 | 0.876 |
| P04216_THY1 | | |
| plasma_protein_{—} | 0.130 | 0.362 |
| P05160_F13B | | |
| plasma_protein_{—} | 0.026 | 0.857 |
| P07942_LAMB1 | | |
| plasma_protein_{—} | −0.114 | 0.424 |
| P10809_CH60 | | |
| plasma_protein_{—} | −0.220 | 0.120 |
| P11021_GRP78 | | |
| plasma_protein_{—} | −0.089 | 0.536 |
| P12277_KCRB | | |
| plasma_protein_{—} | −0.038 | 0.791 |
| P13639_EF2 | | |
| plasma_protein_{—} | 0.375 | 0.007 |
| P13797_PLST | | |
| plasma_protein_{—} | −0.228 | 0.108 |
| P14618_KPYM | | |
| plasma_protein_{—} | −0.253 | 0.073 |
| P14625_ENPL | | |
| plasma_protein_{—} | 0.058 | 0.684 |
| P17980_PRS6A | | |
| plasma_protein_{—} | 0.037 | 0.795 |
| P25786_PSA1 | | |
| plasma_protein_{—} | −0.266 | 0.060 |
| P28066_PSA5 | | |
| plasma_protein_{—} | 0.161 | 0.259 |
| P28838_AMPL | | |
| plasma_protein_{—} | 0.195 | 0.171 |
| P33176_KINH | | |
| plasma_protein_{—} | −0.064 | 0.657 |
| P34913_HYES | | |
| plasma_protein_{—} | −0.284 | 0.044 |
| P35052_GPC1 | | |
| plasma_protein_{—} | −0.113 | 0.429 |
| P35520_CBS | | |
| plasma_protein_{—} | −0.077 | 0.593 |
| P35908_K22E | | |
| plasma_protein_{—} | −0.188 | 0.185 |
| P38606_VATA | | |
| plasma_protein_{—} | −0.311 | 0.027 |
| P45974_UBP5 | | |
| plasma_protein_{—} | 0.017 | 0.908 |
| P48740_MASP1 | | |
| plasma_protein_{—} | −0.286 | 0.042 |
| P55290_CAD13 | | |
| plasma_protein_{—} | −0.111 | 0.437 |
| P60174_TPIS | | |
| plasma_protein_{—} | 0.048 | 0.740 |
| P60983_GMFB | | |
| plasma_protein_{—} | −0.063 | 0.660 |
| P62263_RS14 | | |
| plasma_protein_{—} | −0.249 | 0.078 |
| P62993_GRB2 | | |
| plasma_protein_{—} | −0.096 | 0.505 |
| P80188_NGAL | | |
| plasma_protein_{—} | −0.207 | 0.145 |
| Q00610_CLH1 | | |
| plasma_protein_{—} | −0.166 | 0.244 |
| Q12931_TRAP1 | | |
| plasma_protein_{—} | −0.239 | 0.092 |
| Q13177_PAK2 | | |
| plasma_protein_{—} | −0.131 | 0.358 |
| Q13283_G3BP1 | | |
| plasma_protein_{—} | −0.215 | 0.129 |
| Q14112_NID2 | | |
| plasma_protein_{—} | −0.060 | 0.674 |
| Q14204_DYHC1 | | |
| plasma_protein_{—} | 0.095 | 0.506 |
| Q14258_TRI25 | | |
| plasma_protein_{—} | 0.041 | 0.773 |
| Q14789_GOGB1 | | |
| plasma_protein_{—} | 0.133 | 0.350 |
| Q27J81_INF2 | | |
| plasma_protein_{—} | −0.212 | 0.135 |
| Q5KU26_COL12 | | |
| plasma_protein_{—} | 0.200 | 0.160 |
| Q6NZI2_PTRF | | |
| plasma_protein_{—} | −0.152 | 0.286 |
| Q7Z7G0_TARSH | | |
| plasma_protein_{—} | 0.235 | 0.097 |
| Q86UK5_LBN | | |
| plasma_protein_{—} | 0.155 | 0.278 |
| Q86VI3_IQGA3 | | |
| plasma_protein_{—} | 0.131 | 0.360 |
| Q8NCW5_NNRE | | |
| plasma_protein_{—} | −0.218 | 0.124 |
| Q93091_RNAS6 | | |
| plasma_protein_{—} | 0.142 | 0.321 |
| Q96KN2_CNDP1 | | |
| plasma_protein_{—} | −0.020 | 0.889 |
| Q9BRF8_CPPED | | |
| plasma_protein_{—} | 0.046 | 0.751 |
| Q9BXR6_FHR5 | | |
| plasma_protein_{—} | −0.023 | 0.873 |
| Q9Y244_POMP | | |
| tissue_protein_{—} | −0.082 | 0.577 |
| A0A075B6S5_KV127 | | |
| tissue_protein_{—} | 0.088 | 0.549 |
| A0A0C4DH35_HV335 | | |
| tissue_protein_{—} | −0.019 | 0.899 |
| A0A0C4DH36_HV338 | | |
| tissue_protein_{—} | −0.314 | 0.028 |
| A0AV96_RBM47 | | |
| tissue_protein_{—} | −0.111 | 0.448 |
| A0FGR8_ESYT2 | | |
| tissue_protein_{—} | −0.299 | 0.037 |
| A0MZ66_SHOT1 | | |
| tissue_protein_{—} | 0.301 | 0.036 |
| A1X283_SPD2B | | |
| tissue_protein_{—} | 0.235 | 0.104 |
| A6NMY6_AXA2L | | |
| tissue_protein_{—} | −0.095 | 0.518 |
| A6NNZ2_TBB8B | | |
| tissue_protein_{—} | 0.003 | 0.983 |
| O00154_BACH | | |
| tissue_protein_{—} | −0.315 | 0.027 |
| O00178_GTPB1 | | |
| tissue_protein_{—} | −0.185 | 0.203 |
| O00182_LEG9 | | |
| tissue_protein_{—} | 0.114 | 0.436 |
| O00186_STXB3 | | |
| tissue_protein_{—} | −0.156 | 0.283 |
| O00194_RB27B | | |
| tissue_protein_{—} | 0.069 | 0.636 |
| O00214_LEG8 | | |
| tissue_protein_{—} | −0.196 | 0.177 |
| O00267_SPT5H | | |
| tissue_protein_{—} | 0.039 | 0.789 |
| O00291_HIP1 | | |
| tissue_protein_{—} | −0.185 | 0.204 |
| O00299_CLIC1 | | |
| tissue_protein_{—} | −0.203 | 0.161 |
| O00391_QSOX1 | | |
| tissue_protein_{—} | −0.147 | 0.315 |
| O00400_ACATN | | |
| tissue_protein_{—} | 0.169 | 0.247 |
| O00469_PLOD2 | | |
| tissue_protein_{—} | −0.154 | 0.290 |
| O00483_NDUA4 | | |
| tissue_protein_{—} | 0.127 | 0.386 |
| O00499_BIN1 | | |
| tissue_protein_{—} | −0.111 | 0.447 |
| O00515_LAD1 | | |
| tissue_protein_{—} | 0.086 | 0.555 |
| O00541_PESC | | |
| tissue_protein_{—} | 0.183 | 0.209 |
| O00629_IMA3 | | |
| tissue_protein_{—} | 0.275 | 0.056 |
| O00754_MA2B1 | | |
| tissue_protein_{—} | −0.160 | 0.271 |
| O14498_ISLR | | |
| tissue_protein_{—} | −0.231 | 0.110 |
| O14531_DPYL4 | | |
| tissue_protein_{—} | −0.102 | 0.485 |
| O14556_G3PT | | |
| tissue_protein_{—} | −0.008 | 0.955 |
| O14558_HSPB6 | | |
| tissue_protein_{—} | −0.150 | 0.302 |
| O14732_IMPA2 | | |
| tissue_protein_{—} | −0.176 | 0.225 |
| O14745_NHRF1 | | |
| tissue_protein_{—} | −0.147 | 0.312 |
| O14786_NRP1 | | |
| tissue_protein_{—} | −0.191 | 0.189 |
| O14874_BCKD | | |
| tissue_protein_{—} | −0.264 | 0.067 |
| O14907_TX1B3 | | |
| tissue_protein_{—} | −0.154 | 0.290 |
| O14972_VP26C | | |
| tissue_protein_{—} | 0.127 | 0.385 |
| O14974_MYPT1 | | |
| tissue_protein_{—} | −0.083 | 0.571 |
| O15027_SC16A | | |
| tissue_protein_{—} | −0.107 | 0.465 |
| O15067_PUR4 | | |
| tissue_protein_{—} | −0.274 | 0.057 |
| O15144_ARPC2 | | |
| tissue_protein_{—} | −0.126 | 0.388 |
| O15162_PLS1 | | |
| tissue_protein_{—} | −0.018 | 0.900 |
| O15173_PGRC2 | | |
| tissue_protein_{—} | 0.061 | 0.675 |
| O15195_VILL | | |
| tissue_protein_{—} | 0.008 | 0.958 |
| O15230_LAMA5 | | |
| tissue_protein_{—} | −0.274 | 0.057 |
| O15269_SPTC1 | | |
| tissue_protein_{—} | −0.180 | 0.215 |
| O15305_PMM2 | | |
| tissue_protein_{—} | 0.110 | 0.450 |
| O15355_PPM1G | | |
| tissue_protein_{—} | −0.243 | 0.092 |
| O15371_EIF3D | | |
| tissue_protein_{—} | −0.177 | 0.223 |
| O15382_BCAT2 | | |
| tissue_protein_{—} | 0.114 | 0.436 |
| O15460_P4HA2 | | |
| tissue_protein_{—} | −0.086 | 0.555 |
| O15484_CAN5 | | |
| tissue_protein_{—} | −0.169 | 0.245 |
| O15511_ARPC5 | | |
| tissue_protein_{—} | −0.202 | 0.163 |
| O15533_TPSN | | |
| tissue_protein_{—} | −0.037 | 0.801 |
| O43175_SERA | | |
| tissue_protein_{—} | −0.069 | 0.636 |
| O43252_PAPS1 | | |
| tissue_protein_{—} | −0.163 | 0.262 |
| O43324_MCA3 | | |
| tissue_protein_{—} | −0.015 | 0.918 |
| O43402_EMC8 | | |
| tissue_protein_{—} | −0.174 | 0.233 |
| O43414_ERI3 | | |
| tissue_protein_{—} | −0.260 | 0.071 |
| O43464_HTRA2 | | |
| tissue_protein_{—} | 0.160 | 0.271 |
| O43493_TGON2 | | |
| tissue_protein_{—} | −0.003 | 0.983 |
| O43598_DNPH1 | | |
| tissue_protein_{—} | −0.062 | 0.674 |
| O43615_TIM44 | | |
| tissue_protein_{—} | −0.027 | 0.854 |
| O43660_PLRG1 | | |
| tissue_protein_{—} | −0.160 | 0.271 |
| O43704_ST1B1 | | |
| tissue_protein_{—} | 0.347 | 0.015 |
| O43765_SGTA | | |
| tissue_protein_{—} | 0.051 | 0.728 |
| O43772_MCAT | | |
| tissue_protein_{—} | −0.218 | 0.131 |
| O43776_SYNC | | |
| tissue_protein_{—} | −0.089 | 0.542 |
| O43795_MYO1B | | |
| tissue_protein_{—} | 0.000 | 1.000 |
| O43826_G6PT1 | | |
| tissue_protein_{—} | −0.143 | 0.328 |
| O43837_IDH3B | | |
| tissue_protein_{—} | −0.003 | 0.982 |
| O60216_RAD21 | | |
| tissue_protein_{—} | 0.131 | 0.368 |
| O60218_AK1BA | | |
| tissue_protein_{—} | −0.039 | 0.789 |
| O60234_GMFG | | |
| tissue_protein_{—} | −0.159 | 0.274 |
| O60240_PLIN1 | | |
| tissue_protein_{—} | −0.124 | 0.398 |
| O60271_JIP4 | | |
| tissue_protein_{—} | −0.206 | 0.155 |
| O60341_KDM1A | | |
| tissue_protein_{—} | −0.160 | 0.271 |
| O60437_PEPL | | |
| tissue_protein_{—} | −0.029 | 0.846 |
| O60488_ACSL4 | | |
| tissue_protein_{—} | −0.237 | 0.100 |
| O60504_VINEX | | |
| tissue_protein_{—} | 0.291 | 0.043 |
| O60687_SRPX2 | | |
| tissue_protein_{—} | −0.140 | 0.338 |
| O60704_TPST2 | | |
| tissue_protein_{—} | −0.043 | 0.769 |
| O60762_DPM1 | | |
| tissue_protein_{—} | −0.303 | 0.034 |
| O60763_USO1 | | |
| tissue_protein_{—} | −0.013 | 0.927 |
| O60784_TOM1 | | |
| tissue_protein_{—} | 0.098 | 0.502 |
| O60825_F262 | | |
| tissue_protein_{—} | −0.010 | 0.948 |
| O60831_PRAF2 | | |
| tissue_protein_{—} | −0.133 | 0.364 |
| O60841_IF2P | | |
| tissue_protein_{—} | 0.112 | 0.445 |
| O60869_EDF1 | | |
| tissue_protein_{—} | −0.122 | 0.404 |
| O60888_CUTA | | |
| tissue_protein_{—} | −0.088 | 0.549 |
| O75165_DJC13 | | |
| tissue_protein_{—} | −0.020 | 0.892 |
| O75339_CILP1 | | |
| tissue_protein_{—} | −0.286 | 0.046 |
| O75369_FLNB | | |
| tissue_protein_{—} | −0.246 | 0.089 |
| O75380_NDUS6 | | |
| tissue_protein_{—} | −0.444 | 0.001 |
| O75396_SC22B | | |
| tissue_protein_{—} | −0.304 | 0.034 |
| O75431_MTX2 | | |
| tissue_protein_{—} | −0.249 | 0.084 |
| O75489_NDUS3 | | |
| tissue_protein_{—} | −0.319 | 0.025 |
| O75494_SRS10 | | |
| tissue_protein_{—} | −0.045 | 0.759 |
| O75521_ECI2 | | |
| tissue_protein_{—} | −0.116 | 0.428 |
| O75594_PGRP1 | | |
| tissue_protein_{—} | −0.004 | 0.981 |
| O75629_CREG1 | | |
| tissue_protein_{—} | −0.162 | 0.266 |
| O75674_TM1L1 | | |
| tissue_protein_{—} | −0.235 | 0.103 |
| O75688_PPM1B | | |
| tissue_protein_{—} | −0.165 | 0.257 |
| O75695_XRP2 | | |
| tissue_protein_{—} | −0.089 | 0.541 |
| O75764_TCEA3 | | |
| tissue_protein_{—} | −0.121 | 0.408 |
| O75822_EIF3J | | |
| tissue_protein_{—} | 0.002 | 0.991 |
| O75830_SPI2 | | |
| tissue_protein_{—} | −0.232 | 0.108 |
| O75891_AL1L1 | | |
| tissue_protein_{—} | −0.003 | 0.981 |
| O75923_DYSF | | |
| tissue_protein_{—} | −0.013 | 0.927 |
| O75935_DCTN3 | | |
| tissue_protein_{—} | −0.329 | 0.021 |
| O75947_ATP5H | | |
| tissue_protein_{—} | −0.084 | 0.564 |
| O75954_TSN9 | | |
| tissue_protein_{—} | −0.043 | 0.769 |
| O76003_GLRX3 | | |
| tissue_protein_{—} | −0.263 | 0.068 |
| O76094_SRP72 | | |
| tissue_protein_{—} | 0.036 | 0.804 |
| O76095_JTB | | |
| tissue_protein_{—} | −0.084 | 0.565 |
| O94819_KBTBB | | |
| tissue_protein_{—} | −0.237 | 0.101 |
| O94874_UFL1 | | |
| tissue_protein_{—} | −0.185 | 0.204 |
| O94875_SRBS2 | | |
| tissue_protein_{—} | −0.259 | 0.073 |
| O94905_ERLN2 | | |
| tissue_protein_{—} | 0.006 | 0.966 |
| O94919_ENDD1 | | |
| tissue_protein_{—} | −0.253 | 0.079 |
| O94925_GLSK | | |
| tissue_protein_{—} | −0.266 | 0.065 |
| O95070_YIF1A | | |
| tissue_protein_{—} | 0.003 | 0.981 |
| O95139_NDUB6 | | |
| tissue_protein_{—} | −0.015 | 0.916 |
| O95154_ARK73 | | |
| tissue_protein_{—} | −0.094 | 0.519 |
| O95159_ZFPL1 | | |
| tissue_protein_{—} | 0.204 | 0.160 |
| O95249_GOSR1 | | |
| tissue_protein_{—} | −0.188 | 0.196 |
| O95299_NDUAA | | |
| tissue_protein_{—} | −0.052 | 0.721 |
| O95302_FKBP9 | | |
| tissue_protein_{—} | −0.165 | 0.256 |
| O95340_PAPS2 | | |
| tissue_protein_{—} | −0.111 | 0.448 |
| O95379_TFIP8 | | |
| tissue_protein_{—} | −0.155 | 0.289 |
| O95486_SC24A | | |
| tissue_protein_{—} | −0.198 | 0.173 |
| O95571_ETHE1 | | |
| tissue_protein_{—} | −0.110 | 0.450 |
| O95573_ACSL3 | | |
| tissue_protein_{—} | −0.088 | 0.549 |
| O95671_ASML | | |
| tissue_protein_{—} | 0.117 | 0.423 |
| O95674_CDS2 | | |
| tissue_protein_{—} | −0.145 | 0.319 |
| O95716_RAB3D | | |
| tissue_protein_{—} | −0.289 | 0.044 |
| O95782_AP2A1 | | |
| tissue_protein_{—} | 0.019 | 0.894 |
| O95822_DCMC | | |
| tissue_protein_{—} | −0.188 | 0.196 |
| O95831_AIFM1 | | |
| tissue_protein_{—} | 0.024 | 0.867 |
| O95857_TSN13 | | |
| tissue_protein_{—} | 0.011 | 0.939 |
| O95870_ABHGA | | |
| tissue_protein_{—} | 0.216 | 0.137 |
| O95965_ITGBL | | |
| tissue_protein_{—} | 0.015 | 0.916 |
| O95967_FBLN4 | | |
| tissue_protein_{—} | −0.030 | 0.837 |
| O95980_RECK | | |
| tissue_protein_{—} | −0.175 | 0.228 |
| O96000_NDUBA | | |
| tissue_protein_{—} | −0.111 | 0.446 |
| O96005_CLPT1 | | |
| tissue_protein_{—} | −0.170 | 0.244 |
| P00167_CYB5 | | |
| tissue_protein_{—} | −0.243 | 0.092 |
| P00325_ADH1B | | |
| tissue_protein_{—} | −0.265 | 0.066 |
| P00338_LDHA | | |
| tissue_protein_{—} | −0.308 | 0.031 |
| P00352_AL1A1 | | |
| tissue_protein_{—} | −0.163 | 0.263 |
| P00568_KAD1 | | |
| tissue_protein_{—} | −0.015 | 0.917 |
| P00742_FA10 | | |
| tissue_protein_{—} | 0.065 | 0.659 |
| P00915_CAH1 | | |
| tissue_protein_{—} | 0.137 | 0.348 |
| P00918_CAH2 | | |
| tissue_protein_{—} | −0.152 | 0.296 |
| P00995_ISK1 | | |
| tissue_protein_{—} | −0.018 | 0.900 |
| P01033_TIMP1 | | |
| tissue_protein_{—} | 0.093 | 0.524 |
| P01275_GLUC | | |
| tissue_protein_{—} | −0.142 | 0.332 |
| P01591_IGJ | | |
| tissue_protein_{—} | −0.232 | 0.109 |
| P01599_KV117 | | |
| tissue_protein_{—} | 0.028 | 0.850 |
| P01619_KV320 | | |
| tissue_protein_{—} | −0.191 | 0.188 |
| P01700_LV147 | | |
| tissue_protein_{—} | 0.057 | 0.697 |
| P01705_LV223 | | |
| tissue_protein_{—} | 0.069 | 0.636 |
| P01714_LV319 | | |
| tissue_protein_{—} | 0.179 | 0.218 |
| P01833_PIGR | | |
| tissue_protein_{—} | −0.065 | 0.659 |
| P01903_DRA | | |
| tissue_protein_{—} | −0.021 | 0.884 |
| P01909_DQA1 | | |
| tissue_protein_{—} | −0.069 | 0.639 |
| P01911_DRB | | |
| tissue_protein_{—} | −0.006 | 0.967 |
| P02042_HBD | | |
| tissue_protein_{—} | 0.090 | 0.538 |
| P02100_HBE | | |
| tissue_protein_{—} | 0.116 | 0.428 |
| P02144_MYG | | |
| tissue_protein_{—} | 0.323 | 0.024 |
| P02452_CO1A1 | | |
| tissue_protein_{—} | 0.325 | 0.023 |
| P02458_CO2A1 | | |
| tissue_protein_{—} | 0.315 | 0.027 |
| P02461_CO3A1 | | |
| tissue_protein_{—} | 0.073 | 0.617 |
| P02538_K2C6A | | |
| tissue_protein_{—} | 0.057 | 0.695 |
| P02549_SPTA1 | | |
| tissue_protein_{—} | 0.046 | 0.756 |
| P02654_APOC1 | | |
| tissue_protein_{—} | −0.103 | 0.483 |
| P02656_APOC3 | | |
| tissue_protein_{—} | −0.035 | 0.809 |
| P02730_B3AT | | |
| tissue_protein_{—} | −0.261 | 0.070 |
| P02741_CRP | | |
| tissue_protein_{—} | 0.028 | 0.850 |
| P02745_C1QA | | |
| tissue_protein_{—} | −0.065 | 0.659 |
| P02746_C1QB | | |
| tissue_protein_{—} | −0.058 | 0.690 |
| P02751_FINC | | |
| tissue_protein_{—} | 0.064 | 0.662 |
| P02775_CXCL7 | | |
| tissue_protein_{—} | 0.176 | 0.225 |
| P02786_TFR1 | | |
| tissue_protein_{—} | 0.025 | 0.867 |
| P02792_FRIL | | |
| tissue_protein_{—} | 0.046 | 0.753 |
| P02794_FRIH | | |
| tissue_protein_{—} | 0.012 | 0.933 |
| P03886_NU1M | | |
| tissue_protein_{—} | 0.243 | 0.092 |
| P03905_NU4M | | |
| tissue_protein_{—} | 0.025 | 0.865 |
| P03915_NU5M | | |
| tissue_protein_{—} | 0.048 | 0.746 |
| P03973_SLPI | | |
| tissue_protein_{—} | −0.068 | 0.644 |
| P04040_CATA | | |
| tissue_protein_{—} | 0.022 | 0.883 |
| P04054_PA21B | | |
| tissue_protein_{—} | −0.383 | 0.007 |
| P04075_ALDOA | | |
| tissue_protein_{—} | −0.191 | 0.189 |
| P04083_ANXA1 | | |
| tissue_protein_{—} | −0.022 | 0.883 |
| P04216_THY1 | | |
| tissue_protein_{—} | −0.061 | 0.676 |
| P04259_K2C6B | | |
| tissue_protein_{—} | −0.302 | 0.035 |
| P04439_HLAA | | |
| tissue_protein_{—} | −0.043 | 0.767 |
| P04440_DPB1 | | |
| tissue_protein_{—} | 0.062 | 0.674 |
| P04746_AMYP | | |
| tissue_protein_{—} | −0.319 | 0.026 |
| P04843_RPN1 | | |
| tissue_protein_{—} | −0.034 | 0.817 |
| P05026_AT1B1 | | |
| tissue_protein_{—} | −0.130 | 0.372 |
| P05062_ALDOB | | |
| tissue_protein_{—} | −0.273 | 0.057 |
| P05114_HMGN1 | | |
| tissue_protein_{—} | 0.027 | 0.855 |
| P05121_PAI1 | | |
| tissue_protein_{—} | −0.072 | 0.625 |
| P05161_ISG15 | | |
| tissue_protein_{—} | −0.368 | 0.009 |
| P05162_LEG2 | | |
| tissue_protein_{—} | −0.335 | 0.018 |
| P05362_ICAM1 | | |
| tissue_protein_{—} | −0.236 | 0.103 |
| P05386_RLA1 | | |
| tissue_protein_{—} | −0.262 | 0.069 |
| P05387_RLA2 | | |
| tissue_protein_{—} | −0.477 | 0.001 |
| P05388_RLA0 | | |
| tissue_protein_{—} | −0.011 | 0.941 |
| P05451_REG1A | | |
| tissue_protein_{—} | −0.040 | 0.785 |
| P05455_LA | | |
| tissue_protein_{—} | 0.154 | 0.290 |
| P05543_THBG | | |
| tissue_protein_{—} | 0.038 | 0.793 |
| P05556_ITB1 | | |
| tissue_protein_{—} | 0.351 | 0.013 |
| P05997_CO5A2 | | |
| tissue_protein_{—} | 0.090 | 0.540 |
| P06280_AGAL | | |
| tissue_protein_{—} | −0.111 | 0.448 |
| P06703_S10A6 | | |
| tissue_protein_{—} | −0.059 | 0.686 |
| P06731_CEAM5 | | |
| tissue_protein_{—} | −0.345 | 0.015 |
| P06733_ENOA | | |
| tissue_protein_{—} | −0.143 | 0.327 |
| P06870_KLK1 | | |
| tissue_protein_{—} | 0.064 | 0.662 |
| P07093_GDN | | |
| tissue_protein_{—} | −0.006 | 0.967 |
| P07099_HYEP | | |
| tissue_protein_{—} | −0.233 | 0.107 |
| P07108_ACBP | | |
| tissue_protein_{—} | −0.194 | 0.183 |
| P07148_FABPL | | |
| tissue_protein_{—} | −0.299 | 0.037 |
| P07195_LDHB | | |
| tissue_protein_{—} | −0.052 | 0.723 |
| P07225_PROS | | |
| tissue_protein_{—} | −0.328 | 0.022 |
| P07237_PDIA1 | | |
| tissue_protein_{—} | 0.002 | 0.991 |
| P07311_ACYP1 | | |
| tissue_protein_{—} | −0.020 | 0.891 |
| P07477_TRY1 | | |
| tissue_protein_{—} | −0.022 | 0.883 |
| P07478_TRY2 | | |
| tissue_protein_{—} | −0.157 | 0.282 |
| P07686_HEXB | | |
| tissue_protein_{—} | 0.039 | 0.793 |
| P07711_CATL1 | | |
| tissue_protein_{—} | −0.306 | 0.032 |
| P07737_PROF1 | | |
| tissue_protein_{—} | 0.145 | 0.320 |
| P07738_PMGE | | |
| tissue_protein_{—} | −0.125 | 0.394 |
| P07858_CATB | | |
| tissue_protein_{—} | −0.048 | 0.743 |
| P07902_GALT | | |
| tissue_protein_{—} | −0.422 | 0.003 |
| P07954_FUMH | | |
| tissue_protein_{—} | 0.126 | 0.388 |
| P07996_TSP1 | | |
| tissue_protein_{—} | 0.006 | 0.967 |
| P07998_RNAS1 | | |
| tissue_protein_{—} | 0.335 | 0.018 |
| P08123_CO1A2 | | |
| tissue_protein_{—} | 0.158 | 0.279 |
| P08174_DAF | | |
| tissue_protein_{—} | 0.234 | 0.106 |
| P08185_CBG | | |
| tissue_protein_{—} | 0.035 | 0.809 |
| P08217_CEL2A | | |
| tissue_protein_{—} | −0.108 | 0.461 |
| P08218_CEL2B | | |
| tissue_protein_{—} | −0.003 | 0.983 |
| P08236_BGLR | | |
| tissue_protein_{—} | −0.194 | 0.182 |
| P08240_SRPRA | | |
| tissue_protein_{—} | −0.244 | 0.092 |
| P08243_ASNS | | |
| tissue_protein_{—} | 0.153 | 0.294 |
| P08493_MGP | | |
| tissue_protein_{—} | −0.023 | 0.877 |
| P08519_APOA | | |
| tissue_protein_{—} | −0.228 | 0.116 |
| P08559_ODPA | | |
| tissue_protein_{—} | −0.185 | 0.203 |
| P08575_PTPRC | | |
| tissue_protein_{—} | −0.184 | 0.206 |
| P08579_RU2B | | |
| tissue_protein_{—} | −0.182 | 0.212 |
| P08727_K1C19 | | |
| tissue_protein_{—} | −0.431 | 0.002 |
| P08842_STS | | |
| tissue_protein_{—} | 0.006 | 0.967 |
| P08861_CEL3B | | |
| tissue_protein_{—} | −0.369 | 0.009 |
| P09012_SNRPA | | |
| tissue_protein_{—} | 0.022 | 0.883 |
| P09093_CEL3A | | |
| tissue_protein_{—} | −0.162 | 0.265 |
| P09132_SRP19 | | |
| tissue_protein_{—} | −0.100 | 0.492 |
| P09237_MMP7 | | |
| tissue_protein_{—} | −0.323 | 0.024 |
| P09417_DHPR | | |
| tissue_protein_{—} | −0.128 | 0.382 |
| P09455_RET1 | | |
| tissue_protein_{—} | −0.215 | 0.137 |
| P09493_TPM1 | | |
| tissue_protein_{—} | −0.393 | 0.005 |
| P09496_CLCA | | |
| tissue_protein_{—} | −0.163 | 0.263 |
| P09622_DLDH | | |
| tissue_protein_{—} | 0.123 | 0.399 |
| P09668_CATH | | |
| tissue_protein_{—} | 0.000 | 1.000 |
| P09758_TACD2 | | |
| tissue_protein_{—} | 0.008 | 0.958 |
| P0DOX3_IGD | | |
| tissue_protein_{—} | −0.095 | 0.514 |
| P10321_HLAC | | |
| tissue_protein_{—} | −0.311 | 0.030 |
| P10515_ODP2 | | |
| tissue_protein_{—} | −0.108 | 0.460 |
| P10606_COX5B | | |
| tissue_protein_{—} | −0.249 | 0.084 |
| P10620_MGST1 | | |
| tissue_protein_{—} | −0.058 | 0.690 |
| P11047_LAMC1 | | |
| tissue_protein_{—} | −0.052 | 0.721 |
| P11117_PPAL | | |
| tissue_protein_{—} | −0.409 | 0.003 |
| P11177_ODPB | | |
| tissue_protein_{—} | −0.091 | 0.536 |
| P11182_ODB2 | | |
| tissue_protein_{—} | 0.080 | 0.584 |
| P11233_RALA | | |
| tissue_protein_{—} | −0.031 | 0.834 |
| P11234_RALB | | |
| tissue_protein_{—} | 0.212 | 0.143 |
| P11279_LAMP1 | | |
| tissue_protein_{—} | −0.255 | 0.076 |
| P11310_ACADM | | |
| tissue_protein_{—} | −0.305 | 0.033 |
| P11498_PYC | | |
| tissue_protein_{—} | −0.237 | 0.101 |
| P11586_CITC | | |
| tissue_protein_{—} | −0.014 | 0.923 |
| P12104_FABPI | | |
| tissue_protein_{—} | 0.431 | 0.002 |
| P12107_COBA1 | | |
| tissue_protein_{—} | −0.345 | 0.015 |
| P12268_IMDH2 | | |
| tissue_protein_{—} | −0.382 | 0.007 |
| P12277_KCRB | | |
| tissue_protein_{—} | −0.053 | 0.717 |
| P12532_KCRU | | |
| tissue_protein_{—} | −0.367 | 0.009 |
| P12694_ODBA | | |
| tissue_protein_{—} | 0.093 | 0.523 |
| P12838_DEF4 | | |
| tissue_protein_{—} | 0.014 | 0.923 |
| P12931_SRC | | |
| tissue_protein_{—} | −0.012 | 0.933 |
| P13611_CSPG2 | | |
| tissue_protein_{—} | −0.257 | 0.075 |
| P13674_P4HA1 | | |
| tissue_protein_{—} | −0.206 | 0.155 |
| P13716_HEM2 | | |
| tissue_protein_{—} | −0.142 | 0.332 |
| P13797_PLST | | |
| tissue_protein_{—} | −0.357 | 0.012 |
| P13804_ETFA | | |
| tissue_protein_{—} | 0.317 | 0.026 |
| P13942_COBA2 | | |
| tissue_protein_{—} | −0.133 | 0.362 |
| P14091_CATE | | |
| tissue_protein_{—} | −0.138 | 0.346 |
| P14406_CX7A2 | | |
| tissue_protein_{—} | −0.215 | 0.137 |
| P14543_NID1 | | |
| tissue_protein_{—} | −0.215 | 0.137 |
| P14618_KPYM | | |
| tissue_protein_{—} | −0.058 | 0.690 |
| P14927_QCR7 | | |
| tissue_protein_{—} | −0.003 | 0.983 |
| P15085_CBPA1 | | |
| tissue_protein_{—} | −0.268 | 0.063 |
| P15090_FABP4 | | |
| tissue_protein_{—} | 0.093 | 0.523 |
| P15104_GLNA | | |
| tissue_protein_{—} | −0.138 | 0.343 |
| P15144_AMPN | | |
| tissue_protein_{—} | −0.093 | 0.523 |
| P15170_ERF3A | | |
| tissue_protein_{—} | −0.254 | 0.078 |
| P15289_ARSA | | |
| tissue_protein_{—} | −0.164 | 0.261 |
| P15502_ELN | | |
| tissue_protein_{—} | 0.205 | 0.159 |
| P15927_RFA2 | | |
| tissue_protein_{—} | −0.032 | 0.827 |
| P15941_MUC1 | | |
| tissue_protein_{—} | 0.270 | 0.060 |
| P16035_TIMP2 | | |
| tissue_protein_{—} | 0.188 | 0.197 |
| P16144_ITB4 | | |
| tissue_protein_{—} | −0.073 | 0.617 |
| P16157_ANK1 | | |
| tissue_protein_{—} | −0.314 | 0.028 |
| P16219_ACADS | | |
| tissue_protein_{—} | −0.206 | 0.155 |
| P16401_H15 | | |
| tissue_protein_{—} | 0.185 | 0.203 |
| P16402_H13 | | |
| tissue_protein_{—} | −0.108 | 0.460 |
| P16422_EPCAM | | |
| tissue_protein_{—} | 0.020 | 0.889 |
| P16519_NEC2 | | |
| tissue_protein_{—} | −0.059 | 0.688 |
| P16671_CD36 | | |
| tissue_protein_{—} | −0.043 | 0.768 |
| P16930_FAAA | | |
| tissue_protein_{—} | −0.255 | 0.077 |
| P16949_STMN1 | | |
| tissue_protein_{—} | −0.050 | 0.732 |
| P17026_ZNF22 | | |
| tissue_protein_{—} | −0.172 | 0.237 |
| P17096_HMGA1 | | |
| tissue_protein_{—} | −0.007 | 0.964 |
| P17301_ITA2 | | |
| tissue_protein_{—} | −0.031 | 0.830 |
| P17538_CTRB1 | | |
| tissue_protein_{—} | −0.311 | 0.030 |
| P17693_HLAG | | |
| tissue_protein_{—} | 0.018 | 0.900 |
| P17813_EGLN | | |
| tissue_protein_{—} | −0.246 | 0.088 |
| P17931_LEG3 | | |
| tissue_protein_{—} | −0.276 | 0.055 |
| P18031_PTN1 | | |
| tissue_protein_{—} | −0.128 | 0.381 |
| P18065_IBP2 | | |
| tissue_protein_{—} | 0.045 | 0.759 |
| P18283_GPX2 | | |
| tissue_protein_{—} | −0.054 | 0.713 |
| P18583_SON | | |
| tissue_protein_{—} | −0.037 | 0.801 |
| P18621_RL17 | | |
| tissue_protein_{—} | −0.018 | 0.902 |
| P19075_TSN8 | | |
| tissue_protein_{—} | −0.172 | 0.236 |
| P19404_NDUV2 | | |
| tissue_protein_{—} | −0.162 | 0.267 |
| P19838_NFKB1 | | |
| tissue_protein_{—} | 0.080 | 0.583 |
| P19961_AMY2B | | |
| tissue_protein_{—} | −0.280 | 0.051 |
| P19971_TYPH | | |
| tissue_protein_{—} | −0.099 | 0.497 |
| P20020_AT2B1 | | |
| tissue_protein_{—} | −0.082 | 0.577 |
| P20142_PEPC | | |
| tissue_protein_{—} | −0.300 | 0.036 |
| P20290_BTF3 | | |
| tissue_protein_{—} | −0.285 | 0.047 |
| P20292_AL5AP | | |
| tissue_protein_{—} | 0.135 | 0.356 |
| P20591_MX1 | | |
| tissue_protein_{—} | 0.011 | 0.942 |
| P20702_ITAX | | |
| tissue_protein_{—} | 0.397 | 0.005 |
| P20908_CO5A1 | | |
| tissue_protein_{—} | −0.119 | 0.414 |
| P20962_PTMS | | |
| tissue_protein_{—} | −0.138 | 0.343 |
| P21266_GSTM3 | | |
| tissue_protein_{—} | −0.023 | 0.873 |
| P21283_VATC1 | | |
| tissue_protein_{—} | −0.268 | 0.063 |
| P21333_FLNA | | |
| tissue_protein_{—} | −0.240 | 0.097 |
| P21397_AOFA | | |
| tissue_protein_{—} | −0.277 | 0.054 |
| P21399_ACOC | | |
| tissue_protein_{—} | −0.067 | 0.649 |
| P21741_MK | | |
| tissue_protein_{—} | 0.065 | 0.659 |
| P21810_PGS1 | | |
| tissue_protein_{—} | −0.039 | 0.793 |
| P21953_ODBB | | |
| tissue_protein_{—} | −0.280 | 0.051 |
| P21980_TGM2 | | |
| tissue_protein_{—} | −0.202 | 0.164 |
| P22033_MUTA | | |
| tissue_protein_{—} | −0.328 | 0.021 |
| P22059_OSBP1 | | |
| tissue_protein_{—} | 0.141 | 0.332 |
| P22090_RS4Y1 | | |
| tissue_protein_{—} | 0.003 | 0.982 |
| P22492_H1T | | |
| tissue_protein_{—} | −0.345 | 0.015 |
| P22695_QCR2 | | |
| tissue_protein_{—} | −0.037 | 0.802 |
| P22748_CAH4 | | |
| tissue_protein_{—} | 0.145 | 0.321 |
| P23142_FBLN1 | | |
| tissue_protein_{—} | 0.002 | 0.989 |
| P23193_TCEA1 | | |
| tissue_protein_{—} | −0.222 | 0.126 |
| P23588_IF4B | | |
| tissue_protein_{—} | −0.210 | 0.148 |
| P23786_CPT2 | | |
| tissue_protein_{—} | 0.003 | 0.983 |
| P24043_LAMA2 | | |
| tissue_protein_{—} | −0.252 | 0.080 |
| P24534_EF1B | | |
| tissue_protein_{—} | −0.348 | 0.014 |
| P24539_AT5F1 | | |
| tissue_protein_{—} | 0.105 | 0.473 |
| P24557_THAS | | |
| tissue_protein_{—} | −0.047 | 0.747 |
| P24593_IBP5 | | |
| tissue_protein_{—} | −0.248 | 0.086 |
| P24752_THIL | | |
| tissue_protein_{—} | −0.195 | 0.178 |
| P24821_TENA | | |
| tissue_protein_{—} | 0.327 | 0.022 |
| P25067_CO8A2 | | |
| tissue_protein_{—} | −0.238 | 0.100 |
| P25325_THTM | | |
| tissue_protein_{—} | −0.065 | 0.659 |
| P25774_CATS | | |
| tissue_protein_{—} | 0.073 | 0.620 |
| P25940_CO5A3 | | |
| tissue_protein_{—} | −0.025 | 0.862 |
| P26006_ITA3 | | |
| tissue_protein_{—} | −0.251 | 0.082 |
| P26440_IVD | | |
| tissue_protein_{—} | −0.212 | 0.143 |
| P26583_HMGB2 | | |
| tissue_protein_{—} | −0.305 | 0.033 |
| P26885_FKBP2 | | |
| tissue_protein_{—} | −0.116 | 0.429 |
| P27169_PON1 | | |
| tissue_protein_{—} | −0.050 | 0.733 |
| P27449_VATL | | |
| tissue_protein_{—} | 0.469 | 0.001 |
| P27658_CO8A1 | | |
| tissue_protein_{—} | 0.331 | 0.020 |
| P27701_CD82 | | |
| tissue_protein_{—} | −0.198 | 0.173 |
| P27816_MAP4 | | |
| tissue_protein_{—} | −0.103 | 0.480 |
| P27918_PROP | | |
| tissue_protein_{—} | −0.308 | 0.031 |
| P28062_PSB8 | | |
| tissue_protein_{—} | −0.226 | 0.119 |
| P28065_PSB9 | | |
| tissue_protein_{—} | 0.162 | 0.265 |
| P28068_DMB | | |
| tissue_protein_{—} | −0.226 | 0.118 |
| P28072_PSB6 | | |
| tissue_protein_{—} | −0.040 | 0.784 |
| P28074_PSB5 | | |
| tissue_protein_{—} | −0.031 | 0.834 |
| P28161_GSTM2 | | |
| tissue_protein_{—} | −0.088 | 0.546 |
| P28288_ABCD3 | | |
| tissue_protein_{—} | 0.205 | 0.159 |
| P28289_TMOD1 | | |
| tissue_protein_{—} | −0.277 | 0.054 |
| P28331_NDUS1 | | |
| tissue_protein_{—} | 0.146 | 0.316 |
| P28799_GRN | | |
| tissue_protein_{—} | −0.206 | 0.155 |
| P29466_CASP1 | | |
| tissue_protein_{—} | −0.345 | 0.015 |
| P29692_EF1D | | |
| tissue_protein_{—} | −0.091 | 0.534 |
| P29972_AQP1 | | |
| tissue_protein_{—} | −0.222 | 0.125 |
| P30038_AL4A1 | | |
| tissue_protein_{—} | −0.122 | 0.405 |
| P30039_PBLD | | |
| tissue_protein_{—} | −0.254 | 0.078 |
| P30041_PRDX6 | | |
| tissue_protein_{—} | −0.194 | 0.182 |
| P30043_BLVRB | | |
| tissue_protein_{—} | 0.103 | 0.481 |
| P30047_GFRP | | |
| tissue_protein_{—} | −0.391 | 0.005 |
| P30048_PRDX3 | | |
| tissue_protein_{—} | −0.165 | 0.258 |
| P30086_PEBP1 | | |
| tissue_protein_{—} | −0.069 | 0.640 |
| P30711_GSTT1 | | |
| tissue_protein_{—} | −0.289 | 0.044 |
| P31040_SDHA | | |
| tissue_protein_{—} | −0.082 | 0.578 |
| P31947_1433S | | |
| tissue_protein_{—} | 0.068 | 0.644 |
| P31949_S10AB | | |
| tissue_protein_{—} | −0.234 | 0.106 |
| P32119_PRDX2 | | |
| tissue_protein_{—} | −0.125 | 0.393 |
| P32320_CDD | | |
| tissue_protein_{—} | −0.099 | 0.499 |
| P32322_P5CR1 | | |
| tissue_protein_{—} | −0.157 | 0.281 |
| P32455_GBP1 | | |
| tissue_protein_{—} | −0.263 | 0.068 |
| P32929_CGL | | |
| tissue_protein_{—} | −0.135 | 0.354 |
| P33121_ACSL1 | | |
| tissue_protein_{—} | −0.207 | 0.154 |
| P33241_LSP1 | | |
| tissue_protein_{—} | −0.042 | 0.776 |
| P34913_HYES | | |
| tissue_protein_{—} | −0.292 | 0.042 |
| P34931_HS71L | | |
| tissue_protein_{—} | −0.245 | 0.090 |
| P34949_MPI | | |
| tissue_protein_{—} | 0.283 | 0.049 |
| P35052_GPC1 | | |
| tissue_protein_{—} | 0.143 | 0.327 |
| P35442_TSP2 | | |
| tissue_protein_{—} | −0.028 | 0.849 |
| P35542_SAA4 | | |
| tissue_protein_{—} | 0.320 | 0.025 |
| P35555_FBN1 | | |
| tissue_protein_{—} | −0.274 | 0.057 |
| P35579_MYH9 | | |
| tissue_protein_{—} | −0.105 | 0.474 |
| P35580_MYH10 | | |
| tissue_protein_{—} | −0.302 | 0.035 |
| P35611_ADDA | | |
| tissue_protein_{—} | 0.143 | 0.326 |
| P35754_GLRX1 | | |
| tissue_protein_{—} | 0.015 | 0.918 |
| P35813_PPM1A | | |
| tissue_protein_{—} | −0.007 | 0.964 |
| P35858_ALS | | |
| tissue_protein_{—} | −0.299 | 0.037 |
| P35900_K1C20 | | |
| tissue_protein_{—} | 0.097 | 0.505 |
| P36551_HEM6 | | |
| tissue_protein_{—} | −0.335 | 0.018 |
| P36776_LONM | | |
| tissue_protein_{—} | −0.231 | 0.110 |
| P36873_PP1G | | |
| tissue_protein_{—} | 0.041 | 0.777 |
| P36952_SPB5 | | |
| tissue_protein_{—} | 0.151 | 0.301 |
| P36955_PEDF | | |
| tissue_protein_{—} | 0.087 | 0.554 |
| P36980_FHR2 | | |
| tissue_protein_{—} | 0.232 | 0.109 |
| P37235_HPCL1 | | |
| tissue_protein_{—} | −0.357 | 0.012 |
| P38117_ETFB | | |
| tissue_protein_{—} | −0.021 | 0.888 |
| P39210_MPV17 | | |
| tissue_protein_{—} | −0.311 | 0.030 |
| P40121_CAPG | | |
| tissue_protein_{—} | 0.056 | 0.703 |
| P40199_CEAM6 | | |
| tissue_protein_{—} | −0.097 | 0.506 |
| P40306_PSB10 | | |
| tissue_protein_{—} | −0.178 | 0.222 |
| P40313_CTRL | | |
| tissue_protein_{—} | −0.329 | 0.021 |
| P40925_MDHC | | |
| tissue_protein_{—} | −0.161 | 0.271 |
| P41091_IF2G | | |
| tissue_protein_{—} | 0.322 | 0.024 |
| P41223_BUD31 | | |
| tissue_protein_{—} | −0.231 | 0.111 |
| P42224_STAT1 | | |
| tissue_protein_{—} | 0.119 | 0.414 |
| P42226_STAT6 | | |
| tissue_protein_{—} | 0.149 | 0.307 |
| P42356_PI4KA | | |
| tissue_protein_{—} | −0.391 | 0.005 |
| P42765_THIM | | |
| tissue_protein_{—} | 0.021 | 0.888 |
| P42785_PCP | | |
| tissue_protein_{—} | −0.121 | 0.407 |
| P42858_HD | | |
| tissue_protein_{—} | −0.159 | 0.276 |
| P43155_CACP | | |
| tissue_protein_{—} | −0.124 | 0.394 |
| P43897_EFTS | | |
| tissue_protein_{—} | −0.182 | 0.210 |
| P45954_ACDSB | | |
| tissue_protein_{—} | 0.166 | 0.255 |
| P45973_CBX5 | | |
| tissue_protein_{—} | −0.118 | 0.419 |
| P46379_BAG6 | | |
| tissue_protein_{—} | 0.131 | 0.371 |
| P46734_MP2K3 | | |
| tissue_protein_{—} | −0.335 | 0.019 |
| P46736_BRCC3 | | |
| tissue_protein_{—} | −0.123 | 0.399 |
| P46926_GNPI1 | | |
| tissue_protein_{—} | 0.036 | 0.807 |
| P46952_3HAO | | |
| tissue_protein_{—} | −0.098 | 0.501 |
| P46977_STT3A | | |
| tissue_protein_{—} | −0.045 | 0.760 |
| P47895_AL1A3 | | |
| tissue_protein_{—} | −0.394 | 0.005 |
| P48047_ATPO | | |
| tissue_protein_{—} | 0.096 | 0.512 |
| P48061_SDF1 | | |
| tissue_protein_{—} | 0.144 | 0.325 |
| P48163_MAOX | | |
| tissue_protein_{—} | −0.063 | 0.668 |
| P48304_REGIB | | |
| tissue_protein_{—} | 0.096 | 0.511 |
| P48506_GSH1 | | |
| tissue_protein_{—} | 0.083 | 0.569 |
| P48651_PTSS1 | | |
| tissue_protein_{—} | 0.175 | 0.228 |
| P48681_NEST | | |
| tissue_protein_{—} | −0.296 | 0.039 |
| P48728_GCST | | |
| tissue_protein_{—} | −0.326 | 0.022 |
| P48735_IDHP | | |
| tissue_protein_{—} | −0.160 | 0.271 |
| P48739_PIPNB | | |
| tissue_protein_{—} | 0.051 | 0.726 |
| P48960_AGRE5 | | |
| tissue_protein_{—} | 0.194 | 0.181 |
| P49069_CAMLG | | |
| tissue_protein_{—} | 0.125 | 0.393 |
| P49137_MAPK2 | | |
| tissue_protein_{—} | 0.185 | 0.203 |
| P49356_FNTB | | |
| tissue_protein_{—} | −0.268 | 0.063 |
| P49368_TCPG | | |
| tissue_protein_{—} | −0.177 | 0.223 |
| P49406_RM19 | | |
| tissue_protein_{—} | −0.076 | 0.603 |
| P49407_ARRB1 | | |
| tissue_protein_{—} | −0.246 | 0.088 |
| P49419_AL7A1 | | |
| tissue_protein_{—} | −0.226 | 0.118 |
| P49589_SYCC | | |
| tissue_protein_{—} | 0.187 | 0.198 |
| P49747_COMP | | |
| tissue_protein_{—} | −0.200 | 0.168 |
| P49748_ACADV | | |
| tissue_protein_{—} | 0.200 | 0.169 |
| P49756_RBM25 | | |
| tissue_protein_{—} | 0.120 | 0.411 |
| P49757_NUMB | | |
| tissue_protein_{—} | −0.132 | 0.367 |
| P49792_RBP2 | | |
| tissue_protein_{—} | −0.138 | 0.343 |
| P49821_NDUV1 | | |
| tissue_protein_{—} | 0.208 | 0.151 |
| P49902_5NTC | | |
| tissue_protein_{—} | 0.026 | 0.861 |
| P49959_MRE11 | | |
| tissue_protein_{—} | −0.114 | 0.436 |
| P49961_ENTP1 | | |
| tissue_protein_{—} | −0.022 | 0.880 |
| P50120_RET2 | | |
| tissue_protein_{—} | −0.342 | 0.016 |
| P50213_IDH3A | | |
| tissue_protein_{—} | −0.129 | 0.376 |
| P50440_GATM | | |
| tissue_protein_{—} | −0.071 | 0.629 |
| P50453_SPB9 | | |
| tissue_protein_{—} | −0.243 | 0.092 |
| P50454_SERPH | | |
| tissue_protein_{—} | −0.184 | 0.206 |
| P50552_VASP | | |
| tissue_protein_{—} | −0.302 | 0.035 |
| P50895_BCAM | | |
| tissue_protein_{—} | 0.118 | 0.418 |
| P51114_FXR1 | | |
| tissue_protein_{—} | −0.375 | 0.008 |
| P51571_SSRD | | |
| tissue_protein_{—} | 0.339 | 0.017 |
| P51636_CAV2 | | |
| tissue_protein_{—} | −0.065 | 0.656 |
| P51687_SUOX | | |
| tissue_protein_{—} | −0.085 | 0.562 |
| P51689_ARSD | | |
| tissue_protein_{—} | −0.175 | 0.228 |
| P51690_ARSL | | |
| tissue_protein_{—} | 0.102 | 0.487 |
| P51888_PRELP | | |
| tissue_protein_{—} | 0.073 | 0.617 |
| P52294_IMA5 | | |
| tissue_protein_{—} | 0.200 | 0.169 |
| P52735_VAV2 | | |
| tissue_protein_{—} | −0.146 | 0.317 |
| P52758_RIDA | | |
| tissue_protein_{—} | −0.283 | 0.048 |
| P52788_SPSY | | |
| tissue_protein_{—} | 0.075 | 0.610 |
| P52790_HXK3 | | |
| tissue_protein_{—} | −0.028 | 0.850 |
| P52943_CRIP2 | | |
| tissue_protein_{—} | −0.126 | 0.388 |
| P53597_SUCA | | |
| tissue_protein_{—} | 0.092 | 0.528 |
| P53680_AP2S1 | | |
| tissue_protein_{—} | 0.006 | 0.966 |
| P54315_LIPR1 | | |
| tissue_protein_{—} | −0.388 | 0.006 |
| P54652_HSP72 | | |
| tissue_protein_{—} | −0.029 | 0.842 |
| P54687_BCAT1 | | |
| tissue_protein_{—} | −0.142 | 0.332 |
| P54709_AT1B3 | | |
| tissue_protein_{—} | 0.023 | 0.877 |
| P54802_ANAG | | |
| tissue_protein_{—} | −0.323 | 0.024 |
| P54819_KAD2 | | |
| tissue_protein_{—} | −0.331 | 0.020 |
| P54886_P5CS | | |
| tissue_protein_{—} | 0.305 | 0.033 |
| P55001_MFAP2 | | |
| tissue_protein_{—} | 0.064 | 0.662 |
| P55008_AIF1 | | |
| tissue_protein_{—} | −0.139 | 0.342 |
| P55160_NCKPL | | |
| tissue_protein_{—} | −0.284 | 0.048 |
| P55265_DSRAD | | |
| tissue_protein_{—} | 0.030 | 0.840 |
| P55899_FCGRN | | |
| tissue_protein_{—} | −0.192 | 0.186 |
| P56192_SYMC | | |
| tissue_protein_{—} | 0.037 | 0.801 |
| P56199_ITA1 | | |
| tissue_protein_{—} | 0.212 | 0.144 |
| P56545_CTBP2 | | |
| tissue_protein_{—} | −0.019 | 0.899 |
| P57735_RAB25 | | |
| tissue_protein_{—} | −0.081 | 0.582 |
| P57737_CORO7 | | |
| tissue_protein_{—} | −0.177 | 0.223 |
| P57772_SELB | | |
| tissue_protein_{—} | −0.086 | 0.556 |
| P60059_SC61G | | |
| tissue_protein_{—} | −0.247 | 0.087 |
| P60891_PRPS1 | | |
| tissue_protein_{—} | −0.074 | 0.614 |
| P61011_SRP54 | | |
| tissue_protein_{—} | −0.266 | 0.064 |
| P61158_ARP3 | | |
| tissue_protein_{—} | −0.314 | 0.028 |
| P61160_ARP2 | | |
| tissue_protein_{—} | 0.092 | 0.528 |
| P61201_CSN2 | | |
| tissue_protein_{—} | −0.252 | 0.080 |
| P61221_ABCE1 | | |
| tissue_protein_{—} | −0.103 | 0.480 |
| P61225_RAP2B | | |
| tissue_protein_{—} | −0.059 | 0.685 |
| P61289_PSME3 | | |
| tissue_protein_{—} | −0.225 | 0.121 |
| P61513_RL37A | | |
| tissue_protein_{—} | 0.289 | 0.044 |
| P61601_NCALD | | |
| tissue_protein_{—} | −0.080 | 0.585 |
| P61626_LYSC | | |
| tissue_protein_{—} | −0.009 | 0.949 |
| P61769_B2MG | | |
| tissue_protein_{—} | −0.080 | 0.585 |
| P61803_DAD1 | | |
| tissue_protein_{—} | 0.000 | 1.000 |
| P61964_WDR5 | | |
| tissue_protein_{—} | −0.153 | 0.294 |
| P62070_RRAS2 | | |
| tissue_protein_{—} | −0.289 | 0.044 |
| P62258_1433E | | |
| tissue_protein_{—} | −0.008 | 0.956 |
| P62714_PP2AB | | |
| tissue_protein_{—} | −0.014 | 0.921 |
| P62745_RHOB | | |
| tissue_protein_{—} | −0.025 | 0.865 |
| P63172_DYLT1 | | |
| tissue_protein_{—} | −0.019 | 0.899 |
| P63208_SKP1 | | |
| tissue_protein_{—} | −0.347 | 0.015 |
| P63241_IF5A1 | | |
| tissue_protein_{—} | 0.082 | 0.577 |
| P68871_HBB | | |
| tissue_protein_{—} | −0.156 | 0.285 |
| P69891_HBG1 | | |
| tissue_protein_{—} | −0.185 | 0.203 |
| P69892_HBG2 | | |
| tissue_protein_{—} | 0.185 | 0.203 |
| P78539_SRPX | | |
| tissue_protein_{—} | 0.011 | 0.940 |
| P78559_MAP1A | | |
| tissue_protein_{—} | −0.148 | 0.311 |
| P79483_DRB3 | | |
| tissue_protein_{—} | −0.006 | 0.967 |
| P80188_NGAL | | |
| tissue_protein_{—} | −0.175 | 0.228 |
| P80303_NUCB2 | | |
| tissue_protein_{—} | 0.174 | 0.231 |
| P81605_DCD | | |
| tissue_protein_{—} | −0.078 | 0.592 |
| P82673_RT35 | | |
| tissue_protein_{—} | 0.177 | 0.223 |
| P82675_RT05 | | |
| tissue_protein_{—} | −0.101 | 0.491 |
| P82979_SARNP | | |
| tissue_protein_{—} | −0.232 | 0.109 |
| P83436_COG7 | | |
| tissue_protein_{—} | −0.019 | 0.898 |
| P83916_CBX1 | | |
| tissue_protein_{—} | 0.028 | 0.850 |
| P98088_MUC5A | | |
| tissue_protein_{—} | 0.114 | 0.436 |
| P98095_FBLN2 | | |
| tissue_protein_{—} | −0.208 | 0.152 |
| P98179_RBM3 | | |
| tissue_protein_{—} | −0.044 | 0.762 |
| Q00013_EM55 | | |
| tissue_protein_{—} | −0.246 | 0.088 |
| Q00341_VIGLN | | |
| tissue_protein_{—} | 0.028 | 0.850 |
| Q01085_TIAR | | |
| tissue_protein_{—} | −0.193 | 0.185 |
| Q01105_SET | | |
| tissue_protein_{—} | −0.370 | 0.009 |
| Q01130_SRSF2 | | |
| tissue_protein_{—} | −0.185 | 0.204 |
| Q01484_ANK2 | | |
| tissue_protein_{—} | −0.206 | 0.155 |
| Q01813_PFKAP | | |
| tissue_protein_{—} | 0.111 | 0.449 |
| Q01995_TAGL | | |
| tissue_protein_{—} | 0.024 | 0.872 |
| Q02318_CP27A | | |
| tissue_protein_{—} | 0.100 | 0.496 |
| Q02487_DSC2 | | |
| tissue_protein_{—} | −0.322 | 0.024 |
| Q02818_NUCB1 | | |
| tissue_protein_{—} | −0.235 | 0.105 |
| Q03001_DYST | | |
| tissue_protein_{—} | −0.023 | 0.873 |
| Q03519_TAP2 | | |
| tissue_protein_{—} | 0.288 | 0.044 |
| Q03591_FHR1 | | |
| tissue_protein_{—} | 0.320 | 0.025 |
| Q03692_COAA1 | | |
| tissue_protein_{—} | −0.123 | 0.399 |
| Q04695_K1C17 | | |
| tissue_protein_{—} | −0.359 | 0.011 |
| Q05315_LEG10 | | |
| tissue_protein_{—} | −0.188 | 0.195 |
| Q05655_KPCD | | |
| tissue_protein_{—} | −0.114 | 0.436 |
| Q05682_CALD1 | | |
| tissue_protein_{—} | −0.171 | 0.241 |
| Q05707_COEA1 | | |
| tissue_protein_{—} | −0.135 | 0.354 |
| Q06210_GFPT1 | | |
| tissue_protein_{—} | −0.090 | 0.540 |
| Q06278_AOXA | | |
| tissue_protein_{—} | 0.034 | 0.817 |
| Q06828_FMOD | | |
| tissue_protein_{—} | 0.135 | 0.354 |
| Q07092_COGA1 | | |
| tissue_protein_{—} | −0.262 | 0.069 |
| Q07812_BAX | | |
| tissue_protein_{—} | −0.020 | 0.889 |
| Q07866_KLC1 | | |
| tissue_protein_{—} | −0.120 | 0.411 |
| Q07954_LRP1 | | |
| tissue_protein_{—} | 0.000 | 1.000 |
| Q08170_SRSF4 | | |
| tissue_protein_{—} | 0.018 | 0.900 |
| Q08380_LG3BP | | |
| tissue_protein_{—} | −0.073 | 0.617 |
| Q08830_FGL1 | | |
| tissue_protein_{—} | −0.115 | 0.432 |
| Q09028_RBBP4 | | |
| tissue_protein_{—} | 0.046 | 0.752 |
| Q0VAF6_SYCN | | |
| tissue_protein_{—} | −0.101 | 0.492 |
| Q10469_MGAT2 | | |
| tissue_protein_{—} | 0.013 | 0.931 |
| Q10472_GALT1 | | |
| tissue_protein_{—} | 0.332 | 0.020 |
| Q12768_WASC5 | | |
| tissue_protein_{—} | 0.106 | 0.468 |
| Q12805_FBLN3 | | |
| tissue_protein_{—} | −0.128 | 0.382 |
| Q12882_DPYD | | |
| tissue_protein_{—} | −0.080 | 0.585 |
| Q12884_SEPR | | |
| tissue_protein_{—} | −0.231 | 0.111 |
| Q12904_AIMP1 | | |
| tissue_protein_{—} | −0.120 | 0.413 |
| Q12965_MYO1E | | |
| tissue_protein_{—} | −0.358 | 0.012 |
| Q13033_STRN3 | | |
| tissue_protein_{—} | −0.289 | 0.044 |
| Q13057_COASY | | |
| tissue_protein_{—} | 0.205 | 0.157 |
| Q13123_RED | | |
| tissue_protein_{—} | −0.238 | 0.100 |
| Q13144_EI2BE | | |
| tissue_protein_{—} | −0.145 | 0.321 |
| Q13162_PRDX4 | | |
| tissue_protein_{—} | −0.279 | 0.052 |
| Q13177_PAK2 | | |
| tissue_protein_{—} | −0.093 | 0.527 |
| Q13190_STX5 | | |
| tissue_protein_{—} | 0.160 | 0.272 |
| Q13228_SBP1 | | |
| tissue_protein_{—} | −0.157 | 0.280 |
| Q13283_G3BP1 | | |
| tissue_protein_{—} | 0.155 | 0.287 |
| Q13303_KCAB2 | | |
| tissue_protein_{—} | −0.098 | 0.504 |
| Q13308_PTK7 | | |
| tissue_protein_{—} | −0.225 | 0.120 |
| Q13310_PABP4 | | |
| tissue_protein_{—} | 0.302 | 0.035 |
| Q13361_MFAP5 | | |
| tissue_protein_{—} | −0.277 | 0.054 |
| Q13423_NNTM | | |
| tissue_protein_{—} | −0.002 | 0.991 |
| Q13428_TCOF | | |
| tissue_protein_{—} | −0.211 | 0.145 |
| Q13435_SF3B2 | | |
| tissue_protein_{—} | −0.116 | 0.429 |
| Q13438_OS9 | | |
| tissue_protein_{—} | −0.103 | 0.480 |
| Q13442_HAP28 | | |
| tissue_protein_{—} | 0.166 | 0.254 |
| Q13443_ADAM9 | | |
| tissue_protein_{—} | −0.137 | 0.347 |
| Q13445_TMED1 | | |
| tissue_protein_{—} | 0.072 | 0.625 |
| Q13451_FKBP5 | | |
| tissue_protein_{—} | 0.149 | 0.308 |
| Q13492_PICAL | | |
| tissue_protein_{—} | −0.172 | 0.237 |
| Q13505_MTX1 | | |
| tissue_protein_{—} | 0.298 | 0.038 |
| Q13526_PIN1 | | |
| tissue_protein_{—} | −0.444 | 0.001 |
| Q13561_DCTN2 | | |
| tissue_protein_{—} | −0.268 | 0.063 |
| Q13576_IQGA2 | | |
| tissue_protein_{—} | −0.090 | 0.538 |
| Q13595_TRA2A | | |
| tissue_protein_{—} | −0.069 | 0.635 |
| Q13619_CUL4A | | |
| tissue_protein_{—} | −0.305 | 0.033 |
| Q13724_MOGS | | |
| tissue_protein_{—} | 0.042 | 0.775 |
| Q13751_LAMB3 | | |
| tissue_protein_{—} | 0.069 | 0.637 |
| Q13753_LAMC2 | | |
| tissue_protein_{—} | −0.332 | 0.020 |
| Q13825_AUHM | | |
| tissue_protein_{—} | −0.022 | 0.882 |
| Q13884_SNTB1 | | |
| tissue_protein_{—} | −0.141 | 0.332 |
| Q13885_TBB2A | | |
| tissue_protein_{—} | 0.287 | 0.046 |
| Q13907_IDI1 | | |
| tissue_protein_{—} | −0.245 | 0.090 |
| Q14011_CIRBP | | |
| tissue_protein_{—} | −0.005 | 0.975 |
| Q14118_DAG1 | | |
| tissue_protein_{—} | −0.083 | 0.569 |
| Q14141_SEPT6 | | |
| tissue_protein_{—} | −0.208 | 0.152 |
| Q14165_MLEC | | |
| tissue_protein_{—} | −0.010 | 0.948 |
| Q14166_TTL12 | | |
| tissue_protein_{—} | −0.135 | 0.354 |
| Q14192_FHL2 | | |
| tissue_protein_{—} | 0.039 | 0.790 |
| Q14194_DPYL1 | | |
| tissue_protein_{—} | −0.294 | 0.040 |
| Q14195_DPYL3 | | |
| tissue_protein_{—} | −0.204 | 0.161 |
| Q14197_ICT1 | | |
| tissue_protein_{—} | −0.394 | 0.005 |
| Q14247_SRC8 | | |
| tissue_protein_{—} | 0.138 | 0.343 |
| Q14314_FGL2 | | |
| tissue_protein_{—} | −0.107 | 0.464 |
| Q14318_FKBP8 | | |
| tissue_protein_{—} | −0.130 | 0.373 |
| Q14376_GALE | | |
| tissue_protein_{—} | −0.163 | 0.263 |
| Q14554_PDIA5 | | |
| tissue_protein_{—} | 0.021 | 0.888 |
| Q14558_KPRA | | |
| tissue_protein_{—} | −0.166 | 0.253 |
| Q14573_ITPR3 | | |
| tissue_protein_{—} | 0.052 | 0.720 |
| Q14728_MFS10 | | |
| tissue_protein_{—} | 0.246 | 0.088 |
| Q14766_LTBP1 | | |
| tissue_protein_{—} | 0.262 | 0.069 |
| Q14767_LTBP2 | | |
| tissue_protein_{—} | −0.019 | 0.895 |
| Q14839_CHD4 | | |
| tissue_protein_{—} | −0.152 | 0.296 |
| Q15006_EMC2 | | |
| tissue_protein_{—} | 0.011 | 0.940 |
| Q15020_SART3 | | |
| tissue_protein_{—} | 0.041 | 0.777 |
| Q15036_SNX17 | | |
| tissue_protein_{—} | −0.003 | 0.983 |
| Q15041_AR6P1 | | |
| tissue_protein_{—} | −0.093 | 0.527 |
| Q15043_S39AE | | |
| tissue_protein_{—} | −0.308 | 0.031 |
| Q15046_SYK | | |
| tissue_protein_{—} | 0.036 | 0.808 |
| Q15052_ARHG6 | | |
| tissue_protein_{—} | 0.100 | 0.494 |
| Q15063_POSTN | | |
| tissue_protein_{—} | −0.193 | 0.183 |
| Q15067_ACOX1 | | |
| tissue_protein_{—} | 0.015 | 0.916 |
| Q15080_NCF4 | | |
| tissue_protein_{—} | −0.020 | 0.891 |
| Q15113_PCOC1 | | |
| tissue_protein_{—} | −0.039 | 0.792 |
| Q15125_EBP | | |
| tissue_protein_{—} | −0.268 | 0.063 |
| Q15149_PLEC | | |
| tissue_protein_{—} | −0.118 | 0.419 |
| Q15172_2A5A | | |
| tissue_protein_{—} | −0.038 | 0.796 |
| Q15287_RNPS1 | | |
| tissue_protein_{—} | −0.071 | 0.630 |
| Q15370_ELOB | | |
| tissue_protein_{—} | 0.239 | 0.099 |
| Q15386_UBE3C | | |
| tissue_protein_{—} | −0.015 | 0.920 |
| Q15424_SAFB1 | | |
| tissue_protein_{—} | −0.162 | 0.267 |
| Q15436_SC23A | | |
| tissue_protein_{—} | −0.231 | 0.111 |
| Q15437_SC23B | | |
| tissue_protein_{—} | −0.232 | 0.109 |
| Q15477_SKIV2 | | |
| tissue_protein_{—} | −0.140 | 0.337 |
| Q15582_BGH3 | | |
| tissue_protein_{—} | −0.199 | 0.171 |
| Q15642_CIP4 | | |
| tissue_protein_{—} | 0.061 | 0.679 |
| Q15738_NSDHL | | |
| tissue_protein_{—} | −0.117 | 0.424 |
| Q15746_MYLK | | |
| tissue_protein_{—} | −0.185 | 0.203 |
| Q15758_AAAT | | |
| tissue_protein_{—} | −0.216 | 0.136 |
| Q15836_VAMP3 | | |
| tissue_protein_{—} | −0.100 | 0.495 |
| Q15843_NEDD8 | | |
| tissue_protein_{—} | −0.258 | 0.073 |
| Q16134_ETFD | | |
| tissue_protein_{—} | 0.028 | 0.849 |
| Q16186_ADRM1 | | |
| tissue_protein_{—} | −0.121 | 0.409 |
| Q16222_UAP1 | | |
| tissue_protein_{—} | 0.191 | 0.189 |
| Q16270_IBP7 | | |
| tissue_protein_{—} | −0.269 | 0.061 |
| Q16527_CSRP2 | | |
| tissue_protein_{—} | 0.067 | 0.647 |
| Q16537_2A5E | | |
| tissue_protein_{—} | 0.205 | 0.157 |
| Q16610_ECM1 | | |
| tissue_protein_{—} | −0.144 | 0.325 |
| Q16643_DREB | | |
| tissue_protein_{—} | 0.003 | 0.983 |
| Q16658_FSCN1 | | |
| tissue_protein_{—} | −0.306 | 0.032 |
| Q16698_DECR | | |
| tissue_protein_{—} | 0.189 | 0.193 |
| Q16769_QPCT | | |
| tissue_protein_{—} | −0.154 | 0.290 |
| Q16775_GLO2 | | |
| tissue_protein_{—} | 0.075 | 0.610 |
| Q16787_LAMA3 | | |
| tissue_protein_{—} | −0.058 | 0.690 |
| Q16822_PCKGM | | |
| tissue_protein_{—} | −0.265 | 0.066 |
| Q16891_MIC60 | | |
| tissue_protein_{—} | −0.040 | 0.786 |
| Q27J81_INF2 | | |
| tissue_protein_{—} | −0.092 | 0.529 |
| Q2TAA5_ALG11 | | |
| tissue_protein_{—} | −0.071 | 0.628 |
| Q2TAY7_SMU1 | | |
| tissue_protein_{—} | 0.194 | 0.183 |
| Q2UY09_COSA1 | | |
| tissue_protein_{—} | −0.009 | 0.949 |
| Q32P28_P3H1 | | |
| tissue_protein_{—} | −0.179 | 0.218 |
| Q38SD2_LRRK1 | | |
| tissue_protein_{—} | −0.137 | 0.347 |
| Q3LXA3_TKFC | | |
| tissue_protein_{—} | −0.325 | 0.022 |
| Q4G0N4_NAKD2 | | |
| tissue_protein_{—} | −0.151 | 0.301 |
| Q53EL6_PDCD4 | | |
| tissue_protein_{—} | 0.339 | 0.017 |
| Q53FT3_HIKES | | |
| tissue_protein_{—} | 0.026 | 0.857 |
| Q53FZ2_ACSM3 | | |
| tissue_protein_{—} | −0.247 | 0.087 |
| Q53GG5_PDLI3 | | |
| tissue_protein_{—} | −0.169 | 0.245 |
| Q53GQ0_DHB12 | | |
| tissue_protein_{—} | 0.246 | 0.089 |
| Q53H82_LACB2 | | |
| tissue_protein_{—} | 0.066 | 0.651 |
| Q56VL3_OCAD2 | | |
| tissue_protein_{—} | −0.166 | 0.255 |
| Q5J8M3_EMC4 | | |
| tissue_protein_{—} | −0.022 | 0.880 |
| Q5JSL3_DOC11 | | |
| tissue_protein_{—} | −0.107 | 0.465 |
| Q5JVF3_PCID2 | | |
| tissue_protein_{—} | 0.073 | 0.616 |
| Q5SRE7_PHYD1 | | |
| tissue_protein_{—} | 0.174 | 0.232 |
| Q5T440_CAF17 | | |
| tissue_protein_{—} | −0.159 | 0.276 |
| Q5T5P2_SKT | | |
| tissue_protein_{—} | −0.065 | 0.658 |
| Q5VWZ2_LYPL1 | | |
| tissue_protein_{—} | 0.025 | 0.867 |
| Q5VZ46_K1614 | | |
| tissue_protein_{—} | −0.110 | 0.450 |
| Q5VZF2_MBNL2 | | |
| tissue_protein_{—} | −0.168 | 0.248 |
| Q5W0U4_BSPRY | | |
| tissue_protein_{—} | −0.160 | 0.273 |
| Q5XKE5_K2C79 | | |
| tissue_protein_{—} | −0.182 | 0.211 |
| Q5XKP0_MIC13 | | |
| tissue_protein_{—} | −0.198 | 0.172 |
| Q5ZPR3_CD276 | | |
| tissue_protein_{—} | −0.186 | 0.202 |
| Q68CZ2_TENS3 | | |
| tissue_protein_{—} | −0.350 | 0.014 |
| Q68EM7_RHG17 | | |
| tissue_protein_{—} | 0.338 | 0.018 |
| Q6FHJ7_SFRP4 | | |
| tissue_protein_{—} | 0.203 | 0.162 |
| Q6GMV2_SMYD5 | | |
| tissue_protein_{—} | 0.068 | 0.644 |
| Q6GPI1_CTRB2 | | |
| tissue_protein_{—} | −0.144 | 0.325 |
| Q6JBY9_CPZIP | | |
| tissue_protein_{—} | −0.102 | 0.487 |
| Q6NUK1_SCMC1 | | |
| tissue_protein_{—} | −0.162 | 0.265 |
| Q6NUM9_RETST | | |
| tissue_protein_{—} | 0.228 | 0.116 |
| Q6P179_ERAP2 | | |
| tissue_protein_{—} | 0.082 | 0.577 |
| Q6P1A2_MBOA5 | | |
| tissue_protein_{—} | −0.028 | 0.846 |
| Q6P2E9_EDC4 | | |
| tissue_protein_{—} | −0.133 | 0.364 |
| Q6P4A8_PLBL1 | | |
| tissue_protein_{—} | −0.038 | 0.795 |
| Q6P4E1_GOLM2 | | |
| tissue_protein_{—} | −0.049 | 0.739 |
| Q6P5R6_RL22L | | |
| tissue_protein_{—} | −0.076 | 0.601 |
| Q6PGP7_TTC37 | | |
| tissue_protein_{—} | 0.070 | 0.632 |
| Q6PI78_TMM65 | | |
| tissue_protein_{—} | −0.044 | 0.765 |
| Q6PKGO_LARP1 | | |
| tissue_protein_{—} | 0.305 | 0.033 |
| Q6UVY6_MOXD1 | | |
| tissue_protein_{—} | −0.103 | 0.480 |
| Q6UW15_REG3G | | |
| tissue_protein_{—} | −0.167 | 0.251 |
| Q6UWP7_LCLT1 | | |
| tissue_protein_{—} | −0.077 | 0.599 |
| Q6UX06_OLFM4 | | |
| tissue_protein_{—} | −0.121 | 0.407 |
| Q6UX71_PXDC2 | | |
| tissue_protein_{—} | −0.139 | 0.340 |
| Q6UXG2_ELAP1 | | |
| tissue_protein_{—} | −0.124 | 0.395 |
| Q6UXH1_CREL2 | | |
| tissue_protein_{—} | 0.267 | 0.063 |
| Q6UXI9_NPNT | | |
| tissue_protein_{—} | 0.032 | 0.825 |
| Q6UXV4_MIC27 | | |
| tissue_protein_{—} | −0.155 | 0.288 |
| Q6VY07_PACS1 | | |
| tissue_protein_{—} | −0.194 | 0.181 |
| Q6XQN6_PNCB | | |
| tissue_protein_{—} | 0.106 | 0.467 |
| Q6Y7W6_GGYF2 | | |
| tissue_protein_{—} | −0.048 | 0.741 |
| Q6YHK3_CD109 | | |
| tissue_protein_{—} | −0.154 | 0.291 |
| Q6YN16_HSDL2 | | |
| tissue_protein_{—} | 0.349 | 0.014 |
| Q6ZMP0_THSD4 | | |
| tissue_protein_{—} | −0.108 | 0.460 |
| Q709C8_VP13C | | |
| tissue_protein_{—} | 0.023 | 0.875 |
| Q712K3_UB2R2 | | |
| tissue_protein_{—} | −0.172 | 0.237 |
| Q71UM5_RS27L | | |
| tissue_protein_{—} | 0.135 | 0.354 |
| Q75N90_FBN3 | | |
| tissue_protein_{—} | −0.060 | 0.683 |
| Q7L0Y3_TM10C | | |
| tissue_protein_{—} | −0.282 | 0.049 |
| Q7L5N1_CSN6 | | |
| tissue_protein_{—} | −0.221 | 0.127 |
| Q7L5N7_PCAT2 | | |
| tissue_protein_{—} | −0.320 | 0.025 |
| Q7LG56_RIR2B | | |
| tissue_protein_{—} | 0.029 | 0.843 |
| Q7Z3B1_NEGR1 | | |
| tissue_protein_{—} | −0.171 | 0.241 |
| Q7Z3D6_GLUCM | | |
| tissue_protein_{—} | −0.413 | 0.003 |
| Q7Z3U7_MON2 | | |
| tissue_protein_{—} | −0.252 | 0.080 |
| Q7Z406_MYH14 | | |
| tissue_protein_{—} | −0.188 | 0.195 |
| Q7Z4V5_HDGR2 | | |
| tissue_protein_{—} | −0.050 | 0.732 |
| Q7Z739_YTHD3 | | |
| tissue_protein_{—} | 0.198 | 0.173 |
| Q7Z7F7_RM55 | | |
| tissue_protein_{—} | −0.031 | 0.834 |
| Q7Z7H5_TMED4 | | |
| tissue_protein_{—} | −0.013 | 0.927 |
| Q7Z7K6_CENPV | | |
| tissue_protein_{—} | −0.121 | 0.408 |
| Q86SF2_GALT7 | | |
| tissue_protein_{—} | −0.050 | 0.733 |
| Q86TM6_SYVN1 | | |
| tissue_protein_{—} | −0.025 | 0.863 |
| Q86UP6_CUZD1 | | |
| tissue_protein_{—} | −0.021 | 0.888 |
| Q86UU1_PHLB1 | | |
| tissue_protein_{—} | −0.090 | 0.539 |
| Q86V21_AACS | | |
| tissue_protein_{—} | −0.210 | 0.148 |
| Q86VN1_VPS36 | | |
| tissue_protein_{—} | −0.170 | 0.243 |
| Q86VU5_CMTD1 | | |
| tissue_protein_{—} | −0.111 | 0.449 |
| Q86Y39_NDUAB | | |
| tissue_protein_{—} | −0.021 | 0.888 |
| Q86YB8_ERO1B | | |
| tissue_protein_{—} | 0.230 | 0.113 |
| Q8IUR0_TPPC5 | | |
| tissue_protein_{—} | −0.065 | 0.659 |
| Q8IUX7_AEBP1 | | |
| tissue_protein_{—} | −0.189 | 0.195 |
| Q8IV36_HID1 | | |
| tissue_protein_{—} | −0.144 | 0.325 |
| Q8IVF2_AHNK2 | | |
| tissue_protein_{—} | −0.162 | 0.266 |
| Q8IW45_NNRD | | |
| tissue_protein_{—} | −0.330 | 0.020 |
| Q8IWB7_WDFY1 | | |
| tissue_protein_{—} | −0.214 | 0.139 |
| Q8IWE2_NXP20 | | |
| tissue_protein_{—} | 0.228 | 0.115 |
| Q8IXM3_RM41 | | |
| tissue_protein_{—} | 0.125 | 0.393 |
| Q8IXM6_NRM | | |
| tissue_protein_{—} | 0.313 | 0.029 |
| Q8IY17_PLPL6 | | |
| tissue_protein_{—} | −0.035 | 0.813 |
| Q8IYB5_SMAP1 | | |
| tissue_protein_{—} | −0.391 | 0.005 |
| Q8IZ83_A16A1 | | |
| tissue_protein_{—} | −0.333 | 0.019 |
| Q8IZQ5_SELH | | |
| tissue_protein_{—} | −0.032 | 0.827 |
| Q8N128_F177A | | |
| tissue_protein_{—} | −0.088 | 0.549 |
| Q8N1F7_NUP93 | | |
| tissue_protein_{—} | −0.169 | 0.244 |
| Q8N1G4_LRC47 | | |
| tissue_protein_{—} | −0.075 | 0.610 |
| Q8N1S5_S39AB | | |
| tissue_protein_{—} | −0.068 | 0.641 |
| Q8N2K0_ABD12 | | |
| tissue_protein_{—} | 0.153 | 0.295 |
| Q8N2S1_LTBP4 | | |
| tissue_protein_{—} | 0.032 | 0.827 |
| Q8N392_RHG18 | | |
| tissue_protein_{—} | 0.020 | 0.892 |
| Q8N3C0_ASCC3 | | |
| tissue_protein_{—} | −0.131 | 0.368 |
| Q8N3D4_EH1L1 | | |
| tissue_protein_{—} | 0.149 | 0.307 |
| Q8N3V7_SYNPO | | |
| tissue_protein_{—} | −0.088 | 0.549 |
| Q8N4T8_CBR4 | | |
| tissue_protein_{—} | −0.282 | 0.050 |
| Q8N5M9_JAGN1 | | |
| tissue_protein_{—} | −0.057 | 0.697 |
| Q8N6H7_ARFG2 | | |
| tissue_protein_{—} | −0.242 | 0.094 |
| Q8N6L1_KTAP2 | | |
| tissue_protein_{—} | −0.143 | 0.328 |
| Q8N766_EMC1 | | |
| tissue_protein_{—} | 0.055 | 0.709 |
| Q8N983_RM43 | | |
| tissue_protein_{—} | −0.015 | 0.920 |
| Q8NB37_GALD1 | | |
| tissue_protein_{—} | −0.183 | 0.208 |
| Q8NBF2_NHLC2 | | |
| tissue_protein_{—} | 0.145 | 0.319 |
| Q8NBJ4_GOLM1 | | |
| tissue_protein_{—} | −0.265 | 0.066 |
| Q8NBJ5_GT251 | | |
| tissue_protein_{—} | −0.135 | 0.353 |
| Q8NBJ7_SUMF2 | | |
| tissue_protein_{—} | 0.004 | 0.981 |
| Q8NBJ9_SIDT2 | | |
| tissue_protein_{—} | −0.160 | 0.272 |
| Q8NC51_PAIRB | | |
| tissue_protein_{—} | −0.119 | 0.414 |
| Q8NCA5_FA98A | | |
| tissue_protein_{—} | −0.277 | 0.054 |
| Q8NE62_CHDH | | |
| tissue_protein_{—} | −0.324 | 0.023 |
| Q8NEW0_ZNT7 | | |
| tissue_protein_{—} | −0.202 | 0.165 |
| Q8NFV4_ABHDB | | |
| tissue_protein_{—} | −0.122 | 0.404 |
| Q8NFZ8_CADM4 | | |
| tissue_protein_{—} | 0.069 | 0.637 |
| Q8TB61_S35B2 | | |
| tissue_protein_{—} | −0.174 | 0.232 |
| Q8TBC4_UBA3 | | |
| tissue_protein_{—} | −0.357 | 0.012 |
| Q8TC07_TBC15 | | |
| tissue_protein_{—} | 0.019 | 0.895 |
| Q8TCD5_NT5C | | |
| tissue_protein_{—} | −0.082 | 0.578 |
| Q8TD55_PKHO2 | | |
| tissue_protein_{—} | −0.168 | 0.248 |
| Q8TDZ2_MICA1 | | |
| tissue_protein_{—} | −0.027 | 0.852 |
| Q8TED1_GPX8 | | |
| tissue_protein_{—} | 0.084 | 0.565 |
| Q8WU76_SCFD2 | | |
| tissue_protein_{—} | −0.060 | 0.684 |
| Q8WUH6_TM263 | | |
| tissue_protein_{—} | −0.170 | 0.242 |
| Q8WUP2_FBLI1 | | |
| tissue_protein_{—} | 0.252 | 0.081 |
| Q8WVC6_DCAKD | | |
| tissue_protein_{—} | −0.251 | 0.082 |
| Q8WVJ2_NUDC2 | | |
| tissue_protein_{—} | −0.173 | 0.235 |
| Q8WVM8_SCFD1 | | |
| tissue_protein_{—} | −0.116 | 0.429 |
| Q8WVV4_POF1B | | |
| tissue_protein_{—} | 0.185 | 0.204 |
| Q8WVV9_HNRLL | | |
| tissue_protein_{—} | 0.413 | 0.003 |
| Q8WWI1_LMO7 | | |
| tissue_protein_{—} | 0.236 | 0.102 |
| Q8WWL7_CCNB3 | | |
| tissue_protein_{—} | −0.065 | 0.656 |
| Q8WWM9_CYGB | | |
| tissue_protein_{—} | −0.260 | 0.071 |
| Q8WWX9_SELM | | |
| tissue_protein_{—} | −0.175 | 0.228 |
| Q8WX93_PALLD | | |
| tissue_protein_{—} | −0.177 | 0.225 |
| Q8WXQ8_CBPA5 | | |
| tissue_protein_{—} | 0.153 | 0.295 |
| Q92485_ASM3B | | |
| tissue_protein_{—} | −0.334 | 0.019 |
| Q92506_DHB8 | | |
| tissue_protein_{—} | −0.270 | 0.060 |
| Q92544_TM9S4 | | |
| tissue_protein_{—} | −0.141 | 0.334 |
| Q92572_AP3S1 | | |
| tissue_protein_{—} | −0.232 | 0.109 |
| Q92575_UBXN4 | | |
| tissue_protein_{—} | 0.088 | 0.548 |
| Q92598_HS105 | | |
| tissue_protein_{—} | −0.255 | 0.077 |
| Q92608_DOCK2 | | |
| tissue_protein_{—} | −0.047 | 0.750 |
| Q92621_NU205 | | |
| tissue_protein_{—} | −0.157 | 0.282 |
| Q92626_PXDN | | |
| tissue_protein_{—} | −0.052 | 0.724 |
| Q92692_NECT2 | | |
| tissue_protein_{—} | −0.212 | 0.143 |
| Q92734_TFG | | |
| tissue_protein_{—} | 0.225 | 0.121 |
| Q92743_HTRA1 | | |
| tissue_protein_{—} | 0.081 | 0.579 |
| Q92820_GGH | | |
| tissue_protein_{—} | −0.228 | 0.116 |
| Q92823_NRCAM | | |
| tissue_protein_{—} | −0.114 | 0.436 |
| Q92896_GSLG1 | | |
| tissue_protein_{—} | −0.050 | 0.734 |
| Q92901_RL3L | | |
| tissue_protein_{—} | −0.260 | 0.071 |
| Q92947_GCDH | | |
| tissue_protein_{—} | 0.205 | 0.158 |
| Q92974_ARHG2 | | |
| tissue_protein_{—} | 0.000 | 1.000 |
| Q93008_USP9X | | |
| tissue_protein_{—} | −0.129 | 0.376 |
| Q93050_VPP1 | | |
| tissue_protein_{—} | 0.216 | 0.136 |
| Q93091_RNAS6 | | |
| tissue_protein_{—} | −0.201 | 0.166 |
| Q969G3_SMCE1 | | |
| tissue_protein_{—} | −0.064 | 0.662 |
| Q969G5_CAVN3 | | |
| tissue_protein_{—} | 0.008 | 0.958 |
| Q969L2_MAL2 | | |
| tissue_protein_{—} | −0.045 | 0.758 |
| Q969S3_ZN622 | | |
| tissue_protein_{—} | −0.105 | 0.474 |
| Q969V3_NCLN | | |
| tissue_protein_{—} | −0.212 | 0.143 |
| Q96A26_F162A | | |
| tissue_protein_{—} | −0.232 | 0.109 |
| Q96A33_CCD47 | | |
| tissue_protein_{—} | −0.176 | 0.228 |
| Q96AB3_ISOC2 | | |
| tissue_protein_{—} | −0.028 | 0.850 |
| Q96AC1_FERM2 | | |
| tissue_protein_{—} | −0.269 | 0.062 |
| Q96AQ6_PBIP1 | | |
| tissue_protein_{—} | 0.025 | 0.867 |
| Q96AY3_FKB10 | | |
| tissue_protein_{—} | −0.006 | 0.966 |
| Q96AZ6_ISG20 | | |
| tissue_protein_{—} | −0.135 | 0.355 |
| Q96B97_SH3K1 | | |
| tissue_protein_{—} | −0.168 | 0.247 |
| Q96BJ3_AIDA | | |
| tissue_protein_{—} | 0.071 | 0.629 |
| Q96BM9_ARL8A | | |
| tissue_protein_{—} | −0.268 | 0.063 |
| Q96BW5_PTER | | |
| tissue_protein_{—} | −0.197 | 0.175 |
| Q96C01_F136A | | |
| tissue_protein_{—} | 0.059 | 0.689 |
| Q96CG8_CTHR1 | | |
| tissue_protein_{—} | −0.200 | 0.168 |
| Q96CN7_ISOC1 | | |
| tissue_protein_{—} | −0.038 | 0.797 |
| Q96D15_RCN3 | | |
| tissue_protein_{—} | 0.061 | 0.675 |
| Q96DE0_NUD16 | | |
| tissue_protein_{—} | −0.089 | 0.544 |
| Q96DN0_ERP27 | | |
| tissue_protein_{—} | −0.189 | 0.193 |
| Q96DZ1_ERLEC | | |
| tissue_protein_{—} | −0.105 | 0.474 |
| Q96EE3_SEH1 | | |
| tissue_protein_{—} | −0.022 | 0.881 |
| Q96EK6_GNA1 | | |
| tissue_protein_{—} | 0.059 | 0.688 |
| Q96EY5_MB12A | | |
| tissue_protein_{—} | 0.134 | 0.359 |
| Q96F85_CNRP1 | | |
| tissue_protein_{—} | 0.106 | 0.470 |
| Q96FQ6_S10AG | | |
| tissue_protein_{—} | −0.258 | 0.074 |
| Q96FV2_SCRN2 | | |
| tissue_protein_{—} | −0.299 | 0.037 |
| Q96FW1_OTUB1 | | |
| tissue_protein_{—} | −0.389 | 0.006 |
| Q96GK7_FAH2A | | |
| tissue_protein_{—} | −0.065 | 0.659 |
| Q96HE7_ERO1A | | |
| tissue_protein_{—} | 0.048 | 0.746 |
| Q96HF1_SFRP2 | | |
| tissue_protein_{—} | −0.145 | 0.320 |
| Q96HR9_REEP6 | | |
| tissue_protein_{—} | −0.231 | 0.110 |
| Q96HY6_DDRGK | | |
| tissue_protein_{—} | −0.177 | 0.224 |
| Q96I15_SCLY | | |
| tissue_protein_{—} | 0.067 | 0.649 |
| Q96I59_SYNM | | |
| tissue_protein_{—} | −0.357 | 0.012 |
| Q96I99_SUCB2 | | |
| tissue_protein_{—} | −0.003 | 0.981 |
| Q96IY4_CBPB2 | | |
| tissue_protein_{—} | −0.268 | 0.063 |
| Q96KP4_CNDP2 | | |
| tissue_protein_{—} | −0.193 | 0.184 |
| Q96KR1_ZFR | | |
| tissue_protein_{—} | 0.020 | 0.889 |
| Q96LD4_TRI47 | | |
| tissue_protein_{—} | −0.263 | 0.068 |
| Q96LJ7_DHRS1 | | |
| tissue_protein_{—} | −0.025 | 0.866 |
| Q96M27_PRRC1 | | |
| tissue_protein_{—} | 0.301 | 0.036 |
| Q96M96_FGD4 | | |
| tissue_protein_{—} | −0.298 | 0.037 |
| Q96MW5_COG8 | | |
| tissue_protein_{—} | 0.167 | 0.252 |
| Q96MX0_CKLF3 | | |
| tissue_protein_{—} | −0.128 | 0.381 |
| Q96NB2_SFXN2 | | |
| tissue_protein_{—} | 0.425 | 0.002 |
| Q96P44_COLA1 | | |
| tissue_protein_{—} | −0.244 | 0.092 |
| Q96PE7_MCEE | | |
| tissue_protein_{—} | −0.140 | 0.336 |
| Q96PU8_QKI | | |
| tissue_protein_{—} | −0.118 | 0.421 |
| Q96QR8_PURB | | |
| tissue_protein_{—} | −0.132 | 0.368 |
| Q96RQ9_OXLA | | |
| tissue_protein_{—} | 0.239 | 0.098 |
| Q96S06_LMF1 | | |
| tissue_protein_{—} | −0.298 | 0.037 |
| Q96S97_MYADM | | |
| tissue_protein_{—} | −0.194 | 0.181 |
| Q96SQ9_CP2S1 | | |
| tissue_protein_{—} | −0.135 | 0.354 |
| Q99439_CNN2 | | |
| tissue_protein_{—} | −0.082 | 0.576 |
| Q99442_SEC62 | | |
| tissue_protein_{—} | −0.292 | 0.042 |
| Q99497_PARK7 | | |
| tissue_protein_{—} | 0.014 | 0.926 |
| Q99523_SORT | | |
| tissue_protein_{—} | −0.122 | 0.402 |
| Q99613_EIF3C | | |
| tissue_protein_{—} | −0.235 | 0.103 |
| Q99685_MGLL | | |
| tissue_protein_{—} | 0.129 | 0.376 |
| Q99715_COCA1 | | |
| tissue_protein_{—} | −0.248 | 0.086 |
| Q99798_ACON | | |
| tissue_protein_{—} | −0.151 | 0.301 |
| Q99805_TM9S2 | | |
| tissue_protein_{—} | −0.074 | 0.613 |
| Q99816_TS101 | | |
| tissue_protein_{—} | 0.129 | 0.376 |
| Q99943_PLCA | | |
| tissue_protein_{—} | −0.166 | 0.253 |
| Q99969_RARR2 | | |
| tissue_protein_{—} | −0.102 | 0.487 |
| Q9BQ69_MACD1 | | |
| tissue_protein_{—} | −0.046 | 0.752 |
| Q9BRJ2_RM45 | | |
| tissue_protein_{—} | −0.164 | 0.259 |
| Q9BRJ6_CG050 | | |
| tissue_protein_{—} | 0.114 | 0.435 |
| Q9BRX8_PXL2A | | |
| tissue_protein_{—} | −0.197 | 0.175 |
| Q9BS40_LXN | | |
| tissue_protein_{—} | −0.130 | 0.373 |
| Q9BSH4_TACO1 | | |
| tissue_protein_{—} | −0.015 | 0.916 |
| Q9BT40_INP5K | | |
| tissue_protein_{—} | −0.298 | 0.037 |
| Q9BT78_CSN4 | | |
| tissue_protein_{—} | −0.069 | 0.639 |
| Q9BTT0_AN32E | | |
| tissue_protein_{—} | −0.042 | 0.777 |
| Q9BTZ2_DHRS4 | | |
| tissue_protein_{—} | −0.376 | 0.008 |
| Q9BUH6_PAXX | | |
| tissue_protein_{—} | −0.050 | 0.735 |
| Q9BUL8_PDC10 | | |
| tissue_protein_{—} | 0.009 | 0.950 |
| Q9BUN8_DERL1 | | |
| tissue_protein_{—} | −0.096 | 0.511 |
| Q9BV10_ALG12 | | |
| tissue_protein_{—} | −0.104 | 0.477 |
| Q9BVA1_TBB2B | | |
| tissue_protein_{—} | −0.283 | 0.049 |
| Q9BVK6_TMED9 | | |
| tissue_protein_{—} | −0.151 | 0.301 |
| Q9BVM4_GGACT | | |
| tissue_protein_{—} | −0.003 | 0.982 |
| Q9BVP2_GNL3 | | |
| tissue_protein_{—} | −0.041 | 0.781 |
| Q9BW91_NUDT9 | | |
| tissue_protein_{—} | −0.091 | 0.534 |
| Q9BWF3_RBM4 | | |
| tissue_protein_{—} | −0.009 | 0.949 |
| Q9BWS9_CHID1 | | |
| tissue_protein_{—} | −0.158 | 0.277 |
| Q9BX68_HINT2 | | |
| tissue_protein_{—} | −0.023 | 0.875 |
| Q9BX97_PLVAP | | |
| tissue_protein_{—} | 0.222 | 0.126 |
| Q9BXJ0_C1QT5 | | |
| tissue_protein_{—} | −0.225 | 0.120 |
| Q9BXJ9_NAA15 | | |
| tissue_protein_{—} | 0.077 | 0.599 |
| Q9BXN1_ASPN | | |
| tissue_protein_{—} | −0.085 | 0.563 |
| Q9BY44_EIF2A | | |
| tissue_protein_{—} | −0.263 | 0.068 |
| Q9BY50_SC11C | | |
| tissue_protein_{—} | 0.149 | 0.308 |
| Q9BYD1_RM13 | | |
| tissue_protein_{—} | −0.098 | 0.502 |
| Q9BYD6_RM01 | | |
| tissue_protein_{—} | −0.131 | 0.371 |
| Q9BZG1_RAB34 | | |
| tissue_protein_{—} | −0.096 | 0.512 |
| Q9BZL1_UBL5 | | |
| tissue_protein_{—} | −0.232 | 0.108 |
| Q9BZQ8_NIBA1 | | |
| tissue_protein_{—} | 0.111 | 0.447 |
| Q9BZV1_UBXN6 | | |
| tissue_protein_{—} | −0.087 | 0.552 |
| Q9C0B1_FTO | | |
| tissue_protein_{—} | 0.238 | 0.100 |
| Q9GZM5_YIPF3 | | |
| tissue_protein_{—} | −0.114 | 0.437 |
| Q9GZT3_SLIRP | | |
| tissue_protein_{—} | −0.242 | 0.094 |
| Q9GZT8_NIF3L | | |
| tissue_protein_{—} | −0.159 | 0.275 |
| Q9GZZ1_NAA50 | | |
| tissue_protein_{—} | −0.197 | 0.175 |
| Q9GZZ9_UBA5 | | |
| tissue_protein_{—} | −0.084 | 0.565 |
| Q9H0D6_XRN2 | | |
| tissue_protein_{—} | 0.048 | 0.745 |
| Q9H0U3_MAGT1 | | |
| tissue_protein_{—} | −0.245 | 0.090 |
| Q9H0U6_RM18 | | |
| tissue_protein_{—} | −0.095 | 0.514 |
| Q9H0W9_CK054 | | |
| tissue_protein_{—} | −0.192 | 0.186 |
| Q9H2D6_TARA | | |
| tissue_protein_{—} | 0.062 | 0.671 |
| Q9H330_TM245 | | |
| tissue_protein_{—} | 0.000 | 1.000 |
| Q9H3H5_GPT | | |
| tissue_protein_{—} | −0.084 | 0.565 |
| Q9H3K2_GHITM | | |
| tissue_protein_{—} | 0.197 | 0.176 |
| Q9H3R0_KDM4C | | |
| tissue_protein_{—} | 0.400 | 0.004 |
| Q9H492_MLP3A | | |
| tissue_protein_{—} | −0.135 | 0.354 |
| Q9H553_ALG2 | | |
| tissue_protein_{—} | 0.012 | 0.934 |
| Q9H583_HEAT1 | | |
| tissue_protein_{—} | −0.194 | 0.183 |
| Q9H6K4_OPA3 | | |
| tissue_protein_{—} | −0.008 | 0.958 |
| Q9H6R3_ACSS3 | | |
| tissue_protein_{—} | 0.104 | 0.478 |
| Q9H6S0_YTDC2 | | |
| tissue_protein_{—} | −0.150 | 0.304 |
| Q9H6U8_ALG9 | | |
| tissue_protein_{—} | −0.178 | 0.220 |
| Q9H6Z4_RANB3 | | |
| tissue_protein_{—} | −0.302 | 0.035 |
| Q9H845_ACAD9 | | |
| tissue_protein_{—} | −0.031 | 0.834 |
| Q9H8H3_MET7A | | |
| tissue_protein_{—} | −0.189 | 0.193 |
| Q9H936_GHC1 | | |
| tissue_protein_{—} | 0.005 | 0.974 |
| Q9H9P8_L2HDH | | |
| tissue_protein_{—} | −0.281 | 0.050 |
| Q9HA77_SYCM | | |
| tissue_protein_{—} | 0.253 | 0.079 |
| Q9HAT2_SIAE | | |
| tissue_protein_{—} | 0.162 | 0.265 |
| Q9HB40_RISC | | |
| tissue_protein_{—} | −0.098 | 0.501 |
| Q9HB71_CYBP | | |
| tissue_protein_{—} | −0.157 | 0.281 |
| Q9HCC0_MCCB | | |
| tissue_protein_{—} | 0.266 | 0.064 |
| Q9HCK8_CHD8 | | |
| tissue_protein_{—} | −0.186 | 0.200 |
| Q9HCN8_SDF2L | | |
| tissue_protein_{—} | −0.186 | 0.202 |
| Q9HCU5_PREB | | |
| tissue_protein_{—} | −0.332 | 0.020 |
| Q9HD20_AT131 | | |
| tissue_protein_{—} | 0.175 | 0.228 |
| Q9HD26_GOPC | | |
| tissue_protein_{—} | 0.094 | 0.522 |
| Q9HD33_RM47 | | |
| tissue_protein_{—} | −0.280 | 0.051 |
| Q9NP81_SYSM | | |
| tissue_protein_{—} | 0.207 | 0.154 |
| Q9NPA8_ENY2 | | |
| tissue_protein_{—} | 0.216 | 0.136 |
| Q9NQ50_RM40 | | |
| tissue_protein_{—} | 0.135 | 0.355 |
| Q9NQT8_KI13B | | |
| tissue_protein_{—} | −0.252 | 0.080 |
| Q9NR12_PDLI7 | | |
| tissue_protein_{—} | −0.262 | 0.069 |
| Q9NR45_SIAS | | |
| tissue_protein_{—} | −0.217 | 0.134 |
| Q9NR50_EI2BG | | |
| tissue_protein_{—} | 0.133 | 0.362 |
| Q9NR99_MXRA5 | | |
| tissue_protein_{—} | −0.019 | 0.895 |
| Q9NRN7_ADPPT | | |
| tissue_protein_{—} | 0.210 | 0.147 |
| Q9NRZ7_PLCC | | |
| tissue_protein_{—} | −0.194 | 0.182 |
| Q9NSD9_SYFB | | |
| tissue_protein_{—} | −0.246 | 0.088 |
| Q9NTX5_ECHD1 | | |
| tissue_protein_{—} | −0.311 | 0.030 |
| Q9NUB1_ACS2L | | |
| tissue_protein_{—} | −0.178 | 0.221 |
| Q9NUL5_SHFL | | |
| tissue_protein_{—} | −0.178 | 0.220 |
| Q9NUP9_LIN7C | | |
| tissue_protein_{—} | −0.104 | 0.478 |
| Q9NUQ6_SPS2L | | |
| tissue_protein_{—} | −0.289 | 0.044 |
| Q9NUQ7_UFSP2 | | |
| tissue_protein_{—} | 0.077 | 0.600 |
| Q9NUQ8_ABCF3 | | |
| tissue_protein_{—} | −0.321 | 0.025 |
| Q9NUV9_GIMA4 | | |
| tissue_protein_{—} | −0.074 | 0.614 |
| Q9NVJ2_ARL8B | | |
| tissue_protein_{—} | −0.183 | 0.207 |
| Q9NVZ3_NECP2 | | |
| tissue_protein_{—} | −0.099 | 0.499 |
| Q9NW15_ANO10 | | |
| tissue_protein_{—} | −0.022 | 0.880 |
| Q9NX14_NDUBB | | |
| tissue_protein_{—} | −0.011 | 0.941 |
| Q9NX40_OCAD1 | | |
| tissue_protein_{—} | −0.086 | 0.559 |
| Q9NX46_ADPRS | | |
| tissue_protein_{—} | −0.326 | 0.022 |
| Q9NYU2_UGGG1 | | |
| tissue_protein_{—} | −0.079 | 0.588 |
| Q9NZ32_ARP10 | | |
| tissue_protein_{—} | 0.015 | 0.916 |
| Q9NZJ7_MTCH1 | | |
| tissue_protein_{—} | 0.003 | 0.983 |
| Q9NZM1_MYOF | | |
| tissue_protein_{—} | 0.009 | 0.949 |
| Q9P016_THYN1 | | |
| tissue_protein_{—} | −0.006 | 0.968 |
| Q9P0J1_PDP1 | | |
| tissue_protein_{—} | −0.191 | 0.189 |
| Q9P2B2_FPRP | | |
| tissue_protein_{—} | −0.302 | 0.035 |
| Q9P2E9_RRBP1 | | |
| tissue_protein_{—} | −0.320 | 0.025 |
| Q9P2J5_SYLC | | |
| tissue_protein_{—} | −0.249 | 0.084 |
| Q9P2R7_SUCB1 | | |
| tissue_protein_{—} | −0.205 | 0.157 |
| Q9P2T1_GMPR2 | | |
| tissue_protein_{—} | −0.331 | 0.020 |
| Q9UBI6_GBG12 | | |
| tissue_protein_{—} | −0.061 | 0.679 |
| Q9UBM7_DHCR7 | | |
| tissue_protein_{—} | −0.279 | 0.053 |
| Q9UBQ7_GRHPR | | |
| tissue_protein_{—} | −0.212 | 0.143 |
| Q9UBR2_CATZ | | |
| tissue_protein_{—} | −0.135 | 0.353 |
| Q9UBS4_DJB11 | | |
| tissue_protein_{—} | −0.199 | 0.170 |
| Q9UBT2_SAE2 | | |
| tissue_protein_{—} | −0.058 | 0.690 |
| Q9UBT3_DKK4 | | |
| tissue_protein_{—} | −0.173 | 0.236 |
| Q9UBV2_SE1L1 | | |
| tissue_protein_{—} | −0.039 | 0.792 |
| Q9UBW8_CSN7A | | |
| tissue_protein_{—} | −0.096 | 0.512 |
| Q9UDY2_ZO2 | | |
| tissue_protein_{—} | −0.139 | 0.341 |
| Q9UDY4_DNJB4 | | |
| tissue_protein_{—} | 0.067 | 0.649 |
| Q9UEU0_VTI1B | | |
| tissue_protein_{—} | 0.153 | 0.294 |
| Q9UG63_ABCF2 | | |
| tissue_protein_{—} | −0.022 | 0.879 |
| Q9UGM3_DMBT1 | | |
| tissue_protein_{—} | −0.123 | 0.399 |
| Q9UGP8_SEC63 | | |
| tissue_protein_{—} | −0.210 | 0.148 |
| Q9UH65_SWP70 | | |
| tissue_protein_{—} | −0.162 | 0.265 |
| Q9UHA4_LTOR3 | | |
| tissue_protein_{—} | −0.185 | 0.203 |
| Q9UHB9_SRP68 | | |
| tissue_protein_{—} | −0.258 | 0.073 |
| Q9UHG3_PCYOX | | |
| tissue_protein_{—} | −0.259 | 0.073 |
| Q9UI09_NDUAC | | |
| tissue_protein_{—} | −0.213 | 0.142 |
| Q9UI10_EI2BD | | |
| tissue_protein_{—} | −0.117 | 0.424 |
| Q9UI14_PRAF1 | | |
| tissue_protein_{—} | 0.300 | 0.036 |
| Q9UIQ6_LCAP | | |
| tissue_protein_{—} | −0.012 | 0.933 |
| Q9UJ72_ANX10 | | |
| tissue_protein_{—} | −0.234 | 0.105 |
| Q9UJZ1_STML2 | | |
| tissue_protein_{—} | −0.087 | 0.551 |
| Q9UK22_FBX2 | | |
| tissue_protein_{—} | 0.012 | 0.937 |
| Q9UKD2_MRT4 | | |
| tissue_protein_{—} | 0.061 | 0.679 |
| Q9UKK3_PARP4 | | |
| tissue_protein_{—} | 0.132 | 0.368 |
| Q9UKV3_ACINU | | |
| tissue_protein_{—} | −0.125 | 0.393 |
| Q9UL12_SARDH | | |
| tissue_protein_{—} | 0.245 | 0.090 |
| Q9ULC3_RAB23 | | |
| tissue_protein_{—} | −0.092 | 0.528 |
| Q9ULC5_ACSL5 | | |
| tissue_protein_{—} | 0.053 | 0.719 |
| Q9ULL5_PRR12 | | |
| tissue_protein_{—} | 0.035 | 0.813 |
| Q9ULP9_TBC24 | | |
| tissue_protein_{—} | −0.221 | 0.127 |
| Q9ULZ3_ASC | | |
| tissue_protein_{—} | 0.038 | 0.797 |
| Q9UM00_TMCO1 | | |
| tissue_protein_{—} | 0.114 | 0.435 |
| Q9UMY4_SNX12 | | |
| tissue_protein_{—} | −0.266 | 0.065 |
| Q9UN37_VPS4A | | |
| tissue_protein_{—} | −0.225 | 0.121 |
| Q9UNF0_PACN2 | | |
| tissue_protein_{—} | −0.224 | 0.122 |
| Q9UNL2_SSRG | | |
| tissue_protein_{—} | −0.346 | 0.015 |
| Q9UNZ2_NSFIC | | |
| tissue_protein_{—} | 0.198 | 0.173 |
| Q9UPU7_TBD2B | | |
| tissue_protein_{—} | 0.174 | 0.231 |
| Q9Y224_RTRAF | | |
| tissue_protein_{—} | −0.057 | 0.697 |
| Q9Y237_PIN4 | | |
| tissue_protein_{—} | −0.004 | 0.981 |
| Q9Y240_CLC11 | | |
| tissue_protein_{—} | −0.268 | 0.063 |
| Q9Y262_EIF3L | | |
| tissue_protein_{—} | −0.222 | 0.126 |
| Q9Y263_PLAP | | |
| tissue_protein_{—} | −0.321 | 0.024 |
| Q9Y282_ERGI3 | | |
| tissue_protein_{—} | −0.150 | 0.305 |
| Q9Y295_DRG1 | | |
| tissue_protein_{—} | −0.146 | 0.316 |
| Q9Y2B0_CNPY2 | | |
| tissue_protein_{—} | 0.157 | 0.282 |
| Q9Y2H5_PKHA6 | | |
| tissue_protein_{—} | −0.010 | 0.945 |
| Q9Y2H6_FND3A | | |
| tissue_protein_{—} | −0.206 | 0.156 |
| Q9Y2Q9_RT28 | | |
| tissue_protein_{—} | −0.234 | 0.106 |
| Q9Y2S7_PDIP2 | | |
| tissue_protein_{—} | −0.300 | 0.036 |
| Q9Y2T2_AP3M1 | | |
| tissue_protein_{—} | −0.098 | 0.504 |
| Q9Y2Y8_PRG3 | | |
| tissue_protein_{—} | −0.109 | 0.456 |
| Q9Y315_DEOC | | |
| tissue_protein_{—} | −0.156 | 0.285 |
| Q9Y365_STA10 | | |
| tissue_protein_{—} | −0.224 | 0.121 |
| Q9Y371_SHLB1 | | |
| tissue_protein_{—} | −0.135 | 0.353 |
| Q9Y383_LC7L2 | | |
| tissue_protein_{—} | −0.186 | 0.200 |
| Q9Y385_UB2J1 | | |
| tissue_protein_{—} | −0.245 | 0.090 |
| Q9Y3A6_TMED5 | | |
| tissue_protein_{—} | 0.013 | 0.931 |
| Q9Y3B8_ORN | | |
| tissue_protein_{—} | 0.129 | 0.376 |
| Q9Y3D6_FIS1 | | |
| tissue_protein_{—} | −0.069 | 0.635 |
| Q9Y3D9_RT23 | | |
| tissue_protein_{—} | −0.120 | 0.410 |
| Q9Y3E5_PTH2 | | |
| tissue_protein_{—} | −0.213 | 0.141 |
| Q9Y3P9_RBGP1 | | |
| tissue_protein_{—} | −0.183 | 0.208 |
| Q9Y450_HBS1L | | |
| tissue_protein_{—} | 0.151 | 0.299 |
| Q9Y4E8_UBP15 | | |
| tissue_protein_{—} | −0.063 | 0.668 |
| Q9Y5S9_RBM8A | | |
| tissue_protein_{—} | −0.018 | 0.902 |
| Q9Y5Y2_NUBP2 | | |
| tissue_protein_{—} | −0.329 | 0.021 |
| Q9Y678_COPG1 | | |
| tissue_protein_{—} | 0.118 | 0.419 |
| Q9Y6A9_SPCS1 | | |
| tissue_protein_{—} | −0.170 | 0.242 |
| Q9Y6B6_SAR1B | | |
| tissue_protein_{—} | −0.160 | 0.272 |
| Q9Y6C2_EMIL1 | | |
| tissue_protein_{—} | −0.126 | 0.387 |
| Q9Y6M9_NDUB9 | | |
| tissue_protein_{—} | −0.142 | 0.331 |
| Q9Y6Y8_S23IP | | |
| plasma_lipid_{—} | −0.009 | 0.952 |
| species_conc_{—} | | |
| CE(14:0) | | |
| plasma_lipid_{—} | −0.032 | 0.826 |
| species_conc_{—} | | |
| CE(14:1) | | |
| plasma_lipid_{—} | 0.037 | 0.795 |
| species_conc_{—} | | |
| CE(15:0) | | |
| plasma_lipid_{—} | −0.032 | 0.826 |
| species_conc_{—} | | |
| CE(16:0) | | |
| plasma_lipid_{—} | −0.078 | 0.589 |
| species_conc_{—} | | |
| CE(16:1) | | |
| plasma_lipid_{—} | 0.046 | 0.749 |
| species_conc_{—} | | |
| CE(17:0) | | |
| plasma_lipid_{—} | 0.078 | 0.589 |
| species_conc_{—} | | |
| CE(18:0) | | |
| plasma_lipid_{—} | −0.052 | 0.719 |
| species_conc_{—} | | |
| CE(18:1) | | |
| plasma_lipid_{—} | 0.075 | 0.603 |
| species_conc_{—} | | |
| CE(18:2) | | |
| plasma_lipid_{—} | −0.121 | 0.399 |
| species_conc_{—} | | |
| CE(18:3) | | |
| plasma_lipid_{—} | −0.009 | 0.952 |
| species_conc_{—} | | |
| CE(18:4) | | |
| plasma_lipid_{—} | −0.080 | 0.575 |
| species_conc_{—} | | |
| CE(20:3) | | |
| plasma_lipid_{—} | −0.034 | 0.810 |
| species_conc_{—} | | |
| CE(20:4) | | |
| plasma_lipid_{—} | 0.040 | 0.779 |
| species_conc_{—} | | |
| CE(20:5) | | |
| plasma_lipid_{—} | 0.046 | 0.749 |
| species_conc_{—} | | |
| CE(22:5) | | |
| plasma_lipid_{—} | 0.080 | 0.575 |
| species_conc_{—} | | |
| CE(22:6) | | |
| plasma_lipid_{—} | −0.138 | 0.335 |
| species_conc_{—} | | |
| CER(24:0) | | |
| plasma_lipid_{—} | −0.247 | 0.081 |
| species_conc_{—} | | |
| DAG(16:0/16:0) | | |
| plasma_lipid_{—} | 0.149 | 0.296 |
| species_conc_{—} | | |
| DAG(16:0/18:0) | | |
| plasma_lipid_{—} | −0.359 | 0.010 |
| species_conc_{—} | | |
| DAG(16:0/18:1) | | |
| plasma_lipid_{—} | −0.233 | 0.101 |
| species_conc_{—} | | |
| DAG(16:0/18:2) | | |
| plasma_lipid_{—} | −0.327 | 0.019 |
| species_conc_{—} | | |
| DAG(16:1/18:1) | | |
| plasma_lipid_{—} | −0.327 | 0.019 |
| species_conc_{—} | | |
| DAG(18:0/18:1) | | |
| plasma_lipid_{—} | −0.345 | 0.013 |
| species_conc_{—} | | |
| DAG(18:1/18:1) | | |
| plasma_lipid_{—} | −0.319 | 0.023 |
| species_conc_{—} | | |
| DAG(18:1/18:2) | | |
| plasma_lipid_{—} | 0.230 | 0.105 |
| species_conc_{—} | | |
| FFA(12:0) | | |
| plasma_lipid_{—} | 0.052 | 0.719 |
| species_conc_{—} | | |
| FFA(14:0) | | |
| plasma_lipid_{—} | 0.052 | 0.719 |
| species_conc_{—} | | |
| FFA(14:1) | | |
| plasma_lipid_{—} | 0.043 | 0.764 |
| species_conc_{—} | | |
| FFA(15:0) | | |
| plasma_lipid_{—} | 0.009 | 0.952 |
| species_conc_{—} | | |
| FFA(16:0) | | |
| plasma_lipid_{—} | 0.000 | 1.000 |
| species_conc_{—} | | |
| FFA(16:1) | | |
| plasma_lipid_{—} | 0.037 | 0.795 |
| species_conc_{—} | | |
| FFA(17:0) | | |
| plasma_lipid_{—} | 0.055 | 0.704 |
| species_conc_{—} | | |
| FFA(18:0) | | |
| plasma_lipid_{—} | 0.026 | 0.857 |
| species_conc_{—} | | |
| FFA(18:1) | | |
| plasma_lipid_{—} | 0.103 | 0.470 |
| species_conc_{—} | | |
| FFA(18:2) | | |
| plasma_lipid_{—} | 0.167 | 0.243 |
| species_conc_{—} | | |
| FFA(18:3) | | |
| plasma_lipid_{—} | 0.037 | 0.795 |
| species_conc_{—} | | |
| FFA(20:0) | | |
| plasma_lipid_{—} | 0.037 | 0.795 |
| species_conc_{—} | | |
| FFA(20:1) | | |
| plasma_lipid_{—} | 0.069 | 0.631 |
| species_conc_{—} | | |
| FFA(20:2) | | |
| plasma_lipid_{—} | 0.161 | 0.260 |
| species_conc_{—} | | |
| FFA(20:3) | | |
| plasma_lipid_{—} | −0.009 | 0.952 |
| species_conc_{—} | | |
| FFA(20:4) | | |
| plasma_lipid_{—} | 0.057 | 0.689 |
| species_conc_{—} | | |
| FFA(20:5) | | |
| plasma_lipid_{—} | −0.023 | 0.873 |
| species_conc_{—} | | |
| FFA(22:0) | | |
| plasma_lipid_{—} | −0.009 | 0.952 |
| species_conc_{—} | | |
| FFA(22:1) | | |
| plasma_lipid_{—} | 0.086 | 0.548 |
| species_conc_{—} | | |
| FFA(22:2) | | |
| plasma_lipid_{—} | 0.020 | 0.889 |
| species_conc_{—} | | |
| FFA(22:4) | | |
| plasma_lipid_{—} | 0.169 | 0.235 |
| species_conc_{—} | | |
| FFA(22:5) | | |
| plasma_lipid_{—} | 0.230 | 0.105 |
| species_conc_{—} | | |
| FFA(22:6) | | |
| plasma_lipid_{—} | −0.023 | 0.873 |
| species_conc_{—} | | |
| FFA(24:0) | | |
| plasma_lipid_{—} | 0.138 | 0.335 |
| species_conc_{—} | | |
| FFA(24:1) | | |
| plasma_lipid_{—} | 0.092 | 0.521 |
| species_conc_{—} | | |
| LCER(16:0) | | |
| plasma_lipid_{—} | −0.026 | 0.857 |
| species_conc_{—} | | |
| LPC(16:0) | | |
| plasma_lipid_{—} | −0.049 | 0.734 |
| species_conc_{—} | | |
| LPC(16:1) | | |
| plasma_lipid_{—} | 0.022 | 0.881 |
| species_conc_{—} | | |
| LPC(17:0) | | |
| plasma_lipid_{—} | 0.204 | 0.151 |
| species_conc_{—} | | |
| LPC(18:0) | | |
| plasma_lipid_{—} | 0.078 | 0.589 |
| species_conc_{—} | | |
| LPC(18:1) | | |
| plasma_lipid_{—} | 0.164 | 0.251 |
| species_conc_{—} | | |
| LPC(18:2) | | |
| plasma_lipid_{—} | −0.043 | 0.764 |
| species_conc_{—} | | |
| LPC(20:3) | | |
| plasma_lipid_{—} | −0.009 | 0.952 |
| species_conc_{—} | | |
| LPC(20:4) | | |
| plasma_lipid_{—} | 0.000 | 1.000 |
| species_conc_{—} | | |
| LPE(18:1) | | |
| plasma_lipid_{—} | 0.112 | 0.434 |
| species_conc_{—} | | |
| PC(14:0/18:1) | | |
| plasma_lipid_{—} | 0.106 | 0.458 |
| species_conc_{—} | | |
| PC(14:0/18:2) | | |
| plasma_lipid_{—} | 0.057 | 0.689 |
| species_conc_{—} | | |
| PC(14:0/20:4) | | |
| plasma_lipid_{—} | 0.169 | 0.235 |
| species_conc_{—} | | |
| PC(16:0/14:0) | | |
| plasma_lipid_{—} | −0.006 | 0.968 |
| species_conc_{—} | | |
| PC(16:0/16:0) | | |
| plasma_lipid_{—} | −0.083 | 0.561 |
| species_conc_{—} | | |
| PC(16:0/16:1) | | |
| plasma_lipid_{—} | −0.003 | 0.984 |
| species_conc_{—} | | |
| PC(16:0/18:0) | | |
| plasma_lipid_{—} | −0.227 | 0.110 |
| species_conc_{—} | | |
| PC(16:0/18:1) | | |
| plasma_lipid_{—} | −0.164 | 0.251 |
| species_conc_{—} | | |
| PC(16:0/18:2) | | |
| plasma_lipid_{—} | −0.043 | 0.764 |
| species_conc_{—} | | |
| PC(16:0/18:3) | | |
| plasma_lipid_{—} | −0.201 | 0.157 |
| species_conc_{—} | | |
| PC(16:0/20:2) | | |
| plasma_lipid_{—} | −0.253 | 0.074 |
| species_conc_{—} | | |
| PC(16:0/20:3) | | |
| plasma_lipid_{—} | −0.207 | 0.146 |
| species_conc_{—} | | |
| PC(16:0/20:4) | | |
| plasma_lipid_{—} | 0.049 | 0.734 |
| species_conc_{—} | | |
| PC(16:0/20:5) | | |
| plasma_lipid_{—} | −0.187 | 0.190 |
| species_conc_{—} | | |
| PC(16:0/22:4) | | |
| plasma_lipid_{—} | −0.198 | 0.163 |
| species_conc_{—} | | |
| PC(16:0/22:5) | | |
| plasma_lipid_{—} | −0.040 | 0.779 |
| species_conc_{—} | | |
| PC(16:0/22:6) | | |
| plasma_lipid_{—} | −0.126 | 0.377 |
| species_conc_{—} | | |
| PC(17:0/18:1) | | |
| plasma_lipid_{—} | −0.138 | 0.335 |
| species_conc_{—} | | |
| PC(17:0/18:2) | | |
| plasma_lipid_{—} | −0.161 | 0.260 |
| species_conc_{—} | | |
| PC(17:0/20:4) | | |
| plasma_lipid_{—} | −0.055 | 0.704 |
| species_conc_{—} | | |
| PC(18:0/16:1) | | |
| plasma_lipid_{—} | −0.078 | 0.589 |
| species_conc_{—} | | |
| PC(18:0/18:1) | | |
| plasma_lipid_{—} | 0.017 | 0.905 |
| species_conc_{—} | | |
| PC(18:0/18:2) | | |
| plasma_lipid_{—} | 0.034 | 0.810 |
| species_conc_{—} | | |
| PC(18:0/18:3) | | |
| plasma_lipid_{—} | −0.118 | 0.411 |
| species_conc_{—} | | |
| PC(18:0/20:2) | | |
| plasma_lipid_{—} | −0.132 | 0.356 |
| species_conc_{—} | | |
| PC(18:0/20:3) | | |
| plasma_lipid_{—} | −0.146 | 0.305 |
| species_conc_{—} | | |
| PC(18:0/20:4) | | |
| plasma_lipid_{—} | 0.118 | 0.411 |
| species_conc_{—} | | |
| PC(18:0/20:5) | | |
| plasma_lipid_{—} | −0.159 | 0.264 |
| species_conc_{—} | | |
| PC(18:0/22:4) | | |
| plasma_lipid_{—} | −0.043 | 0.764 |
| species_conc_{—} | | |
| PC(18:0/22:5) | | |
| plasma_lipid_{—} | 0.057 | 0.689 |
| species_conc_{—} | | |
| PC(18:0/22:6) | | |
| plasma_lipid_{—} | −0.123 | 0.388 |
| species_conc_{—} | | |
| PC(18:1/16:1) | | |
| plasma_lipid_{—} | −0.017 | 0.905 |
| species_conc_{—} | | |
| PC(18:1/18:1) | | |
| plasma_lipid_{—} | 0.011 | 0.936 |
| species_conc_{—} | | |
| PC(18:1/18:2) | | |
| plasma_lipid_{—} | −0.169 | 0.235 |
| species_conc_{—} | | |
| PC(18:1/20:3) | | |
| plasma_lipid_{—} | −0.164 | 0.251 |
| species_conc_{—} | | |
| PC(18:1/20:4) | | |
| plasma_lipid_{—} | 0.204 | 0.151 |
| species_conc_{—} | | |
| PC(18:1/20:5) | | |
| plasma_lipid_{—} | 0.052 | 0.719 |
| species_conc_{—} | | |
| PC(18:1/22:6) | | |
| plasma_lipid_{—} | −0.066 | 0.645 |
| species_conc_{—} | | |
| PC(18:2/16:1) | | |
| plasma_lipid_{—} | 0.060 | 0.674 |
| species_conc_{—} | | |
| PC(18:2/18:2) | | |
| plasma_lipid_{—} | −0.244 | 0.084 |
| species_conc_{—} | | |
| PC(18:2/20:3) | | |
| plasma_lipid_{—} | −0.014 | 0.920 |
| species_conc_{—} | | |
| PC(18:2/20:4) | | |
| plasma_lipid_{—} | −0.250 | 0.077 |
| species_conc_{—} | | |
| PE(16:0/18:1) | | |
| plasma_lipid_{—} | −0.324 | 0.020 |
| species_conc_{—} | | |
| PE(16:0/18:2) | | |
| plasma_lipid_{—} | −0.129 | 0.366 |
| species_comp_{—} | | |
| CE(16:0) | | |
| plasma_lipid_{—} | −0.063 | 0.660 |
| species_comp_{—} | | |
| CE(16:1) | | |
| plasma_lipid_{—} | −0.072 | 0.617 |
| species_comp_{—} | | |
| CE(18:1) | | |
| plasma_lipid_{—} | 0.141 | 0.325 |
| species_comp_{—} | | |
| CE(18:2) | | |
| plasma_lipid_{—} | −0.192 | 0.176 |
| species_comp_{—} | | |
| CE(18:3) | | |
| plasma_lipid_{—} | −0.086 | 0.548 |
| species_comp_{—} | | |
| CE(20:4) | | |
| plasma_lipid_{—} | 0.078 | 0.589 |
| species_comp_{—} | | |
| CE(20:5) | | |
| plasma_lipid_{—} | −0.069 | 0.631 |
| species_comp_{—} | | |
| CER(16:0) | | |
| plasma_lipid_{—} | −0.212 | 0.134 |
| species_comp_{—} | | |
| CER(18:0) | | |
| plasma_lipid_{—} | −0.069 | 0.631 |
| species_comp_{—} | | |
| CER(20:0) | | |
| plasma_lipid_{—} | 0.023 | 0.873 |
| species_comp_{—} | | |
| CER(22:0) | | |
| plasma_lipid_{—} | 0.011 | 0.936 |
| species_comp_{—} | | |
| CER(24:0) | | |
| plasma_lipid_{—} | 0.092 | 0.521 |
| species_comp_{—} | | |
| CER(24:1) | | |
| plasma_lipid_{—} | 0.120 | 0.403 |
| species_comp_{—} | | |
| DAG(14:0/14:0) | | |
| plasma_lipid_{—} | 0.055 | 0.703 |
| species_comp_{—} | | |
| DAG(14:0/18:1) | | |
| plasma_lipid_{—} | −0.019 | 0.897 |
| species_comp_{—} | | |
| DAG(16:0/16:0) | | |
| plasma_lipid_{—} | −0.158 | 0.267 |
| species_comp_{—} | | |
| DAG(16:0/16:1) | | |
| plasma_lipid_{—} | 0.385 | 0.005 |
| species_comp_{—} | | |
| DAG(16:0/18:0) | | |
| plasma_lipid_{—} | −0.353 | 0.011 |
| species_comp_{—} | | |
| DAG(16:0/18:1) | | |
| plasma_lipid_{—} | −0.080 | 0.575 |
| species_comp_{—} | | |
| DAG(16:0/18:2) | | |
| plasma_lipid_{—} | −0.218 | 0.124 |
| species_comp_{—} | | |
| DAG(16:1/18:1) | | |
| plasma_lipid_{—} | −0.052 | 0.719 |
| species_comp_{—} | | |
| DAG(16:1/18:2) | | |
| plasma_lipid_{—} | −0.152 | 0.286 |
| species_comp_{—} | | |
| DAG(18:0/18:1) | | |
| plasma_lipid_{—} | −0.201 | 0.157 |
| species_comp_{—} | | |
| DAG(18:1/18:1) | | |
| plasma_lipid_{—} | −0.118 | 0.411 |
| species_comp_{—} | | |
| DAG(18:1/18:2) | | |
| plasma_lipid_{—} | −0.101 | 0.483 |
| species_comp_{—} | | |
| DAG(18:1/20:3) | | |
| plasma_lipid_{—} | −0.141 | 0.325 |
| species_comp_{—} | | |
| DAG(18:1/20:4) | | |
| plasma_lipid_{—} | 0.156 | 0.275 |
| species_comp_{—} | | |
| DAG(18:1/20:5) | | |
| plasma_lipid_{—} | 0.069 | 0.631 |
| species_comp_{—} | | |
| DAG(18:1/22:6) | | |
| plasma_lipid_{—} | 0.149 | 0.296 |
| species_comp_{—} | | |
| DAG(18:2/18:3) | | |
| plasma_lipid_{—} | −0.126 | 0.377 |
| species_comp_{—} | | |
| DAG(18:2/20:4) | | |
| plasma_lipid_{—} | 0.333 | 0.017 |
| species_comp_{—} | | |
| DAG(20:0/20:0) | | |
| plasma_lipid_{—} | 0.017 | 0.903 |
| species_comp_{—} | | |
| DCER(22:0) | | |
| plasma_lipid_{—} | −0.094 | 0.512 |
| species_comp_{—} | | |
| DCER(24:0) | | |
| plasma_lipid_{—} | −0.095 | 0.508 |
| species_comp_{—} | | |
| DCER(24:1) | | |
| plasma_lipid_{—} | −0.158 | 0.268 |
| species_comp_{—} | | |
| FFA(16:0) | | |
| plasma_lipid_{—} | −0.020 | 0.889 |
| species_comp_{—} | | |
| FFA(16:1) | | |
| plasma_lipid_{—} | 0.069 | 0.631 |
| species_comp_{—} | | |
| FFA(18:0) | | |
| plasma_lipid_{—} | −0.141 | 0.325 |
| species_comp_{—} | | |
| FFA(18:1) | | |
| plasma_lipid_{—} | 0.267 | 0.058 |
| species_comp_{—} | | |
| FFA(18:2) | | |
| plasma_lipid_{—} | 0.029 | 0.841 |
| species_comp_{—} | | |
| FFA(20:5) | | |
| plasma_lipid_{—} | −0.413 | 0.003 |
| species_comp_{—} | | |
| HCER(16:0) | | |
| plasma_lipid_{—} | −0.210 | 0.139 |
| species_comp_{—} | | |
| HCER(18:0) | | |
| plasma_lipid_{—} | −0.148 | 0.300 |
| species_comp_{—} | | |
| HCER(20:0) | | |
| plasma_lipid_{—} | 0.218 | 0.124 |
| species_comp_{—} | | |
| HCER(22:0) | | |
| plasma_lipid_{—} | −0.085 | 0.554 |
| species_comp_{—} | | |
| HCER(22:1) | | |
| plasma_lipid_{—} | −0.060 | 0.674 |
| species_comp_{—} | | |
| HCER(24:0) | | |
| plasma_lipid_{—} | −0.029 | 0.841 |
| species_comp_{—} | | |
| HCER(24:1) | | |
| plasma_lipid_{—} | −0.063 | 0.660 |
| species_comp_{—} | | |
| LCER(16:0) | | |
| plasma_lipid_{—} | −0.192 | 0.178 |
| species_comp_{—} | | |
| LCER(24:0) | | |
| plasma_lipid_{—} | 0.164 | 0.250 |
| species_comp_{—} | | |
| LCER(24:1) | | |
| plasma_lipid_{—} | −0.356 | 0.010 |
| species_comp_{—} | | |
| LPC(16:0) | | |
| plasma_lipid_{—} | 0.258 | 0.067 |
| species_comp_{—} | | |
| LPC(18:0) | | |
| plasma_lipid_{—} | 0.161 | 0.260 |
| species_comp_{—} | | |
| LPC(18:1) | | |
| plasma_lipid_{—} | 0.189 | 0.183 |
| species_comp_{—} | | |
| LPC(18:2) | | |
| plasma_lipid_{—} | −0.086 | 0.548 |
| species_comp_{—} | | |
| LPC(20:4) | | |
| plasma_lipid_{—} | −0.066 | 0.645 |
| species_comp_{—} | | |
| LPE(16:0) | | |
| plasma_lipid_{—} | −0.164 | 0.251 |
| species_comp_{—} | | |
| LPE(18:0) | | |
| plasma_lipid_{—} | 0.086 | 0.548 |
| species_comp_{—} | | |
| LPE(18:1) | | |
| plasma_lipid_{—} | 0.172 | 0.227 |
| species_comp_{—} | | |
| LPE(18:2) | | |
| plasma_lipid_{—} | 0.011 | 0.936 |
| species_comp_{—} | | |
| LPE(20:3) | | |
| plasma_lipid_{—} | −0.017 | 0.905 |
| species_comp_{—} | | |
| LPE(20:4) | | |
| plasma_lipid_{—} | −0.080 | 0.575 |
| species_comp_{—} | | |
| PC(16:0/18:1) | | |
| plasma_lipid_{—} | −0.063 | 0.660 |
| species_comp_{—} | | |
| PC(16:0/18:2) | | |
| plasma_lipid_{—} | −0.198 | 0.163 |
| species_comp_{—} | | |
| PC(16:0/20:3) | | |
| plasma_lipid_{—} | −0.141 | 0.325 |
| species_comp_{—} | | |
| PC(16:0/20:4) | | |
| plasma_lipid_{—} | 0.121 | 0.399 |
| species_comp_{—} | | |
| PC(16:0/20:5) | | |
| plasma_lipid_{—} | 0.089 | 0.535 |
| species_comp_{—} | | |
| PC(16:0/22:6) | | |
| plasma_lipid_{—} | 0.195 | 0.170 |
| species_comp_{—} | | |
| PC(18:0/18:2) | | |
| plasma_lipid_{—} | −0.049 | 0.734 |
| species_comp_{—} | | |
| PC(18:0/20:3) | | |
| plasma_lipid_{—} | −0.046 | 0.749 |
| species_comp_{—} | | |
| PC(18:0/20:4) | | |
| plasma_lipid_{—} | 0.181 | 0.204 |
| species_comp_{—} | | |
| PC(18:0/22:6) | | |
| plasma_lipid_{—} | 0.098 | 0.496 |
| species_comp_{—} | | |
| PC(18:1/18:2) | | |
| plasma_lipid_{—} | −0.210 | 0.140 |
| species_comp_{—} | | |
| PE(16:0/18:1) | | |
| plasma_lipid_{—} | −0.299 | 0.033 |
| species_comp_{—} | | |
| PE(16:0/18:2) | | |
| plasma_lipid_{—} | 0.032 | 0.826 |
| class_conc_CE | | |
| plasma_lipid_{—} | −0.164 | 0.251 |
| class_conc_CER | | |
| plasma_lipid_{—} | −0.333 | 0.017 |
| class_conc_DAG | | |
| plasma_lipid_{—} | 0.060 | 0.674 |
| class_conc_FFA | | |
| plasma_lipid_{—} | 0.212 | 0.134 |
| class_conc_HCER | | |
| plasma_lipid_{—} | 0.103 | 0.470 |
| class_conc_LCER | | |
| plasma_lipid_{—} | 0.086 | 0.548 |
| class_conc_LPC | | |
| plasma_lipid_{—} | −0.020 | 0.889 |
| class_conc_LPE | | |
| plasma_lipid_{—} | −0.167 | 0.243 |
| class_conc_PC | | |
| plasma_lipid_{—} | −0.049 | 0.734 |
| class_conc_PE | | |
| plasma_lipid_{—} | −0.078 | 0.589 |
| class_conc_SM | | |
| plasma_lipid_{—} | −0.299 | 0.033 |
| class_conc_TAG | | |
| plasma_lipid_{—} | 0.192 | 0.176 |
| class_comp_CE | | |
| plasma_lipid_{—} | 0.118 | 0.411 |
| class_comp_FFA | | |
| plasma_lipid_{—} | 0.299 | 0.033 |
| class_comp_LPC | | |
| plasma_lipid_{—} | −0.164 | 0.251 |
| class_comp_PC | | |
| plasma_lipid_{—} | 0.118 | 0.411 |
| class_comp_SM | | |
| plasma_lipid_{—} | −0.238 | 0.092 |
| class_comp_TAG | | |
| plasma_lipid_{—} | −0.009 | 0.952 |
| fatty_acid_conc_{—} | | |
| CE(FA14:0) | | |
| plasma_lipid_{—} | −0.032 | 0.826 |
| fatty_acid_conc_{—} | | |
| CE(FA14:1) | | |
| plasma_lipid_{—} | 0.037 | 0.795 |
| fatty_acid_conc_{—} | | |
| CE(FA15:0) | | |
| plasma_lipid_{—} | −0.032 | 0.826 |
| fatty_acid_conc_{—} | | |
| CE(FA16:0) | | |
| plasma_lipid_{—} | −0.078 | 0.589 |
| fatty_acid_conc_{—} | | |
| CE(FA16:1) | | |
| plasma_lipid_{—} | 0.046 | 0.749 |
| fatty_acid_conc_{—} | | |
| CE(FA17:0) | | |
| plasma_lipid_{—} | 0.078 | 0.589 |
| fatty_acid_conc_{—} | | |
| CE(FA18:0) | | |
| plasma_lipid_{—} | −0.052 | 0.719 |
| fatty_acid_conc_{—} | | |
| CE(FA18:1) | | |
| plasma_lipid_{—} | 0.075 | 0.603 |
| fatty_acid_conc_{—} | | |
| CE(FA18:2) | | |
| plasma_lipid_{—} | −0.121 | 0.399 |
| fatty_acid_conc_{—} | | |
| CE(FA18:3) | | |
| plasma_lipid_{—} | −0.009 | 0.952 |
| fatty_acid_conc_{—} | | |
| CE(FA18:4) | | |
| plasma_lipid_{—} | −0.080 | 0.575 |
| fatty_acid_conc_{—} | | |
| CE(FA20:3) | | |
| plasma_lipid_{—} | −0.034 | 0.810 |
| fatty_acid_conc_{—} | | |
| CE(FA20:4) | | |
| plasma_lipid_{—} | 0.040 | 0.779 |
| fatty_acid_conc_{—} | | |
| CE(FA20:5) | | |
| plasma_lipid_{—} | 0.046 | 0.749 |
| fatty_acid_conc_{—} | | |
| CE(FA22:5) | | |
| plasma_lipid_{—} | 0.080 | 0.575 |
| fatty_acid_conc_{—} | | |
| CE(FA22:6) | | |
| plasma_lipid_{—} | −0.138 | 0.335 |
| fatty_acid_conc_{—} | | |
| CER(FA24:0) | | |
| plasma_lipid_{—} | −0.052 | 0.719 |
| fatty_acid_conc_{—} | | |
| DAG(FA14:0) | | |
| plasma_lipid_{—} | −0.241 | 0.088 |
| fatty_acid_conc_{—} | | |
| DAG(FA16:0) | | |
| plasma_lipid_{—} | −0.218 | 0.124 |
| fatty_acid_conc_{—} | | |
| DAG(FA16:1) | | |
| plasma_lipid_{—} | −0.052 | 0.719 |
| fatty_acid_conc_{—} | | |
| DAG(FA18:0) | | |
| plasma_lipid_{—} | −0.356 | 0.010 |
| fatty_acid_conc_{—} | | |
| DAG(FA18:1) | | |
| plasma_lipid_{—} | −0.299 | 0.033 |
| fatty_acid_conc_{—} | | |
| DAG(FA18:2) | | |
| plasma_lipid_{—} | −0.276 | 0.050 |
| fatty_acid_conc_{—} | | |
| DAG(FA20:4) | | |
| plasma_lipid_{—} | 0.230 | 0.105 |
| fatty_acid_conc_{—} | | |
| FFA(FA12:0) | | |
| plasma_lipid_{—} | 0.052 | 0.719 |
| fatty_acid_conc_{—} | | |
| FFA(FA14:0) | | |
| plasma_lipid_{—} | 0.052 | 0.719 |
| fatty_acid_conc_{—} | | |
| FFA(FA14:1) | | |
| plasma_lipid_{—} | 0.043 | 0.764 |
| fatty_acid_conc_{—} | | |
| FFA(FA15:0) | | |
| plasma_lipid_{—} | 0.009 | 0.952 |
| fatty_acid_conc_{—} | | |
| FFA(FA16:0) | | |
| plasma_lipid_{—} | 0.000 | 1.000 |
| fatty_acid_conc_{—} | | |
| FFA(FA16:1) | | |
| plasma_lipid_{—} | 0.037 | 0.795 |
| fatty_acid_conc_{—} | | |
| FFA(FA17:0) | | |
| plasma_lipid_{—} | 0.055 | 0.704 |
| fatty_acid_conc_{—} | | |
| FFA(FA18:0) | | |
| plasma_lipid_{—} | 0.026 | 0.857 |
| fatty_acid_conc_{—} | | |
| FFA(FA18:1) | | |
| plasma_lipid_{—} | 0.103 | 0.470 |
| fatty_acid_conc_{—} | | |
| FFA(FA18:2) | | |
| plasma_lipid_{—} | 0.167 | 0.243 |
| fatty_acid_conc_{—} | | |
| FFA(FA18:3) | | |
| plasma_lipid_{—} | 0.037 | 0.795 |
| fatty_acid_conc_{—} | | |
| FFA(FA20:0) | | |
| plasma_lipid_{—} | 0.037 | 0.795 |
| fatty_acid_conc_{—} | | |
| FFA(FA20:1) | | |
| plasma_lipid_{—} | 0.069 | 0.631 |
| fatty_acid_conc_{—} | | |
| FFA(FA20:2) | | |
| plasma_lipid_{—} | 0.161 | 0.260 |
| fatty_acid_conc_{—} | | |
| FFA(FA20:3) | | |
| plasma_lipid_{—} | −0.009 | 0.952 |
| fatty_acid_conc_{—} | | |
| FFA(FA20:4) | | |
| plasma_lipid_{—} | 0.057 | 0.689 |
| fatty_acid_conc_{—} | | |
| FFA(FA20:5) | | |
| plasma_lipid_{—} | −0.023 | 0.873 |
| fatty_acid_conc_{—} | | |
| FFA(FA22:0) | | |
| plasma_lipid_{—} | −0.009 | 0.952 |
| fatty_acid_conc_{—} | | |
| FFA(FA22:1) | | |
| plasma_lipid_{—} | 0.086 | 0.548 |
| fatty_acid_conc_{—} | | |
| FFA(FA22:2) | | |
| plasma_lipid_{—} | 0.020 | 0.889 |
| fatty_acid_conc_{—} | | |
| FFA(FA22:4) | | |
| plasma_lipid_{—} | 0.169 | 0.235 |
| fatty_acid_conc_{—} | | |
| FFA(FA22:5) | | |
| plasma_lipid_{—} | 0.230 | 0.105 |
| fatty_acid_conc_{—} | | |
| FFA(FA22:6) | | |
| plasma_lipid_{—} | −0.023 | 0.873 |
| fatty_acid_conc_{—} | | |
| FFA(FA24:0) | | |
| plasma_lipid_{—} | 0.138 | 0.335 |
| fatty_acid_conc_{—} | | |
| FFA(FA24:1) | | |
| plasma_lipid_{—} | 0.092 | 0.521 |
| fatty_acid_conc_{—} | | |
| LCER(FA16:0) | | |
| plasma_lipid_{—} | −0.026 | 0.857 |
| fatty_acid_conc_{—} | | |
| LPC(FA16:0) | | |
| plasma_lipid_{—} | −0.049 | 0.734 |
| fatty_acid_conc_{—} | | |
| LPC(FA16:1) | | |
| plasma_lipid_{—} | 0.022 | 0.881 |
| fatty_acid_conc_{—} | | |
| LPC(FA17:0) | | |
| plasma_lipid_{—} | 0.204 | 0.151 |
| fatty_acid_conc_{—} | | |
| LPC(FA18:0) | | |
| plasma_lipid_{—} | 0.078 | 0.589 |
| fatty_acid_conc_{—} | | |
| LPC(FA18:1) | | |
| plasma_lipid_{—} | 0.164 | 0.251 |
| fatty_acid_conc_{—} | | |
| LPC(FA18:2) | | |
| plasma_lipid_{—} | −0.043 | 0.764 |
| fatty_acid_conc_{—} | | |
| LPC(FA20:3) | | |
| plasma_lipid_{—} | −0.009 | 0.952 |
| fatty_acid_conc_{—} | | |
| LPC(FA20:4) | | |
| plasma_lipid_{—} | 0.000 | 1.000 |
| fatty_acid_conc_{—} | | |
| LPE(FA18:1) | | |
| plasma_lipid_{—} | 0.135 | 0.345 |
| fatty_acid_conc_{—} | | |
| PC(FA14:0) | | |
| plasma_lipid_{—} | −0.103 | 0.470 |
| fatty_acid_conc_{—} | | |
| PC(FA15:0) | | |
| plasma_lipid_{—} | −0.207 | 0.146 |
| fatty_acid_conc_{—} | | |
| PC(FA16:0) | | |
| plasma_lipid_{—} | −0.115 | 0.422 |
| fatty_acid_conc_{—} | | |
| PC(FA16:1) | | |
| plasma_lipid_{—} | −0.123 | 0.388 |
| fatty_acid_conc_{—} | | |
| PC(FA17:0) | | |
| plasma_lipid_{—} | −0.075 | 0.603 |
| fatty_acid_conc_{—} | | |
| PC(FA18:0) | | |
| plasma_lipid_{—} | −0.175 | 0.219 |
| fatty_acid_conc_{—} | | |
| PC(FA18:1) | | |
| plasma_lipid_{—} | −0.095 | 0.508 |
| fatty_acid_conc_{—} | | |
| PC(FA18:2) | | |
| plasma_lipid_{—} | 0.006 | 0.968 |
| fatty_acid_conc_{—} | | |
| PC(FA18:3) | | |
| plasma_lipid_{—} | −0.099 | 0.489 |
| fatty_acid_conc_{—} | | |
| PC(FA20:0) | | |
| plasma_lipid_{—} | 0.011 | 0.936 |
| fatty_acid_conc_{—} | | |
| PC(FA20:1) | | |
| plasma_lipid_{—} | −0.152 | 0.286 |
| fatty_acid_conc_{—} | | |
| PC(FA20:2) | | |
| plasma_lipid_{—} | −0.218 | 0.124 |
| fatty_acid_conc_{—} | | |
| PC(FA20:3) | | |
| plasma_lipid_{—} | −0.187 | 0.190 |
| fatty_acid_conc_{—} | | |
| PC(FA20:4) | | |
| plasma_lipid_{—} | 0.046 | 0.749 |
| fatty_acid_conc_{—} | | |
| PC(FA20:5) | | |
| plasma_lipid_{—} | −0.187 | 0.190 |
| fatty_acid_conc_{—} | | |
| PC(FA22:4) | | |
| plasma_lipid_{—} | −0.146 | 0.305 |
| fatty_acid_conc_{—} | | |
| PC(FA22:5) | | |
| plasma_lipid_{—} | −0.037 | 0.795 |
| fatty_acid_conc_{—} | | |
| PC(FA22:6) | | |
| plasma_lipid_{—} | −0.069 | 0.631 |
| fatty_acid_conc_{—} | | |
| PE(FA16:0) | | |
| plasma_lipid_{—} | −0.092 | 0.521 |
| fatty_acid_conc_{—} | | |
| PE(FA18:0) | | |
| plasma_lipid_{—} | 0.029 | 0.841 |
| fatty_acid_conc_{—} | | |
| PE(FA18:1) | | |
| plasma_lipid_{—} | −0.069 | 0.631 |
| fatty_acid_conc_{—} | | |
| PE(FA18:2) | | |
| plasma_lipid_{—} | −0.149 | 0.296 |
| fatty_acid_conc_{—} | | |
| PE(FA20:3) | | |
| plasma_lipid_{—} | 0.000 | 1.000 |
| fatty_acid_conc_{—} | | |
| PE(FA20:4) | | |
| plasma_lipid_{—} | 0.035 | 0.805 |
| fatty_acid_conc_{—} | | |
| PE(FA20:5) | | |
| plasma_lipid_{—} | −0.100 | 0.483 |
| fatty_acid_conc_{—} | | |
| PE(FA22:4) | | |
| plasma_lipid_{—} | 0.168 | 0.239 |
| fatty_acid_conc_{—} | | |
| PE(FA22:5) | | |
| plasma_lipid_{—} | −0.037 | 0.795 |
| fatty_acid_conc_{—} | | |
| PE(FA22:6) | | |
| plasma_lipid_{—} | 0.000 | 1.000 |
| fatty_acid_conc_{—} | | |
| SM(FA14:0) | | |
| plasma_lipid_{—} | −0.009 | 0.952 |
| fatty_acid_conc_{—} | | |
| SM(FA16:0) | | |
| plasma_lipid_{—} | −0.095 | 0.508 |
| fatty_acid_conc_{—} | | |
| SM(FA18:0) | | |
| plasma_lipid_{—} | −0.121 | 0.399 |
| fatty_acid_conc_{—} | | |
| SM(FA18:1) | | |
| plasma_lipid_{—} | −0.181 | 0.204 |
| fatty_acid_conc_{—} | | |
| SM(FA20:0) | | |
| plasma_lipid_{—} | −0.243 | 0.086 |
| fatty_acid_conc_{—} | | |
| SM(FA20:1) | | |
| plasma_lipid_{—} | 0.040 | 0.779 |
| fatty_acid_conc_{—} | | |
| SM(FA22:0) | | |
| plasma_lipid_{—} | −0.233 | 0.101 |
| fatty_acid_conc_{—} | | |
| SM(FA22:1) | | |
| plasma_lipid_{—} | 0.034 | 0.810 |
| fatty_acid_conc_{—} | | |
| SM(FA24:0) | | |
| plasma_lipid_{—} | −0.198 | 0.163 |
| fatty_acid_conc_{—} | | |
| SM(FA24:1) | | |
| plasma_lipid_{—} | 0.023 | 0.873 |
| fatty_acid_conc_{—} | | |
| TAG(FA12:0) | | |
| plasma_lipid_{—} | −0.132 | 0.356 |
| fatty_acid_conc_{—} | | |
| TAG(FA14:0) | | |
| plasma_lipid_{—} | 0.204 | 0.151 |
| fatty_acid_conc_{—} | | |
| TAG(FA14:1) | | |
| plasma_lipid_{—} | 0.204 | 0.151 |
| fatty_acid_conc_{—} | | |
| TAG(FA15:0) | | |
| plasma_lipid_{—} | −0.408 | 0.003 |
| fatty_acid_conc_{—} | | |
| TAG(FA16:0) | | |
| plasma_lipid_{—} | −0.422 | 0.002 |
| fatty_acid_conc_{—} | | |
| TAG(FA16:1) | | |
| plasma_lipid_{—} | 0.212 | 0.134 |
| fatty_acid_conc_{—} | | |
| TAG(FA17:0) | | |
| plasma_lipid_{—} | −0.370 | 0.007 |
| fatty_acid_conc_{—} | | |
| TAG(FA18:0) | | |
| plasma_lipid_{—} | −0.324 | 0.020 |
| fatty_acid_conc_{—} | | |
| TAG(FA18:1) | | |
| plasma_lipid_{—} | −0.347 | 0.013 |
| fatty_acid_conc_{—} | | |
| TAG(FA18:2) | | |
| plasma_lipid_{—} | 0.235 | 0.096 |
| fatty_acid_conc_{—} | | |
| TAG(FA18:3) | | |
| plasma_lipid_{—} | 0.189 | 0.183 |
| fatty_acid_conc_{—} | | |
| TAG(FA20:0) | | |
| plasma_lipid_{—} | −0.264 | 0.061 |
| fatty_acid_conc_{—} | | |
| TAG(FA20:1) | | |
| plasma_lipid_{—} | −0.224 | 0.114 |
| fatty_acid_conc_{—} | | |
| TAG(FA20:2) | | |
| plasma_lipid_{—} | −0.210 | 0.140 |
| fatty_acid_conc_{—} | | |
| TAG(FA20:3) | | |
| plasma_lipid_{—} | −0.379 | 0.006 |
| fatty_acid_conc_{—} | | |
| TAG(FA20:4) | | |
| plasma_lipid_{—} | 0.250 | 0.077 |
| fatty_acid_conc_{—} | | |
| TAG(FA20:5) | | |
| plasma_lipid_{—} | −0.098 | 0.496 |
| fatty_acid_conc_{—} | | |
| TAG(FA22:1) | | |
| plasma_lipid_{—} | −0.227 | 0.109 |
| fatty_acid_conc_{—} | | |
| TAG(FA22:4) | | |
| plasma_lipid_{—} | −0.158 | 0.268 |
| fatty_acid_conc_{—} | | |
| TAG(FA22:5) | | |
| plasma_lipid_{—} | 0.029 | 0.841 |
| fatty_acid_conc_{—} | | |
| TAG(FA22:6) | | |
| plasma_lipid_{—} | −0.129 | 0.366 |
| fatty_acid_comp_{—} | | |
| CE(FA16:0) | | |
| plasma_lipid_{—} | −0.063 | 0.660 |
| fatty_acid_comp_{—} | | |
| CE(FA16:1) | | |
| plasma_lipid_{—} | −0.072 | 0.617 |
| fatty_acid_comp_{—} | | |
| CE(FA18:1) | | |
| plasma_lipid_{—} | 0.141 | 0.325 |
| fatty_acid_comp_{—} | | |
| CE(FA18:2) | | |
| plasma_lipid_{—} | −0.192 | 0.176 |
| fatty_acid_comp_{—} | | |
| CE(FA18:3) | | |
| plasma_lipid_{—} | −0.086 | 0.548 |
| fatty_acid_comp_{—} | | |
| CE(FA20:4) | | |
| plasma_lipid_{—} | 0.078 | 0.589 |
| fatty_acid_comp_{—} | | |
| CE(FA20:5) | | |
| plasma_lipid_{—} | −0.069 | 0.631 |
| fatty_acid_comp_{—} | | |
| CER(FA16:0) | | |
| plasma_lipid_{—} | −0.212 | 0.134 |
| fatty_acid_comp_{—} | | |
| CER(FA18:0) | | |
| plasma_lipid_{—} | −0.069 | 0.631 |
| fatty_acid_comp_{—} | | |
| CER(FA20:0) | | |
| plasma_lipid_{—} | 0.023 | 0.873 |
| fatty_acid_comp_{—} | | |
| CER(FA22:0) | | |
| plasma_lipid_{—} | 0.011 | 0.936 |
| fatty_acid_comp_{—} | | |
| CER(FA24:0) | | |
| plasma_lipid_{—} | 0.092 | 0.521 |
| fatty_acid_comp_{—} | | |
| CER(FA24:1) | | |
| plasma_lipid_{—} | 0.167 | 0.243 |
| fatty_acid_comp_{—} | | |
| DAG(FA14:0) | | |
| plasma_lipid_{—} | 0.100 | 0.483 |
| fatty_acid_comp_{—} | | |
| DAG(FA16:0) | | |
| plasma_lipid_{—} | −0.049 | 0.734 |
| fatty_acid_comp_{—} | | |
| DAG(FA16:1) | | |
| plasma_lipid_{—} | 0.345 | 0.013 |
| fatty_acid_comp_{—} | | |
| DAG(FA18:0) | | |
| plasma_lipid_{—} | −0.388 | 0.005 |
| fatty_acid_comp_{—} | | |
| DAG(FA18:1) | | |
| plasma_lipid_{—} | −0.052 | 0.719 |
| fatty_acid_comp_{—} | | |
| DAG(FA18:2) | | |
| plasma_lipid_{—} | 0.324 | 0.020 |
| fatty_acid_comp_{—} | | |
| DAG(FA20:0) | | |
| plasma_lipid_{—} | −0.129 | 0.366 |
| fatty_acid_comp_{—} | | |
| DAG(FA20:4) | | |
| plasma_lipid_{—} | 0.104 | 0.469 |
| fatty_acid_comp_{—} | | |
| DAG(FA20:5) | | |
| plasma_lipid_{—} | 0.043 | 0.764 |
| fatty_acid_comp_{—} | | |
| DAG(FA22:6) | | |
| plasma_lipid_{—} | 0.017 | 0.903 |
| fatty_acid_comp_{—} | | |
| DCER(FA22:0) | | |
| plasma_lipid_{—} | −0.094 | 0.512 |
| fatty_acid_comp_{—} | | |
| DCER(FA24:0) | | |
| plasma_lipid_{—} | −0.095 | 0.508 |
| fatty_acid_comp_{—} | | |
| DCER(FA24:1) | | |
| plasma_lipid_{—} | −0.158 | 0.268 |
| fatty_acid_comp_{—} | | |
| FFA(FA16:0) | | |
| plasma_lipid_{—} | −0.020 | 0.889 |
| fatty_acid_comp_{—} | | |
| FFA(FA16:1) | | |
| plasma_lipid_{—} | 0.069 | 0.631 |
| fatty_acid_comp_{—} | | |
| FFA(FA18:0) | | |
| plasma_lipid_{—} | −0.141 | 0.325 |
| fatty_acid_comp_{—} | | |
| FFA(FA18:1) | | |
| plasma_lipid_{—} | 0.267 | 0.058 |
| fatty_acid_comp_{—} | | |
| FFA(FA18:2) | | |
| plasma_lipid_{—} | 0.029 | 0.841 |
| fatty_acid_comp_{—} | | |
| FFA(FA20:5) | | |
| plasma_lipid_{—} | −0.413 | 0.003 |
| fatty_acid_comp_{—} | | |
| HCER(FA16:0) | | |
| plasma_lipid_{—} | −0.210 | 0.139 |
| fatty_acid_comp_{—} | | |
| HCER(FA18:0) | | |
| plasma_lipid_{—} | −0.148 | 0.300 |
| fatty_acid_comp_{—} | | |
| HCER(FA20:0) | | |
| plasma_lipid_{—} | 0.218 | 0.124 |
| fatty_acid_comp_{—} | | |
| HCER(FA22:0) | | |
| plasma_lipid_{—} | −0.085 | 0.554 |
| fatty_acid_comp_{—} | | |
| HCER(FA22:1) | | |
| plasma_lipid_{—} | −0.060 | 0.674 |
| fatty_acid_comp_{—} | | |
| HCER(FA24:0) | | |
| plasma_lipid_{—} | −0.029 | 0.841 |
| fatty_acid_comp_{—} | | |
| HCER(FA24:1) | | |
| plasma_lipid_{—} | −0.063 | 0.660 |
| fatty_acid_comp_{—} | | |
| LCER(FA16:0) | | |
| plasma_lipid_{—} | −0.192 | 0.178 |
| fatty_acid_comp_{—} | | |
| LCER(FA24:0) | | |
| plasma_lipid_{—} | 0.164 | 0.250 |
| fatty_acid_comp_{—} | | |
| LCER(FA24:1) | | |
| plasma_lipid_{—} | −0.356 | 0.010 |
| fatty_acid_comp_{—} | | |
| LPC(FA16:0) | | |
| plasma_lipid_{—} | 0.258 | 0.067 |
| fatty_acid_comp_{—} | | |
| LPC(FA18:0) | | |
| plasma_lipid_{—} | 0.161 | 0.260 |
| fatty_acid_comp_{—} | | |
| LPC(FA18:1) | | |
| plasma_lipid_{—} | 0.189 | 0.183 |
| fatty_acid_comp_{—} | | |
| LPC(FA18:2) | | |
| plasma_lipid_{—} | −0.086 | 0.548 |
| fatty_acid_comp_{—} | | |
| LPC(FA20:4) | | |
| plasma_lipid_{—} | −0.066 | 0.645 |
| fatty_acid_comp_{—} | | |
| LPE(FA16:0) | | |
| plasma_lipid_{—} | −0.164 | 0.251 |
| fatty_acid_comp_{—} | | |
| LPE(FA18:0) | | |
| plasma_lipid_{—} | 0.086 | 0.548 |
| fatty_acid_comp_{—} | | |
| LPE(FA18:1) | | |
| plasma_lipid_{—} | 0.172 | 0.227 |
| fatty_acid_comp_{—} | | |
| LPE(FA18:2) | | |
| plasma_lipid_{—} | 0.011 | 0.936 |
| fatty_acid_comp_{—} | | |
| LPE(FA20:3) | | |
| plasma_lipid_{—} | −0.016 | 0.912 |
| fatty_acid_comp_{—} | | |
| LPE(FA20:4) | | |
| plasma_lipid_{—} | −0.235 | 0.096 |
| fatty_acid_comp_{—} | | |
| PC(FA16:0) | | |
| plasma_lipid_{—} | 0.299 | 0.033 |
| fatty_acid_comp_{—} | | |
| PC(FA18:0) | | |
| plasma_lipid_{—} | 0.017 | 0.905 |
| fatty_acid_comp_{—} | | |
| PC(FA18:1) | | |
| plasma_lipid_{—} | 0.078 | 0.589 |
| fatty_acid_comp_{—} | | |
| PC(FA18:2) | | |
| plasma_lipid_{—} | −0.135 | 0.345 |
| fatty_acid_comp_{—} | | |
| PC(FA20:3) | | |
| plasma_lipid_{—} | −0.112 | 0.434 |
| fatty_acid_comp_{—} | | |
| PC(FA20:4) | | |
| plasma_lipid_{—} | 0.121 | 0.399 |
| fatty_acid_comp_{—} | | |
| PC(FA20:5) | | |
| plasma_lipid_{—} | 0.123 | 0.388 |
| fatty_acid_comp_{—} | | |
| PC(FA22:6) | | |
| plasma_lipid_{—} | −0.037 | 0.795 |
| fatty_acid_comp_{—} | | |
| PE(FA16:0) | | |
| plasma_lipid_{—} | −0.316 | 0.024 |
| fatty_acid_comp_{—} | | |
| PE(FA18:0) | | |
| plasma_lipid_{—} | 0.109 | 0.446 |
| fatty_acid_comp_{—} | | |
| PE(FA18:1) | | |
| plasma_lipid_{—} | −0.095 | 0.508 |
| fatty_acid_comp_{—} | | |
| PE(FA18:2) | | |
| plasma_lipid_{—} | −0.138 | 0.335 |
| fatty_acid_comp_{—} | | |
| PE(FA20:3) | | |
| plasma_lipid_{—} | 0.204 | 0.151 |
| fatty_acid_comp_{—} | | |
| PE(FA20:4) | | |
| plasma_lipid_{—} | 0.048 | 0.739 |
| fatty_acid_comp_{—} | | |
| PE(FA20:5) | | |
| plasma_lipid_{—} | 0.172 | 0.226 |
| fatty_acid_comp_{—} | | |
| PE(FA22:5) | | |
| plasma_lipid_{—} | 0.057 | 0.689 |
| fatty_acid_comp_{—} | | |
| PE(FA22:6) | | |
| plasma_lipid_{—} | 0.086 | 0.548 |
| fatty_acid_comp_{—} | | |
| SM(FA14:0) | | |
| plasma_lipid_{—} | 0.123 | 0.388 |
| fatty_acid_comp_{—} | | |
| SM(FA16:0) | | |
| plasma_lipid_{—} | −0.014 | 0.920 |
| fatty_acid_comp_{—} | | |
| SM(FA18:0) | | |
| plasma_lipid_{—} | −0.270 | 0.055 |
| fatty_acid_comp_{—} | | |
| SM(FA20:0) | | |
| plasma_lipid_{—} | 0.267 | 0.058 |
| fatty_acid_comp_{—} | | |
| SM(FA22:0) | | |
| plasma_lipid_{—} | −0.250 | 0.077 |
| fatty_acid_comp_{—} | | |
| SM(FA22:1) | | |
| plasma_lipid_{—} | 0.086 | 0.548 |
| fatty_acid_comp_{—} | | |
| SM(FA24:0) | | |
| plasma_lipid_{—} | −0.083 | 0.561 |
| fatty_acid_comp_{—} | | |
| SM(FA24:1) | | |
| plasma_lipid_{—} | 0.083 | 0.561 |
| fatty_acid_comp_{—} | | |
| TAG(FA14:0) | | |
| plasma_lipid_{—} | 0.324 | 0.020 |
| fatty_acid_comp_{—} | | |
| TAG(FA14:1) | | |
| plasma_lipid_{—} | 0.270 | 0.055 |
| fatty_acid_comp_{—} | | |
| TAG(FA15:0) | | |
| plasma_lipid_{—} | −0.413 | 0.003 |
| fatty_acid_comp_{—} | | |
| TAG(FA16:0) | | |
| plasma_lipid_{—} | −0.362 | 0.009 |
| fatty_acid_comp_{—} | | |
| TAG(FA16:1) | | |
| plasma_lipid_{—} | 0.267 | 0.058 |
| fatty_acid_comp_{—} | | |
| TAG(FA17:0) | | |
| plasma_lipid_{—} | −0.247 | 0.081 |
| fatty_acid_comp_{—} | | |
| TAG(FA18:0) | | |
| plasma_lipid_{—} | −0.276 | 0.050 |
| fatty_acid_comp_{—} | | |
| TAG(FA18:1) | | |
| plasma_lipid_{—} | −0.201 | 0.157 |
| fatty_acid_comp_{—} | | |
| TAG(FA18:2) | | |
| plasma_lipid_{—} | 0.336 | 0.016 |
| fatty_acid_comp_{—} | | |
| TAG(FA18:3) | | |
| plasma_lipid_{—} | −0.300 | 0.032 |
| fatty_acid_comp_{—} | | |
| TAG(FA20:4) | | |
| plasma_lipid_{—} | 0.290 | 0.039 |
| fatty_acid_comp_{—} | | |
| TAG(FA20:5) | | |
| plasma_lipid_{—} | 0.158 | 0.268 |
| fatty_acid_comp_{—} | | |
| TAG(FA22:6) | | |

TABLE 8
CA 19-9 Feature Set

| | # | | | | | ACC | PPV | Sensi- | Speci- |
|---|---|---|---|---|---|---|---|---|---|
| Analytes | Samples | TP | FP | TN | FN | (95% CI) | (95% CI) | tivity | ficity |
| CA 19-9 at | 67 | 13 | 12 | 27 | 15 | 0.59 | 0.52 | 0.46 | 0.69 |
| diagnosis | | | | | | (0.47-0.71) | (0.40-0.64) | | |
| CA 19-9 | 63 | 17 | 15 | 20 | 11 | 0.59 | 0.53 | 0.61 | 0.57 |
| pre-surgery | | | | | | (0.47-0.71) | (0.40-0.65) | | |
| CA 19-9 | 63 | 19 | 15 | 19 | 10 | 0.60 | 0.56 | 0.66 | 0.56 |
| post-surgery | | | | | | (0.48-0.72) | (0.44-0.68) | | |
| CA 19-9 | 59 | 20 | 13 | 18 | 8 | 0.64 | 0.61 | 0.71 | 0.58 |
| pre/post diff | | | | | | (0.52-0.76) | (0.49-0.73) | | |
| CA 19-9 all | 59 | 17 | 12 | 19 | 11 | 0.61 | 0.59 | 0.61 | 0.61 |
| | | | | | | (0.50-0.72) | (0.47-0.71) | | |

TABLE 9

| Tumor Stroma: Neoadjuvant vs Naïve (t-test) | P Value |
|---|---|
| Precent Stroma: Neoadjuvant vs Naïve | 0.0025 |
| Percent Cancer: Neoadjuvant vs. Naïve | 0.0025 |
| Ratio of Cancer to Stroma: Neoadjuvant vs Naïve | 0.0034 |

TABLE 10
Multi-Omic Modeling, Complementarity, and Analyte Importance

| | # | # | | | | | ACC | PPV | Sensi- | Speci- |
|---|---|---|---|---|---|---|---|---|---|---|
| Analytes | Samples | Features | TP | FP | TN | FN | (95% CI) | (95% CI) | tivity | ficity |
| Multi-omic | 39 | 6363 | 26 | 4 | 7 | 2 | 0.85 | 0.87 | 0.93 | 0.64 |
| | | | | | | | (0.73-0.96) | (0.75-0.99) | | |
| w/o | 40 | 4978 | 29 | 6 | 5 | 0 | 0.85 | 0.83 | 1.00 | 0.45 |
| Genomics | | | | | | | (0.74-0.96) | (0.70-0.95) | | |
| w/o | 40 | 5957 | 29 | 6 | 5 | 0 | 0.85 | 0.83 | 1.00 | 0.45 |
| Lipidomics | | | | | | | (0.74-0.96) | (0.70-0.95) | | |
| w/o Comp. | 40 | 5544 | 29 | 6 | 5 | 0 | 0.85 | 0.83 | 1.00 | 0.45 |
| pathology | | | | | | | (0.74-0.96) | (0.70-0.95) | | |
| w/o | 40 | 6032 | 29 | 6 | 5 | 0 | 0.85 | 0.83 | 1.00 | 0.45 |
| Clinical & | | | | | | | (0.74-0.96) | (0.70-0.95) | | |
| Surg. | | | | | | | | | | |
| pathology | | | | | | | | | | |
| w/o | 50 | 905 | 35 | 7 | 7 | 1 | 0.84 | 0.83 | 0.97 | 0.50 |
| Tran- | | | | | | | (0.74-0.94) | (0.72-0.95) | | |
| scriptomics | | | | | | | | | | |
| w/o | 40 | 1865 | 27 | 5 | 6 | 2 | 0.83 | 0.84 | 0.93 | 0.55 |
| Proteomics | | | | | | | (0.71-0.94) | (0.72-0.97) | | |
| All Tissue | 39 | 4715 | 24 | 3 | 8 | 4 | 0.82 | 0.89 | 0.86 | 0.73 |
| analytes | | | | | | | (0.70-0.94) | (0.77-1.00) | | |
| All Plasma | 51 | 994 | 36 | 10 | 4 | 1 | 0.78 | 0.78 | 0.97 | 0.29 |
| analytes | | | | | | | (0.67-0.90) | (0.66-0.90) | | |
| DNA (SNVs, | 71 | 945 | 41 | 14 | 10 | 6 | 0.72 | 0.75 | 0.87 | 0.42 |
| INDELS, | | | | | | | (0.61-0.82) | (0.63-0.86) | | |
| CNVs), | | | | | | | | | | |
| Clinical & | | | | | | | | | | |
| Surg. | | | | | | | | | | |
| pathology, | | | | | | | | | | |
| Comp. | | | | | | | | | | |
| pathology | | | | | | | | | | |
| DNA (SNVs, | 74 | 331 | 47 | 19 | 5 | 3 | 0.70 | 0.71 | 0.94 | 0.21 |
| INDELS, | | | | | | | (0.60-0.81) | (0.60-0.82) | | |
| CNVs), | | | | | | | | | | |
| Clinical & | | | | | | | | | | |
| Surg. | | | | | | | | | | |
| pathology | | | | | | | | | | |

TABLE 11
Proportions of differentially expressed features in each analyte among UPAP Cluster

| | Cluster #1 vs. | Cluster #2 vs. | Cluster #3 vs. | Are differentially expressed |
|---|---|---|---|---|
| | (Cluster #2 , | (Cluster #1 , | (Cluster #1, | features opresent in all 3 |
| Analyte | Cluster #3) | Cluster #3) | Cluster #2) | pairwise comaprisons |
| Clinical & Surgical | 0.30% | 0.30% | 0.90% | NO |
| pathology | | | | |
| RNA gene | 4.40% | 3.70% | 2.60% | NO |
| expressions and | | | | |
| fusions | | | | |
| Computational | 16.50% | 27.70% | 4.90% | YES |
| Pathology | | | | |
| DNA (INDELS, | 1.10% | 0.00% | 0.80% | NO |
| CNVs, SNVs) | | | | |
| Plasma lipids | 3.00% | 1.70% | 5.70% | NO |
| Plasma proteins | 14.40% | 5.10% | 5.80% | NO |
| Tissue protein | 3% | 4.60% | 8.80% | NO |

TABLE 12
Results of Tukey-Kramer test for multiple comparisons of
computational pathology feature means between clusters

| | | | | | | | | | Are the |
|---|---|---|---|---|---|---|---|---|---|
| | | | | | | | | | feature means |
| | | | | | | | | | significantly |
| Clusters | NF40_{—} | NF46_{—} | NF33_{—} | NF18_{—} | NF32_{—} | NF31_{—} | NF49_{—} | NF53_{—} | different? |
| comparison | max | max | max | max | max | 99 | min | min | p < 0.0063 * |
| Cluster #1 vs. | — | — | — | — | — | — | — | — | NO |
| Cluster #2 vs. | | | | | | | | | |
| Cluster #3 | | | | | | | | | |
| Cluster #1 vs. | — | — | — | — | — | x | — | — | YES |
| Cluster #2 | | | | | | | | | |
| Cluster #2 vs. | x | x | x | x | x | x | x | x | YES |
| Cluster #3 | | | | | | | | | |
| Cluster #1 vs. | x | x | x | x | — | — | x | — | YES |
| Cluster #3 | | | | | | | | | |

* significant difference between feature means was established when the p-value from the multiple comparion test was p < 0.05/8 = 0.0063; x means of the feature differ significantly between clusters; — means of the feature do not differ significantly between clusters.
NF-40: large zone size emphasis; NF-46: large zone/high gray emphasis; NF-33: inverse difference inverse difference moment; NF-31: cluster promineance zone size; NF-49: percentage rune percentage: NP-53: (RP); all hemotaxylin staining textures.

TABLE 13A
RNA Gene Signatures for Improved Survival

| | label_patient_survival_{—} | label_patient_survival_{—} | label_patient_survival_{—} | label_patient_survival_{—} |
|---|---|---|---|---|
| Gene | pearson_rho | pearson_pval | spearman_rho | spearman_pval |
| EEF2K | 0.262263039 | 0.048744 | 0.295897 | 0.025432 |
| AC242843.1 | 0.263413974 | 0.047727 | 0.26134 | 0.049573 |
| PHF20 | 0.268600812 | 0.043357 | 0.280778 | 0.034377 |
| FBXL17 | 0.272998897 | 0.039914 | 0.304536 | 0.021264 |
| SFMBT1 | 0.276033977 | 0.037672 | 0.295897 | 0.025432 |
| NCOA2 | 0.278270293 | 0.036087 | 0.274299 | 0.03894 |
| AUTS2 | 0.282670547 | 0.033131 | 0.280778 | 0.034377 |
| CYP20A1 | 0.282791503 | 0.033052 | 0.274299 | 0.03894 |
| TOMM7 | 0.286885431 | 0.030491 | 0.272139 | 0.040569 |
| SH3D19 | 0.287477653 | 0.030134 | 0.300217 | 0.023269 |
| CPD | 0.287560735 | 0.030084 | 0.278618 | 0.035846 |
| NISCH | 0.289705466 | 0.028824 | 0.267819 | 0.043993 |
| PIK3R1 | 0.29052834 | 0.028353 | 0.313176 | 0.01769 |
| CDC42EP3 | 0.29566245 | 0.025554 | 0.315336 | 0.016881 |
| DCN | 0.297501016 | 0.02461 | 0.347733 | 0.008039 |
| ZFAND5 | 0.299014372 | 0.023855 | 0.315336 | 0.016881 |
| GPR137B | 0.299074438 | 0.023825 | 0.276459 | 0.037367 |
| RGS5 | 0.304836325 | 0.02113 | 0.406048 | 0.001725 |
| LRIG3 | 0.305181127 | 0.020977 | 0.317495 | 0.016104 |
| RC3H1 | 0.305654898 | 0.020769 | 0.332614 | 0.011472 |
| ZCCHC24 | 0.313751348 | 0.017471 | 0.313176 | 0.01769 |
| ARHGEF11 | 0.317648593 | 0.01605 | 0.330454 | 0.012054 |
| GTF2IRD2B | 0.323064474 | 0.014239 | 0.326135 | 0.013293 |
| NEDD9 | 0.324386839 | 0.013825 | 0.339094 | 0.009871 |
| SERINC5 | 0.325146169 | 0.013592 | 0.352053 | 0.007239 |
| CPNE3 | 0.333984479 | 0.011116 | 0.330454 | 0.012054 |
| GNAQ | 0.336063825 | 0.010594 | 0.336934 | 0.010382 |
| SLC25A13 | 0.336319128 | 0.010531 | 0.291577 | 0.027761 |
| CACNA1D | 0.338698409 | 0.009963 | 0.321815 | 0.014641 |
| DKK3 | 0.344314045 | 0.008725 | 0.397409 | 0.002206 |
| GON4L | 0.351189711 | 0.007393 | 0.356372 | 0.00651 |
| OPHN1 | 0.352139915 | 0.007224 | 0.347733 | 0.008039 |
| YY1AP1 | 0.364107537 | 0.005364 | 0.38229 | 0.003339 |
| TIPARP | 0.364647249 | 0.005291 | 0.308856 | 0.019407 |
| NIPAL2 | 0.387720097 | 0.002884 | 0.403889 | 0.001835 |
| USP22 | 0.402322038 | 0.001919 | 0.449245 | 0.000456 |
| ABHD2 | 0.411100264 | 0.00149 | 0.367171 | 0.004961 |
| NFE2L2 | 0.414060751 | 0.001365 | 0.425487 | 0.000969 |
| PRKX | 0.417410468 | 0.001236 | 0.421167 | 0.001104 |
| ZNF704 | 0.429563549 | 0.000854 | 0.408208 | 0.001621 |

TABLE 13B
RNA Gene Signatures for Poor Survival

| | label_patient_survival_{—} | label_patient_survival_{—} | label_patient_survival_{—} | label_patient_survival_{—} |
|---|---|---|---|---|
| Gene | pearson_rho | pearson_pval | spearman_rho | spearman_pval |
| NBPF26 | −0.4459 | 0.000509 | −0.44277 | 0.000563 |
| DTX3L | −0.38483 | 0.003119 | −0.39957 | 0.002075 |
| DFFA | −0.38164 | 0.003398 | −0.40173 | 0.001952 |
| PARP9 | −0.37909 | 0.003636 | −0.36501 | 0.005242 |
| PARP14 | −0.37006 | 0.004606 | −0.42981 | 0.000848 |
| SRSF4 | −0.36405 | 0.005372 | −0.38445 | 0.003151 |
| MFN2 | −0.36036 | 0.005895 | −0.32829 | 0.01266 |
| ICAM1 | −0.35556 | 0.006643 | −0.31966 | 0.015357 |
| ANKRD13A | −0.34273 | 0.009059 | −0.38661 | 0.002972 |
| RALB | −0.33369 | 0.011191 | −0.33477 | 0.010915 |
| VAMP3 | −0.32894 | 0.012475 | −0.29806 | 0.02433 |
| DDX60L | −0.32889 | 0.012491 | −0.40605 | 0.001725 |
| TLE3 | −0.32839 | 0.012634 | −0.31102 | 0.018531 |
| MYL6 | −0.32508 | 0.013613 | −0.34125 | 0.009382 |
| YBX1 | −0.32459 | 0.013763 | −0.29374 | 0.026575 |
| MTOR | −0.31301 | 0.017752 | −0.27646 | 0.037367 |
| MSANTD3 | −0.30556 | 0.02081 | −0.31966 | 0.015357 |
| PSMD5 | −0.30321 | 0.021861 | −0.28726 | 0.030266 |
| EHD1 | −0.30246 | 0.022208 | −0.26134 | 0.049573 |
| STAT2 | −0.30139 | 0.022707 | −0.33909 | 0.009871 |
| PRMT3 | −0.30082 | 0.02298 | −0.3175 | 0.016104 |
| ACTN4 | −0.29845 | 0.024134 | −0.28726 | 0.030266 |
| RAB8A | −0.29623 | 0.025258 | −0.32397 | 0.013953 |
| NFKBIZ | −0.29561 | 0.025583 | −0.2851 | 0.031588 |
| VASP | −0.29483 | 0.025989 | −0.26566 | 0.045793 |
| BAZ2A | −0.2932 | 0.026867 | −0.27862 | 0.035846 |
| BCL9L | −0.29283 | 0.027066 | −0.26566 | 0.045793 |
| AP2B1 | −0.29212 | 0.027457 | −0.2851 | 0.031588 |
| NLRC5 | −0.29181 | 0.027631 | −0.2851 | 0.031588 |
| BACH1 | −0.29041 | 0.02842 | −0.2851 | 0.031588 |
| YEATS2 | −0.28299 | 0.032927 | −0.28726 | 0.030266 |
| UBE2S | −0.28197 | 0.033591 | −0.33261 | 0.011472 |
| RNA5SP389 | −0.27148 | 0.041073 | −0.40173 | 0.001952 |
| XAF1 | −0.27132 | 0.041201 | −0.36069 | 0.005846 |
| BMS1 | −0.27063 | 0.041742 | −0.2743 | 0.03894 |
| STAT1 | −0.26967 | 0.042496 | −0.33909 | 0.009871 |
| WDR1 | −0.26768 | 0.044107 | −0.26566 | 0.045793 |
| UBE2Z | −0.26702 | 0.044651 | −0.2851 | 0.031588 |
| KDM1B | −0.26242 | 0.048606 | −0.26566 | 0.045793 |

TABLE 14
Significant Pathways of Gene Signature for Improved and Poor Survival via Enricher

| | P-value | Adjusted P-value | Odds Ratio | Combined Score |
|---|---|---|---|---|
| KEGG 2021 | | | | |
| Growth hormone synthesis, | 1.09E−04 | 0.01858705 | 11.73956852 | 107.1464987 |
| secretion and action | | | | |
| Pancreatic cancer | 2.33E−04 | 0.019930852 | 14.70296296 | 122.9756228 |
| Tight junction | 5.53E−04 | 0.025647561 | 8.139831905 | 61.05699494 |
| Type II diabetes mellitus | 7.97E−04 | 0.025647561 | 18.24785802 | 130.198309 |
| Kaposi sarcoma-associated | 0.001004741 | 0.025647561 | 7.092078781 | 48.95679745 |
| herpesvirus infection | | | | |
| Leukocyte transendothelial | 0.001082589 | 0.025647561 | 9.605333333 | 65.58905847 |
| migration | | | | |
| Pathways in cancer | 0.001170345 | 0.025647561 | 4.179139849 | 28.21110162 |
| AMPK signaling pathway | 0.001309135 | 0.025647561 | 9.105747126 | 60.44749011 |
| Thyroid hormone signaling | 0.001349872 | 0.025647561 | 9.027464387 | 59.65118964 |
| pathway | | | | |
| Vascular smooth muscle | 0.001910074 | 0.032305112 | 8.182739018 | 51.22896513 |
| contraction | | | | |
| WikiPathway 2021 | | | | |
| Interferon type I signaling | 6.14E−05 | 0.006829098 | 21.19573333 | 205.5700083 |
| pathways WP585 | | | | |
| RANKL/RANK signaling | 6.60E−05 | 0.006829098 | 20.77908497 | 200.0222636 |
| pathway WP2018 | | | | |
| Thyroid stimulating hormone | 1.35E−04 | 0.00930959 | 17.08301075 | 152.2235787 |
| (TSH) signaling pathway | | | | |
| WP2032 | | | | |
| CAMKK2 Pathway | 3.25E−04 | 0.011791099 | 25.32682513 | 203.4206339 |
| WP4874 | | | | |
| Type II interferon signaling | 4.18E−04 | 0.011791099 | 23.08862229 | 179.6101791 |
| (IFNG) WP619 | | | | |
| Overview of interferons- | 4.18E−04 | 0.011791099 | 23.08862229 | 179.6101791 |
| mediated signaling pathway | | | | |
| WP4558 | | | | |
| Pancreatic adenocarcinoma | 4.27E−04 | 0.011791099 | 12.44611765 | 96.57333492 |
| pathway WP4263 | | | | |
| EGF/EGFR signaling | 4.56E−04 | 0.011791099 | 8.505766913 | 65.44071376 |
| pathway WP437 | | | | |
| Type III interferon signaling | 6.79E−04 | 0.014470833 | 64.6525974 | 471.6195625 |
| WP2113 | | | | |
| Interleukin-11 Signaling | 6.99E−04 | 0.014470833 | 19.13992298 | 139.0659692 |
| Pathway WP2332 | | | | |
| BioPlanet 2019 | | | | |
| CXCR4 signaling pathway | 6.58E−06 | 0.003118985 | 14.80273973 | 176.6182228 |
| Vascular smooth muscle | 9.63E−05 | 0.016064543 | 12.0586803 | 111.5142935 |
| contraction | | | | |
| Thyroid-stimulating hormone | 0.000134922 | 0.016064543 | 17.08301075 | 152.2235787 |
| signaling pathway | | | | |
| Immune system | 0.000135566 | 0.016064543 | 3.786617443 | 33.72381837 |
| S6K1 signaling | 0.000424753 | 0.032703775 | 86.21212121 | 669.3512057 |
| cAMP receptor, G-protein- | 0.000544712 | 0.032703775 | 73.89239332 | 555.3200357 |
| independent pathways | | | | |
| inferred from amoeba model | | | | |
| Interferon-gamma signaling | 0.000591315 | 0.032703775 | 11.37089606 | 84.52171232 |
| pathway | | | | |
| Adaptive immune system | 0.000631925 | 0.032703775 | 4.16166547 | 30.65790379 |
| Interferon-alpha signaling | 0.000679147 | 0.032703775 | 64.6525974 | 471.6195625 |
| pathway | | | | |
| Interleukin-4 signaling | 0.000768528 | 0.032703775 | 10.5712 | 75.80643478 |
| pathway | | | | |

TABLE 15
Feature Set of Parsimonious Model on TCGA

| | Feature | | Feature | | Feature |
|---|---|---|---|---|---|
| Feature Name | Weight | Feature Name | Weight | Feature Name | Weight |
| pathology_NF17_75% | 0.4155 | rna_expr_ZDHHC7 | 0.1066 | pathology_NF55_mean | 0.0676 |
| pathology_NF28_75% | 0.3840 | rna_expr_WWC2 | 0.1040 | rna_expr_SKIL | 0.0672 |
| pathology_NF8_0.9 | 0.2986 | rna_expr_SERINC5 | 0.1031 | pathology_NF53_min | 0.0661 |
| pathology_NF8_75% | 0.2984 | pathology_NF17_50% | 0.1012 | rna_expr_TLK2 | 0.0658 |
| pathology_NF16_max | 0.2801 | rna_expr_STRN3 | 0.1008 | pathology_NF12_max | 0.0654 |
| pathology_NF19_75% | 0.2738 | pathology_NF59_std | 0.1007 | rna_expr_GNPTAB | 0.0653 |
| pathology_NF55_75% | 0.2644 | rna_expr_MSANTD3 | 0.0993 | rna_expr_PRKAR2A | 0.0652 |
| pathology_NF19_0.9 | 0.2557 | rna_expr_XRN1 | 0.0968 | pathology_NF2_max | 0.0652 |
| rna_expr_NFE2L2 | 0.2554 | rna_expr_RAC1 | 0.0960 | pathology_NF1_0.01 | 0.0646 |
| pathology_NF55_0.9 | 0.2511 | rna_expr_ZC3H11A | 0.0953 | rna_expr_DESI2 | 0.0641 |
| rna_expr_RAB8A | 0.2355 | rna_expr_RGS5 | 0.0953 | rna_expr_GNAQ | 0.0640 |
| pathology_NF42_0.9 | 0.2353 | rna_expr_PRKX | 0.0948 | ma_expr_TUBA1B | 0.0637 |
| pathology_NF44_0.95 | 0.2272 | rna_expr_DHX8 | 0.0928 | rna_expr_WDR3 | 0.0635 |
| pathology_NF42_75% | 0.2143 | pathology_NF19_mean | 0.0920 | rna_expr_DCAF6 | 0.0635 |
| pathology_NF44_75% | 0.2056 | rna_expr_RBFOX2 | 0.0916 | pathology_NF28_mean | 0.0632 |
| pathology_NF44_0.9 | 0.2033 | pathology_NF55_50% | 0.0906 | rna_expr_CDC42EP3 | 0.0627 |
| rna_expr_CPD | 0.1993 | rna_expr_GON4L | 0.0897 | pathology_NF46_50% | 0.0625 |
| pathology_NF19_0.95 | 0.1956 | pathology_NF44_0.99 | 0.0896 | rna_expr_RNF103 | 0.0623 |
| pathology_NF19_50% | 0.1945 | pathology_NF43_0.1 | 0.0889 | pathology_NF62_mean | 0.0622 |
| rna_expr_FBXL17 | 0.1896 | pathology_NF17_0.95 | 0.0878 | rna_expr_ARID1A | 0.0620 |
| pathology_NF30_50% | 0.1875 | rna_expr_JAG1 | 0.0875 | rna_expr_BTRC | 0.0620 |
| pathology_NF17_0.9 | 0.1869 | rna_expr_OXR1 | 0.0872 | rna_expr_ANKRD13A | 0.0615 |
| rna_expr_ABHD2 | 0.1856 | rna_expr_ACTN4 | 0.0863 | pathology_NF63_75% | 0.0613 |
| rna_expr_TOMM7 | 0.1828 | rna_expr_DPY19L4 | 0.0863 | rna_expr_CSNK2A1 | 0.0611 |
| rna_expr_RC3H1 | 0.1817 | pathology_NF37_max | 0.0861 | rna_expr_STAT2 | 0.0609 |
| rna_expr_VPS45 | 0.1808 | pathology_NF30_0.1 | 0.0847 | pathology_NF10_0.05 | 0.0609 |
| pathology_NF62_0.95 | 0.1800 | pathology_NF28_0.95 | 0.0846 | pathology_NF62_0.05 | 0.0608 |
| pathology_NF44_50% | 0.1768 | pathology_NF45_25% | 0.0839 | pathology_NF30_75% | 0.0607 |
| rna_expr_MEX3C | 0.1700 | pathology_NF21_min | 0.0838 | rna_expr_RBM12 | 0.0606 |
| pathology_NF10_50% | 0.1513 | pathology_NF30_25% | 0.0837 | rna_expr_PARP14 | 0.0606 |
| rna_expr_MYL6 | 0.1513 | pathology_NF41_50% | 0.0835 | pathology_NF44_mean | 0.0605 |
| pathology_NF28_50% | 0.1508 | rna_expr_SMC4 | 0.0834 | rna_expr_OPHN1 | 0.060512 |
| pathology_NF62_25% | 0.1484 | rna_expr_XAF1 | 0.0829 | rna_expr_DYNC2H1 | 0.059995 |
| pathology_NF46_25% | 0.1460 | rna_expr_HIVEP2 | 0.0821 | rna_expr_MTF2 | 0.059939 |
| rna_expr_PRMT3 | 0.1452 | rna_expr_NIPAL2 | 0.0809 | rna_expr_SDCBP | 0.059286 |
| rna_expr_DFFA | 0.1450 | rna_expr_BMS1 | 0.0802 | rna_expr_B4GAT1 | 0.05885 |
| pathology_NF4_std | 0.1443 | rna_expr_MPHOSPH9 | 0.0801 | rna_expr_RPTOR | 0.058685 |
| rna_expr_RAB6A | 0.1437 | rna_expr_ANKRD13C | 0.0800 | rna_expr_SORL1 | 0.058382 |
| rna_expr_YBX1 | 0.1435 | rna_expr_EPB41L2 | 0.0789 | rna_expr_FANCC | 0.057976 |
| pathology_NF62_50% | 0.1429 | rna_expr_VPS41 | 0.0788 | rna_expr_MAST2 | 0.057794 |
| rna_expr_NBPF26 | 0.1389 | rna_expr_DTX3L | 0.0788 | pathology_NF42_0.99 | 0.057241 |
| rna_expr_CPNE3 | 0.1382 | rna_expr_TASP1 | 0.0786 | pathology_NF41_25% | 0.056682 |
| pathology_NF42_0.95 | 0.1347 | rna_expr_CTNNB1 | 0.0783 | rna_expr_PARP9 | 0.056678 |
| rna_expr_LRRK1 | 0.1318 | rna_expr_CAND1 | 0.0778 | rna_expr_NPIPB3 | 0.056523 |
| pathology_NF42_50% | 0.1294 | rna_expr_ZFC3H1 | 0.0777 | rna_expr_RNA5SP389 | 0.056113 |
| pathology_NF28_0.9 | 0.1258 | pathology_NF62_0.9 | 0.0774 | pathology_NF62_0.99 | 0.056009 |
| rna_expr_PSMD5 | 0.1252 | rna_expr_DLG1 | 0.0770 | pathology_NF17_mean | 0.055825 |
| pathology_NF8_mean | 0.1241 | rna_expr_CBWD3 | 0.0762 | pathology_NF63_std | 0.055819 |
| rna_expr_GPR137B | 0.1223 | rna_expr_MT-ND6 | 0.0761 | rna_expr_NFKBIZ | 0.055794 |
| pathology_NF8_0.95 | 0.1222 | rna_expr_MPZL1 | 0.0760 | pathology_NF63_0.95 | 0.055466 |
| pathology_NF55_0.95 | 0.1212 | pathology_NF36_min | 0.0750 | rna_expr_ARPC5 | 0.055138 |
| rna_expr_YY1AP1 | 0.1207 | rna_expr_LAMC1 | 0.0744 | pathology_NF55_0.99 | 0.05512 |
| rna_expr_APPBP2 | 0.1203 | pathology_NF19_0.99 | 0.0740 | pathology_NF63_0.9 | 0.054708 |
| rna_expr_SMARCC2 | 0.1198 | rna_expr_DKK3 | 0.0731 | rna_expr_ATAD3A | 0.054513 |
| rna_expr_KCNK1 | 0.1195 | pathology_NF18_max | 0.0729 | rna_expr_IGFBP3 | 0.054346 |
| rna_expr_SRSF4 | 0.1192 | pathology_NF13_min | 0.0720 | pathology_NF45_0.1 | 0.054182 |
| pathology_NF62_75% | 0.1179 | rna_expr_COPA | 0.0719 | pathology_NF8_0.99 | 0.053892 |
| pathology_NF10_mean | 0.1179 | pathology_NF1_0.9 | 0.0719 | rna_expr_PDGFRA | 0.053551 |
| rna_expr_POGZ | 0.1149 | rna_expr_DDX60L | 0.0719 | rna_expr_SP2 | 0.052801 |
| rna_expr_USP22 | 0.1147 | pathology_NF30_mean | 0.0718 | rna_expr_INSR | 0.052481 |
| rna_expr_NEDD9 | 0.1125 | rna_expr_NBR1 | 0.0704 | pathology_NF62_0.01 | 0.052415 |
| rna_expr_LRP1 | 0.1108 | rna_expr_NCOA2 | 0.0695 | pathology_NF51_max | 0.051971 |
| rna_expr_CEP350 | 0.1105 | pathology_NF33_max | 0.0692 | rna_expr_KHDC4 | 0.051875 |
| rna_expr_DCN | 0.1100 | rna_expr_VASP | 0.0681 | rna_expr_ARNT | 0.051814 |
| rna_expr_CDS2 | 0.1098 | pathology_NF63_mean | 0.0679 | rna_expr_BAZ2A | 0.051338 |
| pathology_NF8_50% | 0.1082 | rna_expr_ALB | 0.0678 | rna_expr_C1GALT1 | 0.05062 |
| rna_expr_ZNF704 | 0.1076 | rna_expr_SMC6 | 0.0677 | rna_expr_AP2B1 | 0.050301 |
| | | | | rna_expr_CDC73 | 0.05024 |

TABLE 16
Clinical Data on normal paired samples

| Identifier | Gender | Age | Race |
|---|---|---|---|
| HMN77795 | Male | 66 | Hispanic |
| HMN77796 | Male | 45 | Black |
| HMN77797 | Male | 64 | Black |
| HMN77798 | Male | 45 | Black |
| HMN77799 | Male | 48 | Black |
| HMN77800 | Male | 62 | Hispanic |
| HMN77801 | Male | 44 | Hispanic |
| HMN77802 | Male | 62 | Caucasian |
| HMN77803 | Male | 59 | Black |
| HMN77804 | Male | 57 | Hispanic |
| HMN77806 | Male | 46 | Hispanic |
| HMN77807 | Male | 41 | Black |
| HMN77808 | Male | 43 | Hispanic |
| HMN77809 | Male | 61 | Black |
| HMN77810 | Male | 46 | Black |
| HMN77811 | Male | 54 | Hispanic |
| HMN77812 | Male | 53 | Caucasian |
| HMN77813 | Male | 53 | Caucasian |
| HMN77814 | Male | 71 | Hispanic |
| HMN77815 | Male | 46 | Black |
| HMN77816 | Male | 50 | Black |
| HMN77817 | Male | 40 | Hispanic |
| HMN77818 | Male | 62 | Caucasian |
| HMN77819 | Male | 60 | Other |
| HMN77820 | Male | 61 | Hispanic |
| HMN77821 | Male | 55 | Hispanic |
| HMN77822 | Male | 47 | Caucasian |
| HMN77823 | Male | 64 | Black |
| HMN77825 | Male | 54 | Hispanic |
| HMN77826 | Male | 56 | Hispanic |
| HMN77827 | Male | 43 | Caucasian |
| HMN77828 | Male | 58 | Hispanic |
| HMN77829 | Male | 56 | Caucasian |
| HMN77830 | Male | 61 | Hispanic |
| HMN77832 | Male | 53 | Black |
| HMN77833 | Male | 43 | Caucasian |
| HMN77834 | Male | 66 | Black |
| HMN77836 | Male | 61 | Black |
| HMN77837 | Male | 56 | Black |
| HMN77838 | Male | 57 | Black |
| HMN77839 | Male | 51 | Hispanic |
| HMN77840 | Male | 50 | Hispanic |
| HMN77841 | Male | 59 | Hispanic |
| HMN77842 | Male | 57 | Black |
| HMN77843 | Male | 50 | Black |
| HMN77844 | Female | 47 | Hispanic |
| HMN77845 | Female | 45 | Black |
| HMN77846 | Female | 54 | Hispanic |
| HMN77847 | Female | 46 | Hispanic |
| HMN77848 | Female | 52 | Black |
| HMN77850 | Female | 51 | Black |
| HMN77851 | Female | 58 | Hispanic |
| HMN77858 | Female | 43 | Hispanic |
| HMN77859 | Female | 42 | Black |
| HMN77860 | Female | 50 | Black |
| HMN77861 | Female | 52 | Hispanic |
| HMN77862 | Female | 43 | Hispanic |
| HMN77863 | Female | 57 | Black |
| HMN77864 | Female | 57 | Hispanic |
| HMN77865 | Female | 55 | Caucasian |
| HMN77866 | Female | 58 | Black |
| HMN77867 | Female | 44 | Black |
| HMN77868 | Female | 59 | Black |
| HMN77869 | Female | 53 | Caucasian |
| HMN77870 | Female | 60 | Black |
| HMN77871 | Female | 52 | Caucasian |
| HMN77873 | Female | 64 | Caucasian |
| HMN77874 | Female | 49 | Black |
| HMN77875 | Female | 44 | Black |
| HMN77876 | Female | 44 | Black |
| HMN77877 | Female | 59 | Hispanic |
| HMN77878 | Female | 40 | Caucasian |
| HMN77879 | Female | 47 | Black |
| HMN77880 | Female | 71 | Black |
| HMN77881 | Female | 74 | Hispanic |
| HMN77882 | Female | 53 | Black |
| HMN77883 | Female | 43 | Hispanic |
| HMN77884 | Female | 41 | Caucasian |
| HMN77885 | Female | 55 | Caucasian |
| HMN77887 | Female | 46 | Caucasian |
| HMN77888 | Female | 49 | Caucasian |
| HMN77889 | Female | 55 | Caucasian |
| HMN77890 | Female | 55 | Black |
| HMN77892 | Female | 57 | Hispanic |

TABLE 17
Software Resources Utilized

| Resource | Source | Identifier |
|---|---|---|
| Boto3 (v2.3.4) | AWS Boto3 | boto3.amazonaws.com/v1/documentation/api/latest/index.html |
| CSBDeep (v0.7.2) | Weigert et al., 2018 | csbdeep.bioimagecomputing.com/doc/ |
| dplyr (v1.0.10) | Wickham et al., 2022 | github.com/tidyverse/dplyr |
| Freebayes (v1.3.6) | Garrison et al., 2012 | github.com/freebayes/freebayes |
| H5py (v1.13.2) | The HDF Group | github.com/h5py/h5py |
| Kallisto (v0.46.1) | Brey et al., 2016 | pachterlab.github.io/kallisto/about |
| Keras (v2.9.0) | Tensorflow | github.com/keras-team/keras |
| Matplotlib (v3.5.2) | Hunter et al., 2007 | matplotlib.org/ |
| NumPy (v1.21.0) | Harris et al., 2020 | github.com/numpy/numpy |
| Openslide (v1.2.0) | Goode et al., 2013 | openslide.org/api/python/ |
| Pandas (v1.4.3) | Pandas | github.com/pandas-dev/pandas |
| Pathlib (v3.3) | N/A | docs.python.org/3/library/pathlib.html |
| PIL (v9.2.0) | Clark et al., 2022 | github.com/python-pillow/Pillow |
| Pindel (v0.2.5b9) | Yi et al., 2009 | http://gmt.genome.wustl.edu/packages/pindel/index.html |
| PyEnsembl (v107) | Howe et al., 2021 | github.com/openvax/pyensembl |
| Python (v3.8.13) | N/A | www.python.org/ |
| PyVCF (v0.6.8) | N/A | anaconda.org/bioconda/pyvcf |
| s3fs (v2021.7.2) | Botocore | pypi.org/project/s3fs/ |
| Scikit-learn (v1.1) | Pedregosa et al., 2011 | github.com/scikit-learn/scikit-learn |
| SciPy (v1.9.0) | Virtanen et al., 2020 | github.com/scipy/scipy |
| StarDist (v0.8.3) | Schmidt et al., 2018 | pypi.org/project/stardist/ |
| Tensorflow Data | Breck et al., 2019 | github.com/tensorflow/data-validation |
| Validation (v1.9.0) | | |
| TxImport (v1.24.0) | Soneson et al., 2015 | bioconductor.org/packages/release/bioc/html/tximport.html |
| Varscan (v2.4.2) | Koboldt et al., 2012 | github.com/Jeltje/varscan2 |

TABLE 18A
Frequency of Top Pathology Features

| Analyte | Study Label | Feature | Feature Name | Frequency |
|---|---|---|---|---|
| Pathology | label_deceased | NF1_75% | Solidity (75 percentile) | 1 |
| Pathology | label_deceased | NF16_0.95 | Correlation (95 percentile) | 0.5915493 |
| Pathology | label_deceased | NF46_0.1 | Large Zone/High Gray Emphasis (10 | 0.57746479 |
| | | | percentile) | |
| Pathology | label_deceased | NF59_min | Margination (minimal) | 0.52112676 |
| Pathology | label_deceased | NF2_0.1 | Extent (10 percentile) | 0.52112676 |
| Pathology | label_deceased | NF62_75% | Closest Neighborhood Distance (75 | 0.50704225 |
| | | | percentile) | |
| Pathology | label_deceased | NF47_0.95 | Gray-Level Non-Uniformity (95 | 0.46478873 |
| | | | percentile) | |
| Pathology | label_deceased | NF20_0.05 | Sum variance (5 percentile) | 0.46478873 |
| Pathology | label_deceased | NF37_max | Complexity (maximal) | 0.42253521 |
| Pathology | label_deceased | NF5_0.95 | MinorAxisLength/MajorAxisLength (95 | 0.42253521 |
| | | | percentile) | |
| Pathology | label_deceased | NF32_max | Maximum Probability (maximal) | 0.4084507 |
| Pathology | label_deceased | NF4_0.05 | Eccentricity (5 percentile) | 0.4084507 |
| Pathology | label_deceased | NF37_75% | Complexity (75 percentile) | 0.3943662 |
| Pathology | label_deceased | NF22_std | Entropy (standard deviation) | 0.3943662 |
| Pathology | label_deceased | NF31_mean | Cluster Prominence (Mean) | 0.38028169 |
| Pathology | label_deceased | NF58_0.01 | Homogeneity (1 percentile) | 0.35211268 |
| Pathology | label_deceased | NF1_mean | Solidity (mean) | 0.35211268 |
| Pathology | label_deceased | NF51_75% | LONG RUN EMPHASIS(LRE) (75 | 0.35211268 |
| | | | percentile) | |
| Pathology | label_deceased | NF10_0.05 | Skewness (5 percentile) | 0.32394366 |
| Pathology | label_deceased | NF39_0.01 | Small Zone Size Emphasis (1 percentile) | 0.29577465 |
| Pathology | label_deceased | NF50_0.05 | SHORT RUN EMPHASIS (SRE) (5 | 0.29577465 |
| | | | percentile) | |
| Pathology | label_deceased | NF35_25% | Contrast (25 percentile) | 0.28169014 |
| Pathology | label_deceased | NF12_0.01 | Energy (1 percentile) | 0.28169014 |
| Pathology | label_deceased | NF40_25% | Large Zone Size Emphasis (25 percentile) | 0.26760563 |
| Pathology | label_deceased | NF3_0.05 | EquivDiameter (5 percentile) | 0.25352113 |
| Pathology | label_deceased | NF51_50% | LONG RUN EMPHASIS(LRE) (50 | 0.25352113 |
| | | | percentile) | |
| Pathology | label_deceased | NF43_0.99 | Small Zone/Low Gray Emphasis (99 | 0.23943662 |
| | | | percentile) | |
| Pathology | label_deceased | NF11_75% | Kurtosis (75 percentile) | 0.23943662 |
| Pathology | label_deceased | NF13_0.05 | Entropy (5 percentile) | 0.22535211 |
| Pathology | label_deceased | NF6_max | Area (Maximal) | 0.21126761 |
| Pathology | label_deceased | NF8_std | Mean (Standard Deviation) | 0.18309859 |
| Pathology | label_deceased | NF4_std | Eccentricity (Standard Deviation) | 0.16901409 |
| Pathology | label_deceased | NF16_25% | Correlation (25 percentile) | 0.15492958 |
| Pathology | label_deceased | NF42_std | High Gray-Level Zone Emphasis | 0.15492958 |
| | | | (Standard Deviation) | |
| Pathology | label_deceased | NF13_0.9 | Entropy (90 percentile) | 0.14084507 |
| Pathology | label_deceased | NF5_75% | MinorAxisLength/MajorAxisLength (75 | 0.14084507 |
| | | | percentile) | |
| Pathology | label_deceased | NF4_25% | Eccentricity (25 percentile) | 0.14084507 |
| Pathology | label_deceased | NF50_75% | SHORT RUN EMPHASIS (SRE) (75 | 0.12676056 |
| | | | percentile) | |
| Pathology | label_deceased | NF2_75% | Extent (75 percentile) | 0.11267606 |
| Pathology | label_deceased | NF21_std | Sum Entropy (Standard Deviation) | 0.09859155 |
| Pathology | label_deceased | NF39_50% | Small Zone Size Emphasis (50 percentile) | 0.09859155 |
| Pathology | label_deceased | NF7_0.05 | Perimeter (5 percentile) | 0.09859155 |
| Pathology | label_deceased | NF1_25% | Solidity (25 percentile) | 0.09859155 |
| Pathology | label_deceased | NF1_0.9 | Solidity (90 percentile) | 0.09859155 |
| Pathology | label_deceased | NF52_0.95 | GRAY LEVEL NON-UNIFORMITY | 0.08450704 |
| | | | (GLN) (95 percentile) | |
| Pathology | label_deceased | NF4_0.95 | Eccentricity (95 percentile) | 0.08450704 |
| Pathology | label_deceased | NF1_max | Solidity (Maximal) | 0.08450704 |
| Pathology | label_deceased | NF14_75% | Angular second moment (75 percentile) | 0.08450704 |
| Pathology | label_deceased | NF9_max | Variance (Maximal) | 0.07042254 |
| Pathology | label_deceased | NF26_0.95 | Information Correlation 2 (95 percentile) | 0.07042254 |
| Pathology | label_deceased | NF1_min | Solidity (Minimal) | 0.07042254 |
| Pathology | label_deceased | NF4_mean | Eccentricity (Mean) | 0.05633803 |
| Pathology | label_deceased | NF6_75% | Area (75 percentile) | 0.05633803 |
| Pathology | label_deceased | NF5_0.05 | MinorAxisLength/MajorAxisLength (5 | 0.05633803 |
| | | | percentile) | |
| Pathology | label_deceased | NF9_0.9 | Variance (90 percentile) | 0.05633803 |
| Pathology | label_deceased | NF63_50% | Average Distance to 5 Closest Neighbors | 0.05633803 |
| | | | (50 percentile) | |
| Pathology | label_recurred | NF4_std | Eccentricity (Standard Deviation) | 0.9859 |
| Pathology | label_recurred | NF38_min | Texture Strength (Minimal) | 0.9577 |
| Pathology | label_recurred | NF19_min | Sum average (Minimal) | 0.8732 |
| Pathology | label_recurred | NF36_min | Busyness (Minimal) | 0.8169 |
| Pathology | label_recurred | NF5_std | MinorAxisLength/MajorAxisLength | 0.7606 |
| | | | (Standard Deviation) | |
| Pathology | label_recurred | NF63_min | Average Distance to 5 Closest Neighbors | 0.7042 |
| | | | (Minimal) | |
| Pathology | label_recurred | NF37_std | Complexity (Standard Deviation) | 0.5915 |
| Pathology | label_recurred | NF59_0.01 | Margination (1 percentile) | 0.5352 |
| Pathology | label_recurred | NF62_min | Closest Neighborhood Distance | 0.3662 |
| | | | (Minimal) | |
| Pathology | label_recurred | NF1_0.9 | Solidity (90 percentile) | 0.2817 |
| Pathology | label_recurred | NF28_min | Autocorrelation (Minimal) | 0.2676 |
| Pathology | label_recurred | NF36_max | Busyness (Maximal) | 0.2535 |
| Pathology | label_recurred | NF48_min | Zone Size Non-Uniformity (Minimal) | 0.2535 |
| Pathology | label_recurred | NF37_0.01 | Complexity (1 percentile) | 0.2394 |
| Pathology | label_recurred | NF41_min | Low Gray-Level Zone Emphasis | 0.2254 |
| | | | (Minimal) | |
| Pathology | label_recurred | NF13_min | Entropy (Minimal) | 0.2113 |
| Pathology | label_recurred | NF1_0.99 | Solidity (99 percentile) | 0.2113 |
| Pathology | label_recurred | NF1_0.01 | Solidity (1 percentile) | 0.1972 |
| Pathology | label_recurred | NF46_0.1 | Large Zone/High Gray Emphasis (10 | 0.1972 |
| | | | percentile) | |
| Pathology | label_recurred | NF26_0.99 | Information Correlation 2 (99 percentile) | 0.1831 |
| Pathology | label_recurred | NF60_50% | Clumping (50 percentile) | 0.1549 |
| Pathology | label_recurred | NF23_min | Difference Variance (Minimal) | 0.1549 |
| Pathology | label_recurred | NF10_max | Skewness (Maximal) | 0.1549 |
| Pathology | label_recurred | NF62_0.95 | Closest Neighborhood Distance (95 | 0.1408 |
| | | | percentile) | |
| Pathology | label_recurred | NF20_max | Sum variance (Maximal) | 0.1408 |
| Pathology | label_recurred | NF59_max | Margination (Maximal) | 0.1268 |
| Pathology | label_recurred | NF40_max | Large Zone Size Emphasis (Maximal) | 0.1268 |
| Pathology | label_recurred | NF46_25% | Large Zone/High Gray Emphasis (25 | 0.1127 |
| | | | percentile) | |
| Pathology | label_recurred | NF45_25% | Large Zone/Low Gray Emphasis (25 | 0.0986 |
| | | | percentile) | |
| Pathology | label_recurred | NF46_0.99 | Large Zone/High Gray Emphasis (99 | 0.0986 |
| | | | percentile) | |
| Pathology | label_recurred | NF37_0.05 | Complexity (5 percentile) | 0.0986 |
| Pathology | label_recurred | NF17_min | Sum of squares variance (Minimal) | 0.0986 |
| Pathology | label_recurred | NF62_0.9 | Closest Neighborhood Distance (90 | 0.0986 |
| | | | percentile) | |
| Pathology | label_recurred | NF2_max | Extent (Maximal) | 0.0986 |
| Pathology | label_recurred | NF40_0.99 | Large Zone Size Emphasis (99 percentile) | 0.0845 |
| Pathology | label_recurred | NF45_min | Large Zone/Low Gray Emphasis | 0.0845 |
| | | | (Minimal) | |
| Pathology | label_recurred | NF38_max | Texture Strength (Maximal) | 0.0845 |
| Pathology | label_recurred | NF63_0.95 | Average Distance to 5 Closest Neighbors | 0.0845 |
| | | | (95 percentile) | |
| Pathology | label_recurred | NF23_max | Difference Variance (Maximal) | 0.0845 |
| Pathology | label_recurred | NF42_0.05 | High Gray-Level Zone Emphasis (5 | 0.0845 |
| | | | percentile) | |
| Pathology | label_recurred | NF46_min | Large Zone/High Gray Emphasis | 0.0845 |
| | | | (Minimal) | |
| Pathology | label_recurred | NF51_max | LONG RUN EMPHASIS(LRE) | 0.0845 |
| | | | (Maximal) | |
| Pathology | label_recurred | NF46_max | Large Zone/High Gray Emphasis | 0.0845 |
| | | | (Maximal) | |
| Pathology | label_recurred | NF50_min | SHORT RUN EMPHASIS (SRE) | 0.0845 |
| | | | (Minimal) | |
| Pathology | label_recurred | NF9_min | Variance (Minimal) | 0.0704 |
| Pathology | label_recurred | NF54_max | RUN LENGTH NON-UNIFORMITY | 0.0704 |
| | | | (RLN) (Maximal) | |
| Pathology | label_recurred | NF14_max | Angular second moment (Maximal) | 0.0704 |
| Pathology | label_recurred | NF12_max | Energy (Maximal) | 0.0704 |
| Pathology | label_recurred | NF63_0.1 | Average Distance to 5 Closest Neighbors | 0.0704 |
| | | | (10 percentile) | |
| Pathology | label_recurred | NF51_std | LONG RUN EMPHASIS(LRE) | 0.0563 |
| | | | (Standard Deviation) | |
| Pathology | label_recurred | NF7_0.01 | Perimeter (1 percentile) | 0.0563 |
| Pathology | label_recurred | NF48_0.01 | Zone Size Non-Uniformity (1 percentile) | 0.0563 |
| Pathology | label_recurred | NF31_0.99 | Cluster Prominence (99 percentile) | 0.0563 |
| Pathology | label_recurred | NF8_min | Mean (Minimal) | 0.0563 |
| Pathology | label_recurred | NF7_max | Perimeter (Maximal) | 0.0563 |
| Pathology | label_recurred | NF52_0.01 | GRAY LEVEL NON-UNIFORMITY | 0.0563 |
| | | | (GLN) (1 percentile) | |
| Pathology | label_recurred | NF10_25% | Skewness (25 percentile) | 0.0563 |
| Pathology | label_recurred | NF52_max | GRAY LEVEL NON-UNIFORMITY | 0.0563 |
| | | | (GLN) (Maximal) | |
| Pathology | label_recurred | NF49_min | Zone Size Percentage (Minimal) | 0.0563 |
| Pathology | label_recurred | NF16_0.01 | Correlation (1 percentile) | 0.0563 |
| Pathology | label_recurred | NF38_0.99 | Texture Strength (99 percentile) | 0.0563 |
| Pathology | label_recurred | NF37_max | Complexity (Maximal) | 0.0563 |
| Pathology | label_recurred | NF52_min | GRAY LEVEL NON-UNIFORMITY | 0.0563 |
| | | | (GLN) (Minimal) | |
| Pathology | label_recurred | NF57_0.1 | Heterogeneity (10 percentile) | 0.0563 |
| Pathology | label_recurred | NF46_0.05 | Large Zone/High Gray Emphasis (5 | 0.0563 |
| | | | percentile) | |

TABLE 18B
Complete Computational Pathology Features to Endpoints
Survival

| | Spearman | Spearman | | Spearman | Spearman | | Spearman | Spearman |
|---|---|---|---|---|---|---|---|---|
| | rho | p-value | | rho | p-value | | rho | p-value |
| NF40_25% | 0.052 | 0.667 | NF40_max | −0.093 | 0.441 | NF38_0.99 | 0.007 | 0.954 |
| NF39_0.01 | −0.155 | 0.198 | NF54_min | 0.053 | 0.658 | NF16_75% | 0.217 | 0.070 |
| NF62_75% | −0.330 | 0.005 | NF48_std | −0.184 | 0.124 | NF22_min | 0.156 | 0.194 |
| NF58_0.01 | 0.129 | 0.282 | NF19_max | −0.146 | 0.224 | NF41_0.01 | 0.240 | 0.043 |
| NF46_0.1 | −0.264 | 0.026 | NF53_mean | −0.013 | 0.917 | NF50_0.1 | −0.087 | 0.470 |
| NF16_0.95 | 0.187 | 0.118 | NF24_mean | −0.170 | 0.156 | NF24_0.9 | −0.134 | 0.267 |
| NF59_min | 0.070 | 0.560 | NF13_75% | −0.100 | 0.407 | NF59_0.99 | −0.228 | 0.056 |
| NF22_std | −0.089 | 0.463 | NF9_50% | −0.025 | 0.834 | NF43_50% | 0.288 | 0.015 |
| NF37_75% | −0.198 | 0.097 | NF21_25% | −0.145 | 0.228 | NF62_50% | −0.388 | 0.001 |
| NF20_0.05 | −0.135 | 0.262 | NF41_0.05 | 0.260 | 0.028 | NF60_0.1 | −0.144 | 0.231 |
| NF50_0.05 | −0.086 | 0.477 | NF22_max | −0.243 | 0.041 | NF50_0.99 | −0.007 | 0.954 |
| NF32_max | −0.150 | 0.210 | NF43_std | 0.180 | 0.133 | NF59_50% | −0.342 | 0.004 |
| NF1_75% | −0.417 | 0.000 | NF16_min | −0.100 | 0.407 | NF11_0.99 | 0.013 | 0.917 |
| NF47_0.95 | −0.222 | 0.063 | NF11_0.01 | 0.060 | 0.616 | NF3_0.9 | −0.217 | 0.069 |
| NF51_75% | 0.025 | 0.834 | NF24_0.99 | −0.063 | 0.600 | NF13_25% | −0.094 | 0.434 |
| NF35_25% | −0.070 | 0.560 | NF13_min | 0.157 | 0.190 | NF29_0.05 | −0.184 | 0.124 |
| NF43_0.99 | 0.226 | 0.058 | NF11_max | −0.069 | 0.568 | NF26_50% | 0.308 | 0.009 |
| NF42_std | −0.020 | 0.871 | NF39_0.05 | −0.159 | 0.186 | NF3_0.1 | −0.259 | 0.029 |
| NF51_50% | 0.042 | 0.727 | NF24_std | 0.228 | 0.056 | NF55_25% | −0.236 | 0.047 |
| NF31_mean | −0.115 | 0.338 | NF8_0.1 | −0.255 | 0.032 | NF63_75% | −0.284 | 0.016 |
| NF37_max | −0.027 | 0.825 | NF59_0.9 | −0.283 | 0.017 | NF36_0.01 | −0.044 | 0.718 |
| NF2_0.1 | −0.353 | 0.003 | NF14_std | −0.149 | 0.215 | NF1_std | 0.401 | 0.001 |
| NF12_0.01 | 0.138 | 0.252 | NF18_0.9 | 0.090 | 0.455 | NF61_min | | |
| NF10_0.05 | 0.229 | 0.055 | NF32_0.99 | −0.205 | 0.086 | NF15_25% | −0.176 | 0.143 |
| NF52_0.95 | −0.202 | 0.090 | NF46_0.99 | −0.214 | 0.073 | NF43_mean | 0.285 | 0.016 |
| NF9_max | −0.070 | 0.560 | NF54_0.9 | −0.218 | 0.068 | NF26_0.01 | 0.239 | 0.045 |
| NF3_0.05 | −0.272 | 0.022 | NF46_std | −0.172 | 0.153 | NF36_0.99 | 0.010 | 0.935 |
| NF8_std | 0.083 | 0.492 | NF42_min | −0.121 | 0.315 | NF35_75% | −0.032 | 0.789 |
| NF1_mean | −0.418 | 0.000 | NF1_0.01 | −0.420 | 0.000 | NF11_std | −0.001 | 0.991 |
| NF11_75% | −0.001 | 0.991 | NF51_std | −0.207 | 0.084 | NF5_min | −0.052 | 0.667 |
| NF26_0.95 | 0.097 | 0.421 | NF28_std | −0.038 | 0.753 | NF41_max | 0.120 | 0.321 |
| NF14_75% | 0.150 | 0.210 | NF2_min | −0.035 | 0.771 | NF52_75% | −0.266 | 0.025 |
| NF13_0.05 | −0.127 | 0.293 | NF13_std | 0.076 | 0.529 | NF5_0.05 | −0.238 | 0.046 |
| NF6_max | −0.024 | 0.843 | NF15_std | 0.107 | 0.375 | NF6_0.01 | −0.309 | 0.009 |
| NF6_75% | −0.221 | 0.063 | NF20_mean | −0.067 | 0.576 | NF12_max | −0.141 | 0.242 |
| NF7_0.05 | −0.141 | 0.242 | NF42_max | −0.149 | 0.215 | NF28_0.9 | −0.270 | 0.023 |
| NF13_0.9 | −0.107 | 0.375 | NF13_0.95 | −0.101 | 0.401 | NF48_0.9 | −0.219 | 0.066 |
| NF39_50% | −0.103 | 0.394 | NF22_0.95 | −0.217 | 0.070 | NF50_std | 0.080 | 0.506 |
| NF34_50% | 0.335 | 0.004 | NF54_25% | −0.298 | 0.012 | NF53_0.9 | −0.017 | 0.889 |
| NF50_75% | −0.049 | 0.684 | NF22_0.01 | −0.204 | 0.088 | NF51_max | −0.120 | 0.321 |
| NF25_0.05 | −0.118 | 0.327 | NF49_std | 0.118 | 0.327 | NF17_0.1 | −0.252 | 0.034 |
| NF16_25% | 0.146 | 0.224 | NF15_0.01 | −0.205 | 0.086 | NF32_0.1 | 0.146 | 0.224 |
| NF21_std | 0.128 | 0.288 | NF16_50% | 0.184 | 0.124 | NF59_std | −0.044 | 0.718 |
| NF1_min | −0.207 | 0.084 | NF8_max | −0.135 | 0.262 | NF44_0.05 | −0.246 | 0.039 |
| NF11_0.05 | −0.024 | 0.843 | NF30_min | 0.134 | 0.267 | NF23_75% | −0.141 | 0.242 |
| NF9_0.9 | −0.070 | 0.560 | NF19_0.1 | −0.259 | 0.029 | NF11_50% | −0.007 | 0.954 |
| NF19_25% | −0.252 | 0.034 | NF27_min | | | NF37_0.95 | −0.181 | 0.130 |
| | −0.290 | 0.014 | NF46_0.01 | −0.201 | 0.093 | NF6_0.1 | −0.260 | 0.029 |
| NF9_0.99 | −0.093 | 0.441 | NF14_25% | 0.200 | 0.095 | NF7_50% | −0.239 | 0.045 |
| NF1_max | 0.003 | 0.981 | NF24_0.1 | −0.191 | 0.110 | NF18_0.95 | 0.100 | 0.407 |
| NF61_0.9 | −0.060 | 0.616 | NF23_25% | −0.180 | 0.133 | NF62_max | −0.013 | 0.917 |
| NF51_0.99 | −0.180 | 0.133 | NF5_mean | −0.301 | 0.011 | NF31_0.9 | −0.100 | 0.407 |
| NF45_75% | 0.301 | 0.011 | NF31_50% | −0.096 | 0.428 | NF21_75% | −0.132 | 0.272 |
| NF63_50% | −0.312 | 0.008 | NF44_0.01 | −0.212 | 0.075 | NF6_mean | −0.256 | 0.031 |
| NF9_75% | −0.027 | 0.825 | NF50_25% | −0.072 | 0.552 | NF10_min | 0.142 | 0.237 |
| NF2_std | 0.319 | 0.007 | NF12_75% | 0.089 | 0.463 | NF60_min | | |
| NF48_50% | −0.280 | 0.018 | NF13_0.99 | −0.153 | 0.202 | NF2_0.9 | −0.238 | 0.045 |
| NF1_25% | −0.399 | 0.001 | NF16_max | 0.172 | 0.153 | NF44_0.99 | −0.285 | 0.016 |
| NF12_25% | 0.104 | 0.388 | NF35_0.05 | −0.105 | 0.381 | NF57_50% | −0.082 | 0.495 |
| NF6_50% | −0.295 | 0.013 | NF10_50% | 0.259 | 0.029 | NF33_0.95 | 0.112 | 0.350 |
| NF63_min | −0.290 | 0.014 | NF46_0.9 | −0.238 | 0.046 | NF13_max | −0.015 | 0.898 |
| NF7_0.99 | −0.103 | 0.394 | NF10_25% | 0.266 | 0.025 | NF47_0.05 | −0.156 | 0.194 |
| NF61_max | 0.073 | 0.544 | NF5_0.9 | −0.297 | 0.012 | NF15_0.99 | −0.034 | 0.780 |
| NF39_0.95 | −0.049 | 0.684 | NF50_max | 0.086 | 0.477 | NF25_75% | −0.266 | 0.025 |
| NF28_25% | −0.225 | 0.059 | NF9_0.1 | −0.091 | 0.448 | NF9_std | −0.082 | 0.499 |
| NF20_0.01 | −0.104 | 0.388 | NF35_0.01 | −0.146 | 0.224 | NF30_0.9 | 0.280 | 0.018 |
| NF12_0.05 | 0.097 | 0.421 | NF52_50% | −0.290 | 0.014 | NF37_0.01 | −0.224 | 0.061 |
| NF57_0.1 | −0.144 | 0.231 | NF38_0.95 | 0.114 | 0.344 | NF7_0.01 | −0.153 | 0.202 |
| NF40_0.95 | −0.091 | 0.448 | NF62_25% | −0.387 | 0.001 | NF24_0.95 | −0.129 | 0.282 |
| NF29_0.9 | −0.100 | 0.407 | NF4_0.99 | 0.255 | 0.032 | NF53_0.01 | 0.132 | 0.272 |
| NF34_max | −0.034 | 0.780 | NF18_0.01 | 0.001 | 0.991 | NF26_max | 0.010 | 0.935 |
| NF16_mean | 0.165 | 0.170 | NF45_0.9 | 0.283 | 0.017 | NF50_mean | −0.056 | 0.641 |
| NF24_min | −0.183 | 0.127 | NF14_min | 0.329 | 0.005 | NF26_0.05 | 0.267 | 0.024 |
| NF46_max | −0.097 | 0.421 | NF63_0.05 | −0.277 | 0.019 | NF51_0.9 | −0.020 | 0.871 |
| NF27_25% | | | NF33_25% | 0.077 | 0.521 | NF44_25% | −0.232 | 0.052 |
| NF41_std | 0.128 | 0.288 | NF17_0.95 | −0.255 | 0.032 | NF53_25% | −0.044 | 0.718 |
| NF19_0.01 | −0.129 | 0.282 | NF30_0.01 | 0.231 | 0.053 | NF49_25% | −0.080 | 0.506 |
| NF52_0.05 | −0.128 | 0.288 | NF25_0.9 | −0.269 | 0.024 | NF60_mean | −0.077 | 0.521 |
| NF31_25% | −0.117 | 0.332 | NF33_0.01 | −0.004 | 0.972 | NF53_75% | −0.037 | 0.758 |
| NF23_0.95 | −0.114 | 0.344 | NF55_min | −0.090 | 0.455 | NF20_0.1 | −0.111 | 0.356 |
| NF12_0.99 | 0.070 | 0.560 | NF49_0.01 | −0.100 | 0.407 | NF45_25% | 0.335 | 0.004 |
| NF23_0.1 | −0.208 | 0.082 | NF9_25% | −0.052 | 0.667 | NF50_0.01 | −0.063 | 0.600 |
| NF36_min | −0.190 | 0.113 | NF20_std | −0.073 | 0.544 | NF36_75% | −0.052 | 0.667 |
| NF39_25% | −0.135 | 0.262 | NF19_std | 0.083 | 0.492 | NF14_50% | 0.184 | 0.124 |
| NF33_0.9 | 0.112 | 0.350 | NF8_0.05 | −0.240 | 0.043 | NF54_0.01 | −0.235 | 0.049 |
| NF4_std | −0.381 | 0.001 | NF34_std | 0.160 | 0.182 | NF43_0.95 | 0.250 | 0.035 |
| NF16_0.9 | 0.215 | 0.072 | NF17_max | −0.148 | 0.219 | NF7_std | −0.051 | 0.675 |
| NF6_std | −0.149 | 0.215 | NF17_25% | −0.224 | 0.061 | NF62_0.1 | −0.398 | 0.001 |
| NF23_min | −0.312 | 0.008 | NF59_0.01 | −0.250 | 0.035 | NF34_mean | 0.340 | 0.004 |
| NF59_mean | −0.329 | 0.005 | NF63_0.9 | −0.249 | 0.036 | NF39_max | 0.024 | 0.843 |
| NF55_0.99 | −0.243 | 0.041 | NF31_std | −0.128 | 0.288 | NF29_min | −0.186 | 0.121 |
| NF6_0.95 | −0.182 | 0.129 | NF39_0.1 | −0.152 | 0.206 | NF42_0.9 | −0.297 | 0.012 |
| NF31_0.1 | −0.135 | 0.262 | NF1_0.05 | −0.398 | 0.001 | NF35_0.1 | −0.091 | 0.448 |
| NF62_0.01 | −0.383 | 0.001 | NF31_0.99 | −0.163 | 0.174 | NF20_0.99 | −0.107 | 0.375 |
| NF1_50% | −0.375 | 0.001 | NF32_75% | 0.006 | 0.963 | NF43_0.01 | 0.255 | 0.032 |
| NF32_0.9 | −0.125 | 0.298 | NF31_min | −0.179 | 0.136 | NF57_0.05 | −0.137 | 0.253 |
| NF23_0.01 | −0.242 | 0.042 | NF63_std | −0.135 | 0.262 | NF60_0.05 | −0.137 | 0.253 |
| NF5_0.99 | −0.273 | 0.021 | NF60_75% | | | NF22_0.9 | −0.219 | 0.066 |
| NF55_0.1 | −0.253 | 0.033 | NF32_min | 0.160 | 0.182 | NF34_75% | 0.347 | 0.003 |
| NF15_min | −0.238 | 0.046 | NF49_mean | −0.067 | 0.576 | NF18_mean | 0.076 | 0.529 |
| NF54_mean | −0.240 | 0.043 | NF58_0.95 | 0.137 | 0.253 | NF34_0.1 | 0.242 | 0.042 |
| NF50_0.95 | 0.018 | 0.880 | NF38_mean | 0.218 | 0.068 | NF26_min | 0.097 | 0.421 |
| NF7_0.95 | −0.115 | 0.338 | NF6_0.99 | −0.146 | 0.224 | NF63_0.95 | −0.221 | 0.064 |
| NF4_0.05 | 0.337 | 0.004 | NF47_50% | −0.298 | 0.012 | NF24_max | 0.098 | 0.414 |
| NF58_0.1 | 0.059 | 0.625 | NF33_mean | 0.083 | 0.492 | NF2_max | 0.150 | 0.213 |
| NF55_max | −0.165 | 0.170 | NF41_50% | 0.329 | 0.005 | NF25_max | −0.179 | 0.136 |
| NF41_0.99 | 0.181 | 0.130 | NF8_min | 0.003 | 0.981 | NF63_0.01 | −0.301 | 0.011 |
| NF60_50% | 0.130 | 0.279 | NF7_min | −0.164 | 0.172 | NF38_25% | 0.228 | 0.056 |
| NF45_max | 0.035 | 0.771 | NF53_0.05 | 0.010 | 0.935 | NF5_0.95 | −0.337 | 0.004 |
| NF21_mean | −0.142 | 0.237 | NF4_25% | 0.325 | 0.006 | NF13_0.1 | −0.121 | 0.315 |
| num_nuclei | 0.038 | 0.753 | NF41_25% | 0.309 | 0.009 | NF27_mean | | |
| NF50_50% | −0.059 | 0.625 | NF29_max | 0.077 | 0.521 | NF39_0.99 | 0.038 | 0.753 |
| NF22_25% | −0.247 | 0.037 | NF5_max | −0.139 | 0.247 | NF60_max | | |
| NF8_50% | −0.283 | 0.017 | NF14_mean | 0.094 | 0.434 | NF31_0.05 | −0.132 | 0.272 |
| NF33_75% | 0.114 | 0.344 | NF34_0.01 | 0.236 | 0.047 | NF19_50% | −0.260 | 0.028 |
| NF57_min | | | NF25_0.1 | −0.202 | 0.090 | NF27_0.01 | | |
| NF30_mean | 0.235 | 0.049 | NF9_0.95 | −0.124 | 0.304 | NF18_75% | 0.098 | 0.414 |
| NF45_0.05 | 0.302 | 0.010 | NF42_75% | −0.301 | 0.011 | NF4_min | 0.139 | 0.247 |
| NF10_0.1 | 0.253 | 0.033 | NF43_max | 0.031 | 0.798 | NF14_0.99 | −0.120 | 0.321 |
| NF20_min | −0.129 | 0.282 | NF48_0.95 | −0.207 | 0.084 | NF18_0.05 | 0.013 | 0.917 |
| NF56_mean | −0.260 | 0.028 | NF18_std | 0.075 | 0.537 | NF39_mean | −0.117 | 0.332 |
| NF40_0.9 | −0.021 | 0.861 | NF25_0.95 | −0.259 | 0.029 | NF2_0.95 | −0.141 | 0.242 |
| NF60_std | 0.111 | 0.356 | NF53_0.99 | 0.049 | 0.684 | NF59_max | 0.046 | 0.701 |
| NF50_min | 0.083 | 0.492 | NF35_std | 0.041 | 0.736 | NF48_25% | −0.295 | 0.012 |
| NF58_max | | | NF44_mean | −0.273 | 0.021 | NF51_0.01 | −0.038 | 0.753 |
| NF47_25% | −0.297 | 0.012 | NF37_mean | −0.221 | 0.064 | NF9_0.01 | −0.089 | 0.463 |
| NF42_50% | −0.273 | 0.021 | NF56_0.01 | −0.056 | 0.641 | NF61_75% | −0.063 | 0.600 |
| NF58_min | 0.032 | 0.789 | NF27_0.95 | | | NF17_0.01 | −0.150 | 0.210 |
| NF58_0.99 | | | NF15_50% | −0.153 | 0.202 | NF10_max | 0.107 | 0.375 |
| NF2_0.99 | 0.112 | 0.353 | NF61_50% | −0.085 | 0.480 | NF31_75% | −0.089 | 0.463 |
| NF63_0.99 | −0.165 | 0.170 | NF57_0.95 | −0.072 | 0.552 | NF6_min | −0.113 | 0.350 |
| NF55_50% | −0.259 | 0.029 | NF29_0.1 | −0.169 | 0.159 | NF49_0.9 | −0.034 | 0.775 |
| NF43_0.1 | 0.285 | 0.016 | NF38_75% | 0.267 | 0.024 | NF49_0.95 | −0.007 | 0.954 |
| NF52_min | 0.089 | 0.463 | NF3_max | −0.024 | 0.843 | NF28_min | −0.056 | 0.641 |
| NF4_mean | 0.301 | 0.011 | NF45_0.01 | 0.273 | 0.021 | NF39_min | −0.179 | 0.136 |
| NF52_mean | −0.260 | 0.028 | NF29_25% | −0.153 | 0.202 | NF53_0.1 | −0.021 | 0.861 |
| NF33_0.1 | 0.025 | 0.834 | NF17_mean | −0.259 | 0.029 | NF36_max | −0.142 | 0.237 |
| NF19_0.99 | −0.255 | 0.032 | NF11_0.9 | −0.015 | 0.898 | NF20_75% | −0.024 | 0.843 |
| NF34_0.05 | 0.240 | 0.043 | NF29_mean | −0.143 | 0.233 | NF47_75% | −0.253 | 0.033 |
| NF16_0.05 | 0.034 | 0.780 | NF55_0.9 | −0.278 | 0.019 | NF25_std | −0.127 | 0.293 |
| NF30_25% | 0.202 | 0.090 | NF52_std | −0.177 | 0.139 | NF38_min | −0.060 | 0.616 |
| NF38_0.01 | 0.132 | 0.272 | NF24_75% | −0.156 | 0.194 | NF23_0.05 | −0.228 | 0.056 |
| NF23_50% | −0.165 | 0.170 | NF1_0.9 | −0.422 | 0.000 | NF56_0.95 | −0.202 | 0.090 |
| NF24_0.01 | −0.190 | 0.113 | NF4_0.9 | 0.269 | 0.024 | NF62_min | −0.095 | 0.430 |
| NF27_0.99 | | | NF45_0.99 | 0.117 | 0.332 | NF29_0.95 | −0.100 | 0.407 |
| NF52_0.9 | −0.202 | 0.090 | NF35_mean | −0.062 | 0.608 | NF18_min | −0.107 | 0.375 |
| NF61_25% | −0.064 | 0.598 | NF53_max | 0.065 | 0.588 | NF30_0.1 | 0.208 | 0.082 |
| NF45_0.1 | 0.285 | 0.016 | NF36_0.95 | 0.004 | 0.972 | NF57_0.9 | −0.059 | 0.625 |
| NF49_0.1 | −0.084 | 0.484 | NF2_50% | −0.358 | 0.002 | NF32_std | −0.225 | 0.059 |
| NF57_max | −0.032 | 0.789 | NF38_50% | 0.284 | 0.016 | NF47_std | −0.195 | 0.102 |
| NF36_mean | −0.070 | 0.560 | NF21_max | 0.058 | 0.633 | NF17_75% | −0.290 | 0.014 |
| NF40_mean | −0.059 | 0.625 | NF19_75% | −0.288 | 0.015 | NF39_0.9 | −0.051 | 0.675 |
| NF40_0.99 | −0.197 | 0.100 | NF22_0.99 | −0.201 | 0.093 | NF28_max | −0.134 | 0.267 |
| NF49_max | 0.053 | 0.662 | NF35_0.95 | −0.013 | 0.917 | NF44_std | 0.001 | 0.991 |
| NF22_0.1 | −0.291 | 0.014 | NF30_0.95 | 0.195 | 0.102 | NF49_0.99 | −0.001 | 0.991 |
| NF47_0.99 | −0.174 | 0.146 | NF2_75% | −0.328 | 0.005 | NF22_75% | −0.221 | 0.064 |
| NF43_min | 0.229 | 0.055 | NF18_0.99 | 0.066 | 0.584 | NF3_75% | −0.221 | 0.063 |
| NF35_max | 0.030 | 0.807 | NF28_0.01 | −0.132 | 0.272 | NF48_75% | −0.222 | 0.063 |
| NF18_50% | 0.083 | 0.492 | NF5_0.01 | −0.255 | 0.032 | NF4_max | 0.052 | 0.667 |
| NF44_max | −0.170 | 0.156 | NF1_0.99 | −0.134 | 0.267 | NF37_min | −0.125 | 0.298 |
| NF49_75% | −0.067 | 0.576 | NF34_min | 0.112 | 0.350 | NF49_min | 0.050 | 0.679 |
| NF61_0.01 | | | NF45_50% | 0.321 | 0.006 | NF28_mean | −0.267 | 0.024 |
| NF15_max | −0.020 | 0.871 | NF23_mean | −0.165 | 0.170 | NF13_50% | −0.096 | 0.428 |
| NF27_max | | | NF35_0.9 | −0.024 | 0.843 | NF46_0.95 | −0.233 | 0.050 |
| NF23_max | −0.013 | 0.917 | NF52_max | −0.083 | 0.492 | NF33_50% | 0.097 | 0.421 |
| NF25_mean | −0.290 | 0.014 | NF56_0.9 | −0.202 | 0.090 | NF29_0.99 | −0.007 | 0.954 |
| NF40_50% | 0.045 | 0.709 | NF23_0.99 | −0.062 | 0.608 | NF53_min | 0.098 | 0.414 |
| NF30_75% | 0.266 | 0.025 | NF58_0.05 | 0.072 | 0.552 | NF52_0.01 | −0.056 | 0.641 |
| NF54_50% | −0.271 | 0.022 | NF33_0.99 | 0.069 | 0.568 | NF58_25% | 0.055 | 0.650 |
| NF9_mean | −0.063 | 0.600 | NF62_0.99 | −0.208 | 0.082 | NF7_0.9 | −0.134 | 0.267 |
| NF3_min | −0.113 | 0.350 | NF40_0.1 | 0.024 | 0.843 | NF48_0.99 | −0.197 | 0.100 |
| NF41_75% | 0.295 | 0.012 | NF31_0.01 | −0.139 | 0.247 | NF15_mean | −0.136 | 0.257 |
| NF3_std | −0.142 | 0.237 | NF36_50% | −0.108 | 0.369 | NF47_max | −0.098 | 0.414 |
| NF9_0.05 | −0.112 | 0.350 | NF54_0.95 | −0.193 | 0.107 | NF56_min | 0.089 | 0.463 |
| NF12_0.1 | 0.110 | 0.363 | NF13_mean | −0.098 | 0.414 | NF62_mean | −0.329 | 0.005 |
| NF60_0.9 | | | NF37_0.9 | −0.198 | 0.097 | NF2_mean | −0.349 | 0.003 |
| NF48_min | −0.056 | 0.641 | NF20_0.9 | −0.067 | 0.576 | NF60_0.99 | | |
| NF8_0.9 | −0.301 | 0.011 | NF11_0.95 | 0.001 | 0.991 | NF24_50% | −0.172 | 0.153 |
| NF22_50% | −0.247 | 0.037 | NF45_0.95 | 0.232 | 0.052 | NF26_mean | 0.281 | 0.018 |
| NF22_0.05 | −0.295 | 0.012 | NF46_25% | −0.266 | 0.025 | NF5_25% | −0.284 | 0.016 |
| NF58_75% | 0.059 | 0.623 | NF32_mean | −0.100 | 0.407 | NF47_min | 0.011 | 0.930 |
| NF18_max | −0.059 | 0.625 | NF56_0.1 | −0.190 | 0.113 | NF41_mean | 0.302 | 0.010 |
| NF55_std | −0.024 | 0.843 | NF49_0.05 | −0.087 | 0.470 | NF37_0.1 | −0.233 | 0.050 |
| NF24_0.05 | −0.188 | 0.116 | NF37_std | 0.048 | 0.692 | NF46_75% | −0.266 | 0.025 |
| NF28_0.05 | −0.239 | 0.045 | NF23_std | 0.089 | 0.463 | NF63_0.1 | −0.284 | 0.016 |
| NF32_0.05 | 0.152 | 0.206 | NF17_0.05 | −0.239 | 0.045 | NF20_25% | −0.077 | 0.521 |
| NF44_min | −0.142 | 0.237 | NF40_0.05 | −0.015 | 0.898 | NF13_0.01 | −0.127 | 0.293 |
| NF50_0.9 | −0.006 | 0.963 | NF61_0.05 | −0.137 | 0.253 | NF42_25% | −0.240 | 0.043 |
| NF35_50% | −0.060 | 0.616 | NF33_max | −0.046 | 0.701 | NF21_0.01 | −0.193 | 0.107 |
| NF57_25% | −0.059 | 0.623 | NF38_0.9 | 0.156 | 0.194 | NF57_mean | −0.070 | 0.560 |
| NF48_0.01 | −0.239 | 0.045 | NF45_min | 0.285 | 0.016 | NF37_0.05 | −0.274 | 0.021 |
| NF15_0.95 | −0.110 | 0.363 | NF53_0.95 | 0.027 | 0.825 | NF62_std | −0.149 | 0.215 |
| NF41_min | 0.300 | 0.011 | NF12_0.95 | 0.100 | 0.407 | NF62_0.05 | −0.390 | 0.001 |
| NF25_25% | −0.287 | 0.015 | NF32_50% | 0.082 | 0.499 | NF10_0.9 | 0.273 | 0.021 |
| NF60_0.01 | | | NF62_0.9 | −0.307 | 0.009 | NF55_0.01 | −0.160 | 0.182 |
| NF28_0.95 | −0.255 | 0.032 | NF8_25% | −0.262 | 0.028 | NF52_25% | −0.278 | 0.019 |
| NF51_25% | 0.039 | 0.744 | NF2_0.05 | −0.321 | 0.006 | NF19_0.05 | −0.226 | 0.058 |
| NF19_0.9 | −0.278 | 0.019 | NF40_min | −0.044 | 0.718 | NF36_25% | −0.128 | 0.288 |
| NF62_0.95 | −0.297 | 0.012 | NF20_0.95 | −0.103 | 0.394 | NF43_0.9 | 0.288 | 0.015 |
| NF8_0.99 | −0.276 | 0.020 | NF56_std | −0.177 | 0.139 | NF21_0.99 | −0.180 | 0.133 |
| NF43_0.05 | 0.270 | 0.023 | NF21_0.1 | −0.173 | 0.149 | NF25_0.01 | 0.021 | 0.861 |
| NF36_0.05 | −0.131 | 0.277 | NF38_0.1 | 0.197 | 0.100 | NF44_0.9 | −0.291 | 0.014 |
| NF25_min | −0.045 | 0.709 | NF63_mean | −0.280 | 0.018 | NF11_min | 0.007 | 0.954 |
| NF34_0.95 | 0.297 | 0.012 | NF31_0.95 | −0.122 | 0.309 | NF61_std | −0.079 | 0.514 |
| NF47_mean | −0.270 | 0.023 | NF40_std | −0.169 | 0.159 | NF27_75% | | |
| NF28_0.99 | −0.253 | 0.033 | NF59_0.1 | −0.298 | 0.012 | NF33_std | 0.110 | 0.363 |
| NF21_0.95 | −0.184 | 0.124 | NF61_mean | −0.079 | 0.514 | NF30_0.99 | 0.097 | 0.421 |
| NF55_75% | −0.298 | 0.012 | NF5_75% | −0.325 | 0.006 | NF41_0.1 | 0.264 | 0.026 |
| NF2_25% | −0.357 | 0.002 | NF4_75% | 0.285 | 0.016 | NF42_0.99 | −0.262 | 0.028 |
| NF41_0.95 | 0.214 | 0.073 | NF48_max | −0.163 | 0.174 | NF15_0.05 | −0.204 | 0.088 |
| NF14_0.05 | 0.210 | 0.079 | NF8_75% | −0.297 | 0.012 | NF52_0.99 | −0.143 | 0.233 |
| NF39_std | 0.202 | 0.090 | NF10_0.01 | 0.152 | 0.206 | NF8_mean | −0.287 | 0.015 |
| NF46_mean | −0.271 | 0.022 | NF25_0.99 | −0.218 | 0.068 | NF22_mean | −0.249 | 0.036 |
| NF48_0.05 | −0.292 | 0.013 | NF30_0.05 | 0.181 | 0.130 | NF17_min | −0.021 | 0.861 |
| NF12_0.9 | 0.101 | 0.401 | NF16_0.1 | 0.077 | 0.521 | NF58_0.9 | 0.144 | 0.231 |
| NF8_0.01 | −0.135 | 0.262 | NF3_0.01 | −0.309 | 0.009 | NF35_0.99 | −0.058 | 0.633 |
| NF51_mean | −0.024 | 0.843 | NF15_0.9 | −0.110 | 0.363 | NF59_0.05 | −0.264 | 0.026 |
| NF21_0.9 | −0.169 | 0.159 | NF56_75% | −0.266 | 0.025 | NF44_0.1 | −0.242 | 0.042 |
| NF35_min | −0.157 | 0.190 | NF25_50% | −0.312 | 0.008 | NF8_0.95 | −0.281 | 0.018 |
| NF14_0.9 | 0.115 | 0.338 | NF27_std | | | NF6_0.05 | −0.272 | 0.022 |
| NF44_50% | −0.266 | 0.025 | NF36_0.9 | −0.010 | 0.935 | NF10_mean | 0.285 | 0.016 |
| NF7_75% | −0.155 | 0.198 | NF4_0.1 | 0.297 | 0.012 | NF57_0.01 | | |
| NF56_25% | −0.278 | 0.019 | NF55_0.95 | −0.273 | 0.021 | NF12_std | −0.030 | 0.807 |
| NF3_50% | −0.295 | 0.013 | NF4_0.95 | 0.238 | 0.046 | NF38_max | 0.070 | 0.560 |
| NF10_0.95 | 0.231 | 0.053 | NF55_0.05 | −0.250 | 0.035 | NF30_max | 0.104 | 0.388 |
| NF53_50% | −0.039 | 0.744 | NF42_0.1 | −0.255 | 0.032 | NF31_max | −0.125 | 0.298 |
| NF54_0.05 | −0.267 | 0.024 | NF47_0.9 | −0.235 | 0.049 | NF26_0.99 | −0.020 | 0.871 |
| NF51_0.05 | 0.000 | 1.000 | NF34_25% | 0.270 | 0.023 | NF63_25% | −0.314 | 0.008 |
| NF54_max | −0.136 | 0.257 | NF10_75% | 0.262 | 0.028 | NF26_std | −0.249 | 0.036 |
| NF59_25% | −0.330 | 0.005 | NF27_0.9 | | | NF55_mean | −0.278 | 0.019 |
| NF33_0.05 | 0.032 | 0.789 | NF34_0.99 | 0.162 | 0.178 | NF1_0.1 | −0.382 | 0.001 |
| NF21_50% | −0.134 | 0.267 | NF33_min | −0.087 | 0.470 | NF27_0.1 | | |
| NF19_0.95 | −0.267 | 0.024 | NF18_25% | 0.067 | 0.576 | NF30_std | −0.037 | 0.762 |
| NF38_std | 0.022 | 0.852 | NF7_25% | −0.248 | 0.037 | NF41_0.9 | 0.239 | 0.045 |
| NF7_mean | −0.181 | 0.130 | NF56_max | −0.083 | 0.492 | NF12_mean | 0.090 | 0.455 |
| NF26_25% | 0.288 | 0.015 | NF51_0.95 | −0.079 | 0.514 | NF40_0.01 | 0.006 | 0.963 |
| NF4_0.01 | 0.280 | 0.018 | NF12_50% | 0.107 | 0.375 | NF10_0.99 | 0.153 | 0.202 |
| NF44_0.95 | −0.298 | 0.012 | NF58_mean | 0.070 | 0.560 | NF44_75% | −0.290 | 0.014 |
| NF28_50% | −0.259 | 0.029 | NF61_0.1 | −0.140 | 0.245 | NF61_0.95 | −0.067 | 0.576 |
| NF42_0.05 | −0.263 | 0.027 | NF57_0.99 | −0.129 | 0.282 | NF10_std | 0.098 | 0.414 |
| NF17_0.9 | −0.264 | 0.026 | NF12_min | 0.034 | 0.780 | NF56_0.05 | −0.128 | 0.288 |
| NF48_mean | −0.252 | 0.034 | NF59_75% | −0.335 | 0.004 | NF36_0.1 | −0.153 | 0.202 |
| NF42_0.01 | −0.186 | 0.121 | NF27_50% | | | NF60_0.95 | | |
| NF47_0.01 | −0.015 | 0.898 | NF1_0.95 | −0.330 | 0.005 | NF42_mean | −0.278 | 0.019 |
| NF45_std | 0.065 | 0.592 | NF16_0.99 | 0.082 | 0.499 | NF37_25% | −0.257 | 0.030 |
| NF29_0.01 | −0.174 | 0.146 | NF46_min | −0.028 | 0.816 | NF57_std | −0.077 | 0.521 |
| NF21_0.05 | −0.177 | 0.139 | NF7_0.1 | −0.188 | 0.116 | NF16_0.01 | −0.028 | 0.816 |
| NF26_0.9 | 0.200 | 0.095 | NF29_50% | −0.143 | 0.233 | NF40_75% | 0.037 | 0.762 |
| NF46_50% | −0.252 | 0.034 | NF37_50% | −0.232 | 0.052 | NF18_0.1 | 0.028 | 0.816 |
| NF29_75% | −0.118 | 0.327 | NF39_75% | −0.090 | 0.455 | NF19_min | 0.011 | 0.926 |
| NF29_std | 0.218 | 0.068 | NF14_0.95 | 0.077 | 0.521 | NF37_0.99 | −0.122 | 0.309 |
| NF54_0.1 | −0.295 | 0.012 | NF27_0.05 | | | NF51_min | −0.023 | 0.852 |
| NF54_75% | −0.232 | 0.052 | NF20_max | −0.077 | 0.521 | NF3_25% | −0.308 | 0.009 |
| NF17_std | −0.035 | 0.771 | NF34_0.9 | 0.305 | 0.010 | NF58_50% | 0.082 | 0.495 |
| NF36_std | 0.020 | 0.871 | NF43_25% | 0.291 | 0.014 | NF5_std | 0.136 | 0.257 |
| NF17_0.99 | −0.253 | 0.033 | NF53_std | −0.093 | 0.441 | NF51_0.1 | 0.008 | 0.944 |
| NF28_75% | −0.285 | 0.016 | NF23_0.9 | −0.134 | 0.267 | NF52_0.1 | −0.190 | 0.113 |
| NF54_std | −0.162 | 0.178 | NF30_50% | 0.219 | 0.066 | NF19_mean | −0.271 | 0.022 |
| NF32_0.01 | 0.177 | 0.139 | NF21_min | 0.120 | 0.321 | NF58_std | −0.077 | 0.521 |
| NF47_0.1 | −0.200 | 0.095 | NF26_75% | 0.295 | 0.012 | NF5_0.1 | −0.269 | 0.024 |
| NF14_max | −0.143 | 0.233 | NF42_0.95 | −0.290 | 0.014 | NF4_50% | 0.309 | 0.009 |
| NF2_0.01 | −0.321 | 0.006 | NF61_0.99 | −0.075 | 0.537 | NF63_max | −0.086 | 0.477 |
| NF56_0.99 | −0.143 | 0.233 | NF17_50% | −0.255 | 0.032 | NF49_50% | −0.073 | 0.544 |
| NF20_50% | −0.041 | 0.736 | NF3_0.95 | −0.182 | 0.129 | NF32_0.95 | −0.187 | 0.118 |
| NF9_min | −0.127 | 0.293 | NF11_25% | −0.042 | 0.727 | NF15_0.1 | −0.191 | 0.110 |
| NF60_25% | −0.064 | 0.598 | NF57_75% | −0.055 | 0.650 | NF26_0.1 | 0.263 | 0.027 |
| NF6_0.9 | −0.217 | 0.069 | NF59_0.95 | −0.255 | 0.032 | NF6_25% | −0.308 | 0.009 |
| NF38_0.05 | 0.184 | 0.124 | NF43_75% | 0.297 | 0.012 | NF28_0.1 | −0.259 | 0.029 |
| NF11_mean | −0.010 | 0.935 | NF3_0.99 | −0.146 | 0.224 | NF5_50% | −0.309 | 0.009 |
| NF45_mean | 0.267 | 0.024 | NF14_0.01 | 0.218 | 0.068 | NF3_mean | −0.262 | 0.028 |
| NF24_25% | −0.186 | 0.121 | NF14_0.1 | 0.215 | 0.072 | | | |
| NF54_0.99 | −0.159 | 0.186 | NF15_75% | −0.125 | 0.298 | | | |
| NF32_25% | 0.128 | 0.288 | NF46_0.05 | −0.235 | 0.049 | | | |
| NF11_0.1 | −0.065 | 0.592 | NF16_std | 0.104 | 0.388 | | | |
| NF48_0.1 | −0.302 | 0.010 | NF7_max | 0.112 | 0.350 | | | |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

What is claimed is:

1. A computer-implemented method comprising:
determining available medical tests at a medical institution, the available medical tests being at least a subset of known medical tests performed at various medical institutions;
selecting, from the available medical tests, selected medical tests based on a trained parsimonious model for pancreatic cancer;
obtaining one or more biological samples from a subject for the selected medical tests;
assaying the one or more biological samples via the selected medical tests to obtain one or more factors; and
prognosticating the subject as having a higher likelihood of survival, the subject as having a higher likelihood of recurrence, or a combination thereof based on the trained parsimonious model and the one or more factors.

2. The method of claim 1, further comprising weighting each factor of the one or more factors based on the selected medical tests.

3. The method of claim 1, further comprising selecting a pancreatic cancer treatment method from among a plurality of pancreatic cancer treatment methods based on the trained parsimonious model and the one or more factors.

4. The method of claim 1, further comprising administering the pancreatic cancer treatment method.

5. A computer-implemented method comprising:
processing a plurality of analytes from a plurality of individuals with cancer to obtain a plurality of features;
training one or more machine learning models with single-omic and multi-omic combinations of the plurality of features to predict binary survival and disease recurrence outcomes of the plurality of individuals;
evaluating the one or more machine learning models for positive predictive value and accuracy in predicting the survival and disease recurrence outcomes and feature proportions; and
recursively eliminating features from the plurality of features based on the evaluating of the one or more machine learning models to develop a parsimonious machine learning model for predicting survival and disease recurrence outcome.

6. The method of claim 5, wherein the plurality of analytes are derived from serum, plasma, blood and/or tissue samples subjected to targeted NGS DNA sequencing, whole transcriptome RNA sequencing, paired tissue proteomics, unpaired serum proteomics, lipidomics, surgical pathology, and/or computational pathology.

7. The method of claim 5, wherein the plurality of analytes include plasma or serum or blood proteins, RNA fusions, tissue proteins, plasma or serum lipids, RNA gene expressions, CNVs, INDELS, SNVs, and or tumor nuclei characteristics.

8. The method of claim 5, wherein the feature proportions evaluated using a leave-one-patient-out cross-validation strategy.

9. The method of claim 5, wherein the one or more machine learning models Support Vector Machine (SVM), Principal Component Analysis (PCA)+Logistic Regression, L1-Normalized SVM, L1-Normalized Random Forest, 5-hidden-layer Deep Neural Network, Recursive Feature Elimination (RFE) Logistic Regression and/or RFE Random Forest.

10. A system comprising:
memory storing computer-executable instructions; and
one or more processors, the one or more processors being configured to execute the computer-executable instructions to:
determine available medical tests at a medical institution, the available medical tests being at least a subset of known medical tests performed at various medical institutions;
select, from the available medical tests, selected medical tests based on a trained parsimonious model for pancreatic cancer;
obtain one or more biological samples from a subject for the selected medical tests;
assay the one or more biological samples via the selected medical tests to obtain one or more factors; and
prognosticate the subject as having a higher likelihood of survival, the subject as having a higher likelihood of recurrence, or a combination thereof based on the trained parsimonious model and the one or more factors.

11. The system of claim 10, wherein the one or more processors are configured to execute the computer-executable instructions to weight each factor of the one or more factors based on the selected medical tests.

12. The system of claim 10, wherein the one or more processors are configured to execute the computer-executable instructions to select a pancreatic cancer treatment method from among a plurality of pancreatic cancer treatment methods based on the trained parsimonious model and the one or more factors.

13. The system of claim 10, wherein the one or more processors are configured to execute the computer-executable instructions to cause, at least on part, an administering of the pancreatic cancer treatment.

14. A system comprising:
memory storing computer-executable instructions; and
one or more processors, the one or more processors being configured to execute the computer-executable instructions to:
receive a plurality of features from a plurality of analytes obtained from a plurality of individuals with cancer;
train one or more machine learning models with single-omic and multi-omic combinations of the plurality of features to predict binary survival and disease recurrence outcomes of the plurality of individuals;
evaluate the one or more machine learning models for positive predictive value and accuracy in predicting the survival and disease recurrence outcomes and feature weights; and
recursively eliminate features from the plurality of features based on the evaluating of the one or more machine learning models to develop a parsimonious machine learning model for predicting survival and disease recurrence outcome.

15. The system of claim 14, wherein the plurality of analytes are derived from serum, plasma or blood, and tissue tumor samples subjected to targeted NGS DNA sequencing, whole transcriptome RNA sequencing, paired tissue proteomics, unpaired serum proteomics, lipidomics, surgical pathology, and/or computational pathology.

16. The system of claim 14, wherein the plurality of analytes include plasma, serum, or blood proteins, RNA fusions, tissue proteins, plasma or serum lipids, RNA gene expressions, CNVs, INDELS, SNVs, and tumor nuclei characteristics.

17. The system of claim 14, wherein the feature weights are evaluated using a leave-one-patient-out cross-validation strategy.

18. The system of claim 14, wherein the one or more machine learning models comprise Support Vector Machine (SVM), Principal Component Analysis (PCA)+Logistic Regression, L1-Normalized SVM, L1-Normalized Random Forest, 5-hidden-layer Deep Neural Network, Recursive Feature Elimination (RFE) Logistic Regression or RFE Random Forest.

19. A method of prognosticating prostate cancer in a subject, comprising:
assaying a plurality of analytes to detect a presence of a plurality of features,
wherein the plurality of analytes
(i) are derived from serum, plasma, blood and/or tissue samples subjected to targeted NGS DNA sequencing, whole transcriptome RNA sequencing, paired tissue proteomics, unpaired serum proteomics, lipidomics, surgical pathology, computational pathology, or a combination thereof, or
(ii) include plasma, serum, or blood proteins, RNA fusions, tissue proteins, plasma or serum lipids, RNA gene expressions, CNVs, INDELS, SNVs, tumor nuclei characteristic, or a combination thereof, or
(iii) both (i) and (ii),
wherein the plurality of features are selected from Tables 4A-4C, Tables 5A-5B, Tables 6A-6B, Tables 7A-7B, Table 8, Table 9, Tables 13A-13B, Table 14, Table 15, Tables 18A-18B or a combination thereof, and
prognosticate the subject as having a higher likelihood of survival or the subject as having a lower likelihood of recurrence based on presence of the plurality of features, or
prognosticate the subject as having a lower likelihood of survival or the subject as having a higher likelihood of recurrence based on presence of the plurality of features.

20. The method of claim 19, further comprising selecting a pancreatic cancer treatment method from among a plurality of pancreatic cancer treatment methods based on the likelihood of survival or the likelihood of recurrent.

21. The method of claim 19, further comprising administering the pancreatic cancer treatment method.

22. The method of claim 19, wherein the plurality of features comprises at least 250 features.

23. The method of claim 19, wherein the plurality of features comprises at least 500 features.

24. The method of claim 19, wherein the plurality of analytes comprise at least four analytes.

25. The method of claim 24, wherein the at least four analytes comprises proteins (plasma, serum, or blood protein), lipids (plasma or serum), pathology and clinical.

26. The method of claim 19, wherein the plurality of features are selected from Table 15.
